(19) United States
(12) Patent Application Publication
(10) Pub. No.: US 2026/0201329 A1
(43) Pub. Date: Jul. 16, 2026

(54) GUIDE RNAS THAT TARGET TRAC GENE AND METHODS OF USE

(71) Applicant: Life Edit Therapeutics, Inc., Durham, NC (US)

(72) Inventors: Philip Borden, Durham, NC (US); Michael Coyle, Chapel Hill, NC (US); Alexandra Briner Crawley, Cary, NC (US); Drew Kelso, Holly Springs, NC (US); Michael Vanden Oever, New Hill, NC (US)

(73) Assignee: Life Edit Therapeutics, Inc., Durham, NC (US)

(21) Appl. No.: 19/138,529

(22) PCT Filed: Dec. 15, 2023
(86) PCT No.: PCT/IB2023/062826

§ 371 Date: Jun. 12, 2025

Related U.S. Application Data
(60) Provisional application No. 63/387,889, filed on Dec. 16, 2022.

(51) Int. Cl. C12N 5/0783; C07K 14/725; C12N 9/12; C12N 9/22; C12N 9/78; C12N 15/11

(52) U.S. Cl. CPC C12N 5/0636; C07K 14/7051; C12N 9/1276; C12N 9/226; C12N 9/78; C12N 15/111; C07K 2319/09; C07K 2319/80; C12N 2310/20; C12N 2310/321; C12N 2310/3231; C12N 2310/531; C12N 2510/00; C12Y 207/07049; C12Y 305/03

(57) ABSTRACT

Compositions and methods for binding to a target sequence in a T cell receptor alpha chain constant (TRAC) gene are provided. Compositions include CRISPR RNAs, guide RNAs, and nucleic acid molecules encoding the same. Vectors and host cells comprising the nucleic acid molecules are also provided. Further provided are RNA-guided nuclease (RGN) systems for binding a target sequence in a TRAC gene, wherein the RGN system comprises an RNA-guided nuclease polypeptide and one or more guide RNAs. The compositions find use in cleaving or modifying a target sequence of an TRAC gene, and/or modifying the expression of an TRAC gene.

### CROSS-REFERENCE TO RELATED APPLICATIONS

This application claims priority to U.S. Provisional Application No. 63/387,889, filed Dec. 16, 2022, which is incorporated by reference herein in its entirety.

### REFERENCE TO A SEQUENCE LISTING SUBMITTED ELECTRONICALLY AS AN XML FILE

The instant application contains a Sequence Listing which has been submitted in xml format via USPTO Patent Center and is hereby incorporated by reference in its entirety. Said xml copy, created on Dec. 7, 2023, is named L103438_1360PCT_0253_3_SL, and is 1.15 MB in size.

### FIELD OF THE INVENTION

The present invention relates to the field of molecular biology and gene editing.

### BACKGROUND OF THE INVENTION

T cells are white blood cells that function in the adaptive immune system to attack and destroy foreign molecules, pathogens, and/or tumors. This function of T cells is helped in part by the presence of T cell receptor (TCR) molecules on their surface. TCRs can bind fragments of foreign peptides presented by cells that have encountered a foreign entity such as a virus, and this interaction allows T cells to detect and act against foreign molecules. The most common type of TCR is composed of an alpha chain and a beta chain. Each of the alpha and beta chain contain variable and constant regions, and the variable region functions in binding an antigen. There is a single gene encoding the T cell receptor alpha chain constant (TRAC) region.

The presence of endogenous TCRs, however, may pose a challenge in developing donor-derived T cells that are desired for targeting pathogens or tumors in a recipient. The TCR may cause attack on non-targeted recipient tissues, termed graft-versus-host disease (GvHD). The ability to reduce or knock out a TCR component, such as TRAC, would be invaluable in preventing or reducing GvHD, among other advantages.

Targeted genome editing or modification is rapidly becoming an important tool for basic and applied research, as it allows modification of genomes such as cutting nucleic acids, deleting nucleic acids, inserting nucleic acids, substituting nucleotides in nucleic acids, and regulating gene expression at specific locations in a genome, along with many other possible modifications. Initial efforts in genome editing involved designing nucleases, proteins that are able to edit nucleic acids, to recognize and bind specifically to a target nucleic acid sequence to be edited. However, engineering nucleases takes considerable time and experimentation to obtain ones effective for editing of a particular sequence. Genome editing systems that use RNA-guided nucleases, such as the Clustered Regularly Interspaced Short Palindromic Repeats (CRISPR)-associated (Cas) proteins of the CRISPR-Cas bacterial system, function by complexing a nuclease with a guide RNA. The hybridization of the guide RNA to a particular target sequence allows editing at a specific location in a genome. Thus, genome editing systems that use RNA-guided nucleases can be less costly and more efficient for editing of genome sequences, as nucleic acids typically can be easier to design and re-design as compared to a nuclease.

Thus, regulation of expression of TRAC would benefit from development of RNA-guided nuclease systems that are able to target specific regions of the TRAC gene for binding, cleavage, and/or modification.

### BRIEF SUMMARY OF THE INVENTION

Compositions and methods for binding a target sequence in the T cell receptor alpha chain constant (TRAC) gene are provided. The compositions find use in modifying the TRAC gene at specific regions. Compositions comprise CRISPR RNAs (crRNAs), trans-activating CRISPR RNAs (tracrRNAs), single guide RNAs (sgRNAs), dual guide RNA (dgRNAs), RNA-guided nuclease (RGN) polypeptides, nucleic acid molecules encoding the same, compositions comprising the same, and vectors and host cells comprising the nucleic acid molecules. Also provided are RGN systems and ribonucleoprotein complexes for binding a target sequence in the TRAC gene, wherein the RGN system and ribonucleoprotein complex comprises an RGN polypeptide and one or more guide RNAs. Thus, methods disclosed herein are drawn to binding a target sequence in the TRAC gene, and in some embodiments, cleaving or modifying the target sequence in the TRAC gene. The TRAC gene can be modified, for example, to be knocked out as a result of non-homologous end joining after cleavage of a target sequence. In some embodiments, a target sequence in the TRAC gene is cleaved and a donor polynucleotide inserted at the cleavage site.

In one aspect, the present disclosure provides a guide RNA (gRNA) comprising a CRISPR RNA (crRNA) and a trans-activating CRISPR RNA (tracrRNA), wherein the crRNA comprises (i) a crRNA repeat; and (ii) a spacer, wherein the tracrRNA comprises: (iii) an anti-repeat; and (iv) a tail, wherein the gRNA comprises a first stem loop formed by hybridization of the crRNA repeat and the anti-repeat, wherein the spacer is capable of hybridizing to a target sequence in a T cell receptor alpha chain constant (TRAC) gene, wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, and 104. In some aspects, the target sequence in a TRAC gene that the spacer hybrids to comprises a target strand and a non-target strand.

In some embodiments of the above aspect, the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 to 5 nucleotides. In some embodiments of the above aspect, the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103.

In some embodiments of the above aspect, the gRNA is a single guide RNA (sgRNA) comprising the crRNA and the tracrRNA linked by a linker, wherein the sgRNA comprises a backbone and the spacer, and wherein the backbone of the sgRNA comprises the crRNA repeat, the linker, and the tracrRNA. In some embodiments, the linker has a nucleotide sequence set forth as AAAG, GAAA, ACUU, or CAAAGG. In some embodiments, the linker has a nucleotide sequence set forth as AAAG. In some embodiments of the above aspect, the backbone of the sgRNA comprises a total length of at least 85, 90, 95, 100, 105, 110, 115, or 120 nucleotides. In some embodiments of the above aspect, the backbone of the sgRNA comprises a total length of at most 85, 90, 95, 100, 105, 110, 115, or 120 nucleotides. In some embodiments of the above aspect, the backbone of the sgRNA comprises a total length of 86 to 98 nucleotides. In some embodiments of the above aspect, the backbone of the sgRNA comprises a total length of 94 nucleotides. In some embodiments of the above aspect, the backbone of the sgRNA has a nucleotide sequence having at least 80% sequence identity, at least 90% sequence identity, at least 95% sequence identity, or 100% sequence identity to any one of SEQ ID NOs: 124-134.

In some embodiments of the above aspect, the first stem loop comprises a first stem and a second stem, and wherein the first stem of the first stem loop comprises a total length of at least 3, 4, 5, 6, 7, 8, 9, 10, or 11 base pairs (bp). In some embodiments of the above aspect, the first stem of the first stem loop comprises a total length of at most 3, 4, 5, 6, 7, 8, 9, 10, or 11 bp. In some embodiments, the first stem of the first stem loop comprises a total length of 6 bp. In some embodiments, the first stem of the first stem loop comprises a total length of 3 bp.

In some embodiments of the above aspect, the tail of the tracrRNA comprises a total length of at least 1, 2, 3, 4, 5, 6, or 7 nucleotides. In some embodiments of the above aspect, the tail of the tracrRNA comprises a total length of at most 1, 2, 3, 4, 5, 6, or 7 nucleotides. In some embodiments, the tail of the tracrRNA comprises a total length of 3 nucleotides. In some embodiments, the tail of the tracrRNA comprises a total length of 1 nucleotide.

In some embodiments of the above aspect, the gRNA further comprises a second stem loop most proximal to the tail, wherein the second stem loop comprises a first stem and a second stem. In some embodiments of the above aspect, the first stem of the second stem loop comprises a total length of at least 1, 2, 3, 4, 5, or 6 bp. In some embodiments of the above aspect, the first stem of the second stem loop comprises a total length of at most 1, 2, 3, 4, 5, or 6 bp. In some embodiments, the first stem of the second stem loop comprises a total length of 5 bp.

In some embodiments of the above aspect, the first stem of the first stem loop comprises a total length of 6 bp, the tail of the tracrRNA comprises a total length of 3 nucleotides, and the first stem of the second stem loop comprises a total length of 5 bp.

In some embodiments of the above aspect, the gRNA is a dual guide RNA (dgRNA). In some embodiments of the above aspect, the crRNA repeat of the dgRNA comprises a total length of at least 10, 11, 12, 13, 14, 15, 16, 17, 18, 19, 20, or 21 nucleotides. In some embodiments of the above aspect, the crRNA repeat of the dgRNA comprises a total length of at most 10, 11, 12, 13, 14, 15, 16, 17, 18, 19, 20, or 21 nucleotides. In some embodiments, the crRNA repeat of the dgRNA comprises a total length of 13 nucleotides. In some embodiments, the crRNA repeat of the dgRNA comprises a total length of 16 nucleotides. In some embodiments, the crRNA repeat of the dgRNA comprises a total length of 21 nucleotides. In some embodiments of the above aspect, the tracrRNA of the dgRNA comprises a total length of at least 65, 70, 75, 80, or 85 nucleotides. In some embodiments of the above aspect, the tracrRNA of the dgRNA comprises a total length of at most 65, 70, 75, 80, or 85 nucleotides. In some embodiments, the tracrRNA of the dgRNA comprises a total length of 74 nucleotides. In some embodiments, the tracrRNA of the dgRNA comprises a total length of 77 nucleotides.

In some embodiments of the above aspect, the gRNA comprises a total length of at least 85, 90, 95, 100, 105, 110, 115, 120, 125, 130, or 135 nucleotides. In some embodiments of the above aspect, the gRNA comprises a total length of at most 85, 90, 95, 100, 105, 110, 115, 120, 125, 130, or 135 nucleotides. In some embodiments of the above aspect, the gRNA comprises a total length of 106 to 135 nucleotides. In some embodiments of the above aspect, the gRNA comprises a total length of 117 to 119 nucleotides.

In some embodiments of the above aspect, the gRNA is capable of targeting a bound RNA-guided nuclease (RGN) polypeptide to the target sequence in the TRAC gene. In some embodiments of the above aspect, the RGN polypeptide is capable of recognizing a consensus protospacer adjacent motif (PAM) having the nucleotide sequence set forth as NNNNCC or NNRNCC. In some embodiments of the above aspect, the RGN polypeptide is capable of recognizing a full protospacer adjacent motif (PAM) having the nucleotide sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG.

In some embodiments of the above aspect, the RGN polypeptide has an amino acid sequence having at least 80% sequence identity to SEQ ID NO: 105; and wherein the target sequence and the spacer are selected from the group consisting of: a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 1 to 5 nucleotides; and b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 1 to 5 nucleotides.

In some embodiments of the above aspect, the spacer has the nucleotide sequence set forth as SEQ ID NO: 7 or 9.

In some embodiments of the above aspect, the RGN polypeptide has the amino acid sequence set forth as SEQ ID NO: 105. In some embodiments of the above aspect, the crRNA repeat has the nucleotide sequence set forth as SEQ ID NO: 106 or a nucleotide sequence that differs in length and/or sequence from SEQ ID NO: 106 by 1 to 8 nucleotides. In some embodiments of the above aspect, the crRNA repeat has the nucleotide sequence set forth as any one of SEQ ID NOs: 106, 109-112, 328, 331, and 334. In some embodiments of the above aspect, the crRNA has the nucleotide sequence set forth as any one of SEQ ID NOs: 136-197. In some embodiments of the above aspect, the tracrRNA has a nucleotide sequence having at least 80% sequence identity, at least 90% sequence identity, or at least 95% sequence identity to SEQ ID NO: 107. In some embodiments, the tracrRNA has a nucleotide sequence set forth as SEQ ID NO: 107. In some embodiments of the above aspect, the tracrRNA has a nucleotide sequence that differs in length from SEQ ID NO: 107 by 1 to 16 nucleotides. In some embodiments, the tracrRNA has a nucleotide sequence that is 8 nucleotides shorter than SEQ ID NO: 107. In some embodiments, the tracrRNA has a nucleotide sequence that is 11 nucleotides shorter than SEQ ID NO: 107. In some embodiments of the above aspect, the tracrRNA has the nucleotide sequence set forth as any one of SEQ ID NOs: 107, 114-123, 329, 332, and 335.

In some embodiments of the above aspect, the RGN polypeptide has an amino acid sequence having at least 80% sequence identity, at least 90% sequence identity, at least 95% sequence identity, or 100% sequence identity to SEQ ID NO: 327 or 330. In some embodiments, the RGN polypeptide has an amino acid sequence set forth as SEQ ID NO: 327 or 330. In some embodiments of the above aspect, the RGN polypeptide has an amino acid sequence having at least 80% sequence identity, at least 90% sequence identity, at least 95% sequence identity, or 100% sequence identity to SEQ ID NO: 333. In some embodiments, the RGN polypeptide has an amino acid sequence set forth as SEQ ID NO: 333. In some embodiments of the above aspect, the gRNA has a nucleotide sequence set forth as any one of SEQ ID NOs: 198-200, 202-213, 215-233, 235-241, and 243-259. In some embodiments, the gRNA has a nucleotide sequence set forth as SEQ ID NO: 204 or 205.

In some embodiments of the above aspect, the gRNA comprises at least one chemical modification. In some embodiments, the at least one chemical modification comprises a bridged nucleic acid (BNA) modification; 2′-O-methyl(2′-O-Me) modification; 2′-O-methoxy-ethyl(2′MOE) modification; 2′-fluoro (2′-F) modification; 2′F-4′Cα-OMe modification; 2′,4′-di-Cα-OMe modification; 2′-O-methyl 3′phosphorothioate (MS) modification; 2′-O-methyl 3′thiophosphonoacetate (MSP) modification; 2′-O-methyl 3′phosphonoacetate (MP) modification; phosphorothioate (PS) modification; or a combination thereof. In some embodiments, the BNA comprises a 2′,4′ BNA modification. In some embodiments, the 2′,4′ BNA modification is selected from the group consisting of: locked nucleic acid (LNA) modification, BNANC[N-Me] modification, 2′-O,4′-C-ethylene bridged nucleic acid (2′,4′-ENA) modification, and S-constrained ethyl (cEt) modification. In some embodiments, the 2′,4′ BNA is a LNA modification. In some embodiments, the 2′,4′ BNA is a cEt modification. In some embodiments, the at least one chemical modification comprises a BNA modification, 2′-O-Me modification, PS modification, or a combination thereof.

In some embodiments, the at least one chemical modification comprises 2′-O-methyl 3′phosphorothioate (MS) modifications at the 3 terminal nucleotides at the 5′ region and at the 3 terminal nucleotides at the 3′ region of the gRNA. In some embodiments of the above aspect, the crRNA repeat has the nucleotide sequence set forth as any one of SEQ ID NOs: 430, 432-435, 583, 585, and 587. In some embodiments of the above aspect, the crRNA has the nucleotide sequence set forth as any one of SEQ ID NOs: 459-520. In some embodiments of the above aspect, the tracrRNA has the nucleotide sequence set forth as any one of SEQ ID NOs: 431, 437-446, 584, 586, and 588. In some embodiments of the above aspect, the gRNA has the nucleotide sequence set forth as any one of SEQ ID NOs: 521-523, 525-536, 538-556, 558-564, and 566-582.

In some embodiments of the above aspect, the gRNA further comprises an extension comprising an edit template for reverse transcriptase (RT) editing.

In another aspect, the present disclosure provides a guide RNA (gRNA) comprising a CRISPR RNA (crRNA) and a trans-activating CRISPR RNA (tracrRNA), wherein the crRNA comprises: (i) a crRNA repeat; and (ii) a spacer, wherein the tracrRNA comprises: (iii) an anti-repeat; and (iv) a tail, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103, or has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 to 5 nucleotides.

In some embodiments of the above gRNA aspect, the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103. In some embodiments of the above gRNA aspect, the spacer is capable of hybridizing to a target sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, and 104.

In another aspect, the present disclosure provides a nucleic acid molecule comprising a CRISPR RNA (crRNA) or encoding a crRNA, wherein the crRNA comprises a spacer and a crRNA repeat, wherein the spacer is capable of hybridizing to a target sequence in a T cell receptor alpha chain constant (TRAC) gene, and wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, and 104.

In some embodiments of the nucleic acid molecule aspect, the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 to 5 nucleotides. In some embodiments, the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103.

In some embodiments of the nucleic acid molecule aspect, the crRNA is capable of binding a trans-activating CRISPR RNA (tracrRNA) to form a guide RNA (gRNA), wherein the tracrRNA comprises an anti-repeat and a tail. In some embodiments of the nucleic acid molecule aspect, the gRNA is a single guide RNA (sgRNA) comprising the crRNA and the tracrRNA linked by a linker, wherein the sgRNA comprises a backbone and the spacer, and wherein the backbone of the sgRNA comprises the crRNA repeat, the linker, and the tracrRNA. In some embodiments, the backbone of the sgRNA comprises a total length of 86 to 98 nucleotides. In some embodiments, the backbone of the sgRNA comprises a total length of 94 nucleotides. In some embodiments of the nucleic acid molecule aspect, the backbone of the sgRNA has a nucleotide sequence having at least 80% sequence identity, at least 90% sequence identity, at least 95% sequence identity, or 100% sequence identity to any one of SEQ ID NOs: 124-134.

In some embodiments of the nucleic acid molecule aspect, the gRNA comprises a first stem loop comprising a first stem and a second stem formed by hybridization of the crRNA repeat and the anti-repeat, wherein the first stem of the first stem loop comprises a total length of at least 3, 4, 5, 6, 7, 8, 9, 10, or 11 bp. In some embodiments of the nucleic acid molecule aspect, the gRNA comprises a first stem loop comprising a first stem and a second stem formed by hybridization of the crRNA repeat and the anti-repeat, wherein the first stem of the first stem loop comprises a total length of at most 3, 4, 5, 6, 7, 8, 9, 10, or 11 bp. In some embodiments, the first stem of the first stem loop comprises a total length of 6 bp. In some embodiments, the first stem of the first stem loop comprises a total length of 3 bp.

In some embodiments of the nucleic acid molecule aspect, the tail of the tracrRNA comprises a total length of at least 1, 2, 3, 4, 5, 6, or 7 nucleotides. In some embodiments of the nucleic acid molecule aspect, the tail of the tracrRNA comprises a total length of at most 1, 2, 3, 4, 5, 6, or 7 nucleotides. In some embodiments, the tail of the tracrRNA comprises a total length of 3 nucleotides. In some embodiments, the tail of the tracrRNA comprises a total length of 1 nucleotide.

In some embodiments of the nucleic acid molecule aspect, the gRNA further comprises a second stem loop most proximal to the tail, wherein the second stem loop comprises a first stem and a second stem. In some embodiments of the nucleic acid molecule aspect, the first stem of the second stem loop comprises a total length of at least 1, 2, 3, 4, 5, or 6 bp. In some embodiments of the nucleic acid molecule aspect, the first stem of the second stem loop comprises a total length of at most 1, 2, 3, 4, 5, or 6 bp. In some embodiments, the first stem of the second stem loop comprises a total length of 5 bp.

In some embodiments of the nucleic acid molecule aspect, the first stem of the first stem loop comprises a total length of 6 bp, the tail of the tracrRNA comprises a total length of 3 nucleotides, and the first stem of the second stem loop comprises a total length of 5 bp.

In some embodiments of the nucleic acid molecule aspect, the gRNA is a dual guide RNA (dgRNA). In some embodiments of the above aspect, the crRNA repeat comprises a total length of at least 10, 11, 12, 13, 14, 15, 16, 17, 18, 19, 20, or 21 nucleotides. In some embodiments of the nucleic acid molecule aspect, the crRNA repeat comprises a total length of at most 10, 11, 12, 13, 14, 15, 16, 17, 18, 19, 20, or 21 nucleotides. In some embodiments, the crRNA repeat comprises a total length of 13 nucleotides. In some embodiments, the crRNA repeat comprises a total length of 16 nucleotides. In some embodiments, the crRNA repeat comprises a total length of 21 nucleotides. In some embodiments of the nucleic acid molecule aspect, the tracrRNA comprises a total length of at least 65, 70, 75, 80, or 85 nucleotides. In some embodiments of the nucleic acid molecule aspect, the tracrRNA comprises a total length of at most 65, 70, 75, 80, or 85 nucleotides. In some embodiments, the tracrRNA comprises a total length of 74 nucleotides. In some embodiments, the tracrRNA comprises a total length of 77 nucleotides.

In some embodiments of the nucleic acid molecule aspect, the gRNA comprises a total length of at least 85, 90, 95, 100, 105, 110, 115, 120, 125, 130, or 135 nucleotides. In some embodiments of the nucleic acid molecule aspect, the gRNA comprises a total length of at most 85, 90, 95, 100, 105, 110, 115, 120, 125, 130, or 135 nucleotides. In some embodiments, the gRNA comprises a total length of 106 to 135 nucleotides. In some embodiments, the gRNA comprises a total length of 117 to 119 nucleotides. In some embodiments, the gRNA is capable of targeting a bound RNA-guided nuclease (RGN) polypeptide to a target sequence.

In some embodiments of the nucleic acid molecule aspect, the gRNA is capable of binding to an RGN polypeptide capable of recognizing a consensus protospacer adjacent motif (PAM) having the nucleotide sequence set forth as NNNNCC or NNRNCC. In some embodiments of the nucleic acid molecule aspect, the gRNA is capable of binding to an RGN polypeptide capable of recognizing a full protospacer adjacent motif (PAM) having the nucleotide sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG.

In some embodiments of the nucleic acid molecule aspect, the RGN polypeptide comprises an amino acid sequence having at least 80% sequence identity to SEQ ID NO: 105; and wherein the target sequence and the spacer are selected from the group consisting of: a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 1 to 5 nucleotides; and b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 1 to 5 nucleotides. In some embodiments, the spacer has the nucleotide sequence set forth as SEQ ID NO: 7 or 9.

In some embodiments of the nucleic acid molecule aspect, the RGN polypeptide comprises an amino acid sequence set forth as SEQ ID NO: 105. In some embodiments of the nucleic acid molecule aspect, the crRNA repeat has the nucleotide sequence set forth as SEQ ID NO: 106 or a nucleotide sequence that differs in length and/or sequence from SEQ ID NO: 106 by 1 to 8 nucleotides. In some embodiments of the nucleic acid molecule aspect, the crRNA repeat has the nucleotide sequence set forth as any one of SEQ ID NOs: 106, 109-112, 328, 331, and 334. In some embodiments of the nucleic acid molecule aspect, the crRNA has the nucleotide sequence set forth as any one of SEQ ID NOs: 136-197. In some embodiments of the nucleic acid molecule aspect, the tracrRNA has a nucleotide sequence having at least 80% sequence identity, at least 90% sequence identity, or at least 95% sequence identity to SEQ ID NO: 107. In some embodiments, the tracrRNA has a nucleotide sequence set forth as SEQ ID NO: 107. In some embodiments of the nucleic acid molecule aspect, the tracrRNA has a nucleotide sequence that differs in length from SEQ ID NO: 107 by 1 to 16 nucleotides. In some embodiments, the tracrRNA has a nucleotide sequence that is 8 nucleotides shorter than SEQ ID NO: 107. In some embodiments, the tracrRNA has a nucleotide sequence that is 11 nucleotides shorter than SEQ ID NO: 107. In some embodiments of the nucleic acid molecule aspect, the tracrRNA has the nucleotide sequence set forth as any one of SEQ ID NOs: 107, 114-123, 329, 332, and 335.

In some embodiments of the nucleic acid molecule aspect, the RGN polypeptide has an amino acid sequence having at least 80% sequence identity, at least 90% sequence identity, at least 95% sequence identity, or 100% sequence identity to SEQ ID NO: 327 or 330. In some embodiments, the RGN polypeptide has an amino acid sequence set forth as SEQ ID NO: 327 or 330. In some embodiments of the nucleic acid molecule aspect, the RGN polypeptide has an amino acid sequence having at least 80% sequence identity, at least 90% sequence identity, at least 95% sequence identity, or 100% sequence identity to SEQ ID NO: 333. In some embodiments, the RGN polypeptide has an amino acid sequence set forth as SEQ ID NO: 333. In some embodiments of the nucleic acid molecule aspect, the gRNA has a nucleotide sequence set forth as any one of SEQ ID NOs: 198-200, 202-213, 215-233, 235-241, and 243-259. In some embodiments, the gRNA has a nucleotide sequence set forth as SEQ ID NO: 204 or 205.

In some embodiments of the nucleic acid molecule aspect, the gRNA comprises at least one chemical modification. In some embodiments, the at least one chemical modification comprises a bridged nucleic acid (BNA) modification; 2′-O-methyl(2′-O-Me) modification; 2′-O-methoxy-ethyl (2′MOE) modification; 2′-fluoro (2′-F) modification; 2′F-4′Cα-OMe modification; 2′,4′-di-Cα-OMe modification; 2′-O-methyl 3′phosphorothioate (MS) modification; 2′-O-methyl 3′thiophosphonoacetate (MSP) modification; 2′-O-methyl 3′phosphonoacetate (MP) modification; phosphorothioate (PS) modification; or a combination thereof. In some embodiments, the BNA comprises a 2′,4′ BNA modification. In some embodiments, the 2′,4′ BNA modification is selected from the group consisting of: locked nucleic acid (LNA) modification, BNANC[N-Me] modification, 2′-O,4′-C-ethylene bridged nucleic acid (2′,4′-ENA) modification, and S-constrained ethyl (cEt) modification. In some embodiments, the 2′,4′ BNA is a LNA modification. In some embodiments, the 2′,4′ BNA is a cEt modification. In some embodiments, the at least one chemical modification comprises a BNA modification, 2′-O-Me modification, PS modification, or a combination thereof.

In some embodiments, the at least one chemical modification comprises 2′-O-methyl 3′phosphorothioate (MS) modifications at the 3 terminal nucleotides at the 5′ region and at the 3 terminal nucleotides at the 3′ region of the gRNA. In some embodiments of the nucleic acid molecule aspect, the crRNA repeat has the nucleotide sequence set forth as any one of SEQ ID NOs: 430, 432-435, 583, 585, and 587. In some embodiments of the nucleic acid molecule aspect, the crRNA has the nucleotide sequence set forth as any one of SEQ ID NOs: 459-520. In some embodiments of the nucleic acid molecule aspect, the tracrRNA has the nucleotide sequence set forth as any one of SEQ ID NOs: 431, 437-446, 584, 586, and 588. In some embodiments of the nucleic acid molecule aspect, the gRNA has the nucleotide sequence set forth as any one of SEQ ID NOs: 521-523, 525-536, 538-556, 558-564, and 566-582.

In some embodiments of the nucleic acid molecule aspect, the gRNA further comprises an extension comprising an edit template for reverse transcriptase (RT) editing.

In yet another aspect, the present disclosure provides a nucleic acid molecule comprising a CRISPR RNA (crRNA) or encoding a crRNA, wherein the crRNA comprises a spacer and a crRNA repeat, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103, or has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 to 5 nucleotides.

In some embodiments of the above nucleic acid molecule aspect, the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103.

In some embodiments of the above nucleic acid molecule aspect, the spacer is capable of hybridizing to a target sequence, and wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, and 104.

In still another aspect, the present disclosure provides a vector comprising the nucleic acid molecule as described hereinabove, wherein the nucleic acid molecule comprises a polynucleotide encoding the crRNA. In some embodiments of the vector aspect, the nucleic acid molecule further comprises a heterologous promoter operably linked to the polynucleotide encoding the crRNA. In some embodiments, the heterologous promoter is an RNA polymerase III (pol III) promoter. In some embodiments of the vector aspect, the vector further comprises a nucleic acid molecule encoding an RGN polypeptide, wherein the crRNA is capable of binding a tracrRNA to form a guide RNA, wherein the guide RNA is capable of binding to the RGN polypeptide. In some embodiments of the vector aspect, the vector further comprises a promoter operably linked to the nucleic acid molecule encoding the RGN polypeptide.

In yet another aspect, the present disclosure provides a vector comprising the nucleic acid molecule as described hereinabove, wherein the nucleic acid molecule comprises a polynucleotide encoding the crRNA, and wherein the vector further comprises a polynucleotide encoding the tracrRNA. In some embodiments of the vector aspect, the polynucleotide encoding the crRNA and the polynucleotide encoding the tracrRNA are operably linked to the same promoter and are encoded as a sgRNA. In some embodiments of the vector aspect, the polynucleotide encoding the crRNA and the polynucleotide encoding the tracrRNA are operably linked to separate promoters. In some embodiments of the vector aspect, the vector further comprises a nucleic acid molecule encoding an RGN polypeptide, wherein the crRNA is capable of binding the tracrRNA to form a guide RNA, wherein the guide RNA is capable of binding to the RGN polypeptide. In some embodiments of the vector aspect, the vector further comprises a promoter operably linked to the nucleic acid molecule encoding the RGN polypeptide.

In another aspect, the present disclosure provides a cell comprising the gRNA, the nucleic acid molecule, or the vector as described hereinabove.

In another aspect, the present disclosure provides an RNA-guided nuclease (RGN) system for binding a target sequence within a T cell receptor alpha chain constant (TRAC) gene, wherein the RGN system comprises: a) one or more gRNAs as described hereinabove, or one or more polynucleotides comprising one or more nucleotide sequences encoding the one or more gRNAs as described hereinabove; and b) an RGN polypeptide, or a polynucleotide comprising a nucleotide sequence encoding the RGN polypeptide; wherein the one or more guide RNAs are capable of forming a complex with the RGN polypeptide to direct the RGN polypeptide to bind to the target sequence.

In some embodiments of the RGN system aspect, the RGN polypeptide is capable of recognizing a consensus protospacer adjacent motif (PAM) having the nucleotide sequence set forth as NNNNCC or NNRNCC. In some embodiments of the RGN system aspect, the RGN polypeptide is capable of recognizing a full PAM having a nucleotide sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG. In some embodiments of the RGN system aspect, the RGN polypeptide comprises an amino acid sequence set forth as SEQ ID NO: 105. In some embodiments of the RGN system aspect, the RGN polypeptide has an amino acid sequence set forth as SEQ ID NO: 327. In some embodiments of the RGN system aspect, the RGN polypeptide has an amino acid sequence set forth as SEQ ID NO: 330. In some embodiments of the RGN system aspect, the RGN polypeptide has an amino acid sequence set forth as SEQ ID NO: 333.

In some embodiments of the RGN system aspect, the polynucleotide comprising a nucleotide sequence encoding the RGN polypeptide comprises an mRNA. In some embodiments of the RGN system aspect, the polynucleotide comprising a nucleotide sequence encoding the RGN polypeptide is codon optimized for expression in a mammalian cell. In some embodiments of the RGN system aspect, at least one of the one or more nucleotide sequences encoding the one or more gRNAs and the nucleotide sequence encoding the RGN polypeptide is operably linked to a promoter heterologous to the nucleotide sequence. In some embodiments of the RGN system aspect, the one or more nucleotide sequences encoding the one or more gRNAs and the nucleotide sequence encoding the RGN polypeptide are located on one vector. In some embodiments of the RGN system aspect, the RGN polypeptide is nuclease inactive or is a nickase. In some embodiments of the RGN system aspect, the RGN polypeptide is fused to a base-editing polypeptide. In some embodiments, the base-editing polypeptide comprises a deaminase. In some embodiments of the RGN system aspect, the RGN polypeptide is fused to a RT editing polypeptide. In some embodiments, the RT editing polypeptide comprises a DNA polymerase. In some embodiments, the DNA polymerase comprises a reverse transcriptase. In some embodiments of the RGN system aspect, the gRNA further comprises an extension comprising an edit template for RT editing. In some embodiments of the RGN system aspect, the RGN polypeptide comprises one or more nuclear localization signals.

In still another aspect, the present disclosure provides a ribonucleoprotein (RNP) complex comprising the one or more gRNA and the RGN polypeptide of the RGN system as described hereinabove.

In still another aspect, the present disclosure provides a cell comprising the RGN system or the RNP complex as described hereinabove. In some embodiments, the cell is a eukaryotic cell. In some embodiments, the eukaryotic cell is a mammalian cell. In some embodiments, the mammalian cell is a human cell. In some embodiments, the mammalian cell or human cell is a T cell or an induced pluripotent stem cell.

In another aspect, the present disclosure provides a method for binding a target sequence within a TRAC gene, comprising delivering the RGN system or the RNP complex as described hereinabove to the target sequence or a cell comprising the target sequence. In some embodiments of the method for binding a target sequence within a TRAC gene aspect, cleavage or modification of the target sequence occurs.

In another aspect, the present disclosure provides a method for assembling an RNA-guided nuclease (RGN) ribonucleoprotein complex, the method comprising combining under conditions suitable for formation of the complex: a) the guide RNA as described hereinabove; and b) an RGN polypeptide that binds the guide RNA. In some embodiments of the method for assembling aspect, the RGN polypeptide is capable of recognizing a consensus protospacer adjacent motif (PAM) having the nucleotide sequence set forth as NNNNCC or NNRNCC. In some embodiments of the method for assembling aspect, the complex directs cleavage of the target sequence. In some embodiments, the cleavage generates a double-stranded break. In some embodiments, wherein the cleavage generates a single-stranded break.

In another aspect, the present disclosure provides a method for binding a target sequence within a TRAC gene, the method comprising: a) combining under conditions suitable for formation of a ribonucleoprotein (RNP) complex: i) the guide RNA as described hereinabove; and ii) an RGN polypeptide that binds the guide RNA; thereby assembling an RNP complex; and b) contacting the target sequence or a cell comprising the target sequence with the assembled RNP complex; wherein the guide RNA hybridizes to the target sequence, thereby directing binding of the RNP complex to the target sequence. In some embodiments of the method for binding a target sequence within a TRAC gene aspect, the RGN polypeptide is capable of recognizing a consensus protospacer adjacent motif (PAM) having the nucleotide sequence set forth as NNNNCC or NNRNCC. In some embodiments of the method for binding a target sequence within a TRAC gene aspect, the RGN polypeptide is capable of recognizing a full protospacer adjacent motif (PAM) having the nucleotide sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG. In some embodiments of the method for binding a target sequence within a TRAC gene aspect, the RGN polypeptide comprises an amino acid sequence set forth as SEQ ID NO: 105. In some embodiments of the method for binding a target sequence within a TRAC gene aspect, the RGN polypeptide has an amino acid sequence set forth as SEQ ID NO: 327. In some embodiments of the method for binding a target sequence within a TRAC gene aspect, the RGN polypeptide has an amino acid sequence set forth as SEQ ID NO: 330. In some embodiments of the method for binding a target sequence within a TRAC gene aspect, the RGN polypeptide has an amino acid sequence set forth as SEQ ID NO: 333.

In some embodiments of the method for binding a target sequence within a TRAC gene aspect, the method is performed in vitro or ex vivo. In some embodiments of the method for binding a target sequence within a TRAC gene aspect, the RGN polypeptide is capable of cleaving the target sequence, thereby allowing for the cleaving and/or modifying of the target sequence. In some embodiments, the cleaving generates a double-stranded break. In some embodiments, the cleaving generates a single-stranded break. In some embodiments, the cleaving results in insertion of a heterologous sequence within the target sequence.

In some embodiments of the method for binding a target sequence within a TRAC gene aspect, the RGN polypeptide is nuclease inactive or is a nickase. In some embodiments of the method for binding a target sequence within a TRAC gene aspect, the RGN polypeptide is fused to a base-editing polypeptide. In some embodiments, the base-editing polypeptide comprises a deaminase. In some embodiments of the method for binding a target sequence within a TRAC gene aspect, the RGN polypeptide is fused to a RT editing polypeptide. In some embodiments, the RT editing polypeptide comprises a DNA polymerase. In some embodiments, the DNA polymerase comprises a reverse transcriptase. In some embodiments of the method for binding a target sequence within a TRAC gene aspect, the gRNA further comprises an extension comprising an edit template for RT editing.

In a further aspect, the present disclosure provides a method for modulating expression of a T cell receptor alpha chain (TRAC) gene in a population of cells, comprising delivering the RGN system described hereinabove or the RNP complex described hereinabove to the population of cells, wherein the population of cells comprises the target sequence, and wherein TRAC gene expression is modulated as compared to TRAC gene expression in a control population of cells.

In some embodiments of the method for modulating expression of a TRAC gene aspect, cleavage or modification of the target sequence occurs. In some embodiments, cleavage or modification of the target sequence is detected by sequencing. In some embodiments, TRAC gene expression is measured by quantitative PCR, microarray, RNA-seq, flow cytometry, immunoblot, enzyme-linked immunosorbent assay (ELISA), protein immunoprecipitation, immunostaining, high performance liquid chromatography (HPLC), liquid chromatography-mass spectrometry (LC/MS), mass spectrometry, or a combination thereof.

In some embodiments of the method for modulating expression of a TRAC gene aspect, TRAC gene expression is decreased. In some embodiments, the decrease in TRAC gene expression comprises decrease in TRAC mRNA and/or TRAC protein level. In some embodiments, the decrease in TRAC protein level is measured by flow cytometry for detection of CD3+ cells. In some embodiments, a decrease in CD3+ cells as compared to a level of CD3+ cells in the control population of cells is indicative of the decrease in TRAC protein level. In some embodiments, the decrease in CD3+ cells is 30% to 100%. In some embodiments, the decrease in CD3+ cells is 50% to 100%.

In some embodiments of the method for modulating expression of a TRAC gene aspect, cleavage or modification of the target sequence occurs at a rate of 40% to 100%. In some embodiments, cleavage or modification of the target sequence occurs at a rate of 80% to 100%.

In some embodiments of the method for modulating expression of a TRAC gene aspect, the control population of cells has not been subjected to the delivering.

In some embodiments of the method for modulating expression of a TRAC gene aspect, the population of cells comprises T cells.

### BRIEF DESCRIPTION OF THE FIGURES

FIG. 1 shows consistent editing with a TRAC guide RNA at higher doses of ribonucleoprotein (RNP) complex of guide RNA (gRNA) and APG07433.1 RGN. The pmol indicate the RNA-guided nuclease (RGN) amount and the ratio is RGN: guide RNA. For the guide used, the dose of RNP complex and RGN protein: guide RNA ratio are from left to right: 90 μmol 1:2, 90 μmol 1:3, 120 μmol 1:2, and 120 μmol 1:3.

FIG. 2 shows that a TRAC guide RNA has >70% editing at TRAC in cells from different donors using the APG07433.1 RGN. 60 μmol of RGN was used. For the guide used, the donor and RGN protein: guide RNA ratio are from left to right: Donor 1 (F) 1:2, Donor 1 (F) 1:3, Donor 2 (M) 1:2, Donor 2 (M) 1:3, Donor 3 (F) 1:2, and Donor 3 (F) 1:3.

FIG. 3 shows consistent high TRAC editing using APG07433.1 RGN, as measured by knockdown of the CD3 surface marker in cells from different donors. The % CD3+ cells was measured using flow cytometry. For each graph and each of the control or RNP complex dose, the donors are from left to right: Donor 1, Donor 2, and Donor 3.

FIG. 4 shows two TRAC guide RNAs having robust editing at TRAC in cells from different donors and across a range of RNP complex doses. For each guide used, the dose of RNP complex is from left to right: 20 μmol, 40 μmol, 60 μmol, and 80 μmol.

FIG. 5 shows performance of guide RNAs with two different spacers (1880 and 1881) in TRAC editing, with indicated backbone variant and spacer length as compared to guide RNA with native backbone and 25 nt spacer (‘Full Length’). The APG07433.1 RGN was used. TRAC editing was measured by knockdown of the CD3 surface marker in cells. The M backbone has: a deletion of 10 nt in the first stem of stem loop 1 formed by hybridization of the crRNA repeat and anti-repeat; a deletion of 2 nt in stem loop 3 most proximal to the tail of the guide RNA; and a deletion of 4 nt from the tail of the guide RNA; as compared to the native APG07433.1 backbone. The 94bb has a deletion of 16 nt in the first stem of stem loop 1, as compared to the native APG07433.1 backbone. ‘25’, ‘24’, and ‘23’ indicate the spacer length in nucleotides. The control indicates conditions without RGN and gRNA, where cells are mixed with nucleofection solution but do not go through the nucleofection process. The highest editing for the 1880 TRAC guide RNA was observed with a 24 nt spacer and a 94 nt backbone (118 nt total length of guide), and the highest editing for the 1881 TRAC guide RNA was observed with a 23 nt spacer and the M backbone (117 nt total length of guide). For each backbone variant, the % CD3+ cells with the 1880 spacer is on the left, and the % CD3+ cells with the 1881 spacer is on the right.

FIG. 6 shows that 2 truncated guide RNAs (shortened in spacer and backbone) were effective at editing 2 TRAC target sites across a dose range of RNP complex of guide RNA and APG07433.1 RGN and across multiple donors, where TRAC editing was measured by knockdown of the CD3 surface marker in cells. All 3 donors showed over 95% knockdown with both guides on average at highest dose. Knockdown was dose-dependent. SGN3156 is a TRAC guide RNA with the 754 24 nt spacer and the 94 nt backbone. SGN6286 is a TRAC guide RNA with the 755 23 nt spacer and the M backbone. Note that ‘754’ and ‘1880’ refer to the same 24 nt TRAC spacer sequence herein. Similarly, ‘755’ and ‘1881’ refer to the same 23 nt TRAC spacer sequence herein. For each guide used, the dose of RNP complex is from left to right: control, 20 μmol, 40 μmol, 60 μmol, and 80 μmol. The control indicates conditions without RGN and gRNA, where cells are mixed with nucleofection solution but do not go through the nucleofection process.

FIG. 7 shows the effectiveness of SGN3156 and SGN6286 truncated TRAC guide RNAs as percent editing, across a dose range of RNP complex of guide RNA and APG07433.1 RGN and across multiple donors. For each guide used, the dose of RNP complex is from left to right: 20 μmol, 40 μmol, 60 μmol, and 80 μmol.

FIG. 8 shows that the SGN3156 and SGN6286 truncated TRAC guide RNAs showed equal or slightly improved editing as compared to the original guide RNA with native backbone and 25 nt spacer. For each guide used, the dose of RNP complex is from left to right: 20 μmol, 40 μmol, 60 μmol, and 80 μmol.

FIG. 9 shows that cell viability was at or above 80% for most samples, across multiple donors, and across a dose range of RNP complex of truncated TRAC guide RNA and APG07433.1 RGN. For each guide used, the dose of RNP complex is from left to right: 20 μmol, 40 μmol, 60 μmol, and 80 μmol.

FIG. 10 shows that the 2 lead SGN3156 and SGN6286 truncated TRAC guide RNAs had no significant off-target modifications. For each on target site or predicted in silico off target site, the % insertions/deletions (indel) for edited is on the left, and the % indel for control is on the right. The control indicates conditions without RGN and gRNA, where cells are mixed with nucleofection solution but do not go through the nucleofection process.

### DETAILED DESCRIPTION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended embodiments. Although specific terms are employed herein, they are used in a generic and descriptive sense only and not for purposes of limitation.

### I. Overview

RNA-guided nuclease (RGN) systems allow for the targeted manipulation of specific site(s) within a genome and are useful in the context of gene targeting for therapeutic and research applications. In a variety of organisms, including mammals, RGN systems have been used for genome engineering by stimulating non-homologous end joining and homologous recombination, for example. The compositions and methods described herein are useful for modifying a T cell receptor alpha chain constant (TRAC) gene.

The RGN systems disclosed herein can bind, cleave, and/or modify target sequences in the TRAC gene. Modification of the TRAC gene can include reducing or eliminating expression of TRAC. The guide RNAs of the disclosed RGN systems can be engineered to be shorter than their native lengths and still maintain editing efficiencies of >60%.

The ability to reduce or knock out a TCR component, such as TRAC, would be valuable in situations where eliminating endogenous TCRs are desired, such as in preventing or reducing donor T cell reactivity against recipient tissues in adoptive T cell transfer, and/or engineering T cells with a heterologous TCR or chimeric antigen receptor.

### II. Guide RNA

The present disclosure provides guide RNAs, components thereof, and polynucleotides encoding the same that target an associated RNA-guided nuclease (RGN) to a target nucleotide sequence in the TRAC gene. The term “guide RNA” is known in the art and generally refers to an RNA molecule (or a group of RNA molecules collectively) that can bind to an RNA-guided nuclease (RGN) and aid in targeting the RGN to a specific location within a target polynucleotide (e.g., a DNA or an mRNA molecule). The guide RNA can comprise a nucleotide sequence (i.e., a spacer) having sufficient complementarity with a target nucleotide sequence to hybridize with the target sequence and direct sequence-specific binding of an RGN to the target nucleotide sequence. In some embodiments, when the target nucleotide sequence is double-stranded as is the case with DNA, the target nucleotide sequence comprises a non-target strand (which comprises the PAM sequence) and the target strand, which hybridizes with the spacer of the guide RNA. In these embodiments, the guide RNA has sufficient complementarity with the target strand of a double-stranded target sequence (e.g., target DNA sequence of a TRAC gene) such that the guide RNA hybridizes with the target strand and directs sequence-specific binding of an associated RGN to the target sequence (e.g., target DNA sequence of a TRAC gene). Therefore, in some embodiments, a guide RNA includes a spacer that is identical to the sequence of the non-target strand except that uracil (U) replaces thymidine (T) in the guide RNA.

An RGN's respective guide RNA is one or more RNA molecules (generally, one or two), that can bind to the RGN and guide the RGN to bind to a particular target sequence, and in those embodiments wherein the RGN has nickase or nuclease activity, also cleave the target strand and/or the non-target strand. In general, a guide RNA comprises a CRISPR RNA (crRNA) and a trans-activating CRISPR RNA (tracrRNA).

The term “guide RNA” also encompasses, collectively, a group of two or more RNA molecules, where the crRNA and the tracrRNA are located in separate RNA molecules. Native guide RNAs that comprise both a crRNA and a tracrRNA generally comprise two separate RNA molecules that hybridize to each other through the repeat sequence of the crRNA and the anti-repeat sequence of the tracrRNA. In certain embodiments, the crRNA and tracrRNA are linked together by a multi-nucleotide linker (e.g., a four-nucleotide linker) to form a single guide RNA molecule, wherein the crRNA and the tracrRNA hybridize to each other through the repeat sequence of the crRNA and the anti-repeat sequence of the tracrRNA. Thus, a guide RNA encompasses a single-guide RNA (sgRNA), where the crRNA and the tracrRNA are located in the same RNA molecule or strand. A total length of a guide RNA refers to the length of the spacer and backbone in a sgRNA, or length of the crRNA and tracrRNA in a dgRNA.

A guide RNA of the disclosure can comprise at least one chemical modification. The at least one chemical modification includes: a bridged nucleic acid (BNA) modification; 2′-O-methyl(2′-O-Me) modification; 2′-O-methoxy-ethyl(2′MOE) modification; 2′-fluoro (2′-F) modification; 2′F-4′Cα-OMe modification; 2′,4′-di-Cα-OMe modification; 2′-O-methyl 3′phosphorothioate (MS) modification; 2′-O-methyl 3′thiophosphonoacetate (MSP) modification; 2′-O-methyl 3′phosphonoacetate (MP) modification; and phosphorothioate (PS) modification; or a combination thereof. In some embodiments, the BNA comprises a 2′,4′ BNA modification. In some embodiments, the 2′,4′ BNA modification is selected from the group consisting of: locked nucleic acid (LNA) modification, BNA^{NC}[N-Me] modification, 2′-O,4′-C-ethylene bridged nucleic acid (2′,4′-ENA) modification, and S-constrained ethyl (cEt) modification. In some embodiments, the 2′,4′ BNA is a LNA modification. In some embodiments, the 2′,4′ BNA is a cEt modification. In some embodiments, the at least one chemical modification comprises a BNA modification, 2′-O-Me modification, or PS modification. Chemical modifications of spacers, crRNA repeats, crRNAs, tracrRNAs, and guide RNAs are described in International application no. PCT/IB2023/058418, filed Aug. 25, 2023, which is hereby incorporated by reference in its entirety herein. The at least one chemical modification can comprise 2′-O-methyl 3′phosphorothioate (MS) modifications at the 3 terminal nucleotides at the 5′ region and at the 3 terminal nucleotides at the 3′ region of the guide RNA. As used herein, a “5′ region” of an RNA molecule disclosed herein includes the first nucleotide, the first 2 nucleotides, the first 3 nucleotides, the first 4 nucleotides, or the first 5 nucleotides of the 5′ end of the RNA molecule. As used herein, a “3′ region” of an RNA molecule disclosed herein includes the first nucleotide, the first 2 nucleotides, the first 3 nucleotides, the first 4 nucleotides, or the first 5 nucleotides of the 3′ end of the RNA molecule. In some embodiments, a 3′ region of a crRNA in the context of a single guide RNA includes the first nucleotide, the first 2 nucleotides, the first 3 nucleotides, the first 4 nucleotides, or the first 5 nucleotides from the tracrRNA or the linker that joins the crRNA and the tracrRNA of the single guide RNA.

As used herein, the term “crRNA” refers to an RNA molecule or portion thereof that includes a spacer, which is the nucleotide sequence that hybridizes with the target strand of a target sequence, and a CRISPR repeat (i.e. a crRNA repeat) that comprises a nucleotide sequence that forms a structure, either on its own or in concert with a hybridized tracrRNA, that is recognized by the RGN molecule. As used herein, the term “tracrRNA” or “transactivating crRNA” refers to an RNA molecule that comprises an anti-repeat sequence that has sufficient complementarity to hybridize to at least a portion of the CRISPR repeat of a crRNA to form a structure that is recognized by an RGN molecule. In some embodiments, additional secondary structure(s) (e.g., stem-loops) within the tracrRNA molecule is required for binding to an RGN.

The present invention provides CRISPR RNAs (crRNAs) or polynucleotides encoding CRISPR RNAs that target an associated RGN to a target sequence in the TRAC gene. A crRNA comprises a spacer and a CRISPR repeat. The “spacer” has a nucleotide sequence that directly hybridizes with the non-target strand of a target sequence (e.g., target DNA sequence in the TRAC gene) of interest. The spacer is engineered to have full or partial complementarity with the target strand of a target sequence of interest. In some embodiments, the spacer can comprise from about 8 nucleotides to about 30 nucleotides, or more. For example, the spacer can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the spacer is 8, 9, 10, 11, 12, 13, 14, 15, 16, 17, 18, 19, 20, 21, 22, 23, 24, 25, 26, 27, 28, 29, 30, or more nucleotides in length. In some embodiments, the spacer is about 10 to about 26 nucleotides in length, or about 12 to about 30 nucleotides in length. In some embodiments, the spacer is about 30 nucleotides in length. In embodiments, the spacer is 30 nucleotides in length. In some embodiments, the degree of complementarity between a spacer and the target strand of a target sequence (e.g., target DNA sequence), when optimally aligned using a suitable alignment algorithm, is between 50% and 99% or more, including but not limited to about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In embodiments, the degree of complementarity between a spacer and the target strand of a target sequence (e.g., target DNA sequence), when optimally aligned using a suitable alignment algorithm, is 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more. The spacer can be identical in sequence to the non-target strand of a target sequence. In some of those embodiments wherein the target sequence is a target DNA sequence, the spacer can be identical in sequence to the non-target strand of the target DNA sequence, with the exception of the thymidines (Ts) in the target strand being replaced by uracils (Us) in the spacer. In some embodiments, the spacer is free of secondary structure, which can be predicted using any suitable polynucleotide folding algorithm known in the art, including but not limited to mFold (see, e.g., Zuker and Stiegler (1981) Nucleic Acids Res. 9:133-148) and RNAfold (see, e.g., Gruber et al. (2008) Cell 106(1): 23-24). A spacer can comprise at least one chemical modification. In some embodiments, a spacer as part of a guide RNA comprises 2′-O-methyl 3′phosphorothioate (MS) modifications at the 3 terminal nucleotides at the 5′ region of the spacer.

The presently disclosed crRNAs comprise a spacer capable of targeting a bound RGN polypeptide to a target sequence in the T cell receptor alpha chain constant (TRAC) gene, wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, and 104. In some embodiments, a spacer of the disclosure has a nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 or a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 to 5 nucleotides.

In some embodiments, the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 5 nucleotides.

In some embodiments, the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 4 nucleotides.

In some embodiments, the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 3 nucleotides.

In some embodiments, the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 2 nucleotides.

In some embodiments, the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 nucleotide.

In some embodiments, a spacer of the disclosure has a nucleotide sequence set forth as: GCCGUGUACCAGCUGAGAGACUCU (SEQ ID NO: 7), or a nucleotide sequence that differs in length and/or sequence from SEQ ID NO: 7 by 1 to 5 nucleotides. In some embodiments, the spacer has a nucleotide sequence that differs in length and/or sequence from SEQ ID NO: 7 by 5 nucleotides. In some embodiments, the spacer has a nucleotide sequence that differs in length and/or sequence from SEQ ID NO: 7 by 4 nucleotides. In some embodiments, the spacer has a nucleotide sequence that differs in length and/or sequence from SEQ ID NO: 7 by 3 nucleotides. In some embodiments, the spacer has a nucleotide sequence that differs in length and/or sequence from SEQ ID NO: 7 by 2 nucleotides. In some embodiments, the spacer has a nucleotide sequence that differs in length and/or sequence from SEQ ID NO: 7 by 1 nucleotide.

In some embodiments, a spacer of the disclosure has a nucleotide sequence set forth as: AUCCUCUUGUCCCACAGAUAUCC (SEQ ID NO: 9), or a nucleotide sequence that differs in length and/or sequence from SEQ ID NO: 9 by 1 to 5 nucleotides. In some embodiments, the spacer has a nucleotide sequence that differs in length and/or sequence from SEQ ID NO: 9 by 5 nucleotides. In some embodiments, the spacer has a nucleotide sequence that differs in length and/or sequence from SEQ ID NO: 9 by 4 nucleotides. In some embodiments, the spacer has a nucleotide sequence that differs in length and/or sequence from SEQ ID NO: 9 by 3 nucleotides. In some embodiments, the spacer has a nucleotide sequence that differs in length and/or sequence from SEQ ID NO: 9 by 2 nucleotides. In some embodiments, the spacer has a nucleotide sequence that differs in length and/or sequence from SEQ ID NO: 9 by 1 nucleotide.

Along with a spacer, crRNAs further comprise a CRISPR RNA repeat. The CRISPR RNA repeat comprises a nucleotide sequence that forms a structure, either on its own or in concert with a hybridized tracrRNA, that is recognized by the RGN molecule. In some embodiments, the CRISPR RNA repeat can comprise from about 8 nucleotides to about 30 nucleotides, or more. For example, the CRISPR repeat can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the CRISPR repeat is 8, 9, 10, 11, 12, 13, 14, 15, 16, 17, 18, 19, 20, 21, 22, 23, 24, 25, 26, 27, 28, 29, 30, or more nucleotides in length. In some embodiments, the degree of complementarity between a CRISPR repeat and its corresponding tracrRNA antirepeat, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In particular embodiments, the degree of complementarity between a CRISPR repeat and its corresponding tracrRNA antirepeat, when optimally aligned using a suitable alignment algorithm, is 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more.

The CRISPR repeat can comprise the nucleotide sequence of any one of SEQ ID NOs: 106, 109-112, 328, 331, and 334, or an active variant or fragment thereof that when comprised within a guide RNA, is capable of directing the sequence-specific binding of an associated RNA-guided nuclease provided herein to a presently disclosed target DNA sequence within the TRAC gene. In some embodiments, an active CRISPR repeat variant comprises a nucleotide sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to a nucleotide sequence set forth as any one of SEQ ID NOs: 106, 109-112, 328, 331, and 334. In some embodiments, an active CRISPR repeat fragment comprises at least 5, 6, 7, 8, 9, 10, 11, 12, 13, 14, 15, 16, 17, 18, 19, 20, 21, or 22 contiguous nucleotides of a nucleotide sequence set forth as any one of SEQ ID NOs: 106, 109-112, 328, 331, and 334. In some embodiments, the CRISPR repeat comprises a nucleotide sequence that differs in length and/or sequence from SEQ ID NO: 106 by 1 to 8 nucleotides. In some embodiments, the CRISPR repeat comprises a nucleotide sequence that differs in length and/or sequence from SEQ ID NO: 106 by 8 nucleotides. In some embodiments, the CRISPR repeat comprises a nucleotide sequence that differs in length and/or sequence from SEQ ID NO: 106 by 7 nucleotides. In some embodiments, the CRISPR repeat comprises a nucleotide sequence that differs in length and/or sequence from SEQ ID NO: 106 by 6 nucleotides. In some embodiments, the CRISPR repeat comprises a nucleotide sequence that differs in length and/or sequence from SEQ ID NO: 106 by 5 nucleotides. In some embodiments, the CRISPR repeat comprises a nucleotide sequence that differs in length and/or sequence from SEQ ID NO: 106 by 4 nucleotides. In some embodiments, the CRISPR repeat comprises a nucleotide sequence that differs in length and/or sequence from SEQ ID NO: 106 by 3 nucleotides. In some embodiments, the CRISPR repeat comprises a nucleotide sequence that differs in length and/or sequence from SEQ ID NO: 106 by 2 nucleotides. In some embodiments, the CRISPR repeat comprises a nucleotide sequence that differs in length and/or sequence from SEQ ID NO: 106 by 1 nucleotide. In some embodiments, the CRISPR repeat comprises the nucleotide sequence set forth as: GUCAUAGUUCCAUUAAAGCCA (SEQ ID NO: 106). A CRISPR repeat can comprise at least one chemical modification. In some embodiments, a CRISPR repeat as part of a guide RNA comprises 2′-O-methyl 3′phosphorothioate (MS) modifications at the 3 terminal nucleotides at the 3′ region of the CRISPR repeat. CRISPR repeats comprising 2′-O-methyl 3′phosphorothioate (MS) modifications at the 3 terminal nucleotides at the 3′ region of the CRISPR repeat can have nucleotide sequences set forth as any one of SEQ ID NOs: 430, 432-435, 583, 585, and 587.

The crRNA can be an engineered sequence that is not naturally occurring. In some embodiments, the specific CRISPR repeat is not linked to the engineered spacer in nature and the CRISPR repeat is considered heterologous to the spacer. In some embodiments, the spacer is an engineered sequence that is not naturally occurring.

In some embodiments, the crRNA has the sequence set forth as any one of SEQ ID NOs: 136-197. A crRNA can comprise at least one chemical modification. In some embodiments, a crRNA of the disclosure can comprise 2′-O-methyl 3′phosphorothioate (MS) modifications at the 3 terminal nucleotides at the 5′ region and at the 3 terminal nucleotides at the 3′ region of the crRNA. crRNAs comprising 2′-O-methyl 3′phosphorothioate (MS) modifications at the 3 terminal nucleotides at the 5′ region and at the 3 terminal nucleotides at the 3′ region of the crRNA can have nucleotide sequences set forth as any one of SEQ ID NOs: 459-520.

Generally, presently disclosed guide RNAs comprise a crRNA and a trans-activating CRISPR RNA (tracrRNA), while some presently disclosed compositions and methods utilize RGN polypeptides that do not require a tracrRNA. A tracrRNA molecule comprises a nucleotide sequence comprising a region, referred to herein as the anti-repeat, that has sufficient complementarity to hybridize to a crRNA repeat. In some embodiments, the tracrRNA molecule further comprises a region with secondary structure (e.g., stem-loop). In some embodiments, secondary structure includes nucleotides that are in one of two states, paired or unpaired, where nucleotide or base pairing includes base-base hydrogen bonding interactions (e.g., adenine (A) pairs with uracil (U), cytosine (C) pairs with guanine (G)) between two complementary nucleic acid strands to form a helix. In some embodiments, the combination of one or more helical elements interspersed with unpaired, single-stranded nucleotides constitutes an RNA structure.

A “stem loop” as used herein refers to a form of secondary structure comprising at least one “stem” and at least one “loop”, “bulge”, or “bubble” found in polynucleotides. A stem loop can form intramolecularly (within one molecule, e.g., within a tracrRNA or a sgRNA) or intermolecularly (between two distinct nucleic acids, e.g., in a dual guide RNA by the crRNA repeat of a crRNA and the anti-repeat of a tracrRNA). Stem loops are created when there is at least some complementarity between two nucleic acid sequences to form a paired double helix. The paired double helix region with full complementarity or sometimes including a G: U wobble base pair (or I: U, I: A, or I: C, where I refers to inosine) is referred to as a “stem”. The term “loop”, “bulge”, or “bubble” refers to a single stranded region within the “stem loop” structure where there is no complementarity between nucleotides, excluding G: U wobble base pairs (or I: U, I: A, or I: C, where I refers to inosine). Thus, “loops”, “bulges” and “bubbles” include nucleotides that are not paired. In some embodiments, a “loop” is distinguished from a “bulge” or “bubble” by being located at one end of the “stem loop” structure, while a “bulge” or a “bubble” is located between two “stems” in the “stem loop” structure.

In certain embodiments, a stem loop structure comprises a stem and a loop at one end of the stem. In some embodiments, a stem loop structure comprises a first stem and a second stem with a bubble in between the stems. In some embodiments, a stem loop structure comprises a loop, multiple stems and multiple bubbles in between the stems. In this circumstance, the bubbles in the order of closeness to the loop are referred to as a “first bubble”, a “second bubble”, a “third bubble”, etc., and the stems in the order of closeness to the loop are referred to as a “first stem”, a “second stem”, a “third stem”, etc. In embodiments of dgRNA, the stem loop formed by the crRNA repeat of a crRNA and the anti-repeat of a tracrRNA does not include a loop, and thus the bubbles in the order of closeness to the 5′ end of the tracrRNA (or 3′ end of the crRNA) are referred to as a “first bubble”, a “second bubble”, a “third bubble”, etc., and the stems in the order of closeness to the 5′ end of the tracrRNA (or 3′ end of the crRNA) are referred to as a “first stem”, a “second stem”, a “third stem”, etc.

The term “first stem of a crRNA repeat of a crRNA”, “first stem of a crRNA repeat”, or “first stem of a crRNA” means the region in the crRNA repeat of the crRNA that forms the first stem of a stem loop structure when hybridizing with an anti-repeat of a tracrRNA. The term “second stem of a crRNA repeat of a crRNA”, “second stem of a crRNA repeat”, or “second stem of a crRNA” means the region in the crRNA repeat of the crRNA that forms the second stem of a stem loop structure when hybridizing with an anti-repeat of a tracrRNA. Similarly, the term “first stem of an anti-repeat of a tracrRNA”, “first stem of an anti-repeat”, or “first stem of a tracrRNA” means the region in the anti-repeat of the tracrRNA that forms the first stem of a stem loop structure when hybridizing with a crRNA repeat of a crRNA. The term “second stem of an anti-repeat of a tracrRNA”, “second stem of an anti-repeat”, or “second stem of a tracrRNA” means the region in the anti-repeat of the tracrRNA that forms the second stem of a stem loop structure when hybridizing with a crRNA repeat of a crRNA.

In some embodiments, a stem loop formed intramolecularly is a hairpin stem loop. Base pairings occur in the stem part of a stem loop and typically involve guanine-cytosine base pairing and adenine-uracil (thymidine) base pairing, although guanine-uracil base pairing is possible. Base stacking interactions promote helix formation. The loop part of a stem loop includes bases that are not paired. In some embodiments, a loop is the point at which a nucleic acid strand turns back on itself for nucleotide pairing to create a stem. In some embodiments, loops that are less than three bases long are sterically impossible and do not form. In some embodiments, optimal loop length is about 4-8 bases long. Common loops with four nucleotide sequences such as GAAA, AAAG, ACUU, or UUCG are known as the “tetraloop” and are particularly stable due to the base-stacking interactions of its component nucleotides.

In some embodiments, the region of the tracrRNA that is fully or partially complementary to a crRNA repeat is at the 5′ end of the molecule and the 3′ end of the tracrRNA comprises secondary structure. This region of secondary structure generally comprises several hairpin structures, including the nexus hairpin, which is found adjacent to the anti-repeat. The nexus forms the core of the interactions between the guide RNA and the RGN, and is at the intersection between the guide RNA, the RGN, and the target sequence. The nexus hairpin often has a conserved nucleotide sequence in the base of the hairpin stem, with the motif UNANNC found in many nexus hairpins in tracrRNAs. In embodiments, guide RNAs or RGN systems of the disclosure use tracrRNAs that comprise non-canonical sequences in the base of the hairpin stem of their nexus hairpins, including UNANNG and CNANNC. In some embodiments, a guide RNA or an RGN system of the disclosure uses a tracrRNA that includes, in the base of the nexus hairpin stem, the non-canonical sequence of UNANNG. In some embodiments, a guide RNA or an RGN system of the disclosure uses a tracrRNA that includes, in the base of the nexus hairpin stem, the non-canonical sequence of CNANNC. There are often terminal hairpins at the 3′ end of the tracrRNA that can vary in structure and number, but often comprise a GC-rich Rho-independent transcriptional terminator hairpin followed by a string of U's at the 3′ end. See, for example, Briner et al. (2014) Molecular Cell 56:333-339, Briner and Barrangou (2016) Cold Spring Harb Protoc; doi: 10.1101/pdb.top090902, and U.S. Publication No. 2017/0275648, each of which is herein incorporated by reference in its entirety.

A tracrRNA of the disclosure can include a tail. The term “tail” as used herein refers to the non-complementary region closest to the 3′ end (e.g., within twelve, eleven, ten, nine, eight, seven, six, five nucleotides from the 3′ end) of a tracrRNA of the disclosure. In some embodiments, a tail of a tracrRNA includes 1-12, 1-8, 1-7, or 1-6 nucleotides from the 3′ end of the tracrRNA. In some embodiments, a tail of a tracrRNA includes 1, 2, 3, 4, 5, 6, 7, 8, 9, 10, 11, 12, or more nucleotides from the 3′ end of the tracrRNA.

A tracrRNA of the disclosure can include additional hairpin or stem loop structures in addition to the nexus hairpin. In some embodiments, a tracrRNA includes at least one stem loop. In some embodiments, a tracrRNA includes at least one stem loop proximal to the anti-repeat and at least one stem loop proximal to the 3′ end of the tracrRNA. “Proximal” refers to being within 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides of a region or an end of a nucleic acid molecule. In certain embodiments, “proximal” refers to being within 1 nucleotide, 2 nucleotides, 3 nucleotides, 4 nucleotides, 5 nucleotides, or 6 nucleotides of a region or an end of a nucleic acid molecule. “Most proximal” refers to being the nearest to a region or to an end of a nucleic acid molecule. For example, a stem loop most proximal to the tail of a tracrRNA is the first stem loop nearest the tail of the tracrRNA. “Distal” refers to being at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, or more away from a region or an end of a nucleic acid molecule. In some embodiments, “distal” refers to being at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, or more away from a structure of a nucleic acid molecule (e.g., bubble, loop). For example, nucleotides of the first stem of the anti-repeat of a dual guide RNA distal to the first bubble of the stem loop is nearer to the 3′ terminal nucleotide of the crRNA and the 5′ terminal nucleotide of the tracrRNA than they are to the first bubble. A tracrRNA also forms secondary structure upon hybridizing with its corresponding crRNA. The anti-repeat region of a tracrRNA is fully or partially complementary to the crRNA repeat of a crRNA. In some embodiments, a portion of the anti-repeat of a tracrRNA and a portion of a crRNA repeat hybridize and form a stem. In some embodiments, the crRNA: tracrRNA stem includes at least one nucleotide pair (i.e. base pair) because these portions of the anti-repeat and crRNA repeat are complementary. As described elsewhere herein, a portion of the anti-repeat of a tracrRNA forming a first stem is the first stem of the anti-repeat, a portion of the anti-repeat of a tracrRNA forming a second stem is the second stem of the anti-repeat, a portion of the anti-repeat of a tracrRNA forming a third stem is the third stem of the anti-repeat, etc. As described elsewhere herein, a portion of the crRNA repeat of a crRNA forming a first stem is the first stem of the crRNA repeat, a portion of the crRNA repeat of a crRNA forming a second stem is the second stem of the crRNA repeat, a portion of the crRNA repeat of a crRNA forming a third stem is the third stem of the crRNA repeat, etc. In some embodiments, a portion of the anti-repeat of a tracrRNA and a portion of the crRNA repeat are not complementary with each other and thus do not hybridize to form base pairs. In some embodiments, the region of non-complementarity between the anti-repeat and the crRNA repeat forms a bulge or a bubble. In some embodiments, hybridization of the anti-repeat of a tracrRNA and the crRNA repeat of a crRNA forms a secondary structure that includes at least one stem. In some embodiments, hybridization of the anti-repeat of a tracrRNA and the crRNA repeat of a crRNA forms a secondary structure that includes at least one bubble. In some embodiments, hybridization of the anti-repeat of a tracrRNA and the crRNA repeat of a crRNA forms a secondary structure that includes at least one stem and at least one bubble. In some embodiments, hybridization of the anti-repeat of a tracrRNA and the crRNA repeat of a crRNA forms a secondary structure that includes two stems and one bubble in between.

In some embodiments, the anti-repeat of the tracrRNA that is fully or partially complementary to the CRISPR repeat comprises from about 8 nucleotides to about 30 nucleotides, or more. For example, the region of base pairing between the tracrRNA anti-repeat and the CRISPR repeat can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the region of base pairing between the tracrRNA anti-repeat and the CRISPR repeat is 6, 7, 8, 9, 10, 11, 12, 13, 14, 15, 16, 17, 18, 19, 20, 21, 22, 23, 24, 25, 26, 27, 28, 29, 30, or more nucleotides in length. In some embodiments, the degree of complementarity between a CRISPR repeat and its corresponding tracrRNA anti-repeat, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In some embodiments, the degree of complementarity between a CRISPR repeat and its corresponding tracrRNA anti-repeat, when optimally aligned using a suitable alignment algorithm, is 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more.

In some embodiments, the entire tracrRNA can comprise from about 60 nucleotides to more than about 210 nucleotides. For example, the tracrRNA can be about 60, about 65, about 70, about 75, about 80, about 85, about 90, about 95, about 100, about 105, about 110, about 115, about 120, about 125, about 130, about 135, about 140, about 150, about 160, about 170, about 180, about 190, about 200, about 210, or more nucleotides in length. In some embodiments, the tracrRNA is 60, 65, 70, 75, 80, 85, 90, 95, 100, 105, 110, 115, 120, 125, 130, 135, 140, 150, 160, 170, 180, 190, 200, 210 or more nucleotides in length. In some embodiments, the tracrRNA is about 70 to about 105 nucleotides in length, including about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, and about 105 nucleotides in length. In embodiments, the tracrRNA is 70 to 105 nucleotides in length, including 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, and 105 nucleotides in length.

In some embodiments, the tracrRNA comprises the nucleotide sequence set forth as any one of SEQ ID NOs: 107, 114-123, 329, 332, and 335, or an active variant or fragment thereof that when comprised within a guide RNA is capable of directing the sequence-specific binding of an associated RNA-guided nuclease provided herein to a presently disclosed target sequence within the TRAC gene. In some embodiments, an active tracrRNA sequence variant comprises a nucleotide sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the nucleotide sequence set forth as any one of SEQ ID NOs: 107, 114-123, 329, 332, and 335. In some embodiments, an active tracrRNA sequence fragment comprises at least 5, 10, 15, 20, 25, 30, 35, 40, 45, 50, 55, 60, 65, 70, 75, 80, or more contiguous nucleotides of the nucleotide sequence set forth as any one of SEQ ID NOs: 107, 114-123, 329, 332, and 335. An active tracrRNA sequence fragment differs in length from SEQ ID NO: 107 by 1 to 16 nucleotides. In some embodiments, an active tracrRNA has a nucleotide sequence that is 8 nucleotides shorter than the nucleotide sequence set forth as SEQ ID NO: 107. In some embodiments, an active tracrRNA has a nucleotide sequence that is 11 nucleotides shorter than the nucleotide sequence set forth as SEQ ID NO: 107. An active tracrRNA sequence fragment can comprise the nucleotide sequence set forth as any one of SEQ ID NOs: 107, 114-123, 329, 332, and 335. In some embodiments, an active tracrRNA has at least 80% sequence identity, at least 90% sequence identity, or at least 95% sequence identity to a nucleotide sequence set forth as SEQ ID NO: 107. In some embodiments, an active tracrRNA has the nucleotide sequence set forth as:

(SEQ ID NO: 107)
UGGCUUUGAUGUUUCUAUGAUAAGGGUUUCGACCCGUGGCGUCGGGGAU
CGCCUGCCCAUUGAAAUGGGCUUCUCCCCAUUUAUU.

A tracrRNA can comprise at least one chemical modification. In some embodiments, a tracrRNA of the disclosure can comprise 2′-O-methyl 3′phosphorothioate (MS) modifications at the 3 terminal nucleotides at the 5′ region and at the 3 terminal nucleotides at the 3′ region of the tracrRNA. TracrRNAs comprising 2′-O-methyl 3′phosphorothioate (MS) modifications at the 3 terminal nucleotides at the 5′ region and at the 3 terminal nucleotides at the 3′ region of the tracrRNA can have nucleotide sequences set forth as any one of SEQ ID NOs: 431, 437-446, 584, 586, and 588.

Two polynucleotide sequences can be considered to be substantially complementary when the two sequences hybridize to each other under stringent conditions. The term “hybridize” refers to one molecule binding or associating with another molecule, or regions of one molecule binding or associating with each other. A spacer of a guide RNA and its target sequence are considered to be substantially complementary when the two sequences hybridize to each other sufficiently to allow for the localization to the target sequence of an RGN bound to the guide RNA. Likewise, an RGN is considered to bind to a particular target sequence in a sequence-specific manner if the guide RNA bound to the RGN binds to a target sequence under normal experimental or in vivo conditions. The term “sequence specific” can also refer to the binding of a RGN polypeptide to a target sequence at a greater affinity than binding to a randomized background sequence.

The Tm is the temperature (under defined ionic strength and pH) at which 50% of a complementary target sequence hybridizes to a perfectly matched sequence. For DNA-DNA hybrids, the Tm can be approximated from the equation of Meinkoth and Wahl (1984) Anal. Biochem. 138:267-284: Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, New York).

The guide RNA can be a single guide RNA (sgRNA) or a dual-guide RNA (dgRNA). A single guide RNA comprises the crRNA and tracrRNA on a single molecule of RNA, whereas a dual-guide RNA system comprises a crRNA and a tracrRNA present on two distinct RNA molecules, hybridized to one another through at least a portion of the CRISPR repeat of the crRNA and at least a portion of the tracrRNA (i.e., the anti repeat), which may be fully or partially complementary to the CRISPR repeat of the crRNA. In embodiments wherein the guide RNA is a single guide RNA, the crRNA and tracrRNA are separated by a linker nucleotide sequence. In general, the linker nucleotide sequence is one that does not include complementary bases in order to avoid the formation of secondary structure within or comprising nucleotides of the linker nucleotide sequence. In some embodiments, the linker nucleotide sequence between the crRNA and tracrRNA is at least 3, at least 4, at least 5, at least 6, at least 7, at least 8, at least 9, at least 10, at least 11, at least 12, or more nucleotides in length. In some embodiments, the linker nucleotide sequence of a single guide RNA is at least 4 nucleotides in length. In certain embodiments, the linker nucleotide sequence of a single guide RNA is 4 nucleotides in length. In some embodiments, the linker nucleotide sequence includes a nucleotide sequence set forth as any of AAAG, GAAA, ACUU, and CAAAGG. In certain embodiments, the linker nucleotide sequence includes a nucleotide sequence set forth as AAAG. In some embodiments, the linker nucleotide sequence includes a nucleotide sequence set forth as GAAA. In some embodiments, the linker nucleotide sequence includes a nucleotide sequence set forth as ACUU. In some embodiments, the linker nucleotide sequence includes a nucleotide sequence set forth as CAAAGG.

The single guide RNA or dual-guide RNA can be synthesized chemically or via in vitro transcription. Assays for determining sequence-specific binding between an RGN and a guide RNA are known in the art and include, but are not limited to, in vitro binding assays between an expressed RGN and the guide RNA, which can be tagged with a detectable label (e.g., biotin) and used in a pull-down detection assay in which the guide RNA: RGN complex is captured via the detectable label (e.g., with streptavidin beads). A control guide RNA with an unrelated sequence or structure to the guide RNA can be used as a negative control for non-specific binding of the RGN to RNA. In some embodiments, the guide RNA includes any one of SEQ ID NOs: 198-200, 202-213, 215-233, 235-241, and 243-259. In some embodiments, the guide RNA has at least 80% sequence identity, at least 90% sequence identity, or at least 95% sequence identity, to a nucleotide sequence set forth as SEQ ID NO: 204. In some embodiments, the guide RNA has the nucleotide sequence set forth as: GCCGUGUACCAGCUGAGAGACUCUGUCAUAGUUCCAUAAAGAUGUUUCUAUGAUAAG GGUUUCGACCCGUGGCGUCGGGGAUCGCCUGCCCAUUGAAAUGGGCUUCUCCCCAUUU AUU (SEQ ID NO: 204). In some embodiments, the guide RNA has at least 80% sequence identity, at least 90% sequence identity, or at least 95% sequence identity, to a nucleotide sequence set forth as SEQ ID NO: 205. In some embodiments, the guide RNA has the nucleotide sequence set forth as:

(SEQ ID NO: 205)
AUCCUCUUGUCCCACAGAUAUCCGUCAUAGUUCCAUUAAAAAGUUGAUG
UUUCUAUGAUAAGGGUUUCGACCCGUGGCGUCGGGGAUCGCCUGCCCUU
GAAAGGGCUUCUCCCCAUU.

A guide RNA of the disclosure can comprise at least one chemical modification. In a single guide RNA format, the at least one chemical modification can comprise 2′-O-methyl 3′phosphorothioate (MS) modifications at the 3 terminal nucleotides at the 5′ region and at the 3 terminal nucleotides at the 3′ region of the single guide RNA. In a dual guide RNA format, the at least one chemical modification can comprise 2′-O-methyl 3′phosphorothioate (MS) modifications at the 3 terminal nucleotides at the 5′ region and at the 3 terminal nucleotides at the 3′ region of the crRNA, and can comprise 2′-O-methyl 3′phosphorothioate (MS) modifications at the 3 terminal nucleotides at the 5′ region and/or at the 3 terminal nucleotides at the 3′ region of the tracrRNA. MS modified guide RNAs can have nucleotide sequences set forth as any one of SEQ ID NOs: 521-523, 525-536, 538-556, 558-564, and 566-582.

The guide RNA can be introduced into a target cell or embryo as an RNA molecule. The guide RNA can be transcribed in vitro or chemically synthesized. In some embodiments, a nucleotide sequence encoding the guide RNA is introduced into the cell or embryo. In some embodiments, the nucleotide sequence encoding the guide RNA is operably linked to a promoter (e.g., an RNA polymerase III promoter). The promoter can be a native promoter or heterologous to the guide RNA-encoding nucleotide sequence.

In some embodiments, the guide RNA can be introduced into a target cell or embryo as a ribonucleoprotein complex, as described herein, wherein the guide RNA is bound to an RGN polypeptide.

The guide RNA directs an associated RGN to a particular target nucleotide sequence of interest through hybridization of the guide RNA to the target sequence of interest. The target sequence can be bound (and in some embodiments, cleaved) by an RNA-guided nuclease in vitro or in a cell. A target sequence can comprise DNA, RNA, or a combination of both and can be single-stranded or double-stranded. A target sequence can be genomic DNA (i.e., chromosomal DNA), plasmid DNA, or an RNA molecule (e.g., messenger RNA, ribosomal RNA, transfer RNA, micro RNA, small interfering RNA). In those embodiments wherein the target sequence is a chromosomal sequence, the chromosomal sequence can be a nuclear or mitochondrial chromosomal sequence. In the presently disclosed compositions and methods, the target sequence is within a target nucleic acid molecule that is double-stranded (e.g., a target DNA sequence). More specifically, the target sequence is within the TRAC gene. In some embodiments, the target sequence is unique in the target genome. In some embodiments, the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, and 104.

The target sequence is adjacent to a protospacer adjacent motif (PAM) and the non-target strand of the target sequence is the strand that comprises the PAM. The PAM is immediately adjacent to the target sequence and often comprises Ns, where each “N” represents any nucleotide. In some embodiments, the PAM comprises about 1 to about 10 Ns, including about 1, about 2, about 3, about 4, about 5, about 6, about 7, about 8, about 9, or about 10 Ns. In certain embodiments, a PAM comprises 1 to 10 Ns, including 1, 2, 3, 4, 5, 6, 7, 8, 9, or 10 Ns. The PAM can be 5′ or 3′ of the target sequence on its non-target strand. In some embodiments, the PAM is 3′ of the target sequence on its non-target strand for the presently disclosed guide RNAs and RGN systems. Generally, the PAM is a consensus sequence of about 3-4 nucleotides, but in certain embodiments it can be 2, 3, 4, 5, 6, 7, 8, 9, or more nucleotides in length.

In some embodiments, a PAM sequence adjacent to a presently disclosed target sequence on its non-target strand comprises the consensus sequence set forth as any one of the PAM sequences in Table 1. In some embodiments, a PAM sequence adjacent to the presently disclosed target sequence on its non-target strand includes the sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG. In some embodiments, the PAM sequence is 3′ of the target sequence on its non-target strand.

It is well-known in the art that PAM sequence specificity for a given nuclease enzyme is affected by enzyme concentration (see, e.g., Karvelis et al. (2015) Genome Biol 16:253), which may be modified by altering the promoter used to express the RGN, or the amount of ribonucleoprotein complex delivered to the cell or embryo.

Upon recognizing its corresponding PAM sequence, the RGN can cleave one or both strands of a target sequence at a specific cleavage site. As used herein, a cleavage site is made up of the two particular nucleotides within a target sequence between which the target strand, non-target strand, or both strands of a target sequence are cleaved by an RGN. The cleavage site can comprise the 1^{st} and 2^{nd}, 2^{nd} and 3^{rd}, 3^{rd} and 4^{th}, 4^{th} and 5^{th}, 5^{th} and 6^{th}, 7^{th} and 8^{th}, or 8^{th} and 9^{th} nucleotides from the PAM in either the 5′ or 3′ direction. In some embodiments, the cleavage site may be over 10, 11, 12, 13, 14, 15, 16, 17, 18, 19, or 20 nucleotides from the PAM in either the 5′ or 3′ direction. As RGNs can cleave a target sequence resulting in staggered ends, in certain embodiments, the cleavage site is defined based on the distance of the two nucleotides from the PAM on the non-target strand of the target sequence and, for the target strand, the distance of the two nucleotides from the complement of the PAM.

### III. Length Modifications to Guide RNA

The guide RNAs disclosed herein that are effective in targeting an associated RNA-guided nuclease (RGN) to a target nucleotide sequence in the TRAC gene can be engineered to be shorter than their corresponding native guide RNAs but have comparable efficiencies as their corresponding native guide RNAs in gene editing. A native guide RNA includes a guide RNA that is naturally occurring, for example, a guide RNA from an organism. A guide RNA that is engineered to be shorter than its native guide RNA length can be as effective as its non-engineered counterpart in its ability to bind an associated RGN and cleave and/or modify a target sequence.

A modification (e.g., deletion, truncation) “within” a region of a RNA molecule of the disclosure includes all nucleotides and phosphate backbone in that region, including the first and last nucleotide positions that are considered part of that region.

In some embodiments, a spacer, a crRNA repeat, a crRNA, an anti-repeat, a tracrRNA, a backbone, and/or a guide RNA of the present disclosure are engineered to be truncated or shortened. In some embodiments, a truncated spacer, truncated crRNA repeat, truncated crRNA, truncated anti-repeat, truncated tracrRNA, truncated backbone, and/or truncated guide RNA maintains or enhances gene editing efficiency as compared to the same spacer, crRNA repeat, crRNA, anti-repeat, tracrRNA, backbone, and/or guide RNA prior to its engineering. “Truncation” and “deletion” in the context of engineering a spacer, crRNA repeat, crRNA, anti-repeat, tracrRNA, backbone, or guide RNA, are used interchangeably herein and refer to removal of at least one nucleotide from a reference spacer, crRNA repeat, crRNA, anti-repeat, tracrRNA, backbone, or guide RNA, which might be naturally occurring or synthetic.

An engineered spacer can comprise a truncation of 1 nucleotide (nt), 2 nt, 3 nt, 4 nt, or 5 nt, as compared to the same spacer prior to its engineering. An engineered spacer can comprise a truncation of 1 nt, as compared to the spacer prior to its engineering. An engineered spacer can comprise a truncation of 2 nt, as compared to the spacer prior to its engineering. An engineered spacer can comprise a truncation of 3 nt, as compared to the spacer prior to its engineering. An engineered spacer can comprise a truncation of 4 nt, as compared to the spacer prior to its engineering. An engineered spacer can comprise a truncation of 5 nt, as compared to the spacer prior to its engineering. In some embodiments, a spacer of the disclosure has a nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103. In some embodiments, a spacer as part of a guide RNA comprises 2′-O-methyl 3′phosphorothioate (MS) modifications at the 3 terminal nucleotides at the 5′ region of the spacer.

An engineered crRNA repeat can comprise a truncation of 1 nt, 2 nt, 3 nt, 4 nt, 5 nt, 6 nt, 7 nt, 8 nt, 9 nt, or 10 nt, as compared to the crRNA repeat prior to its engineering. An engineered crRNA repeat can comprise a truncation of 1 nt, 2 nt, 3 nt, 4 nt, 5 nt, 6 nt, 7 nt, 8 nt, 9 nt, or 10 nt from its 3′ terminus as compared to the nucleotide sequence set forth as SEQ ID NO: 106. In some embodiments, an engineered crRNA repeat comprises a truncation of 1 nt from its 3′ terminus as compared to the nucleotide sequence set forth as SEQ ID NO: 106. In some embodiments, an engineered crRNA repeat comprises a truncation of 2 nt from its 3′ terminus as compared to the nucleotide sequence set forth as SEQ ID NO: 106. In some embodiments, an engineered crRNA repeat comprises a truncation of 3 nt from its 3′ terminus as compared to the nucleotide sequence set forth as SEQ ID NO: 106. In some embodiments, an engineered crRNA repeat comprises a truncation of 4 nt from its 3′ terminus as compared to the nucleotide sequence set forth as SEQ ID NO: 106. In some embodiments, an engineered crRNA repeat comprises a truncation of 5 nt from its 3′ terminus as compared to the nucleotide sequence set forth as SEQ ID NO: 106. In some embodiments, an engineered crRNA repeat comprises a truncation of 6 nt from its 3′ terminus as compared to the nucleotide sequence set forth as SEQ ID NO: 106. In some embodiments, an engineered crRNA repeat comprises a truncation of 7 nt from its 3′ terminus as compared to the nucleotide sequence set forth as SEQ ID NO: 106. In some embodiments, an engineered crRNA repeat comprises a truncation of 8 nt from its 3′ terminus as compared to the nucleotide sequence set forth as SEQ ID NO: 106. In some embodiments, an engineered crRNA repeat comprises a truncation of 9 nt from its 3′ terminus as compared to the nucleotide sequence set forth as SEQ ID NO: 106. In some embodiments, an engineered crRNA repeat comprises a truncation of 10 nt from its 3′ terminus as compared to the nucleotide sequence set forth as SEQ ID NO: 106.

In some embodiments, an engineered crRNA repeat has the nucleotide sequence set forth as SEQ ID NO: 106 or that differs in length and/or sequence from SEQ ID NO: 106 by 1 to 8 nucleotides. In some embodiments, an engineered crRNA repeat has a nucleotide sequence that differs in length and/or sequence from SEQ ID NO: 106 by 8 nucleotides. In some embodiments, an engineered crRNA repeat has a nucleotide sequence that differs in length and/or sequence from SEQ ID NO: 106 by 7 nucleotides. In some embodiments, an engineered crRNA repeat has a nucleotide sequence that differs in length and/or sequence from SEQ ID NO: 106 by 6 nucleotides. In some embodiments, an engineered crRNA repeat has a nucleotide sequence that differs in length and/or sequence from SEQ ID NO: 106 by 5 nucleotides. In some embodiments, an engineered crRNA repeat has a nucleotide sequence that differs in length and/or sequence from SEQ ID NO: 106 by 4 nucleotides. In some embodiments, an engineered crRNA repeat has a nucleotide sequence that differs in length and/or sequence from SEQ ID NO: 106 by 3 nucleotides. In some embodiments, an engineered crRNA repeat has a nucleotide sequence that differs in length and/or sequence from SEQ ID NO: 106 by 2 nucleotides. In some embodiments, an engineered crRNA repeat has a nucleotide sequence that differs in length and/or sequence from SEQ ID NO: 106 by 1 nucleotide.

A crRNA repeat can comprise a total length of at least 10, 11, 12, 13, 14, 15, or 16 nucleotides. A crRNA repeat can comprise a total length of at most 10, 11, 12, 13, 14, 15, or 16 nucleotides. In some embodiments, a crRNA repeat can comprise a total length of 13 nucleotides. In some embodiments, a crRNA repeat can comprise a total length of 16 nucleotides. In some embodiments, a crRNA repeat has the nucleotide sequence set forth as any one of SEQ ID NOs: 106, 109-112, 328, 331, and 334. In some embodiments, a crRNA repeat as part of a guide RNA comprises 2′-O-methyl 3′phosphorothioate (MS) modifications at the 3 terminal nucleotides at the 3′ region of the crRNA repeat. MS modified crRNA repeats can have nucleotide sequences set forth as any one of SEQ ID NOs: 430, 432-435, 583, 585, and 587.

An engineered crRNA can comprise a truncation of 1 nt, 2 nt, 3 nt, 4 nt, 5 nt, 6 nt, 7 nt, 8 nt, 9 nt, 10 nt, 11 nt, 12 nt, 13 nt, 14 nt, or 15 nt as compared to the crRNA prior to its engineering. An engineered crRNA can comprise a truncation of 1 nt, 2 nt, 3 nt, 4 nt, or 5 nt from its 5′ terminus. In some embodiments, an engineered crRNA comprises a truncation of 1 nt from its 5′ terminus. In some embodiments, an engineered crRNA comprises a truncation of 2 nt from its 5′ terminus. In some embodiments, an engineered crRNA comprises a truncation of 3 nt from its 5′ terminus. An engineered crRNA can comprise a truncation of 1 nt, 2 nt, 3 nt, 4 nt, 5 nt, 6 nt, 7 nt, 8 nt, 9 nt, 10 nt, 11 nt, or 12 nt from its 3′ terminus. In some embodiments, an engineered crRNA comprises a truncation of 5 nt from its 3′ terminus. In some embodiments, an engineered crRNA comprises a truncation of 8 nt from its 3′ terminus.

A crRNA can have a nucleotide sequence having at least 80% sequence identity to any one of SEQ ID NOs: 136-197. In some embodiments, a crRNA has a nucleotide sequence having at least 90% sequence identity to any one of SEQ ID NOs: 136-197. In some embodiments, a crRNA has a nucleotide sequence having at least 95% sequence identity to any one of SEQ ID NOs: 136-197. In some embodiments, a crRNA has a nucleotide sequence having 100% sequence identity to any one of SEQ ID NOs: 136-197. A crRNA of the disclosure can comprise 2′-O-methyl 3′phosphorothioate (MS) modifications at the 3 terminal nucleotides at the 5′ region and at the 3 terminal nucleotides at the 3′ region of the crRNA. MS modified crRNAs can have nucleotide sequences set forth as any one of SEQ ID NOs: 459-520.

An engineered tracrRNA can comprises a truncation of 1 nt, 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, or more, as compared to the same tracrRNA prior to its engineering. In some embodiments, an engineered tracrRNA comprises a deletion of 1 to 12 nucleotides within the first stem of the anti-repeat, as compared to the tracrRNA prior to its engineering. In some embodiments, an engineered tracrRNA comprises a deletion of 1 nt, 2 nt, 3 nt, 4 nt, 5 nt, 6 nt, 7 nt, 8 nt, 9 nt, 10 nt, 11 nt, or 12 nt within the first stem of the anti-repeat, as compared to the tracrRNA prior to its engineering. In some embodiments, an engineered tracrRNA comprises a deletion of 1 nt, 2 nt, 3 nt, 4 nt, 5 nt, 6 nt, 7 nt, 8 nt, or 9 nt within the first stem of the anti-repeat, as compared to the tracrRNA prior to its engineering.

An engineered tracrRNA can comprise a deletion of nucleotides from the tail, as compared to the tracrRNA prior to its engineering. In some embodiments, an engineered tracrRNA comprises a deletion of 1 to 6 nucleotides from the tail, as compared to the tracrRNA prior to its engineering. In some embodiments, an engineered tracrRNA comprises a deletion of 1 nucleotide, 2 nucleotides, 3 nucleotides, 4 nucleotides, 5 nucleotides, or 6 nucleotides from the tail, as compared to the tracrRNA prior to its engineering.

An engineered tracrRNA can comprise a deletion in a stem loop most proximal to the tail, as compared to the tracrRNA prior to its engineering. In some embodiments, an engineered tracrRNA comprises a deletion of 1 to 4 base pairs (bp), or 2 to 8 nt, within the first stem of the stem-loop most proximal to the tail of the tracrRNA, as compared to the tracrRNA prior to its engineering. In some embodiments, an engineered tracrRNA comprises a deletion of 1 to 3 bp, or 2 to 6 nt, within the first stem of the stem-loop most proximal to the tail of the tracrRNA, as compared to the tracrRNA prior to its engineering. In some embodiments, an engineered tracrRNA comprises a deletion of 1 bp (2 nt), 2 bp (4 nt), or 3 bp (6 nt) within the first stem of the stem-loop most proximal to the tail of the tracrRNA, as compared to the tracrRNA prior to its engineering.

As disclosed herein, a tracrRNA can comprise a total length of at least 65, 70, 75, 80, or 85 nucleotides. A tracrRNA can comprise comprises a total length of at most 65, 70, 75, 80, or 85 nucleotides. In some embodiments, a tracrRNA comprises a total length of 74 nucleotides. In some embodiments, a tracrRNA comprises a total length of 77 nucleotides.

A tail of a tracrRNA can comprise a total length of at least 1, 2, 3, 4, 5, 6, or 7 nucleotides. A tail of a tracrRNA can comprise a total length of at most 1, 2, 3, 4, 5, 6, or 7 nucleotides. In some embodiments, a tail of a tracrRNA comprises a total length of 3 nucleotides. In some embodiments, a tail of a tracrRNA comprises a total length of 1 nucleotide.

A tracrRNA can comprise a nucleotide sequence having at least 80% sequence identity to any one of SEQ ID NOs: 107, 114-123, 329, 332, and 335. In some embodiments, a tracrRNA has a nucleotide sequence having at least 90% sequence identity to any one of SEQ ID NOs: 107, 114-123, 329, 332, and 335. In some embodiments, a tracrRNA has a nucleotide sequence having at least 95% sequence identity to any one of SEQ ID NOs: 107, 114-123, 329, 332, and 335. In some embodiments, a tracrRNA has a nucleotide sequence having 100% sequence identity to any one of SEQ ID NOs: 107, 114-123, 329, 332, and 335. In some embodiments, a tracrRNA as part of a guide RNA comprises 2′-O-methyl 3′phosphorothioate (MS) modifications at the 3 terminal nucleotides at the 5′ region of the tracrRNA and at the 3 terminal nucleotides at the 3′ region of the tracrRNA. MS modified tracrRNAs can have nucleotide sequences set forth as any one of SEQ ID NOs: 431, 437-446, 584, 586, and 588.

A gRNA of the disclosure includes a sgRNA that comprises a backbone, wherein the backbone of the sgRNA comprises a crRNA repeat and a tracrRNA linked by a nucleotide linker. In some embodiments, the linker has a nucleotide sequence set forth as AAAG, GAAA, ACUU, or CAAAGG. In some embodiments, the linker has the nucleotide sequence set forth as AAAG.

Engineered sgRNA backbones disclosed herein can be 2 to 30 nucleotides shorter, as compared to the backbone prior to its engineering. An engineered sgRNA backbone can be 12 to 24 nucleotides shorter, as compared to the backbone prior to its engineering. In some embodiments, an engineered sgRNA backbone is 2 nucleotides, 4 nucleotides, 6 nucleotides, 8 nucleotides, 10 nucleotides, 12 nucleotides, 14 nucleotides, 16 nucleotides, 18 nucleotides, 20 nucleotides, 22 nucleotides, 24 nucleotides, 26 nucleotides, 28 nucleotides, 30 nucleotides, or more shorter, as compared to the backbone prior to its engineering.

An sgRNA backbone of the disclosure can comprise a total length of at least 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 nucleotides. An sgRNA backbone of the disclosure can comprise a total length of at most 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 nucleotides. In some embodiments, the sgRNA backbone comprises a total length of 86 to 98 nucleotides. In some embodiments, the sgRNA backbone comprises a total length of 94 nucleotides. In some embodiments, a sgRNA has the nucleotide sequence set forth as any one of SEQ ID NOs: 124-134.

An sgRNA backbone of the disclosure can have a nucleotide sequence having at least 80% sequence identity to any one of SEQ ID NOs: 124-134. In some embodiments, an sgRNA backbone has a nucleotide sequence having at least 90% sequence identity to any one of SEQ ID NOs: 124-134. In some embodiments, an sgRNA backbone has a nucleotide sequence having at least 95% sequence identity to any one of SEQ ID NOs: 124-134. In some embodiments, an sgRNA backbone has a nucleotide sequence having 100% sequence identity to any one of SEQ ID NOs: 124-134. In some embodiments, a backbone as part of a guide RNA comprises 2′-O-methyl 3′phosphorothioate (MS) modifications at the 3 terminal nucleotides at the 3′ region of the backbone. MS modified backbones can have nucleotide sequences set forth as any one of SEQ ID NOs: 447-457.

A gRNA of the disclosure includes a sgRNA that comprises a spacer and a backbone, wherein the backbone of the sgRNA comprises a crRNA repeat and a tracrRNA linked by a nucleotide linker. In some embodiments, an engineered sgRNA comprises a truncation in the spacer and/or a truncation in the backbone, as compared to the sgRNA prior to its engineering. In some embodiments, an engineered sgRNA comprises a truncation in the spacer, as compared to the sgRNA prior to its engineering. In some embodiments, an engineered sgRNA comprises a truncation in the backbone, as compared to the sgRNA prior to its engineering. In some embodiments, an engineered sgRNA comprises a truncation in the spacer and a truncation in the backbone, as compared to the sgRNA prior to its engineering. In embodiments where an engineered sgRNA comprises a truncation in the backbone, the truncation can be within the first stem of the stem loop formed by hybridization of the crRNA repeat and the anti-repeat, within the first stem of the stem loop most proximal to the tail, and/or within the tail of the tracrRNA.

An engineered sgRNA can comprise a deletion of 1 to 30 total nucleotides, as compared to the sgRNA prior to its engineering. In some embodiments, an engineered sgRNA comprises a deletion of 13 to 25 total nucleotides, as compared to the sgRNA prior to its engineering. In some embodiments, an engineered sgRNA comprises a deletion of 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 total nucleotides, or more, as compared to the sgRNA prior to its engineering.

The first stem of a stem loop formed by hybridization of the crRNA repeat and the anti-repeat of a gRNA can comprise a total length of at least 3, 4, 5, 6, 7, 8, 9, 10, or 11 base pairs (bp), or at least 6, 8, 10, 12, 14, 16, 18, 20, or 22 nt. The first stem of a stem loop formed by hybridization of the crRNA repeat and the anti-repeat of a gRNA can comprise a total length of at most 3, 4, 5, 6, 7, 8, 9, 10, or 11 bp, or at most 6, 8, 10, 12, 14, 16, 18, 20, or 22 nt. In some embodiments, the first stem of a stem loop formed by hybridization of the crRNA repeat and the anti-repeat of a gRNA comprises a total length of 6 bp, or 12 nt. In some embodiments, the first stem of a stem loop formed by hybridization of the crRNA repeat and the anti-repeat of a gRNA comprises a total length of 3 bp, or 6 nt.

The first stem of the stem loop most proximal to the tail in a gRNA can comprise a total length of at least 1, 2, 3, 4, 5, or 6 bp, or at least 2, 4, 6, 8, 10, or 12 nt. The first stem of the stem loop most proximal to the tail in a gRNA can comprise a total length of at most 1, 2, 3, 4, 5, or 6 bp, or at most 2, 4, 6, 8, 10, or 12 nt. In some embodiments, the first stem of the stem loop most proximal to the tail in a gRNA comprises a total length of 5 bp, or 10 nt.

In some embodiments, a gRNA of the disclosure comprises the following: the first stem of the stem loop formed by hybridization of the crRNA repeat and the anti-repeat comprises a total length of 6 bp (12 nt), the tail of the tracrRNA comprises a total length of 3 nucleotides, and the first stem of the stem loop most proximal to the tail comprises a total length of 3 bp (6 nt). In some embodiments, a gRNA of the disclosure comprises a first stem of a stem loop formed by hybridization of the crRNA repeat and the anti-repeat comprising a total length of 13 bp (26 nt).

A total length of a guide RNA can refer to a total length of a sgRNA or of a dgRNA. A gRNA of the disclosure can comprise a total length of at least 85, 90, 95, 100, 105, 110, 115, 120, 125, 130, or 135 nucleotides. A gRNA of the disclosure can comprise a total length of at most 85, 90, 95, 100, 105, 110, 115, 120, 125, 130, or 135 nucleotides. In some embodiments, a gRNA of the disclosure comprises a total length of 106 to 135 nucleotides. In some embodiments, a gRNA of the disclosure comprises a total length of 117 to 119 nucleotides. In embodiments where the gRNA comprises a total length of 117 to 119 nucleotides, the gRNA is a sgRNA. In embodiments where a gRNA comprises a total length of 117 to 119 nucleotides as a sgRNA, the total length of the gRNA as a dgRNA can be 4 to 6 nucleotides fewer, or 111 to 115 nucleotides. In some embodiments, the total length of a gRNA as a dgRNA is 4 to 6 nucleotides fewer, or a number of nucleotides fewer that is equivalent to the length of the linker joining the crRNA and tracrRNA, as compared to the total length of the gRNA as a sgRNA. In some embodiments, a gRNA of the disclosure comprises a total length of 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135 nucleotides, or more. In some embodiments, a sgRNA has the nucleotide sequence set forth as any one of SEQ ID NOs: 198-200, 202-213, 215-233, 235-241, and 243-259. In some embodiments, a sgRNA has at least 80% sequence identity, at least 90% sequence identity, or at least 95% sequence identity, to a nucleotide sequence set forth as SEQ ID NO: 204. In some embodiments, a sgRNA has the nucleotide sequence set forth as SEQ ID NO: 204. In some embodiments, a sgRNA has at least 80% sequence identity, at least 90% sequence identity, or at least 95% sequence identity, to a nucleotide sequence set forth as SEQ ID NO: 205. In some embodiments, a sgRNA has the nucleotide sequence set forth as SEQ ID NO: 205. A sgRNA of the disclosure can comprise 2′-O-methyl 3′phosphorothioate (MS) modifications at the 3 terminal nucleotides at the 5′ region and at the 3 terminal nucleotides at the 3′ region of the sgRNA. MS modified sgRNAs can have nucleotide sequences set forth as any one of SEQ ID NOs: 521-523, 525-536, 538-556, 558-564, and 566-582.

### IV. RNA-Guided Nucleases and Other Nucleases

Provided herein are RNA-guided nuclease systems comprising the presently disclosed guide RNAs targeting the TRAC gene. The term RNA-guided nuclease (RGN) refers to a polypeptide that binds to a particular target sequence (e.g., target DNA sequence) in a sequence-specific manner and is directed to the target sequence by a guide RNA molecule that is complexed with the polypeptide and hybridizes with the target strand of the target sequence (e.g., target DNA sequence). Active fragments or variants thereof of naturally-occurring RGNs maintain binding to a target nucleotide sequence in an RNA-guided sequence-specific manner. Although an RGN can be capable of cleaving the target sequence upon binding, the term RGN also encompasses nuclease-dead RGNs that are capable of binding to, but not cleaving, a target sequence. Cleavage of a target strand and/or non-target strand of a target sequence by an RGN can result in a single- or double-stranded break. RGNs only capable of cleaving a single strand of a double-stranded target nucleic acid molecule are referred to herein as nickases.

The presently disclosed RGN systems comprise an RGN that binds to a TRAC target sequence disclosed herein. In some embodiments, the RGN recognizes a PAM having a consensus nucleotide sequence including NNNNCC 3′ of the target sequence on its non-target strand (where N is A, C, T/U, or G; R is G or A), and active fragments or variants thereof. In some embodiments, the RGN recognizes a PAM having a consensus nucleotide sequence including NNRNCC 3′ of the target sequence on its non-target strand (where N is A, C, T/U, or G; R is G or A), and active fragments or variants thereof. In some embodiments, the active fragment or variant of an RGN recognizing such PAM sequences is capable of binding and in some embodiments, cleaving or nicking a target sequence.

In some embodiments, an RGN, or an active variant or fragment thereof, capable of binding a target sequence adjacent to a PAM consensus sequence (i.e., capable of recognizing the PAM consensus sequence) set forth as NNNNCC or NNRNCC is used in the presently disclosed compositions and methods. In some embodiments, an RGN, or an active variant or fragment thereof, capable of binding a target sequence adjacent to a full PAM sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG is used in the presently disclosed compositions and methods. In some embodiments, the PAM sequence is 3′ of the target sequence on its non-target strand. In some embodiments, the RGN binds to a guide RNA having a sequence set forth as any one of SEQ ID NOs: 198-200, 202-213, 215-233, 235-241, and 243-259. In some embodiments, the RGN binds to a guide RNA having at least 80% sequence identity, at least 90% sequence identity, or at least 95% sequence identity to a nucleotide sequence set forth as SEQ ID NO: 204. In some embodiments, the RGN binds to a guide RNA having at least 80% sequence identity, at least 90% sequence identity, or at least 95% sequence identity to a nucleotide sequence set forth as SEQ ID NO: 205. In some embodiments, the RGN binds to a guide RNA having a sequence set forth as SEQ ID NO: 204 or 205. In some embodiments, the RGN binds to a guide RNA comprising a CRISPR repeat set forth as any one of SEQ ID NOs: 106, 109-112, 328, 331, and 334, or an active variant or fragment thereof, and a tracrRNA set forth as any one of SEQ ID NOs: 107, 114-123, 329, 332, and 335, or an active variant or fragment thereof. In some embodiments, the RGN binds to a guide RNA comprising a CRISPR repeat having the nucleotide sequence set forth as SEQ ID NO: 106 or a nucleotide sequence that differs in length and/or sequence from SEQ ID NO: 106 by 1 to 8 nucleotides. In some embodiments, the RGN binds to a guide RNA comprising a tracrRNA having at least 80% sequence identity, at least 90% sequence identity, or at least 95% sequence identity to a nucleotide sequence set forth as SEQ ID NO: 107. In some embodiments, the RGN binds to a guide RNA comprising a tracrRNA having the nucleotide sequence set forth as SEQ ID NO: 107.

RGNs useful in the presently disclosed compositions and methods can be wild-type RGN sequences derived from bacterial or archaeal species. Alternatively, the RGNs can be variants or fragments of wild-type polypeptides. The wild-type RGN can be modified to alter nuclease activity or alter PAM specificity, for example. In some embodiments, the RGN is not naturally-occurring. RGN systems can be classified into Class 1 or Class 2. The Class 1 and 2 systems are subdivided into types (Types I, II, III, IV, V, VI), with some types further divided into subtypes (e.g., Type II-A, Type II-B, Type II-C, Type V-A, Type V-B). Class 2 systems comprise a single effector nuclease and include Types II, V, and VI.

In certain embodiments, the RGN is a naturally-occurring Type II CRISPR effector protein or an active variant or fragment thereof. As used herein, the term “Type II CRISPR-Cas protein,” “Type II CRISPR-Cas effector protein,” or “Type II RNA-guided nuclease” refers to an RGN that requires a trans-activating RNA (tracrRNA) and comprises two nuclease domains (i.e., RuvC and HNH), each of which is responsible for cleaving a single strand of a double-stranded DNA molecule. A representative type II RGN includes a Streptococcus pyogenes Cas9 protein, such as Streptococcus pyogenes Cas9 (SpCas9 or SpyCas9) or a SpCas9 nickase, the sequences of which are set forth as SEQ ID NOs: 324 and 325, respectively, and are described in U.S. Pat. Nos. 10,000,772 and 8,697,359, each of which is herein incorporated by reference in its entirety. SpCas9 recognizes a NGG PAM sequence 3′ of a target sequence, and some of the disclosed TRAC target sequences could be targeted with an SpCas9 associated with its guide RNA, as indicated in Table 2 in the Examples. Another representative Cas9 ortholog that recognizes a NNNNCC PAM sequence 3′ of a target sequence includes a compact, high-accuracy Neisseria meningitidis Cas9 (Nme2Cas9), the sequence of which is set forth as SEQ ID NO: 326 and described in Edraki et al. Mol Cell. 2019 Feb. 21; 73(4): 714-726.

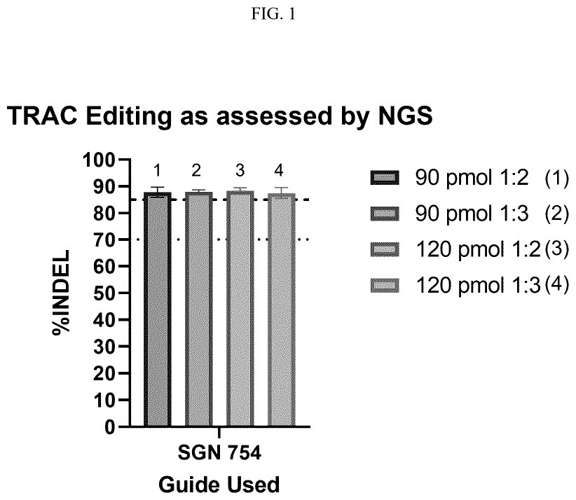

Non-limiting examples of RGN systems useful in the presently disclosed compositions and methods along with corresponding crRNA sequences and tracrRNA sequences (if needed), are presented in Table 1 below and described further in Examples 1-4, and FIGS. 1-10 of the present specification. In certain embodiments, RGN systems of the disclosure comprise an RGN, or a nickase or nuclease-dead variant thereof, listed in Table 1. The guide RNA sequences (crRNA repeat and tracrRNA sequences) that can be used with each RGN of Table 1 are also provided, as well as the consensus PAM sequence (if known). In certain embodiments, an RGN of the disclosure comprises an active variant of an RGN (one able to bind to a nucleic acid molecule in an RNA-guided manner) listed in Table 1 having between 80% and 99% or more sequence identity to any one of the amino acid sequences listed in Table 1, including but not limited to about or more than about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In certain embodiments, an RGN of the disclosure comprises an RGN having 80%, 81%, 82%, 83%, 84%, 85%, 86%, 87%, 88%, 89%, 90%, 91%, 92%, 93%, 94%, 95%, 96%, 97%, 98%, 99%, or more sequence identity to an RGN amino acid sequence disclosed in Table 1. In some embodiments, an RGN of the disclosure comprises a fragment of an RGN listed in Table 1 such as one that differs by as few as 1-15 amino acid residues, as few as 1-10, such as 6-10, as few as 5, as few as 4, as few as 3, as few as 2, or as few as 1 amino acid residue. In certain embodiments, the RGN comprises an N-terminal or a C-terminal truncation, which can comprise at least a deletion of 5, 10, 15, 20, 25, 30, 35, 40, 45, 50, 55, 60 amino acids or more from either the N or C terminus of the polypeptide. In some embodiments, the RGN comprises an internal deletion which can comprise at least a deletion of 1, 2, 3, 4, 5, 6, 7, 8, 9, 10, 11, 12, 13, 14, 15, 16, 17, 18, 19, 20, 25, 30, 35, 40, 45, 50, 55, 60 amino acids or more.

TABLE 1
Non-limiting examples of RNA-guided nucleases and corresponding crRNA repeat
sequences, tracrRNA sequences, and PAM sequences.

| | RGN SEQ | crRNA repeat | tracrRNA | PAM |
|---|---|---|---|---|
| RGN Name | ID NO: | SEQ ID NO: | SEQ ID NO: | sequence |
| APG07433.1 | 105 | 106 | 107 | NNNNCC |
| APG05083.1 | 327 | 328 | 329 | NNNNCC |
| APG07513.1 | 330 | 331 | 332 | NNNNCC |
| APG08290.1 | 333 | 334 | 335 | NNRNCC |
| APG05459.1 | 336 | 337 | 338 | NGG |
| APG01688.1 | 339 | 340 | 341 | NNRNNC |
| APG03128 | 342 | 343 | 344 | NNGGNNC |
| APG05733.1 | 345 | 346 | 347 | NNNNCC |
| APG01658.1 | 348 | 349 | 350 | NNGG |
| APG06498.1 | 351 | 352 | 353 | NNNNCC |
| APG06877.1 | 354 | 355 | 356 | NNNNCC |
| APG09053.1 | 357 | 358 | 359 | NGG |
| APG04293.1 | 360 | 361 | 362 | NNNNC |
| APG06646.1 | 363 | 364 | 365 | NNNNC |
| APG02874 | 366 | 367 | 368 | NNNNCC |
| APG03031 | 369 | 370 | 371 | NNNNCC |
| APG09208 | 372 | 373 | 374 | NNNNC |
| APG09344 | 375 | 376 | 377 | NAG |
| APG07991 | 378 | 379 | 380 | NGG |
| APG01868 | 381 | 382 | 383 | NGG |
| APG02998 | 384 | 385 | 386 | NGG |
| APG07433.1 deletion variant | 387 | 106 | 107 | NNNNCC |
| APG08290.1 deletion variant | 388 | 334 | 335 | NNRNCC |
| LPG10134 | 389 | 390 | 391 | NGG |
| LPG10136 | 392 | 393 | 394 | NNNNCC |
| LPG10138 | 395 | 396 | 397 | NNNNC |
| LPG10139 | 398 | 399 | 400 | NNNNC |
| LPG10141 | 401 | 402 | 403 | NGGNR |
| LPG10145 | 404 | 405 | 406 | NNGG |
| SpyCas9 | 324 | 407 | 408 | NGG |

N = A, C, T/U, or G; R = G or A

Non-limiting examples of RGNs useful in the presently disclosed methods and compositions include APG07433.1 RNA-guided nuclease, the amino acid sequence of which is set forth as:

(SEQ ID NO: 5)
MRELDYRIGLDIGTNSIGWGVIELSWNKDRERYEKVRIVDQGVRMFDRA
EMPKTGASLAEPRRIARSSRRRLNRKSQRKKNIRNLLVQHGVITQEELD
SLYPLSKKSMDIWGIRLDGLDRLLNHFEWARLLIHLAQRRGFKSNRKSE
LKDTETGKVLSSIQLNEKRLSLYRTVGEMWMKDPDFSKYDRKRNSPNEY
VFSVSRAELEKEIVTLFAAQRRFQSPYASKDLQETYLQIWTHQLPFASG
NAILNKVGYCSLLKGKERRIPKATYTFQYFSALDQVNRTRLGPDFQPFT
KEQREIILNNMFQRTDYYKKKTIPEVTYYDIRKWLELDETIQFKGLNYD
PNEELKKIEKKPFINLKAFYEINKVVANYSERTNETFSTLDYDGIGYAL
TVYKTDKDIRSYLKSSHNLPKRCYDDQLIEELLSLSYTKFGHLSLKAIN
HVLSIMQKGNTYKEAVDQLGYDTSGLKKEKRSKFLPPISDEITNPIVKR
ALTQARKVVNAIIRRHGSPHSVHIELARELSKNHDERTKIVSAQDENYK
KNKGAISILSEHGILNPTGYDIVRYKLWKEQGERCAYSLKEIPADTFFN
ELKKERNGAPILEVDHILPYSQSFIDSYHNKVLVYSDENRKKGNRIPYT
YFLETNKDWEAFERYVRSNKFFSKKKREYLLKRAYLPRESELIKERHLN
DTRYASTFLKNFIEQNLQFKEAEDNPRKRRVQTVNGVITAHFRKRWGLE
KDRQETYLHHAMDAIIVACTDHHMVTRVTEYYQIKESNKSVKKPYFPMP
WEGFRDELLSHLASQPIAKKISEELKAGYQSLDYIFVSRMPKRSITGAA
HKQTIMRKGGIDKKGKTIIIERLHLKDIKFDENGDFKMVGKEQDMATYE
AIKQRYLEHGKNSKKAFETPLYKPSKKGTGNLIKRVKVEGQAKSFVREV
NGGVAQNGDLVRVDLFEKDDKYYMVPIYVPDTVCSELPKKVVASSKGYE
QWLTLDNSFTFKFSLYPYDLVRLVKGDEDRFLYFGTLDIDSDRLNFKDV
NKPSKKNEYRYSLKTIEDLEKYEVGVLGDLRLVRKETRRNFH,

and active fragments or variants thereof that retain the ability to bind to a target sequence in an RNA-guided sequence-specific manner. In some embodiments, an active variant of an RGN disclosed herein comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth as SEQ ID NO: 105. In some embodiments, an active fragment of the APG07433.1 RGN comprises at least 50, 100, 150, 200, 250, 300, 350, 400, 450, 500, 550, 600, 650, 700, 750, 800, 850, 900, 950, 1000, 1050 or more contiguous amino acid residues of the amino acid sequence set forth as SEQ ID NO: 105.

In some embodiments, the presently disclosed compositions and methods comprise an RGN capable of binding a target sequence of the disclosure or an RGN having an amino acid sequence set forth as SEQ ID NO: 105, or an active variant or fragment thereof, wherein the RGN is capable of binding a target sequence adjacent to a PAM consensus sequence set forth as NNNNCC. In some embodiments, the PAM sequence is 3′ of the target sequence on its non-target strand. In some embodiments, the RGN binds to a guide RNA having a sequence set forth as any one of SEQ ID NOs: 198-200, 202-213, 215-233, 235-241, 243-259, 521-523, 525-536, 538-556, 558-564, and 566-582. In some embodiments, the RGN binds to a guide RNA comprising a CRISPR repeat set forth as any one of SEQ ID NOs: 106, 109-112, 328, 331, 334, 430, 432-435, 583, 585, and 587, or an active variant or fragment thereof, and a tracrRNA set forth as any one of SEQ ID NOs: 107, 114-123, 329, 332, 335, 431, 437-446, 584, 586, and 588, or an active variant or fragment thereof.

In some embodiments, the presently disclosed compositions and methods comprise an RGN capable of binding a target sequence of the disclosure or an RGN having an amino acid sequence set forth as SEQ ID NO: 105, or an active variant or fragment thereof, wherein the RGN is capable of binding a target sequence adjacent to a full PAM sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG. In some embodiments, the PAM sequence is 3′ of the target sequence on its non-target strand. In some embodiments, the RGN binds to a guide RNA having a sequence set forth as any one of SEQ ID NOs: 198-200, 202-213, 215-233, 235-241, 243-259, 521-523, 525-536, 538-556, 558-564, and 566-582. In some embodiments, the RGN binds to a guide RNA having at least 80% sequence identity, at least 90% sequence identity, or at least 95% sequence identity to a nucleotide sequence set forth as SEQ ID NO: 204. In some embodiments, the RGN binds to a guide RNA having at least 80% sequence identity, at least 90% sequence identity, or at least 95% sequence identity to a nucleotide sequence set forth as SEQ ID NO: 205. In some embodiments, the RGN binds to a guide RNA having a sequence set forth as SEQ ID NO: 204 or 205. In some embodiments, the RGN binds to a guide RNA comprising a CRISPR repeat set forth as any one of SEQ ID NOs: 106, 109-112, 328, 331, 334, 430, 432-435, 583, 585, and 587, or an active variant or fragment thereof, and a tracrRNA set forth as any one of SEQ ID NOs: 107, 114-123, 329, 332, 335, 431, 437-446, 584, 586, and 588, or an active variant or fragment thereof. In some embodiments, the RGN binds to a guide RNA comprising a CRISPR repeat set forth as SEQ ID NO: 106, or an active variant or fragment thereof. In some embodiments, the RGN binds to a guide RNA comprising a tracrRNA set forth as SEQ ID NO: 107, or an active variant or fragment thereof.

RGNs useful in the presently disclosed methods and compositions include APG05083.1 RNA-guided nuclease, the amino acid sequence of which is set forth as SEQ ID NO: 327, and active fragments or variants thereof that retain the ability to bind to a target sequence in an RNA-guided sequence-specific manner. In some embodiments, an active variant of an RGN disclosed herein comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth as SEQ ID NO: 327. In some embodiments, an active fragment of the APG05083.1 RGN comprises at least 50, 100, 150, 200, 250, 300, 350, 400, 450, 500, 550, 600, 650, 700, 750, 800, 850, 900, 950, 1000, 1050 or more contiguous amino acid residues of the amino acid sequence set forth as SEQ ID NO: 327.

In some embodiments, the presently disclosed compositions and methods comprise an RGN capable of binding a target sequence of the disclosure or an RGN having an amino acid sequence set forth as SEQ ID NO: 327, or an active variant or fragment thereof, wherein the RGN is capable of binding a target sequence adjacent to a PAM consensus sequence set forth as NNNNCC. In embodiments, the PAM sequence is 3′ of the target sequence on its non-target strand. In some embodiments, the RGN binds to a guide RNA having a sequence set forth as any one of SEQ ID NOs: 198-200, 202-213, 215-233, 235-241, 243-259, 521-523, 525-536, 538-556, 558-564, and 566-582. In some embodiments, the RGN binds to a guide RNA comprises a CRISPR repeat set forth as any one of SEQ ID NOs: 106, 109-112, 328, 331, 334, 430, 432-435, 583, 585, and 587, or an active variant or fragment thereof, and a tracrRNA set forth as any one of SEQ ID NOs: 107, 114-123, 329, 332, 335, 431, 437-446, 584, 586, and 588, or an active variant or fragment thereof.

In some embodiments, the presently disclosed compositions and methods comprise an RGN capable of binding a target sequence of the disclosure or an RGN having an amino acid sequence set forth as SEQ ID NO: 327, or an active variant or fragment thereof, wherein the RGN is capable of binding a target sequence adjacent to a full PAM sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG. In some embodiments, the PAM sequence is 3′ of the target sequence on its non-target strand. In some embodiments, the RGN binds to a guide RNA having a sequence set forth as any one of SEQ ID NOs: 198-200, 202-213, 215-233, 235-241, 243-259, 521-523, 525-536, 538-556, 558-564, and 566-582. In some embodiments, the RGN binds to a guide RNA comprising a CRISPR repeat set forth as any one of SEQ ID NOs: 106, 109-112, 328, 331, 334, 430, 432-435, 583, 585, and 587, or an active variant or fragment thereof, and a tracrRNA set forth as any one of SEQ ID NOs: 107, 114-123, 329, 332, 335, 431, 437-446, 584, 586, and 588, or an active variant or fragment thereof.

RGNs useful in the presently disclosed methods and compositions include APG07513.1 RNA-guided nuclease, the amino acid sequence of which is set forth as SEQ ID NO: 330, and active fragments or variants thereof that retain the ability to bind to a target sequence in an RNA-guided sequence-specific manner. In some embodiments, an active variant of an RGN disclosed herein comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth as SEQ ID NO: 330. In some embodiments, an active fragment of the APG07513.1 RGN comprises at least 50, 100, 150, 200, 250, 300, 350, 400, 450, 500, 550, 600, 650, 700, 750, 800, 850, 900, 950, 1000, 1050 or more contiguous amino acid residues of the amino acid sequence set forth as SEQ ID NO: 330.

In some embodiments, the presently disclosed compositions and methods comprise an RGN capable of binding a target sequence of the disclosure or an RGN having an amino acid sequence set forth as SEQ ID NO: 330, or an active variant or fragment thereof, wherein the RGN is capable of binding a target sequence adjacent to a PAM consensus sequence set forth as NNNNCC. In embodiments, the PAM sequence is 3′ of the target sequence on its non-target strand. In some embodiments, the RGN binds to a guide RNA having a sequence set forth as any one of SEQ ID NOs: 198-200, 202-213, 215-233, 235-241, 243-259, 521-523, 525-536, 538-556, 558-564, and 566-582. In some embodiments, the RGN binds to a guide RNA comprises a CRISPR repeat set forth as any one of SEQ ID NOs: 106, 109-112, 328, 331, 334, 430, 432-435, 583, 585, and 587, or an active variant or fragment thereof, and a tracrRNA set forth as any one of SEQ ID NOs: 107, 114-123, 329, 332, 335, 431, 437-446, 584, 586, and 588, or an active variant or fragment thereof.

In some embodiments, the presently disclosed compositions and methods comprise an RGN capable of binding a target sequence of the disclosure or an RGN having an amino acid sequence set forth as SEQ ID NO: 330, or an active variant or fragment thereof, wherein the RGN is capable of binding a target sequence adjacent to a full PAM sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG. In some embodiments, the PAM sequence is 3′ of the target sequence on its non-target strand. In some embodiments, the RGN binds to a guide RNA having a sequence set forth as any one of SEQ ID NOs: 198-200, 202-213, 215-233, 235-241, 243-259, 521-523, 525-536, 538-556, 558-564, and 566-582. In some embodiments, the RGN binds to a guide RNA comprising a CRISPR repeat set forth as any one of SEQ ID NOs: 106, 109-112, 328, 331, 334, 430, 432-435, 583, 585, and 587, or an active variant or fragment thereof, and a tracrRNA set forth as any one of SEQ ID NOs: 107, 114-123, 329, 332, 335, 431, 437-446, 584, 586, and 588, or an active variant or fragment thereof.

RGNs useful in the presently disclosed methods and compositions include APG08290.1 RNA-guided nuclease, the amino acid sequence of which is set forth as SEQ ID NO: 333, and active fragments or variants thereof that retain the ability to bind to a target sequence in an RNA-guided sequence-specific manner. In some embodiments, an active variant of an RGN disclosed herein comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth as SEQ ID NO: 333. In some embodiments, an active fragment of the APG08290.1 RGN comprises at least 50, 100, 150, 200, 250, 300, 350, 400, 450, 500, 550, 600, 650, 700, 750, 800, 850, 900, 950, 1000, 1050 or more contiguous amino acid residues of the amino acid sequence set forth as SEQ ID NO: 333.

In some embodiments, the presently disclosed compositions and methods comprise an RGN capable of binding a target sequence of the disclosure or an RGN having an amino acid sequence set forth as SEQ ID NO: 333, or an active variant or fragment thereof, wherein the RGN is capable of binding a target sequence adjacent to a PAM consensus sequence set forth as NNRNCC. In embodiments, the PAM sequence is 3′ of the target sequence on its non-target strand. In some embodiments, the RGN binds to a guide RNA having a sequence set forth as any one of SEQ ID NOs: 198-200, 202-213, 215-233, 235-241, 243-259, 521-523, 525-536, 538-556, 558-564, and 566-582. In some embodiments, the RGN binds to a guide RNA comprises a CRISPR repeat set forth as any one of SEQ ID NOs: 106, 109-112, 328, 331, 334, 430, 432-435, 583, 585, and 587, or an active variant or fragment thereof, and a tracrRNA set forth as any one of SEQ ID NOs: 107, 114-123, 329, 332, 335, 431, 437-446, 584, 586, and 588, or an active variant or fragment thereof. In some embodiments, the RGN binds to a guide RNA comprising a CRISPR repeat set forth as SEQ ID NO: 334, or an active variant or fragment thereof. In some embodiments, the RGN binds to a guide RNA comprising a tracrRNA set forth as SEQ ID NO: 335, or an active variant or fragment thereof.

In some embodiments, the presently disclosed compositions and methods comprise an RGN capable of binding a target sequence of the disclosure or an RGN having an amino acid sequence set forth as SEQ ID NO: 333, or an active variant or fragment thereof, wherein the RGN is capable of binding a target sequence adjacent to a full PAM sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG. In some embodiments, the PAM sequence is 3′ of the target sequence on its non-target strand. In some embodiments, the RGN binds to a guide RNA having a sequence set forth as any one of SEQ ID NOs: 198-200, 202-213, 215-233, 235-241, 243-259, 521-523, 525-536, 538-556, 558-564, and 566-582. In some embodiments, the RGN binds to a guide RNA comprising a CRISPR repeat set forth as any one of SEQ ID NOs: 106, 109-112, 328, 331, 334, 430, 432-435, 583, 585, and 587, or an active variant or fragment thereof, and a tracrRNA set forth as any one of SEQ ID NOs: 107, 114-123, 329, 332, 335, 431, 437-446, 584, 586, and 588, or an active variant or fragment thereof.

In some embodiments, the presently disclosed compositions and methods comprise an RGN capable of binding a target sequence of the disclosure or an RGN having an amino acid sequence set forth as SEQ ID NO: 324, or an active variant or fragment thereof, wherein the RGN is capable of binding a target sequence adjacent to a full PAM sequence set forth as GGGCCCAG. In some embodiments, the PAM sequence is 3′ of the target sequence on its non-target strand. In some embodiments, the RGN binds to a guide RNA comprising a CRISPR repeat set forth as SEQ ID NO: 407, or an active variant or fragment thereof, and a tracrRNA set forth as SEQ ID NO: 408, or an active variant or fragment thereof.

In some embodiments, the presently disclosed compositions and methods comprise an RGN capable of binding a target sequence of the disclosure or an RGN having an amino acid sequence set forth as SEQ ID NO: 404, or an active variant or fragment thereof, wherein the RGN is capable of binding a target sequence adjacent to a full PAM sequence set forth as CAGGCCAA. In some embodiments, the PAM sequence is 3′ of the target sequence on its non-target strand. In some embodiments, the RGN binds to a guide RNA comprising a CRISPR repeat set forth as SEQ ID NO: 405, or an active variant or fragment thereof, and a tracrRNA set forth as SEQ ID NO: 406, or an active variant or fragment thereof.

According to the present invention, the presently disclosed target sequences within the TRAC gene are bound by an RGN. The target strand of the target sequence hybridizes with the guide RNA associated with the RGN. The target strand and/or the non-target strand of the target sequence (e.g., target DNA sequence) can then be subsequently cleaved by the RGN if the polypeptide possesses nuclease activity. The terms “cleave” or “cleavage” refer to the hydrolysis of at least one phosphodiester bond within the backbone of one or both strands of a double-stranded target sequence (e.g., target DNA sequence) that can result in either single-stranded or double-stranded breaks within the target DNA sequence. The cleavage of a presently disclosed target sequence can result in staggered breaks or blunt ends.

In some embodiments, the RGN used in the presently disclosed compositions and methods functions as a nickase, only cleaving a single strand of a double-stranded target sequence (e.g., target DNA sequence). Such RGNs have a single functioning nuclease domain. In some embodiments, the nickase is capable of cleaving the target strand or the non-target strand of the double-stranded target sequence (e.g., target DNA sequence). In embodiments where a nickase is used, in order to effect a double-stranded cleavage of a target sequence within the TRAC gene, two nickases are needed, each of which nicks a single strand within the target sequence. In some embodiments, additional nuclease domains have been mutated such that the nuclease activity is reduced or eliminated.

In some embodiments, the RGN lacks nuclease activity altogether and is referred to herein as nuclease-dead or nuclease inactive. Any method known in the art for introducing mutations into an amino acid sequence, such as PCR-mediated mutagenesis and site-directed mutagenesis, can be used for generating nickases or nuclease-dead RGNs. See, e.g., U.S. Publ. No. 2014/0068797 and U.S. Pat. No. 9,790,490; each of which is incorporated by reference in its entirety.

In some embodiments, nucleases other than RGNs are used in the presently disclosed compositions and methods. These nucleases can bind to additional target sequences of the TRAC gene distinct from the presently disclosed target sequences. As used herein, the term “nuclease” refers to an enzyme that catalyzes the cleavage of phosphodiester bonds between nucleotides in a nucleic acid molecule. In general, the nuclease is an endonuclease, which is capable of cleaving phosphodiester bonds between nucleotides within a nucleic acid molecule. In some embodiments, the sequence-specific nuclease is selected from the group consisting of a meganuclease, a zinc finger nuclease, a TAL-effector DNA binding domain-nuclease fusion protein (TALEN), and an RNA-guided nuclease (RGN) or variants thereof wherein the nuclease activity has been reduced or inhibited.

As used herein, the term “meganuclease” or “homing endonuclease” refers to endonucleases that bind a recognition site within double-stranded DNA that is 12 to 40 bp in length. Non-limiting examples of meganucleases are those that belong to the LAGLIDADG family that comprise the conserved amino acid motif LAGLIDADG (SEQ ID NO: 410). The term “meganuclease” can refer to a dimeric or single-chain meganuclease.

As used herein, the term “zinc finger nuclease” or “ZFN” refers to a chimeric protein comprising a zinc finger DNA-binding domain and a nuclease domain.

As used herein, the term “TAL-effector DNA binding domain-nuclease fusion protein” or “TALEN” refers to a chimeric protein comprising a TAL effector DNA-binding domain and a nuclease domain.

RGNs or nucleases (such as meganucleases, zinc finger nucleases, or TALENs) that lack nuclease activity and therefore, function as a DNA-binding polypeptide, can be used to deliver a fused polypeptide, polynucleotide, or small molecule payload to a particular genomic location. In some embodiments, the RGN polypeptide, guide RNA, or nuclease can be fused to a detectable label to allow for detection of a particular sequence. The detectable label or purification tag can be located at the N-terminus, the C-terminus, or an internal location of the RNA-guided nuclease, either directly or indirectly via a linker peptide. In some embodiments, the RGN component of the fusion protein is a nuclease-dead RGN. In some embodiments, the RGN component of the fusion protein is an RGN with nickase activity.

A detectable label is a molecule that can be visualized or otherwise observed. The detectable label may be fused to the RGN as a fusion protein (e.g., fluorescent protein) or may be a small molecule conjugated to the RGN polypeptide that can be detected visually or by other means. Detectable labels that can be fused to the presently disclosed RGNs as a fusion protein include any detectable protein domain, including but not limited to, a fluorescent protein or a protein domain that can be detected with a specific antibody. Non-limiting examples of fluorescent proteins include green fluorescent proteins (e.g., GFP, EGFP, ZsGreen1) and yellow fluorescent proteins (e.g., YFP, EYFP, ZsYellow1). Non-limiting examples of small molecule detectable labels include radioactive labels, such as ³H and ³⁵S.

RGN polypeptides can also comprise a purification tag, which is any molecule that can be utilized to isolate a protein or fused protein from a mixture (e.g., biological sample, culture medium). Non-limiting examples of purification tags include biotin, myc, maltose binding protein (MBP), glutathione-S-transferase (GST), and 3× FLAG tag.

Alternatively, nuclease-dead RGNs can be targeted to the TRAC gene to alter the expression of the gene. In some embodiments, the binding of a nuclease-dead RGN to a target sequence within the TRAC gene results in the reduction in expression of TRAC by interfering with the binding of RNA polymerase or transcription factors within the targeted genomic region. In some embodiments, the RGN (e.g., a nuclease-dead RGN) or its complexed guide RNA further comprises an expression modulator that, upon binding to a target sequence within the TRAC gene, serves to either repress or activate the expression of the target gene.

In some embodiments, the expression modulator comprises a transcriptional repressor domain, which interacts with transcriptional control elements and/or transcriptional regulatory proteins, such as RNA polymerases and transcription factors, to reduce or terminate transcription of the TRAC gene. Transcriptional repressor domains are known in the art and include, but are not limited to, Sp1-like repressors, IκB, and Krüppel associated box (KRAB) domains.

In some embodiments, the expression modulator comprises a transcriptional activation domain, which interacts with transcriptional control elements and/or transcriptional regulatory proteins, such as RNA polymerases and transcription factors, to increase or activate transcription of the TRAC gene. Transcriptional activation domains are known in the art and include, but are not limited to, a herpes simplex virus VP16 activation domain and an NFAT activation domain.

In some embodiments, the expression modulator modulates the expression of the TRAC sequence through epigenetic mechanisms. In some embodiments, an epigenetic modulator covalently modifies DNA or histone proteins to alter histone structure and/or chromosomal structure without altering the DNA sequence, leading to changes in gene expression (e.g., upregulation or downregulation). Non-limiting examples of epigenetic modifications include acetylation or methylation of lysine residues, arginine methylation, serine and threonine phosphorylation, and lysine ubiquitination and sumoylation of histone proteins, and methylation and hydroxymethylation of cytosine residues in DNA. Non-limiting examples of epigenetic modulators include histone acetyltransferases, histone deacetylases, histone methyltransferases, histone demethylases, DNA methyltransferases, and DNA demethylases.

The nuclease-dead RGNs or an RGN with nickase activity can be targeted to particular genomic locations to modify the sequence of a target polynucleotide through fusion to a base-editing polypeptide, for example a deaminase polypeptide or active variant or fragment thereof, that directly chemically modifies (e.g., deaminates) a nucleobase, resulting in conversion from one nucleobase to another. The base-editing polypeptide can be fused to the RGN at its amino-terminal (N-terminal) or carboxy-terminal (C-terminal) end. Additionally, the base-editing polypeptide may be fused to the RGN via a peptide linker. Fusions of base-editing polypeptides and RGNs are described in International Appl. No. PCT/IB2023/061192, filed Nov. 6, 2023, which is herein incorporated by reference in its entirety. A non-limiting example of a deaminase polypeptide that is useful for such compositions and methods includes a cytosine deaminase or an adenosine deaminase (such as the adenosine deaminase base editor described in Gaudelli et al. (2017) Nature 551:464-471, U.S. Publ. Nos. 2017/0121693 and 2018/0073012, and International Publ. No. WO 2018/027078, or any of the deaminases disclosed in International Publ. No. WO 2020/139783, International Publ. No. WO 2022/056254, International Appl. No. PCT/US2022/021271, filed Mar. 22, 2022, and International Appl. No. PCT/IB2023/061192, filed Nov. 6, 2023, each of which is herein incorporated by reference in its entirety). In some embodiments, the deaminase polypeptide that is useful for such presently disclosed compositions and methods is a deaminase disclosed in Table 17 of International Publ. No. WO 2020/139783, which is incorporated herein by reference in its entirety.

Further, it is known in the art that certain fusion proteins between an RGN and a base-editing enzyme (e.g., cytosine deaminase) may also comprise at least one uracil stabilizing polypeptide that increases the mutation rate of a cytidine, deoxycytidine, or cytosine to a thymidine, deoxythymidine, or thymine in a nucleic acid molecule by a deaminase. Non-limiting examples of uracil stabilizing polypeptides include those disclosed in PCT Publication No. WO 2021/217002 and PCT Publication No. WO 2022/015969, each of which is herein incorporated by reference in its entirety. The disclosed uracil stabilizing polypeptides include USP2, and a uracil glycosylase inhibitor (UGI) domain, which may increase base editing efficiency. Therefore, a fusion protein may comprise an RGN described herein or variant thereof, a deaminase, and optionally at least one uracil stabilizing polypeptide, such as UGI or USP2. In embodiments, the RGN that is fused to the base-editing polypeptide is a nickase that cleaves the DNA strand that is not acted upon by the base-editing polypeptide (e.g., deaminase).

An RGN may be fused to a reverse transcriptase (RT) editing polypeptide (also referred to as prime editing polypeptide). RT editing (also referred to as prime editing) is a versatile and precise genome editing method that directly writes new genetic information into a specified DNA site using a nucleic acid programmable DNA binding protein working in association with a polymerase (described in, e.g., U.S. Pat. No. 11,447,770B1; WO2021072328; WO2021226558; WO2020156575; WO2021042047; U.S. Pat. No. 11,193,123; each incorporated by reference in its entirety herein). The RT editing system uses an RGN that is a nickase, and the system is programmed with a RT editing guide RNA. The RT editing guide RNA is a guide RNA that both specifies the target sequence and provides the template for polymerization of the replacement strand containing the edit by way of an extension engineered onto the guide RNA (e.g., at the 5′ or 3′ end, or at an internal portion of the guide RNA). The RGN nickase/RT editing polypeptide fusion is guided to the target sequence by the RT editing guide RNA and nicks the non-target strand upstream of sequence to be edited and upstream of the PAM, creating a 3′ flap on the non-target strand. The RT editing guide RNA includes a primer binding site (PBS) that is complementary to the 3′ flap of the non-target strand. In some embodiments, a PBS is at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In certain embodiments, the RT editing guide RNA comprises a PBS that is at least 5 (e.g., at least 5, 6, 7, 8, 9, 10, 11, 12, 13, 14, 15, 16, 17, 28, 19, or 20) nucleotides in length. In some embodiments, the RT editing guide RNA may comprise a PBS that is at least 8 nucleotides in length. Hybridrization of the PBS and 3′ flap of the non-target strand allows polymerization of the replacement strand containing the edit using the extension of the RT editing guide RNA as template. The extension of the RT editing guide RNA can be formed from RNA or DNA. In the case of an RNA extension, the polymerase of the RT editor can be an RNA-dependent DNA polymerase (such as a reverse transcriptase). In the case of a DNA extension, the polymerase of the RT editor may be a DNA-dependent DNA polymerase.

The replacement strand containing the desired edit (e.g., a single nucleobase substitution) shares the same sequence as the non-target strand of the target sequence to be edited (with the exception that it includes the desired edit). Through DNA repair and/or replication machinery, the non-target strand of the target sequence is replaced by the newly synthesized replacement strand containing the desired edit. In some cases, RT editing may be thought of as a “search-and-replace” genome editing technology since the RT editors not only search and locate the desired target sequence to be edited, but at the same time, encode a replacement strand containing a desired edit which is installed in place of the corresponding non-target strand of the target sequence. Thus, in some embodiments, a guide RNA of the disclosure comprises an extension comprising an edit template for RT editing. In some embodiments, a RT editing polypeptide that can be fused to an RGN includes a DNA polymerase. In certain embodiments, the DNA polymerase is a reverse transcriptase. In certain embodiments, the RGN is a nickase.

RGNs or other nucleases that are fused to a polypeptide or domain can be separated or joined by a linker. The term “linker,” as used herein, refers to a chemical group or a molecule linking two molecules or moieties, e.g., a binding domain and a cleavage domain of a nuclease. In some embodiments, a linker joins a gRNA binding domain of an RGN and a detectable label or epigenetic modulator. In some embodiments, a linker joins a nuclease-dead RGN and a detectable label or epigenetic modulator. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The presently disclosed compositions and methods can utilize RGNs or other nucleases comprising at least one nuclear localization signal (NLS) to enhance transport of the RGN to the nucleus of a cell. Nuclear localization signals are known in the art and generally comprise a stretch of basic amino acids (see, e.g., Lange et al., J. Biol. Chem. (2007) 282:5101-5105). In some embodiments, the RGN comprises 2, 3, 4, 5, 6 or more nuclear localization signals. The nuclear localization signal(s) can be a heterologous NLS. Non-limiting examples of nuclear localization signals useful for the presently disclosed RGNs are the nuclear localization signals of SV40 Large T-antigen, nucleoplasmin, and c-Myc (see, e.g., Ray et al. (2015) Bioconjug Chem 26(6): 1004-7). In embodiments, the RGN comprises the NLS sequence set forth as SEQ ID NO: 411 or 412. The RGN or other nuclease can comprise one or more NLS sequences at its N-terminus, C-terminus, or both the N-terminus and C-terminus. For example, the RGN can comprise two NLS sequences at the N-terminal region and four NLS sequences at the C-terminal region.

In some embodiments, the presently disclosed compositions and methods utilize RGNs or other nucleases comprising at least one cell-penetrating domain that facilitates cellular uptake of the RGN. Cell-penetrating domains are known in the art and generally comprise stretches of positively charged amino acid residues (i.e., polycationic cell-penetrating domains), alternating polar amino acid residues and non-polar amino acid residues (i.e., amphipathic cell-penetrating domains), or hydrophobic amino acid residues (i.e., hydrophobic cell-penetrating domains) (see, e.g., Milletti F. (2012) Drug Discov Today 17:850-860). A non-limiting example of a cell-penetrating domain is the trans-activating transcriptional activator (TAT) from the human immunodeficiency virus 1.

The nuclear localization signal and/or cell-penetrating domain can be located at the N-terminus, the C-terminus, or in an internal location of the RGN or other nuclease.

### V. Polynucleotides Encoding RNA-Guided Nucleases, Single Guide RNAs, CRISPR RNAs, and/or tracrRNAs

The present disclosure provides polynucleotides comprising or encoding the presently disclosed RGNs, crRNAs, tracrRNAs, and/or sgRNAs. Presently disclosed polynucleotides include those comprising or encoding a crRNA comprising a spacer capable of targeting a bound RGN to a target sequence in the TRAC gene having the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103.

The use of the term “polynucleotide” or “nucleic acid molecule” is not intended to limit the present disclosure to polynucleotides comprising DNA. Those of ordinary skill in the art will recognize that polynucleotides can comprise ribonucleotides (RNA) and combinations of ribonucleotides and deoxyribonucleotides. Such deoxyribonucleotides and ribonucleotides include both naturally occurring molecules and synthetic analogues. These include peptide nucleic acids (PNAs), PNA-DNA chimers, locked nucleic acids (LNAs), and phosphothiorate linked sequences. The polynucleotides disclosed herein also encompass all forms of sequences including, but not limited to, single-stranded forms, double-stranded forms, DNA-RNA hybrids, triplex structures, stem-and-loop structures, and the like.

In some of those embodiments wherein the presently disclosed compositions and methods comprise a nucleic acid molecule encoding an RGN, the nucleic acid molecule is an mRNA (messenger RNA) molecule. An mRNA refers to any polynucleotide which encodes a polypeptide of interest and which is capable of being translated to produce the encoded polypeptide of interest in vitro, in vivo, in situ, or ex vivo. In some embodiments, the basic components of an mRNA molecule include at least a coding region, a 5′UTR, a 3′UTR, a 5′ cap and a poly-A tail. In some embodiments, an mRNA encoding an RGN useful in the presently disclosed methods and compositions can include one or more structural and/or chemical modifications or alterations which impart useful properties to the polynucleotide. For instance, a useful property of an mRNA includes the lack of a substantial induction of the innate immune response of a cell into which the mRNA is introduced. A “structural” feature or modification is one in which two or more linked nucleotides are inserted, deleted, duplicated, inverted or randomized in an mRNA without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. Chemical modifications to mRNA can involve inclusion of 5-methylcytosine, N1-methyl-pseudouridine, pseudouridine, 2-thiouridine, 4-thiouridine, 5-methoxyuridine, 2′Fluoroguanosine, 2′Fluorouridine, 5-bromouridine, 5-(2-carbomethoxyvinyl) uridine, 5-[3(1-E-propenylamino)] uridine, α-thiocytidine, N6-methyladenosine, 5-methylcytidine, N4-acetylcytidine, 5-formylcytidine, or combinations thereof, in an mRNA.

The nucleic acid molecules encoding RGNs can be codon optimized for expression in an organism of interest (e.g., mammal). A “codon-optimized” coding sequence is a polynucleotide coding sequence having its frequency of codon usage designed to mimic the frequency of preferred codon usage or transcription conditions of a particular host cell. Expression in the particular host cell or organism is enhanced as a result of the alteration of one or more codons at the nucleic acid level such that the translated amino acid sequence is not changed. Nucleic acid molecules can be codon optimized, either wholly or in part. Codon tables and other references providing preference information for a wide range of organisms are available in the art (see, e.g., Gaspar et al. (2012) Bioinformatics 28(20): 2683-2684; Komar et al. (1998) Biol. Chem. 379(10): 1295-1300; and Inouye et al. (2015) Protein Expr. Purif. 109:47-54). Non-limiting examples of codon-optimized coding sequences for RGNs useful in the presently disclosed compositions and methods include SEQ ID NO: 108, 428, and 429.

Polynucleotides encoding the RGNs, crRNAs, tracrRNAs, and/or sgRNAs provided herein can be provided in expression cassettes for in vitro expression or expression in a cell, embryo, or organism of interest. The cassette will include 5′ and 3′ regulatory sequences operably linked to a polynucleotide encoding an RGN, a crRNA, a tracrRNA, and/or an sgRNA provided herein that allows for expression of the polynucleotide. The cassette may additionally contain at least one additional gene or genetic element to be co-transformed into the organism. Where additional genes or elements are included, the components are operably linked. The term “operably linked” is intended to mean a functional linkage between two or more elements. For example, an operable linkage between a promoter and a coding region of interest (e.g., region coding for an RGN, a crRNA, a tracrRNA, and/or an sgRNA) is a functional link that allows for expression of the coding region of interest. Operably linked elements may be contiguous or non-contiguous. When used to refer to the joining of two protein coding regions, by operably linked or “operably fused” is intended that the coding regions are in the same reading frame. In some embodiments, polypeptides that are “operably fused” means that the structure and/or biological activity of each individual peptide is also present in the fusion. Alternatively, the additional gene(s) or element(s) can be provided on multiple expression cassettes. For example, the nucleotide sequence encoding a presently disclosed RGN can be present on one expression cassette, whereas the nucleotide sequence encoding a crRNA, a tracrRNA, or a complete guide RNA can be on a separate expression cassette. Such an expression cassette is provided with a plurality of restriction sites and/or recombination sites for insertion of the polynucleotides to be under the transcriptional regulation of the regulatory regions. The expression cassette may additionally contain a selectable marker gene.

The expression cassette will include in the 5′-3′ direction of transcription, a transcriptional (and, in some embodiments, translational) initiation region (i.e., a promoter), an RGN-, crRNA-, tracrRNA- and/or sgRNA-encoding polynucleotide of the disclosure, and a transcriptional (and in some embodiments, translational) termination region (i.e., termination region) functional in the organism of interest. The promoters of the disclosure are capable of directing or driving expression of a coding sequence in a host cell. The regulatory regions (e.g., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention. As used herein, a chimeric gene comprises a coding sequence operably linked to a transcription initiation region that is heterologous to the coding sequence.

Convenient termination regions include ones from simian virus (SV40), human growth hormone (hGH), bovine growth hormone (BGH), and rabbit beta-globin (rbGlob). See also Proudfoot (1991) Cell 64:671-674; Munroe et al. (1990) Gene 91:151-158; Schek et al. (1992) Molecular and Cellular Biology 12(12): 5386-5393; Gil and Proudfoot (1987) Cell 49(3): 399-406; Goodwin and Rottman (1992) The Journal of Biological Chemistry 267(23): 16330-16334; and Lanoix and Acheson (1988) EMBO J. 7(8): 2515-2522.

Additional regulatory signals include, but are not limited to, transcriptional initiation start sites, operators, activators, enhancers, other regulatory elements, ribosomal binding sites, an initiation codon, termination signals, and the like. See, for example, Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, inducible, growth stage-specific, cell type-specific, tissue-preferred, tissue-specific, or other promoters for expression in the organism of interest.

Exemplary constitutive promoters for expression in cells of the present disclosure include: an SV40 early promoter; a mouse mammary tumor virus long terminal repeat (LTR) promoter; adenovirus major late promoter (Ad MLP); a herpes simplex virus (HSV) promoter; a cytomegalovirus (CMV) promoter such as the CMV immediate early promoter region (CMVIE); a rous sarcoma virus (RSV) promoter; a human ubiquitin C promoter (UBC); a human U6 small nuclear promoter (U6); an enhanced U6 promoter; a human H1 promoter from RNA polymerase III (H1); a human elongation factor 1α promoter (EF1A); a human beta-actin promoter (ACTB); a human or mouse phosphoglycerate kinase 1 promoter (PGK); a chicken β-Actin promoter coupled with CMV early enhancer (CAGG); a yeast transcription elongation factor promoter (TEF1); and the like. See, for example, Miyagishi et al. (2002) Nature Biotechnology 20:497-500; Xia et al. (2003) Nucleic Acids Res. 31(17): e100-e100; Pasleau et al. (1985) Gene 38:227-232; Martin-Gallardo et al. (1988) Gene 70:51-56; Oellig and Seliger (1990) J Neurosci Res 26:390-396; Manthorpe et al. (1993) Hum Gene Ther 4:419-431; Yew et al. (1997) Hum Gene Ther 8:575-584; Xu et al. (2001) Gene 272:149-156; Nguyen et al. (2008) J Surg Res 148:60-66; Costa et al. (2005) Nat Meth. 2:259-260; Lam and Truong (2020) ACS Synth. Biol. 9(10): 2625-2631.

Examples of inducible promoters include: stress-regulated promoters such as Hsp70 and Hsp90 promoters (Wurm et al. (1986) Proc. Natl. Acad. Sci. USA. 83:5414-5418; Nover L. Heat Shock Response. CRC Press; Boca Raton, FL, USA: 1991); metal-regulated promoters (Mayo et al. (1982) Cell. 29:99-108; Searle et al. (1985) Mol. Cell. Biol. 5:1480-1489); hormone-responsive promoters including a glucocorticoid-responsive promoter (Hynes et al. (1981) Proc. Natl. Acad. Sci. USA. 78:2038-2042; Klock et al. (1987) Nature. 329:734-736). Chemically regulated promoters from prokaryotes that have been used include isopropyl-beta-D-thiogalactopyranoside (IPTG)-regulated promoters, lactose-regulated promoters, and tetracycline-regulated promoters (see, for example, Gossen et al. (1993) Trends Biochem Sci. 18:471-475; Gossen and Bujard (1992) Proc. Natl Acad. Sci. USA 89:5547-5551; Zhou et al. (2006) Gene Ther. 13:1382-1390). Inducible expression can be obtained using operator systems including AlcR/acetaldehyde, ArgR/L-arginine, BirA/biotinyl-AMP, CymR/cumate, EthR/2-phenylethylbutyrate, HdnoR/6-hydroxynicotine, HucR/uric acid, MphR (A)/macrolides, PIP/Streptogramins, Rex/NADH, RheA/heat, ScbR/SCB1, TraR/3-oxo-C8-HSL, and TtgR/phloretin; see, for example, U.S. Pat. No. 8,728,759B2; U.S. Pat. No. 7,745,592B2; Weber and Fussenegger (2004) Methods Mol. Biol. 267:451-466; Hartenbach et al. (2007) Nucleic Acids Res. 35: e136; Weber et al. (2009) Metab. Eng. 11:117-124; Weber et al. (2008) Proc. Natl. Acad. Sci. USA. 105:9994-9998; Malphettes et al. (2005) Nucleic Acids Res. 33: e107; Kemmer et al. (2010) Nat. Biotechnol. 28:355-360; Weber et al. (2002) Nat. Biotechnol. 20:901-907; Fussenegger et al. (2000) Nat. Biotechnol. 18:1203-1208; Weber et al. (2006) Metab. Eng. 8:273-280; Weber et al. (2003) Nucleic Acids Res. 31: e69; Weber et al. (2003) Nucleic Acids Res. 31: e71; Neddermann et al. (2003) EMBO Rep. 4:159-165; and Gitzinger et al. (2009) Proc. Natl. Acad. Sci. USA. 106:10638-10643. Inducible expression can be obtained using protein-protein interaction systems including: rapamycin-induced interaction between FKBP12 (FK506 binding protein 12) and mTOR (Rivera et al. (1996) Nat. Med. 2:1028-1032; Belshaw et al. (1996) Proc. Natl. Acad. Sci. USA. 93:4604-46077); abscisic acid (ABA)-regulated interaction between PYL1 (abscisic acid receptor) and ABI1 (protein phosphatase 2C56) (Liang et al. (2011) Sci. Signal. 4(164): rs2-rs2); and light-induced protein-protein interaction systems (Wang et al. (2012) Nat. Methods. 9:266-269; Yamada et al. (2018) Cell. Rep. 25:487-500).

Tissue-specific or tissue-preferred promoters can be utilized to target expression of an expression construct within a particular tissue. In embodiments, the tissue-specific or tissue-preferred promoters are active in mammalian tissue. Examples of tissue-specific or tissue-preferred promoters include promoters that initiate transcription preferentially in certain tissues, such as the heart, CNS, or eye. A “tissue specific” promoter is a promoter that initiates transcription only in certain tissues. Unlike constitutive expression of genes, tissue-specific expression is the result of several interacting levels of gene regulation. As such, promoters from homologous or closely related species can be preferable to use to achieve efficient and reliable expression of transgenes in particular tissues. In some embodiments, the expression comprises a tissue-preferred promoter. A “tissue preferred” promoter is a promoter that initiates transcription preferentially, but not necessarily entirely or solely in certain tissues.

In some embodiments, the nucleic acid molecules encoding an RGN, crRNA, tracrRNA, and/or sgRNA comprise a cell type-specific promoter. A “cell type specific” promoter is a promoter that primarily drives expression in certain cell types in one or more organs. Some examples of cells in which cell type specific promoters may be primarily active include, for example, a cytotoxic T cell, a regulatory T cell, or a stem cell. The nucleic acid molecules can also include cell type preferred promoters. A “cell type preferred” promoter is a promoter that primarily drives expression mostly, but not necessarily entirely or solely in certain cell types in one or more organs. Some examples of cells in which cell type preferred promoters may be preferentially active include, for example, lymphocyte, neuron, adipocyte, cardiomyocyte, smooth muscle cell, and photoreceptor cell.

The nucleic acid sequences encoding the RGNs, crRNAs, tracrRNAs, and/or sgRNAs can be operably linked to a promoter sequence that is recognized by a phage RNA polymerase for example, for in vitro mRNA synthesis. In some embodiments, the in vitro-transcribed RNA can be purified for use in the methods described herein. For example, the promoter sequence can be a T7, T3, or SP6 promoter sequence or a variation of a T7, T3, or SP6 promoter sequence. In some embodiments, the expressed protein and/or RNAs can be purified for use in the methods of genome modification described herein.

In embodiments, the polynucleotide encoding the RGN, crRNA, tracrRNA, and/or sgRNA also can be linked to a polyadenylation signal (e.g., SV40 polyA signal and other signals functional in plants) and/or at least one transcriptional termination sequence. Additionally, the sequence encoding the RGN also can be linked to sequence(s) encoding at least one nuclear localization signal, at least one cell-penetrating domain, and/or at least one signal peptide capable of trafficking proteins to particular subcellular locations, as described elsewhere herein.

The polynucleotide encoding the RGN, crRNA, tracrRNA, and/or sgRNA can be present in a vector or multiple vectors. A “vector” refers to a polynucleotide composition for transferring, delivering, or introducing a nucleic acid into a host cell. Suitable vectors include plasmid vectors, phagemids, cosmids, artificial/mini-chromosomes, transposons, and viral vectors (e.g., lentiviral vectors, adeno-associated viral vectors, baculoviral vector). The vector can comprise additional expression control sequences (e.g., enhancer sequences, Kozak sequences, polyadenylation sequences, transcriptional termination sequences), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001.

The vector can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT). Marker genes can include genes that allow selection for growth on a particular nutrient or substance, such as dihydrofolate reductase (DHFR; Simonsen and Levinson (1983) Proc. Natl. Acad. Sci. U.S.A. 80:2495-2499), histidinol dehydrogenase (hisD; Hartman and Mulligan (1988) Proc. Natl. Acad. Sci. U.S.A. 85:8047-8051), puromycin-N-acetyl transferase (PAC or puro; de la Luna et al. (1988) Gene 62:121-126), thymidine kinase (TK; Littlefield (1964) Science 145:709-710), and xanthine-guanine phosphoribosyltransferase (XGPRT or gpt; Mulligan and Berg (1981) Proc. Natl. Acad. Sci. U.S.A. 78:2072-2076).

In some embodiments, the expression cassette or vector comprising the sequence encoding the RGN polypeptide can further comprise a sequence encoding a crRNA and/or a tracrRNA, or the crRNA and tracrRNA combined to create an sgRNA. The sequence(s) encoding the crRNA and/or tracrRNA can be operably linked to at least one transcriptional control sequence for expression of the crRNA and/or tracrRNA in the organism or host cell of interest. For example, the polynucleotide encoding the crRNA and/or tracrRNA can be operably linked to a promoter sequence that is recognized by RNA polymerase III (Pol III). Examples of suitable Pol III promoters include, but are not limited to, mammalian U6, U3, H1, and 7SL RNA promoters and rice U6 and U3 promoters, such as the human U6 promoter set forth as SEQ ID NO: 413, as well as the promoters disclosed in U.S. Provisional Appl. No. 63/209,660, filed Jun. 11, 2021, and International Application No. PCT/US2022/032940, filed Jun. 10, 2022, each of which is herein incorporated by reference in its entirety, including promoters set forth herein as SEQ ID NOs: 414-423.

As indicated, expression constructs comprising nucleotide sequences encoding an RGN, a crRNA, a tracrRNA, and/or an sgRNA can be used to transform organisms of interest. Methods for transformation involve introducing a nucleotide construct into an organism of interest. By “introducing” is intended to introduce the nucleotide construct to the host cell in such a manner that the construct gains access to the interior of the host cell. The methods of the disclosure do not require a particular method for introducing a nucleotide construct to a host organism, only that the nucleotide construct gains access to the interior of at least one cell of the host organism. The host cell can be a eukaryotic or prokaryotic cell. In some embodiments, the eukaryotic host cell is a mammalian cell, an avian cell, or an insect cell. In some embodiments, the eukaryotic cell that comprises or expresses a presently disclosed crRNA, tracrRNA, sgRNA, and/or RGN or that has been modified by a presently disclosed RGN system is a human cell. In some embodiments, the eukaryotic cell that comprises or expresses a presently disclosed crRNA, tracrRNA, sgRNA, and/or RGN or that has been modified by a presently disclosed RGN system is a stem cell, including an induced pluripotent stem cell. In some embodiments, the mammalian or human cell that comprises or expresses a presently disclosed crRNA, tracrRNA, sgRNA, and/or RGN or that has been modified by a presently disclosed RGN system is a lymphocyte. In some embodiments, the lymphocyte includes a cytotoxic T cell or a regulatory T cell.

Methods for introducing nucleotide constructs into host cells are known in the art including, but not limited to, stable transformation methods, transient transformation methods, and virus-mediated methods.

The presently disclosed methods can result in a transformed organism or cell line derived from these transformed cells.

“Transgenic organisms” or “transformed organisms” or “stably transformed” organisms or cells or tissues refers to organisms that have incorporated or integrated a polynucleotide encoding an RGN, a crRNA, a tracrRNA, and/or an sgRNA of the disclosure. It is recognized that other exogenous or endogenous nucleic acid sequences or DNA fragments may also be incorporated into the host cell. Transformation of a host cell may be performed by infection, conjugation, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, and viral mediated, liposome mediated and the like. Viral-mediated introduction of a polynucleotide encoding an RGN, a crRNA, a tracrRNA, and/or an sgRNA includes retroviral, lentiviral, adenoviral, and adeno-associated viral mediated introduction and expression.

Transformation may result in stable or transient incorporation of the nucleic acid into the cell. “Stable transformation” is intended to mean that the nucleotide construct introduced into a host cell integrates into the genome of the host cell and is capable of being inherited by the progeny thereof. “Transient transformation” is intended to mean that a polynucleotide is introduced into the host cell and does not integrate into the genome of the host cell.

In some embodiments, cells that have been transformed may be introduced into an organism. These cells could have originated from the organism, wherein the cells are transformed in an ex vivo approach. These cells can be autologous (originated and returned to the same subject), allogeneic (the donor and recipient subjects are of the same species). In general, the donor and recipient of allogeneic cells are a complete or partial HLA match.

The polynucleotides encoding the RGNs, crRNAs, tracrRNAs, and/or sgRNAs or comprising the crRNAs, tracrRNAs, and/or sgRNAs can also be used to transform any prokaryotic species, including but not limited to, archaea and bacteria (e.g., Bacillus sp., Klebsiella sp. Streptomyces sp., Rhizobium sp., Escherichia sp., Pseudomonas sp., Salmonella sp., Shigella sp., Vibrio sp., Yersinia sp., Mycoplasma sp., Agrobacterium, Lactobacillus sp.).

The polynucleotides encoding the RGNs, crRNAs, tracrRNAs, and/or sgRNAs or comprising the crRNAs, tracrRNAs, and/or sgRNAs can be used to transform any eukaryotic species, including but not limited to animals (e.g., mammals, humans, insects, fish, birds, and reptiles), fungi, amoeba, algae, and yeast.

Conventional viral and non-viral based gene transfer methods can be used to introduce nucleic acids in mammalian, insect, or avian cells or target tissues. Such methods can be used to administer nucleic acids encoding components of an RGN system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1): 31-44 (1995); Haddada et al., in Current Topics in Microbiology and Immunology, Doerfler and Bohm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration). The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids takes advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Viral. 66:2731-2739 (1992); Johann et al., J. Viral. 66:1635-1640 (1992); Sommnerfelt et al., Viral. 176:58-59 (1990); Wilson et al., J. Viral. 63:2374-2378 (1989); Miller et al., J. Viral. 65:2220-2224 (1991); PCT/US94/05700).

In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Katin, Human Gene Therapy 5:793-801 (1994); Muzyczka, 1. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Viral. 63:03822-3828 (1989). Packaging cells are typically used to form virus particles that are capable of infecting a host cell. Such cells include 293 cells, which package adenovirus, and ψJ2 cells or PA317 cells, which package retrovirus.

Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences.

The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In some embodiments, a host cell is transiently or non-transiently transfected with one or more nucleic acid molecules or vectors described herein. In some embodiments, a cell is transfected as it naturally occurs in a subject. In some embodiments, a cell that is transfected is taken from a subject. In embodiments, the cell is derived from cells taken from a subject, such as a cell line. In some embodiments, the cell line may be mammalian, insect, or avian cells. A wide variety of cell lines for tissue culture are known in the art. Examples of cell lines include, but are not limited to, C8161, CCRF-CEM, MOLT, mIMCD-3, NHDF, HeLaS3, Huhl, Huh4, Huh7, HUVEC, HASMC, HEKn, HEKa, MiaPaCell, Panel, PC-3, TFI, CTLL-2, CIR, Rat6, CVI, RPTE, AIO, T24, 182, A375, ARH-77, Calul, SW480, SW620, SKOV3, SK-UT, CaCo2, P388DI, SEM-K2, WEHI-231, HB56, TIB55, lurkat, 145.01, LRMB, Bcl-1, BC-3, IC21, DLD2, Raw264.7, NRK, NRK-52E, MRC5, MEF, Hep G2, HeLa B, HeLa T4. COS, COS-1, COS-6, COS-M6A, BS-C-1 monkey kidney epithelial, BALB/3T3 mouse embryo fibroblast, 3T3 Swiss, 3T3-LI, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-I cells, BEAS-2B, bEnd.3, BHK-21, BR 293, BxPC3, C3H-10TI/2, C6/36, Cal-27, CHO, CHO-7, CHO-IR, CHO-K1, CHO-K2, CHO-T, CHO Dhfr−/−, COR-L23, COR-L23/CPR, COR-L235010, CORL23/R23, COS-7, COV-434, CML TI, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, lurkat, IY cells, K562 cells, Ku812, KCL22, KGI, KYOI, LNCap, Ma-Mel 1-48, MC-38, MCF-7, MCF-10A, MDA-MB-231, MDA-MB-468, MDA-MB-435, MDCKII, MDCKII, MOR/0.2R, MONO-MAC 6, MTD-IA, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THPI cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

In some embodiments, a cell transfected with one or more nucleic acid molecules or vectors described herein is used to establish a new cell line comprising one or more vector-derived sequences. In some embodiments, a cell transiently transfected with the components of an RGN system as described herein (such as by transient transfection of one or more vectors, or transfection with RNA), and modified through the activity of an RGN system, is used to establish a new cell line comprising cells containing the modification but lacking any other exogenous sequence.

In some embodiments, one or more nucleic acid molecules or vectors described herein are used to produce a non-human transgenic animal. In some embodiments, the transgenic animal is a mammal, such as a mouse, rat, hamster, rabbit, cow, or pig. In some embodiments, the transgenic animal is a bird, such as a chicken or a duck. In some embodiments, the transgenic animal is an insect, such as a mosquito or a tick.

### VI. Variants and Fragments of Polypeptides and Polynucleotides

The present disclosure provides active variants and fragments of the presently disclosed crRNAs, tracrRNAs, sgRNA backbones, sgRNAs, and RGNs. An active variant or fragment of a naturally-occurring (i.e., wild-type) RGN binds to a target sequence described herein within the TRAC gene in an RNA-guided sequence-specific manner. In some embodiments, a target sequence described herein includes a target strand having the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, and 104. In some embodiments, the disclosure provides active variants and fragments of an RGN having an amino acid sequence set forth as SEQ ID NO: 105 or 333, as well as active variants and fragments of naturally-occurring CRISPR repeats, including sequences set forth as SEQ ID NOs: 106, 109-112, 328, 331, 334, 430, 432-435, 583, 585, and 587, active variants and fragments of naturally-occurring tracrRNAs, such as any one of the sequences set forth as SEQ ID NOs: 107, 114-123, 329, 332, 335, 431, 437-446, 584, 586, and 588, and active variants and fragments of sgRNAs, such as any one of the sequences set forth as SEQ ID NOs: 198-200, 202-213, 215-233, 235-241, 243-259, 521-523, 525-536, 538-556, 558-564, and 566-582, and polynucleotides encoding the same.

While the activity of a variant or fragment may be altered compared to the polynucleotide or polypeptide of interest, the variant and fragment should retain the functionality of the polynucleotide or polypeptide of interest. For example, a variant or fragment may have increased activity, decreased activity, different spectrum of activity or any other alteration in activity when compared to the polynucleotide or polypeptide of interest.

Fragments and variants of naturally-occurring RGN polypeptides, such as those disclosed herein, will retain sequence-specific, RNA-guided DNA-binding activity. In embodiments, fragments and variants of naturally-occurring RGN polypeptides, such as those disclosed herein, retain nuclease activity (single-stranded or double-stranded).

Fragments and variants of naturally-occurring CRISPR repeats, such as those disclosed herein, will retain the ability, when part of a guide RNA (comprising a tracrRNA), to bind to and guide an RNA-guided nuclease (complexed with the guide RNA) to a target sequence in a sequence-specific manner.

Fragments and variants of naturally-occurring tracrRNAs, such as those disclosed herein, will retain the ability, when part of a guide RNA (comprising a CRISPR RNA), to guide an RNA-guided nuclease (complexed with the guide RNA) to a target sequence in a sequence-specific manner.

Fragments and variants of sgRNA backbones, such as those disclosed herein, will retain the ability, when part of a guide RNA, to guide an RNA-guided nuclease (complexed with the guide RNA) to a target sequence in a sequence-specific manner.

Fragments and variants of sgRNAs, such as those disclosed herein, will retain the ability to guide an RNA-guided nuclease (complexed with the sgRNA) to a target sequence in a sequence-specific manner.

The term “fragment” refers to a portion of a polynucleotide or polypeptide sequence of the disclosure. “Fragments” or “biologically active portions” include polynucleotides comprising a sufficient number of contiguous nucleotides to retain the biological activity (i.e., binding to and directing an RGN in a sequence-specific manner to a target sequence when comprised within a guide RNA). “Fragments” or “biologically active portions” include polypeptides comprising a sufficient number of contiguous amino acid residues to retain the biological activity (i.e., binding to a target sequence in a sequence-specific manner when complexed with a guide RNA). Fragments of the RGN proteins include those that are shorter than the full-length sequences due to the use of an alternate downstream start site. A biologically active portion of an RGN protein can be a polypeptide that comprises, for example, 10, 25, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700 or more contiguous amino acid residues of an RGN that binds a target nucleotide sequence disclosed herein or of SEQ ID NO: 105 or 333. Such biologically active portions can be prepared by recombinant techniques and evaluated for sequence-specific, RNA-guided DNA-binding activity. A biologically active fragment of a CRISPR repeat sequence can comprise at least 8 contiguous nucleotides of any one of SEQ ID NOs: 106, 109-112, 328, 331, 334, 430, 432-435, 583, 585, and 587. A biologically active portion of a CRISPR repeat sequence can be a polynucleotide that comprises, for example, 8, 9, 10, 11, 12, or 13 contiguous nucleotides of any one of SEQ ID NOs: 106, 109-112, 328, 331, 334, 430, 432-435, 583, 585, and 587. A biologically active fragment of a crRNA sequence can comprise at least 20 contiguous nucleotides of any one of SEQ ID NOs: 136-197, and 459-520. A biologically active portion of a crRNA can be a polynucleotide that comprises, for example, 20, 25, 30, 35, 40 or more contiguous nucleotides of any one of SEQ ID NOs: 136-197, and 459-520. A biologically active portion of a tracrRNA can be a polynucleotide that comprises, for example, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80 or more contiguous nucleotides of any one of SEQ ID NOs: 107, 114-123, 329, 332, 335, 431, 437-446, 584, 586, and 588. A biologically active portion of a sgRNA backbone can be a polynucleotide that comprises, for example, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more contiguous nucleotides of any one of SEQ ID NOs: 124-134, and 447-457. A biologically active portion of a sgRNA can be a polynucleotide that comprises, for example, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more contiguous nucleotides of any one of SEQ ID NOs: 198-200, 202-213, 215-233, 235-241, 243-259, 521-523, 525-536, 538-556, 558-564, and 566-582.

In general, “variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” or “wild type” polynucleotide or polypeptide comprises a naturally occurring nucleotide sequence or amino acid sequence, respectively. For polynucleotides, conservative variants include those sequences that, because of the degeneracy of the genetic code, encode the native amino acid sequence of the gene of interest. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode the polypeptide or the polynucleotide of interest. Generally, variants of a particular polynucleotide disclosed herein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide disclosed herein (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides disclosed herein is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

In certain embodiments, the presently disclosed polynucleotides encode an RNA-guided nuclease polypeptide comprising an amino acid sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to an amino acid sequence encoding an RGN that binds a target sequence disclosed herein or an amino acid sequence set forth as SEQ ID NO: 105.

A biologically active variant of an RGN polypeptide of the disclosure may differ by as few as about 1-15 amino acid residues, as few as about 1-10, such as about 6-10, as few as 5, as few as 4, as few as 3, as few as 2, or as few as 1 amino acid residue. In some embodiments, the polypeptides can comprise an N-terminal or a C-terminal truncation, which can comprise at least a deletion of 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700 amino acids or more from either the N or C terminus of the polypeptide.

In some embodiments, the presently disclosed polynucleotides comprise or encode a crRNA repeat comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to the nucleotide sequence set forth as any one of SEQ ID NOs: 106, 109-112, 328, 331, 334, 430, 432-435, 583, 585, and 587.

In some embodiments, the presently disclosed polynucleotides comprise or encode a crRNA comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to the nucleotide sequence set forth as any one of SEQ ID NOs: 136-197, and 459-520.

The presently disclosed polynucleotides can comprise or encode a tracrRNA comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to the nucleotide sequence set forth as any one of SEQ ID NOs: 107, 114-123, 329, 332, 335, 431, 437-446, 584, 586, and 588.

The presently disclosed polynucleotides can comprise or encode an sgRNA backbone comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to the nucleotide sequence set forth as any one of SEQ ID NOs: 124-134, and 447-457.

The presently disclosed polynucleotides can comprise or encode an sgRNA comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to the nucleotide sequence set forth as any one of SEQ ID NOs: 198-200, 202-213, 215-233, 235-241, 243-259, 521-523, 525-536, 538-556, 558-564, and 566-582.

Biologically active variants of a CRISPR repeat, crRNA, tracrRNA, sgRNA backbone, or sgRNA of the disclosure may differ by as few as about 1-15 nucleotides, as few as about 1-10, such as about 6-10, as few as 5, as few as 4, as few as 3, as few as 2, or as few as 1 nucleotide. In some embodiments, the polynucleotides can comprise a 5′ or 3′ truncation, which can comprise at least a deletion of 5, 10, 15, 20, 25, 30, 35, 40, 45, 50, 55, 60, 65, 70, 75, 80, 90, 95, 100, 105, 110 nucleotides or more from either the 5′ or 3′ end of the polynucleotide.

It is recognized that modifications may be made to the RGN polypeptides, CRISPR repeats, crRNAs, tracrRNAs, sgRNA backbones, and sgRNAs provided herein, creating variant proteins and polynucleotides. Changes designed by man may be introduced through the application of site-directed mutagenesis techniques. Alternatively, native, as yet-unknown, or as yet unidentified polynucleotides and/or polypeptides structurally and/or functionally-related to the sequences disclosed herein may also be identified that fall within the scope of the present disclosure. Conservative amino acid substitutions may be made in non-conserved regions that do not alter the function of the RGN proteins. Alternatively, modifications may be made that improve the activity of the RGN.

Variant polynucleotides and proteins also encompass sequences and proteins derived from a mutagenic and recombinogenic procedure such as DNA shuffling. With such a procedure, one or more different RGN proteins disclosed herein (e.g., SEQ ID NO: 105 or 333) is manipulated to create a new RGN protein possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the RGN sequences provided herein and other known RGN genes to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458. A “shuffled” nucleic acid is a nucleic acid produced by a shuffling procedure such as any shuffling procedure set forth herein. Shuffled nucleic acids are produced by recombining (physically or virtually) two or more nucleic acids (or character strings), for example in an artificial, and optionally recursive, fashion. Generally, one or more screening steps are used in shuffling processes to identify nucleic acids of interest; this screening step can be performed before or after any recombination step. In some (but not all) shuffling embodiments, it is desirable to perform multiple rounds of recombination prior to selection to increase the diversity of the pool to be screened. The overall process of recombination and selection are optionally repeated recursively. Depending on context, shuffling can refer to an overall process of recombination and selection, or, alternately, can simply refer to the recombinational portions of the overall process.

As used herein, “sequence identity” or “identity” in the context of two polynucleotides or polypeptide sequences makes reference to the residues in the two sequences that are the same when aligned for maximum correspondence over a specified comparison window. It is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Protein sequences that differ by such conservative substitutions are said to have “sequence similarity” or “similarity”. Means for measuring sequence similarity are well known to those of skill in the art. Typically, this involves scoring a conservative substitution as a partial rather than a full mismatch. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acid substitution matrices and their use in quantifying the similarity between two sequences are well-known in the art and described, e.g., in Dayhoff et al. (1978) “A model of evolutionary change in proteins.” In “Atlas of Protein Sequence and Structure,” Vol. 5, Suppl. 3 (ed. M. O. Dayhoff), pp. 345-352. Natl. Biomed. Res. Found., Washington, D.C. and Henikoff et al. (1992) Proc. Natl. Acad. Sci. USA 89:10915-10919. The BLOSUM62 matrix is often used as a default scoring substitution matrix in sequence alignment protocols. The gap existence penalty is imposed for the introduction of a single amino acid gap in one of the aligned sequences, and the gap extension penalty is imposed for each additional empty amino acid position inserted into an already opened gap. The alignment is defined by the amino acids positions of each sequence at which the alignment begins and ends, and optionally by the insertion of a gap or multiple gaps in one or both sequences, so as to arrive at the highest possible score. While optimal alignment and scoring can be accomplished manually, the process is facilitated by the use of a computer-implemented alignment algorithm, e.g., gapped BLAST 2.0, described in Altschul et al. (1997) Nucleic Acids Res. 25:3389-3402, and made available to the public at the National Center for Biotechnology Information Website (www.ncbi.nlm.nih.gov). Optimal alignments, including multiple alignments, can be prepared using, e.g., PSI-BLAST, available through www.ncbi.nlm.nih.gov and described by Altschul et al. (1997) Nucleic Acids Res. 25:3389-3402.

With respect to an amino acid sequence that is optimally aligned with a reference sequence, an amino acid residue “corresponds to” the position in the reference sequence with which the residue is paired in the alignment. The “position” is denoted by a number that sequentially identifies each amino acid in the reference sequence based on its position relative to the N-terminus. Owing to deletions, insertion, truncations, fusions, etc., that must be taken into account when determining an optimal alignment, in general the amino acid residue number in a test sequence as determined by simply counting from the N-terminal will not necessarily be the same as the number of its corresponding position in the reference sequence. For example, in a case where there is a deletion in an aligned test sequence, there will be no amino acid that corresponds to a position in the reference sequence at the site of deletion. Where there is an insertion in an aligned reference sequence, that insertion will not correspond to any amino acid position in the reference sequence. In the case of truncations or fusions there can be stretches of amino acids in either the reference or aligned sequence that do not correspond to any amino acid in the corresponding sequence.

### VII. RGN Systems and Ribonucleoprotein Complexes for Binding a Target Sequence of Interest and Methods of Making the Same

The present disclosure provides a RGN system for binding a target sequence in the TRAC gene. As used herein, an RGN system comprises at least one RGN polypeptide or a polynucleotide comprising a nucleotide sequence encoding the RGN polypeptide and one or more guide RNAs. The one or more guide RNAs are capable of forming a complex with the RGN polypeptide (ribonucleoprotein complex). The presently disclosed RGN systems comprise: a) one or more guide RNAs, or one or more polynucleotides comprising one or more nucleotide sequences encoding the one or more guide RNAs; and b) an RGN polypeptide or a polynucleotide comprising a nucleotide sequence encoding the RGN polypeptide. The one or more guide RNAs are capable of targeting a bound RGN polypeptide to a target sequence. The one or more guide RNAs are capable of forming a complex with the RGN polypeptide to direct the RGN polypeptide to bind to the target sequence in the TRAC gene. The guide RNA hybridizes to the target strand of a target sequence in the TRAC gene and also forms a complex with the RGN polypeptide, thereby directing the RGN polypeptide to bind to the target sequence. In some embodiments, the target sequence is set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, and 104. In some embodiments, the target sequence within the TRAC gene has the nucleotide sequence set forth as: GCCGTGTACCAGCTGAGAGACTCT (SEQ ID NO: 8). In some embodiments, the target sequence within the TRAC gene has the nucleotide sequence set forth as: ATCCTCTTGTCCCACAGATATCC (SEQ ID NO: 10). In some embodiments, the RGN is capable of recognizing a consensus PAM sequence set forth as NNNNCC or NNRNCC. In some embodiments, the RGN is capable of recognizing a full PAM sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG. In some embodiments, the RGN comprises an amino acid sequence set forth as SEQ ID NO: 105, or an active variant or fragment thereof. In some embodiments, the RGN comprises an amino acid sequence having at least 80% sequence identity, at least 90% sequence identity, or at least 95% sequence identity to SEQ ID NO: 105. In some embodiments, the RGN comprises an amino acid sequence set forth as SEQ ID NO: 333, or an active variant or fragment thereof. In some embodiments, the RGN comprises an amino acid sequence having at least 80% sequence identity, at least 90% sequence identity, or at least 95% sequence identity to SEQ ID NO: 333. In some embodiments, the guide RNA comprises a CRISPR repeat sequence comprising the nucleotide sequence set forth as any one of SEQ ID NOs: 106, 109-112, 328, 331, 334, 430, 432-435, 583, 585, and 587, or an active variant or fragment thereof. In some embodiments, the guide RNA comprises a CRISPR repeat having the nucleotide sequence set forth as SEQ ID NO: 106, or an active variant or fragment thereof. In some embodiments, the guide RNA comprises a crRNA comprising the nucleotide sequence set forth as any one of SEQ ID NOs: 136-197, and 459-520, or an active variant or fragment thereof. In some embodiments, the guide RNA comprises a tracrRNA comprising the nucleotide sequence set forth as any one of SEQ ID NOs: 107, 114-123, 329, 332, 335, 431, 437-446, 584, 586, and 588, or an active variant or fragment thereof. In some embodiments, the guide RNA comprises a tracrRNA having the nucleotide sequence set forth as SEQ ID NO: 107, or an active variant or fragment thereof. In some embodiments, the guide RNA comprises an sgRNA backbone comprising any one of the nucleotide sequences set forth as SEQ ID NOs: 124-134, and 447-457. In some embodiments, the guide RNA comprises an sgRNA comprising any one of the nucleotide sequences set forth as SEQ ID NOs: 198-200, 202-213, 215-233, 235-241, 243-259, 521-523, 525-536, 538-556, 558-564, and 566-582, or an active variant or fragment thereof. In some embodiments, the guide RNA comprises a sgRNA having the nucleotide sequence set forth as SEQ ID NO: 204, or an active variant or fragment thereof. In some embodiments, the guide RNA comprises a sgRNA having the nucleotide sequence set forth as SEQ ID NO: 205, or an active variant or fragment thereof. The guide RNA of the system can be a single guide RNA or a dual-guide RNA. In some embodiments, the system comprises an RNA-guided nuclease that is heterologous to the guide RNA, wherein the RGN and guide RNA are not found complexed to one another (i.e., bound to one another) in nature.

The system for binding a target sequence of interest provided herein can be a ribonucleoprotein complex, which is at least one molecule of an RNA bound to at least one protein. The ribonucleoprotein complexes provided herein comprise at least one guide RNA as the RNA component and an RNA-guided nuclease as the protein component. Such ribonucleoprotein complexes can be purified from a cell or organism that naturally expresses an RGN polypeptide and has been engineered to express a particular guide RNA that is specific for a target sequence of interest (e.g., a target sequence in the TRAC gene). Alternatively, the ribonucleoprotein complex can be purified from a cell or organism that has been transformed with polynucleotides (e.g., an mRNA) that encode an RGN polypeptide and a guide RNA and cultured under conditions to allow for the expression of the RGN polypeptide and guide RNA. In some embodiments, the ribonucleoprotein complex is purified from a cell or organism that has been transformed with a polynucleotide (e.g., an mRNA) that encodes an RGN polypeptide and wherein a synthetically derived gRNA has been introduced. Thus, methods are provided for making an RGN polypeptide or an RGN ribonucleoprotein complex. Such methods comprise culturing a cell comprising a nucleotide sequence encoding an RGN polypeptide, and in some embodiments a nucleotide sequence encoding a guide RNA, under conditions in which the RGN polypeptide (and in some embodiments, the guide RNA) is expressed. The RGN polypeptide or RGN ribonucleoprotein can then be purified from a lysate of the cultured cells. In some embodiments, the nucleotide sequence encoding an RGN polypeptide includes a mRNA (messenger RNA). In some embodiments, methods for assembling an RNP complex comprise combining one or more of the presently disclosed guide RNAs and one or more of the presently disclosed RGN polypeptides under conditions suitable for formation of the RNP complex.

Methods for purifying an RGN polypeptide or RGN ribonucleoprotein complex from a lysate of a biological sample are known in the art (e.g., size exclusion and/or affinity chromatography, 2D-PAGE, HPLC, reversed-phase chromatography, immunoprecipitation). In particular methods, the RGN polypeptide is recombinantly produced and comprises a purification tag to aid in its purification, including but not limited to, glutathione-S-transferase (GST), chitin binding protein (CBP), maltose binding protein, thioredoxin (TRX), poly (NANP), tandem affinity purification (TAP) tag, myc, AcV5, AU1, AU5, E, ECS, E2, FLAG (e.g., 3× FLAG tag), HA, nus, Softag 1, Softag 3, Strep, SBP, Glu-Glu, HSV, KT3, S, S1, T7, V5, VSV-G, 6×His, 10×His, biotin carboxyl carrier protein (BCCP), and calmodulin. Generally, the tagged RGN polypeptide or RGN ribonucleoprotein complex is purified using immobilized metal affinity chromatography. It will be appreciated that other similar methods known in the art may be used, including other forms of chromatography or for example immunoprecipitation, either alone or in combination.

An “isolated” or “purified” polypeptide, or biologically active portion thereof, is substantially or essentially free from components that normally accompany or interact with the polypeptide as found in its naturally occurring environment. Thus, an isolated or purified polypeptide is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals. Similarly, an “isolated” polynucleotide or nucleic acid molecule is removed from its naturally occurring environment. An isolated polynucleotide is substantially free of chemical precursors or other chemicals when chemically synthesized or has been removed from a genomic locus via the breaking of phosphodiester bonds. An isolated polynucleotide can be part of a vector, a composition of matter or can be contained within a cell so long as the cell is not the original environment of the polynucleotide.

Particular methods provided herein for binding and/or cleaving a target sequence of interest involve the use of an in vitro assembled RGN ribonucleoprotein complex. In vitro assembly of an RGN ribonucleoprotein complex can be performed using any method known in the art in which an RGN polypeptide is contacted with a guide RNA under conditions to allow for binding of the RGN polypeptide to the guide RNA. As used herein, “contact”, contacting”, “contacted,” refer to placing the components of a desired reaction together under conditions suitable for carrying out the desired reaction. The RGN polypeptide can be purified from a biological sample, cell lysate, or culture medium, produced via in vitro translation, or chemically synthesized. The guide RNA can be purified from a biological sample, cell lysate, or culture medium, transcribed in vitro, or chemically synthesized. The RGN polypeptide and guide RNA can be brought into contact in solution (e.g., buffered saline solution) to allow for in vitro assembly of the RGN ribonucleoprotein complex.

Some aspects of this disclosure provide kits comprising one or more elements of an RGN system described herein, including: guide RNAs (i.e. crRNAs, tracrRNAs, and/or sgRNAs), RGNs, and/or polynucleotides encoding the same; cells; and complete RGN systems, and in some embodiments another type of nuclease. In some embodiments, the kit includes suitable reagents, buffers, and/or instructions for using one or more elements of an RGN system, e.g., for in vitro or in vivo nucleic acid editing. Reagents may be provided in any suitable container, such as a vial, a bottle, or a tube. Reagents may be used in a process utilizing one or more of the elements of an RGN system. For example, restriction enzymes may be included for cloning of a polynucleotide encoding an RGN or a guide RNA into a vector. In some embodiments, the kit includes instructions regarding the design and use of suitable guide RNAs (i.e. crRNAs, tracrRNAs, and/or sgRNAs) for targeted editing of a nucleic acid sequence. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10.

A kit including one or more elements of an RGN system of the disclosure has utility in a wide variety of applications including modifying (e.g., deleting, inserting, translocating, inactivating, activating) a target polynucleotide in a multiplicity of cell types.

In some embodiments, a kit of the disclosure includes a kit including a composition described herein. In some embodiments, a kit may include: (a) a container containing a composition of the disclosure in lyophilized form and (b) a second container containing an acceptable diluent (e.g., sterile water) for injection. An acceptable diluent can be used for reconstitution or dilution of the lyophilized compound of the disclosure. Optionally associated with such container(s) can be a notice in the form prescribed by a governmental agency regulating the manufacture, use or sale of biological products.

### VIII. Methods of Binding, Cleaving, or Modifying a Target Sequence

The present disclosure provides methods for binding, cleaving, and/or modifying a target sequence in the TRAC gene. The methods include delivering an RGN system comprising at least one guide RNA or a polynucleotide encoding the same, and at least one RGN polypeptide or a polynucleotide encoding the same to the target sequence or a cell or embryo comprising the target sequence. In some embodiments, the target sequence within the TRAC gene has a nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, and 104. In some embodiments, the target sequence within the TRAC gene has the nucleotide sequence set forth as SEQ ID NO: 8. In some embodiments, the target sequence within the TRAC gene has the nucleotide sequence set forth as SEQ ID NO: 10.

In some embodiments, the RGN is capable of recognizing a consensus PAM sequence set forth as NNNNCC or NNRNCC. In some embodiments, the RGN is capable of recognizing a full PAM sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG. The RGN can comprise an amino acid sequence set forth as SEQ ID NO: 105, or an active variant or fragment thereof. In some embodiments, the RGN comprises an amino acid sequence having at least 80% sequence identity, at least 90% sequence identity, or at least 95% sequence identity to SEQ ID NO: 105. In some embodiments, the RGN comprises an amino acid sequence set forth as SEQ ID NO: 333, or an active variant or fragment thereof. In some embodiments, the RGN comprises an amino acid sequence having at least 80% sequence identity, at least 90% sequence identity, or at least 95% sequence identity to SEQ ID NO: 333. The guide RNA can comprise a CRISPR repeat sequence comprising the nucleotide sequence set forth as any one of SEQ ID NOs: 106, 109-112, 328, 331, 334, 430, 432-435, 583, 585, and 587, or an active variant or fragment thereof. In some embodiments, the guide RNA comprises a CRISPR repeat having the nucleotide sequence set forth as SEQ ID NO: 106, or an active variant or fragment thereof. The guide RNA can comprise a crRNA comprising the nucleotide sequence set forth as any one of SEQ ID NOs: 136-197, and 459-520, or an active variant or fragment thereof. The guide RNA can comprise a tracrRNA comprising the nucleotide sequence set forth as any one of SEQ ID NOs: 107, 114-123, 329, 332, 335, 431, 437-446, 584, 586, and 588, or an active variant or fragment thereof. In some embodiments, the guide RNA comprises a tracrRNA having the nucleotide sequence set forth as SEQ ID NO: 107, or an active variant or fragment thereof. The guide RNA can comprise an sgRNA backbone comprising any one of the nucleotide sequences set forth as SEQ ID NOs: 124-134, and 447-457, or an active variant or fragment thereof. The guide RNA can comprise an sgRNA comprising any one of the nucleotide sequences set forth as SEQ ID NOs: 198-200, 202-213, 215-233, 235-241, 243-259, 521-523, 525-536, 538-556, 558-564, and 566-582, or an active variant or fragment thereof. In some embodiments, the guide RNA comprises a sgRNA having the nucleotide sequence set forth as SEQ ID NO: 204, or an active variant or fragment thereof. In some embodiments, the guide RNA comprises a sgRNA having the nucleotide sequence set forth as SEQ ID NO: 205, or an active variant or fragment thereof. The guide RNA of the system can be a single guide RNA or a dual-guide RNA.

The RGN of the system may be a nuclease dead RGN, have nickase activity, or may be a fusion polypeptide. In some embodiments, the RGN fusion protein comprises a polypeptide that recruits members of a functional nucleic acid repair complex, such as a member of the nucleotide excision repair (NER) or transcription coupled-nucleotide excision repair (TC-NER) pathway (Wei et al., 2015, PNAS USA 112(27): E3495-504; Troelstra et al., 1992, Cell 71:939-953; Marnef et al., 2017, J Mol Biol 429(9): 1277-1288), as described in U.S. Provisional Application No. 62/966,203, which was filed on Jan. 27, 2020, and is incorporated by reference in its entirety. In some embodiments, the RGN fusion protein comprises CSB (van den Boom et al., 2004, J Cell Biol 166(1): 27-36; van Gool et al., 1997, EMBO J 16(19): 5955-65; an example of which is set forth as SEQ ID NO: 424), which is a member of the TC-NER (nucleotide excision repair) pathway and functions in the recruitment of other members. In further embodiments, the RGN fusion protein comprises an active domain of CSB, such as the acidic domain of CSB which comprises amino acid residues 356-394 of SEQ ID NO: 424 (Teng et al., 2018, Nat Commun 9(1): 4115).

In certain embodiments, the RGN and/or guide RNA is heterologous to the cell or embryo to which the RGN and/or guide RNA (or polynucleotide(s) encoding at least one of the RGN and guide RNA) are introduced.

In embodiments wherein the method comprises delivering a polynucleotide encoding a guide RNA and/or an RGN polypeptide, the cell or embryo can then be cultured under conditions in which the guide RNA and/or RGN polypeptide are expressed. In some embodiments, the method comprises contacting a target nucleic acid molecule with an RGN ribonucleoprotein complex. The RGN ribonucleoprotein complex may comprise an RGN that is nuclease dead or has nickase activity. In some embodiments, the method comprises introducing into a cell or embryo comprising a target nucleic acid molecule an RGN ribonucleoprotein complex. The RGN ribonucleoprotein complex can be one that has been purified from a biological sample, recombinantly produced and subsequently purified, or in vitro-assembled as described herein. In embodiments wherein the RGN ribonucleoprotein complex that is contacted with the target nucleic acid molecule, or cell or embryo, has been assembled in vitro, the method can further comprise the in vitro assembly of the complex prior to contact with the target nucleic acid molecule, cell or embryo.

A purified or in vitro assembled RGN ribonucleoprotein complex can be introduced into a cell or embryo using any method known in the art, including, but not limited to electroporation. Alternatively, an RGN polypeptide and/or polynucleotide encoding or comprising the guide RNA can be introduced into a cell or embryo using any method known in the art (e.g., electroporation).

Upon delivery to or contact with the target nucleic acid molecule or cell or embryo comprising the target nucleic acid molecule, the guide RNA directs the RGN to bind to the target sequence within the target nucleic acid molecule in a sequence-specific manner. In those embodiments wherein the RGN has nuclease activity, the RGN polypeptide cleaves the target sequence upon binding. The target sequence can subsequently be modified via endogenous repair mechanisms, such as non-homologous end joining, or homology-directed repair with a provided donor polynucleotide.

Methods to measure binding of an RGN polypeptide to a target sequence are known in the art and include chromatin immunoprecipitation assays, gel mobility shift assays, DNA pull-down assays, reporter assays, microplate capture and detection assays. Likewise, methods to measure cleavage or modification of a target nucleic acid molecule comprising a target sequence are known in the art and include in vitro or in vivo cleavage assays wherein cleavage is confirmed using PCR, sequencing, or gel electrophoresis, with or without the attachment of an appropriate label (e.g., radioisotope, fluorescent substance) to the target sequence to facilitate detection of degradation products. Alternatively, the nicking triggered exponential amplification reaction (NTEXPAR) assay can be used (see, e.g., Zhang et al. (2016) Chem. Sci. 7:4951-4957). In vivo cleavage can be evaluated using the Surveyor assay (Guschin et al. (2010) Methods Mol Biol 649:247-256).

In some embodiments, the methods involve the use of only one RGN and only one of the presently disclosed guide RNAs. In some embodiments, the methods involve the use of a single type of RGN complexed with more than one guide RNA. In some embodiments, the methods involve the use of two types of RGNs, each complexed with a guide RNA. The more than one guide RNA can target different regions of a single gene or can target multiple genes. For example, a first guide RNA can target exon 1 in the TRAC gene and a second guide RNA can target intron 1 in the TRAC gene.

In those embodiments wherein a donor polynucleotide is not provided, a double-stranded break introduced by an RGN polypeptide can be repaired by a non-homologous end-joining (NHEJ) repair process. Due to the error-prone nature of NHEJ, repair of the double-stranded break can result in a mutation to the target sequence. In certain embodiments, a “mutation” in reference to a nucleic acid molecule refers to a change in the nucleotide sequence of the nucleic acid molecule, which can be a deletion, insertion, or substitution of one or more nucleotides, or a combination thereof. Mutation of the target nucleic acid molecule comprising a target sequence can result in the expression of an altered protein product or inactivation of a coding sequence.

The methods can comprise integrating a donor polynucleotide into the TRAC gene using an RGN system of the disclosure. In those embodiments wherein a donor polynucleotide is present, the donor sequence in the donor polynucleotide can be integrated into or exchanged with the target nucleotide sequence during the course of repair of the introduced double-stranded break, resulting in the introduction of the exogenous donor sequence. A donor polynucleotide thus comprises a donor sequence that is desired to be introduced into a target sequence of interest (e.g., a target sequence in the TRAC gene). In some embodiments, the donor sequence alters the original target nucleotide sequence such that the newly integrated donor sequence will not be recognized and cleaved by the RGN. Integration of the donor sequence can be enhanced by the inclusion within the donor polynucleotide of flanking sequences, referred to herein as “homology arms” that have substantial sequence identity with the sequences flanking the target nucleotide sequence, allowing for a homology-directed repair process. In some embodiments, homology arms have a length of at least 50 base pairs, at least 100 base pairs, and up to 2000 base pairs or more, and have at least 90%, at least 95%, or more, sequence homology to their corresponding sequence within the target nucleotide sequence. In some embodiments, the donor polynucleotide comprises a nucleotide sequence encoding an engineered T cell receptor, a chimeric antigen receptor, or an antibody.

In those embodiments wherein the RGN polypeptide introduces double-stranded staggered breaks, the donor polynucleotide can comprise a donor sequence flanked by compatible overhangs, allowing for direct ligation of the donor sequence to the cleaved target nucleotide sequence comprising overhangs by a non-homologous repair process during repair of the double-stranded break.

In those embodiments wherein the method involves the use of an RGN that is a nickase (i.e., is only able to cleave a single strand of a double-stranded polynucleotide), the method can comprise introducing two RGN nickases that target identical or overlapping target sequences and cleave different strands of the polynucleotide. For example, an RGN nickase that only cleaves the positive (+) strand of a double-stranded polynucleotide can be introduced along with a second RGN nickase that only cleaves the negative (−) strand of a double-stranded polynucleotide.

In some embodiments, a method is provided for binding a target nucleotide sequence and detecting the target sequence, wherein the method comprises introducing into a cell or embryo at least one guide RNA or a polynucleotide encoding the same, and at least one RGN polypeptide or a polynucleotide encoding the same, expressing the guide RNA and/or RGN polypeptide (if coding sequences are introduced), wherein the RGN polypeptide is a nuclease-dead RGN and further comprises a detectable label, and the method further comprises detecting the detectable label. The detectable label may be fused to the RGN as a fusion protein (e.g., fluorescent protein) or may be a small molecule conjugated to or incorporated within the RGN polypeptide that can be detected visually or by other means.

Also provided herein are methods for modulating the expression of a TRAC gene. In some embodiments, the methods comprise modulating expression of a TRAC gene in a population of cells. In some embodiments, the population of cells comprises T cells. The method can comprise comprising delivering an RGN system or an RNP complex described herein to the population of cells, wherein the population of cells comprises a target sequence within the TRAC gene, and wherein TRAC gene expression is modulated as compared to TRAC gene expression in a control population of cells. In some embodiments, cleavage or modification of the target sequence occurs. Cleavage or modification of the target sequence can be detected by sequencing. TRAC gene expression can be measured by quantitative PCR, microarray, RNA-seq, flow cytometry, immunoblot, enzyme-linked immunosorbent assay (ELISA), protein immunoprecipitation, immunostaining, high performance liquid chromatography (HPLC), liquid chromatography-mass spectrometry (LC/MS), mass spectrometry, or a combination thereof. In some embodiments, TRAC gene expression is decreased. The decrease in TRAC gene expression can comprise a decrease in TRAC mRNA level and/or TRAC protein level. In some embodiments, the decrease in TRAC mRNA level and/or Trac protein level is due to cleavage of the TRAC gene by an RGN system of the disclosure. In some embodiments, the decrease in TRAC protein level is measured by flow cytometry for detection of CD3+ cells. A decrease in CD3+ cells as compared to a level of CD3+ cells in a control population of cells can be indicative of a decrease in TRAC protein level. In some embodiments, the decrease in CD3+ cells is 30% to 100%. In some embodiments, the decrease in CD3+ cells is 50% to 100%. Cleavage or modification of the target sequence can occur at a rate of 40% to 100%, or 60% to 99%, or 70% to 90%. In some embodiments, cleavage or modification of the target sequence can occur at a rate of at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or more. In some embodiments, cleavage or modification of the target sequence occurs at a rate of 80% to 100%. The control population of cells can include a population of cells that has not been subjected to the delivering.

In some embodiments, methods for modulating the expression of a TRAC gene comprise introducing into a cell or embryo at least one guide RNA or a polynucleotide encoding the same, and at least one RGN polypeptide or a polynucleotide encoding the same, expressing the guide RNA and/or RGN polypeptide (if coding sequences are introduced), wherein the RGN polypeptide is a nuclease-dead RGN. In some embodiments, the nuclease-dead RGN is a fusion protein comprising an expression modulator as described herein.

The methods can comprise activation of the TRAC gene using an RGN system of the disclosure. In some embodiments, an RGN system can be targeted to the TRAC gene to increase or activate expression of the gene. The RGN (e.g., a nuclease-dead RGN) or its complexed guide RNA can be operably fused to an expression modulator such that binding of the RGN/guide RNA complex to a target sequence within the TRAC gene serves to increase or activate expression of the TRAC gene. In some embodiments, the expression modulator comprises a transcriptional activation domain, which interacts with transcriptional control elements and/or transcriptional regulatory proteins, such as RNA polymerases and transcription factors, to increase or activate transcription of the TRAC gene. Transcriptional activation domains are known in the art and include, but are not limited to, a herpes simplex virus VP16 activation domain and an NFAT activation domain.

One of ordinary skill in the art will appreciate that any of the presently disclosed methods can be used to target a single target sequence or multiple target sequences in the TRAC gene. Thus, methods comprise the use of a single RGN polypeptide in combination with multiple, distinct guide RNAs, which can target multiple, distinct sequences within the TRAC gene.

In some embodiments, methods of the disclosure are performed ex vivo or in vitro. In some embodiments, methods of the disclosure do not include methods for treatment of the human or animal body by therapy. In some embodiments, methods of the disclosure do not include methods that comprise a process for modifying the germ line genetic identity of human beings or does not comprise a use of human embryos for industrial or commercial purposes.

### IX. Cells Comprising a Polynucleotide Genetic Modification

Provided herein are cells and organisms comprising a target sequence in the TRAC gene that has been modified using a process mediated by an RGN, crRNA, tracrRNA, and/or sgRNA as described herein. In some embodiments, the RGN is capable of recognizing a consensus PAM sequence set forth as NNNNCC or NNRNCC. In some embodiments, the RGN is capable of recognizing a full PAM sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG. The RGN can comprise an amino acid sequence set forth as SEQ ID NO: 105, or an active variant or fragment thereof. In some embodiments, the RGN comprises an amino acid sequence having at least 80% sequence identity, at least 90% sequence identity, or at least 95% sequence identity to SEQ ID NO: 105. In some embodiments, the RGN comprises an amino acid sequence set forth as SEQ ID NO: 333, or an active variant or fragment thereof. In some embodiments, the RGN comprises an amino acid sequence having at least 80% sequence identity, at least 90% sequence identity, or at least 95% sequence identity to SEQ ID NO: 333. The guide RNA can comprise a CRISPR repeat sequence comprising the nucleotide sequence set forth as any one of SEQ ID NOs: 106, 109-112, 328, 331, 334, 430, 432-435, 583, 585, and 587, or an active variant or fragment thereof. In some embodiments, the guide RNA comprises a CRISPR repeat having the nucleotide sequence set forth as SEQ ID NO: 106, or an active variant or fragment thereof. The guide RNA can comprise a crRNA comprising the nucleotide sequence set forth as any one of SEQ ID NOs: 136-197, and 459-520, or an active variant or fragment thereof. The guide RNA can comprise a tracrRNA comprising the nucleotide sequence set forth as any one of SEQ ID NOs: 107, 114-123, 329, 332, 335, 431, 437-446, 584, 586, and 588, or an active variant or fragment thereof. In some embodiments, the guide RNA comprises a tracrRNA having the nucleotide sequence set forth as SEQ ID NO: 107, or an active variant or fragment thereof. The guide RNA can comprise an sgRNA backbone comprising the nucleotide sequence set forth as any one of SEQ ID NOs: 124-134, and 447-457, or an active variant or fragment thereof. The guide RNA can comprise an sgRNA comprising the nucleotide sequences set forth as any one of SEQ ID NOs: 198-200, 202-213, 215-233, 235-241, 243-259, 521-523, 525-536, 538-556, 558-564, and 566-582, or an active variant or fragment thereof. In some embodiments, the guide RNA comprises a sgRNA having the nucleotide sequence set forth as SEQ ID NO: 204, or an active variant or fragment thereof. In some embodiments, the guide RNA comprises a sgRNA having the nucleotide sequence set forth as SEQ ID NO: 205, or an active variant or fragment thereof. The guide RNA of the system can be a single guide RNA or a dual-guide RNA.

The modified cells can be eukaryotic (e.g., mammalian, insect, avian cell) or prokaryotic. Prokaryotic cells can be from species, including but not limited to, archaea and bacteria (e.g., Bacillus sp., Klebsiella sp. Streptomyces sp., Rhizobium sp., Escherichia sp., Pseudomonas sp., Salmonella sp., Shigella sp., Vibrio sp., Yersinia sp., Mycoplasma sp., Agrobacterium, Lactobacillus sp.).

Eukaryotic cells can include cells from animals (e.g., mammals, insects, fish, birds, and reptiles), fungi, amoeba, algae, and yeast. In some embodiments, the cell that is modified by the presently disclosed methods include lymphocytes. In some embodiments, lymphocytes include cytotoxic T cells or regulatory T cells. Cytotoxic T cells recognize and destroy infected, damaged, or cancerous cells and can be identified by various markers including CD8; CD45; CD54; tumor necrosis factor (TNF) alpha, interferon (IFN) gamma, IL-2 CXCR3, and/or TBX21 for Tc1; IL-4, IL-5, CCR4, and/or GATA3 for Tc2; IL-9, IL-10, and/or IRF4 for Tc9; and CCR6, KLRB1, IL-17, IRF4, and/or RORC for Tc17. Regulatory T cells modulate or suppress immune responses by, for example, secreting anti-inflammatory cytokines, expressing inhibitory proteins, and/or inducing apoptosis of effector T cells by cytokine deprivation, and can be identified by various markers including TRAC, IL-2 receptor alpha (IL2RA or CD25), STAT5A, CTLA4, IL-10, and/or transforming growth factor (TGF) beta. Also provided are embryos comprising at least one TRAC gene that has been modified by a process utilizing an RGN, crRNA, tracrRNA, and/or sgRNA as described herein. The genetically modified cells, organisms, and embryos can be heterozygous or homozygous for the modified TRAC gene.

In some embodiments, the chromosomal modification of the cell, organism, or embryo can result in downregulation or abolishment of expression of the TRAC mRNA or protein encoded by the TRAC gene. In embodiments, the chromosomal modification results in the production of a TRAC mRNA that has decreased translation of the TRAC protein as compared to a TRAC mRNA transcribed from a wild-type TRAC gene of a cell, organism, or embryo that has not undergone chromosomal modification. In some embodiments, the chromosomal modification results in the production of a variant TRAC protein product that is less stable or reduced in expression as compared to a TRAC protein encoded by a wild-type TRAC gene of a cell, organism or embryo that has not undergone chromosomal modification. In some embodiments, the expressed variant TRAC protein can have at least one amino acid substitution and/or the addition or deletion of at least one amino acid. The variant TRAC protein encoded by the altered chromosomal sequence can exhibit modified characteristics or activities when compared to the wild-type TRAC protein, including but not limited to altered ability to activate or repress TRAC target genes.

Cells that have been modified may be introduced into an organism. These cells could have originated from the same organism (e.g., person) in the case of autologous cellular transplants, wherein the cells are modified in an ex vivo approach. Alternatively, the cells originated from another organism within the same species (e.g., another person) in the case of allogeneic cellular transplants.

The article “a” and “an” are used herein to refer to one or more than one (i.e., to at least one) of the grammatical object of the article. By way of example, “a polypeptide” means one or more polypeptides.

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended embodiments.

Non-limiting embodiments include:
1. A guide RNA (gRNA) comprising a CRISPR RNA (crRNA) and a trans-activating CRISPR RNA (tracrRNA), wherein the crRNA comprises
(i) a crRNA repeat; and
(ii) a spacer,
   wherein the tracrRNA comprises:
(iii) an anti-repeat; and
(iv) a tail,
   wherein the spacer is capable of hybridizing to a target sequence in a T cell receptor alpha chain constant (TRAC) gene, wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, and 104.
2. The gRNA of embodiment 1, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 to 5 nucleotides.
3. The gRNA of embodiment 2, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 5 nucleotides.
4. The gRNA of embodiment 2, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 4 nucleotides.
5. The gRNA of embodiment 2, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 3 nucleotides.
6. The gRNA of embodiment 2, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 2 nucleotides.
7. The gRNA of embodiment 2, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 nucleotide.
8. The gRNA of embodiment 1, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103.
9. The gRNA of any one of embodiments 1-8, wherein the crRNA repeat has the nucleotide sequence set forth as SEQ ID NO: 106 or a nucleotide sequence that differs in length and/or sequence from SEQ ID NO: 106 by 1 to 8 nucleotides.
10. The gRNA of embodiment 9, wherein the crRNA repeat has a nucleotide sequence that differs in length and/or sequence from SEQ ID NO: 106 by 8 nucleotides.
11. The gRNA of embodiment 9, wherein the crRNA repeat has a nucleotide sequence that differs in length and/or sequence from SEQ ID NO: 106 by 7 nucleotides.
12. The gRNA of embodiment 9, wherein the crRNA repeat has a nucleotide sequence that differs in length and/or sequence from SEQ ID NO: 106 by 6 nucleotides.
13. The gRNA of embodiment 9, wherein the crRNA repeat has a nucleotide sequence that differs in length and/or sequence from SEQ ID NO: 106 by 5 nucleotides.
14. The gRNA of embodiment 9, wherein the crRNA repeat has a nucleotide sequence that differs in length and/or sequence from SEQ ID NO: 106 by 4 nucleotides.
15. The gRNA of embodiment 9, wherein the crRNA repeat has a nucleotide sequence that differs in length and/or sequence from SEQ ID NO: 106 by 3 nucleotides.
16. The gRNA of embodiment 9, wherein the crRNA repeat has a nucleotide sequence that differs in length and/or sequence from SEQ ID NO: 106 by 2 nucleotides.
17. The gRNA of embodiment 9, wherein the crRNA repeat has a nucleotide sequence that differs in length and/or sequence from SEQ ID NO: 106 by 1 nucleotide.
18. The gRNA of embodiment 9, wherein the crRNA repeat has the nucleotide sequence set forth as any one of SEQ ID NOs: 106, 109-112, 328, 331, and 334.
19. The gRNA of any one of embodiments 1-8, wherein the crRNA has a nucleotide sequence having at least 80% sequence identity to SEQ ID NOs: 136-197.
20. The gRNA of embodiment 19, wherein the crRNA has a nucleotide sequence having at least 90% sequence identity to SEQ ID NOs: 136-197.
21. The gRNA of embodiment 19, wherein the crRNA has a nucleotide sequence having at least 95% sequence identity to SEQ ID NOs: 136-197.
22. The gRNA of embodiment 19, wherein the crRNA has the nucleotide sequence set forth as any one of SEQ ID NOs: 136-197.
23. The gRNA of any one of embodiments 1-8, wherein the tracrRNA has a nucleotide sequence having at least 80% sequence identity to SEQ ID NO: 107.
24. The gRNA of embodiment 23, wherein the tracrRNA has a nucleotide sequence having at least 90% sequence identity to SEQ ID NO: 107.
25. The gRNA of embodiment 23, wherein the tracrRNA has a nucleotide sequence having at least 95% sequence identity to SEQ ID NO: 107.
26. The gRNA of any one of embodiments 1-8, wherein the tracrRNA has a nucleotide sequence that differs in length from SEQ ID NO: 107 by 1 to 16 nucleotides.
27. The gRNA of embodiment 26, wherein the tracrRNA has a nucleotide sequence that is 8 nucleotides shorter than SEQ ID NO: 107.
28. The gRNA of embodiment 26, wherein the tracrRNA has a nucleotide sequence that is 11 nucleotides shorter than SEQ ID NO: 107.
29 The gRNA of any one of embodiments 23-28, wherein the tracrRNA has the nucleotide sequence set forth as any one of SEQ ID NOs: 107, 114-123, 329, 332, and 335.
30. The gRNA of any one of embodiments 1-8, wherein the gRNA is a single guide RNA (sgRNA) comprising the crRNA and the tracrRNA linked by a linker, wherein the sgRNA comprises a backbone and the spacer, and wherein the backbone of the sgRNA comprises the crRNA repeat, the linker, and the tracrRNA.
31. The gRNA of embodiment 30, wherein the linker has a nucleotide sequence set forth as AAAG, GAAA, ACUU, or CAAAGG.
32. The gRNA of embodiment 31, wherein the linker has a nucleotide sequence set forth as AAAG.
33. The gRNA of any one of embodiments 30-32, wherein the backbone of the sgRNA comprises a total length of at least 85, 90, 95, 100, 105, 110, 115, or 120 nucleotides.
34. The gRNA of any one of embodiments 30-32, wherein the backbone of the sgRNA comprises a total length of at most 85, 90, 95, 100, 105, 110, 115, or 120 nucleotides.
35. The gRNA of any one of embodiments 30-32, wherein the backbone of the sgRNA comprises a total length of 86 to 98 nucleotides.
36. The gRNA of any one of embodiments 30-32, wherein the backbone of the sgRNA comprises a total length of 94 nucleotides.
37. The gRNA of any one of embodiments 30-32, wherein the backbone of the sgRNA has a nucleotide sequence having at least 80% sequence identity to any one of SEQ ID NOs: 124-134.
38. The gRNA of embodiment 37, wherein the backbone of the sgRNA has a nucleotide sequence having at least 90% sequence identity to any one of SEQ ID NOs: 124-134.
39. The gRNA of embodiment 37, wherein the backbone of the sgRNA has a nucleotide sequence having at least 95% sequence identity to any one of SEQ ID NOs: 124-134.
40. The gRNA of embodiment 37, wherein the backbone of the sgRNA has the nucleotide sequence set forth as any one of SEQ ID NOs: 124-134.
41. The gRNA of any one of embodiments 1-8, wherein the gRNA comprises a first stem loop formed by hybridization of the crRNA repeat and the anti-repeat, wherein the first stem loop comprises a first stem and a second stem, and wherein the first stem of the first stem loop comprises a total length of at least 3, 4, 5, 6, 7, 8, 9, 10, or 11 base pairs (bp).
42. The gRNA of any one of embodiments 1-8, wherein the gRNA comprises a first stem loop formed by hybridization of the crRNA repeat and the anti-repeat, wherein the first stem loop comprises a first stem and a second stem, and wherein the first stem of the first stem loop comprises a total length of at most 3, 4, 5, 6, 7, 8, 9, 10, or 11 bp.
43. The gRNA of embodiment 41 or 42, wherein the first stem of the first stem loop comprises a total length of 6 bp.
44. The gRNA of embodiment 41 or 42, wherein the first stem of the first stem loop comprises a total length of 3 bp.
45. The gRNA of any one of embodiments 1-8, wherein the tail of the tracrRNA comprises a total length of at least 1, 2, 3, 4, 5, 6, or 7 nucleotides.
46. The gRNA of any one of embodiments 1-8, wherein the tail of the tracrRNA comprises a total length of at most 1, 2, 3, 4, 5, 6, or 7 nucleotides.
47. The gRNA of embodiment 45 or 46, wherein the tail of the tracrRNA comprises a total length of 3 nucleotides.
48. The gRNA of embodiment 45 or 46, wherein the tail of the tracrRNA comprises a total length of 1 nucleotide.
49. The gRNA of embodiment 41 or 42, wherein the gRNA further comprises a second stem loop most proximal to the tail, wherein the second stem loop comprises a first stem and a second stem.
50. The gRNA of embodiment 49, wherein the first stem of the second stem loop comprises a total length of at least 1, 2, 3, 4, 5, or 6 bp.
51. The gRNA of embodiment 49, wherein the first stem of the second stem loop comprises a total length of at most 1, 2, 3, 4, 5, or 6 bp.
52. The gRNA of embodiment 50 or 51, wherein the first stem of the second stem loop comprises a total length of 5 bp.
53. The gRNA of any one of embodiments 49-52, wherein the first stem of the first stem loop comprises a total length of 6 bp, the tail of the tracrRNA comprises a total length of 3 nucleotides, and the first stem of the second stem loop comprises a total length of 5 bp.
54. The gRNA of any one of embodiments 1-8, wherein the gRNA is a dual guide RNA (dgRNA).
55. The gRNA of embodiment 54, wherein the crRNA repeat of the dgRNA comprises a total length of at least 10, 11, 12, 13, 14, 15, 16, 17, 18, 19, 20, or 21 nucleotides.
56. The gRNA of embodiment 54, wherein the crRNA repeat of the dgRNA comprises a total length of at most 10, 11, 12, 13, 14, 15, 16, 17, 18, 19, 20, or 21 nucleotides.
57. The gRNA of embodiment 55 or 56, wherein the crRNA repeat of the dgRNA comprises a total length of 13 nucleotides.
58. The gRNA of embodiment 55 or 56, wherein the crRNA repeat of the dgRNA comprises a total length of 16 nucleotides.
59. The gRNA of embodiment 55 or 56, wherein the crRNA repeat of the dgRNA comprises a total length of 21 nucleotides.
60. The gRNA of embodiment 54, wherein the tracrRNA of the dgRNA comprises a total length of at least 65, 70, 75, 80, or 85 nucleotides.
61. The gRNA of embodiment 54, wherein the tracrRNA of the dgRNA comprises a total length of at most 65, 70, 75, 80, or 85 nucleotides.
62. The gRNA of embodiment 60 or 61, wherein the tracrRNA of the dgRNA comprises a total length of 74 nucleotides.
63. The gRNA of embodiment 60 or 61, wherein the tracrRNA of the dgRNA comprises a total length of 77 nucleotides.
64. The gRNA of any one of embodiments 1-63, wherein the gRNA comprises a total length of at least 85, 90, 95, 100, 105, 110, 115, 120, 125, 130, or 135 nucleotides.
65. The gRNA of any one of embodiments 1-63, wherein the gRNA comprises a total length of at most 85, 90, 95, 100, 105, 110, 115, 120, 125, 130, or 135 nucleotides.
66. The gRNA of any one of embodiments 1-63, wherein the gRNA comprises a total length of 106 to 135 nucleotides.
67. The gRNA of embodiment 66, wherein the gRNA comprises a total length of 117 to 119 nucleotides.
68. The gRNA of any one of embodiments 1-67, wherein the gRNA is capable of targeting a bound RNA-guided nuclease (RGN) polypeptide to the target sequence.
69. The gRNA of embodiment 68, wherein the RGN polypeptide is capable of recognizing a consensus protospacer adjacent motif (PAM) having the nucleotide sequence set forth as NNNNCC or NNRNCC.
70. The gRNA of embodiment 69, wherein the RGN polypeptide is capable of recognizing a full PAM having the nucleotide sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG.
71. The gRNA of any one of embodiments 68-70, wherein the RGN polypeptide has an amino acid sequence having at least 80% sequence identity to SEQ ID NO: 105; and wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 1 to 5 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 1 to 5 nucleotides.
72. The gRNA of embodiment 71, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 5 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 5 nucleotides.
73. The gRNA of embodiment 71, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 4 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 4 nucleotides.
74. The gRNA of embodiment 71, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 3 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 3 nucleotides.
75. The gRNA of embodiment 71, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 2 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 2 nucleotides.
76. The gRNA of embodiment 71, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 1 nucleotide; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 1 nucleotide.
77. The gRNA of embodiment 71, wherein the spacer has the nucleotide sequence set forth as SEQ ID NO: 7 or 9.
78. The gRNA of any one of embodiments 71-77, wherein the RGN polypeptide has an amino acid sequence having at least 90% sequence identity to SEQ ID NO: 105.
79. The gRNA of any one of embodiments 71-77, wherein the RGN polypeptide has an amino acid sequence having at least 95% sequence identity to SEQ ID NO: 105.
80. The gRNA of any one of embodiments 71-77, wherein the RGN polypeptide has the amino acid sequence set forth as SEQ ID NO: 105.
81. The gRNA of any one of embodiments 71-80, wherein the RGN polypeptide has an amino acid sequence having at least 80% sequence identity to SEQ ID NO: 333.
82. The gRNA of embodiment 81, wherein the RGN polypeptide has an amino acid sequence having at least 90% sequence identity to SEQ ID NO: 333.
83. The gRNA of embodiment 81, wherein the RGN polypeptide has an amino acid sequence having at least 95% sequence identity to SEQ ID NO: 333.
84. The gRNA of embodiment 81, wherein the RGN polypeptide has the amino acid sequence set forth as SEQ ID NO: 333.
85. The gRNA of any one of embodiments 68-84, wherein the gRNA has a nucleotide sequence having at least 80% sequence identity to any one of SEQ ID NOs: 198-200, 202-213, 215-233, 235-241, and 243-259.
86. The gRNA of embodiment 85, wherein the gRNA has a nucleotide sequence having at least 90% sequence identity to any one of SEQ ID NOs: 198-200, 202-213, 215-233, 235-241, and 243-259.
87. The gRNA of embodiment 85, wherein the gRNA has a nucleotide sequence having at least 95% sequence identity to any one of SEQ ID NOs: 198-200, 202-213, 215-233, 235-241, and 243-259.
88. The gRNA of embodiment 85, wherein the gRNA has the nucleotide sequence set forth as any one of SEQ ID NOs: 198-200, 202-213, 215-233, 235-241, and 243-259.
89. The gRNA of embodiment 88, wherein the gRNA has a nucleotide sequence set forth as SEQ ID NO: 204 or 205.
90. The gRNA of embodiment 68 or 69, wherein the RGN polypeptide has an amino acid sequence having at least 80% sequence identity to SEQ ID NO: 327 or 330.
91. The gRNA of embodiment 90, wherein the RGN polypeptide has an amino acid sequence having at least 90% sequence identity to SEQ ID NO: 327 or 330.
92. The gRNA of embodiment 90, wherein the RGN polypeptide has an amino acid sequence having at least 95% sequence identity to SEQ ID NO: 327 or 330.
93. The gRNA of embodiment 90, wherein the RGN polypeptide has an amino acid sequence set forth as SEQ ID NO: 327 or 330.
94. The gRNA of embodiment 70, wherein the RGN polypeptide is capable of recognizing a full PAM having the nucleotide sequence set forth as GGGCCCAG.
95. The gRNA of embodiment 94, wherein the RGN polypeptide has an amino acid sequence having at least 80% sequence identity to SEQ ID NO: 324.
96. The gRNA of embodiment 95, wherein the RGN polypeptide has an amino acid sequence having at least 90% sequence identity to SEQ ID NO: 324.
97. The gRNA of embodiment 95, wherein the RGN polypeptide has an amino acid sequence having at least 95% sequence identity to SEQ ID NO: 324.
98. The gRNA of embodiment 95, wherein the RGN polypeptide has the amino acid sequence set forth as SEQ ID NO: 324.
99. The gRNA of embodiment 70, wherein the RGN polypeptide is capable of recognizing a full PAM having the nucleotide sequence set forth as CAGGCCAA.
100. The gRNA of embodiment 99, wherein the RGN polypeptide has an amino acid sequence having at least 80% sequence identity to SEQ ID NO: 404.
101. The gRNA of embodiment 100, wherein the RGN polypeptide has an amino acid sequence having at least 90% sequence identity to SEQ ID NO: 404.
102. The gRNA of embodiment 100, wherein the RGN polypeptide has an amino acid sequence having at least 95% sequence identity to SEQ ID NO: 404.
103. The gRNA of embodiment 100, wherein the RGN polypeptide has the amino acid sequence set forth as SEQ ID NO: 404.
104. The gRNA of any one of embodiments 1-103, wherein the gRNA comprises at least one chemical modification.
105. The gRNA of embodiment 104, wherein the at least one chemical modification comprises a bridged nucleic acid (BNA) modification; 2′-O-methyl(2′-O-Me) modification; 2′-O-methoxy-ethyl(2′MOE) modification; 2′-fluoro (2′-F) modification; 2′F-4′Cα-OMe modification; 2′,4′-di-Cα-OMe modification; 2′-O-methyl 3′phosphorothioate (MS) modification; 2′-O-methyl 3′thiophosphonoacetate (MSP) modification; 2′-O-methyl 3′phosphonoacetate (MP) modification phosphorothioate (PS) modification; or a combination thereof.
106. The gRNA of embodiment 105, wherein the at least one chemical modification comprises MS modifications at the 3 terminal nucleotides at the 5′ region and at the 3 terminal nucleotides at the 3′ region of the gRNA.
107. The gRNA of embodiment 106, wherein the crRNA repeat has the nucleotide sequence set forth as any one of SEQ ID NOs: 430, 432-435, 583, 585, and 587.
108. The gRNA of embodiment 106 or 107, wherein the crRNA has the nucleotide sequence set forth as any one of SEQ ID NOs: 459-520.
109. The gRNA of any one of embodiments 106-108, wherein the tracrRNA has the nucleotide sequence set forth as any one of SEQ ID NOs: 431, 437-446, 584, 586, and 588.
110. The gRNA of any one of embodiments 106-109, wherein the gRNA has the nucleotide sequence set forth as any one of SEQ ID NOs: 521-523, 525-536, 538-556, 558-564, and 566-582.
111. The gRNA of embodiment 105, wherein the BNA comprises a 2′,4′ BNA modification.
112. The gRNA of embodiment 111, wherein the 2′,4′ BNA modification is selected from the group consisting of: locked nucleic acid (LNA) modification, BNA^{NC}[N-Me] modification, 2′-O,4′-C-ethylene bridged nucleic acid (2′,4′-ENA) modification, and S-constrained ethyl (cEt) modification.
113. The gRNA of embodiment 112, wherein the 2′,4′ BNA is a LNA modification.
114. The gRNA of embodiment 112, wherein the 2′,4′ BNA is a cEt modification.
115. The gRNA of embodiment 105, wherein the at least one chemical modification comprises a BNA modification, 2′-O-Me modification, PS modification, or a combination thereof.
116. The gRNA of any one of embodiments 1-115, wherein the gRNA further comprises an extension comprising an edit template for reverse transcriptase (RT) editing.
117. A guide RNA (gRNA) comprising a CRISPR RNA (crRNA) and a trans-activating CRISPR RNA (tracrRNA), wherein the crRNA comprises
(i) a crRNA repeat; and
(ii) a spacer, wherein the tracrRNA comprises:
(iii) an anti-repeat; and
(iv) a tail,
   wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103, or has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 to 5 nucleotides.
118. The gRNA of embodiment 117, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 5 nucleotides.
119. The gRNA of embodiment 117, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 4 nucleotides.
120. The gRNA of embodiment 117, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 3 nucleotides.
121. The gRNA of embodiment 117, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 2 nucleotides.
122. The gRNA of embodiment 117, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 nucleotide.
123. The gRNA of embodiment 117, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103.
124. The gRNA of any one of embodiments 117-123, wherein the spacer is capable of hybridizing to a target sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, and 104.
125. The gRNA of any one of embodiments 117-124, wherein the crRNA repeat has the nucleotide sequence set forth as SEQ ID NO: 106 or a nucleotide sequence that differs in length and/or sequence from SEQ ID NO: 106 by 1 to 8 nucleotides.
126. The gRNA of embodiment 125, wherein the crRNA repeat has a nucleotide sequence that differs in length and/or sequence from SEQ ID NO: 106 by 8 nucleotides.
127. The gRNA of embodiment 125, wherein the crRNA repeat has a nucleotide sequence that differs in length and/or sequence from SEQ ID NO: 106 by 7 nucleotides.
128. The gRNA of embodiment 125, wherein the crRNA repeat has a nucleotide sequence that differs in length and/or sequence from SEQ ID NO: 106 by 6 nucleotides.
129. The gRNA of embodiment 125, wherein the crRNA repeat has a nucleotide sequence that differs in length and/or sequence from SEQ ID NO: 106 by 5 nucleotides.
130. The gRNA of embodiment 125, wherein the crRNA repeat has a nucleotide sequence that differs in length and/or sequence from SEQ ID NO: 106 by 4 nucleotides.
131. The gRNA of embodiment 125, wherein the crRNA repeat has a nucleotide sequence that differs in length and/or sequence from SEQ ID NO: 106 by 3 nucleotides.
132. The gRNA of embodiment 125, wherein the crRNA repeat has a nucleotide sequence that differs in length and/or sequence from SEQ ID NO: 106 by 2 nucleotides.
133. The gRNA of embodiment 125, wherein the crRNA repeat has a nucleotide sequence that differs in length and/or sequence from SEQ ID NO: 106 by 1 nucleotide.
134. The gRNA of embodiment 125, wherein the crRNA repeat has the nucleotide sequence set forth as any one of SEQ ID NOs: 106, 109-112, 328, 331, and 334.
135. The gRNA of any one of embodiments 117-125, wherein the crRNA has a nucleotide sequence having at least 80% sequence identity to SEQ ID NOs: 136-197.
136. The gRNA of embodiment 135, wherein the crRNA has a nucleotide sequence having at least 90% sequence identity to SEQ ID NOs: 136-197.
137. The gRNA of embodiment 135, wherein the crRNA has a nucleotide sequence having at least 95% sequence identity to SEQ ID NOs: 136-197.
138. The gRNA of embodiment 135, wherein the crRNA has the nucleotide sequence set forth as any one of SEQ ID NOs: 136-197.
139. The gRNA of any one of embodiments 117-125, wherein the tracrRNA has a nucleotide sequence having at least 80% sequence identity to SEQ ID NO: 107.
140. The gRNA of embodiment 139, wherein the tracrRNA has a nucleotide sequence having at least 90% sequence identity to SEQ ID NO: 107.
141. The gRNA of embodiment 139, wherein the tracrRNA has a nucleotide sequence having at least 95% sequence identity to SEQ ID NO: 107.
142. The gRNA of any one of embodiments 117-125, wherein the tracrRNA has a nucleotide sequence that differs in length from SEQ ID NO: 107 by 1 to 16 nucleotides.
143. The gRNA of embodiment 142, wherein the tracrRNA has a nucleotide sequence that is 8 nucleotides shorter than SEQ ID NO: 107.
144. The gRNA of embodiment 142, wherein the tracrRNA has a nucleotide sequence that is 11 nucleotides shorter than SEQ ID NO: 107.
145. The gRNA of any one of embodiments 139-144, wherein the tracrRNA has the nucleotide sequence set forth as any one of SEQ ID NOs: 107, 114-123, 329, 332, and 335.
146. The gRNA of any one of embodiments 117-124, wherein the gRNA is a single guide RNA (sgRNA) comprising the crRNA and the tracrRNA linked by a linker, wherein the sgRNA comprises a backbone and the spacer, and wherein the backbone of the sgRNA comprises the crRNA repeat, the linker, and the tracrRNA.
147. The gRNA of embodiment 146, wherein the linker has a nucleotide sequence set forth as AAAG, GAAA, ACUU, or CAAAGG.
148. The gRNA of embodiment 147, wherein the linker has a nucleotide sequence set forth as AAAG.
149. The gRNA of any one of embodiments 146-148, wherein the backbone of the sgRNA comprises a total length of at least 85, 90, 95, 100, 105, 110, 115, or 120 nucleotides.
150. The gRNA of any one of embodiments 146-148, wherein the backbone of the sgRNA comprises a total length of at most 85, 90, 95, 100, 105, 110, 115, or 120 nucleotides.
151. The gRNA of any one of embodiments 146-148, wherein the backbone of the sgRNA comprises a total length of 86 to 98 nucleotides.
152. The gRNA of any one of embodiments 146-148, wherein the backbone of the sgRNA comprises a total length of 94 nucleotides.
153. The gRNA of any one of embodiments 146-148, wherein the backbone of the sgRNA has a nucleotide sequence having at least 80% sequence identity to any one of SEQ ID NOs: 124-134.
154. The gRNA of embodiment 153, wherein the backbone of the sgRNA has a nucleotide sequence having at least 90% sequence identity to any one of SEQ ID NOs: 124-134.
155. The gRNA of embodiment 153, wherein the backbone of the sgRNA has a nucleotide sequence having at least 95% sequence identity to any one of SEQ ID NOs: 124-134.
156. The gRNA of embodiment 153, wherein the backbone of the sgRNA has the nucleotide sequence set forth as any one of SEQ ID NOs: 124-134.
157. The gRNA of any one of embodiments 117-124, wherein the gRNA comprises a first stem loop formed by hybridization of the crRNA repeat and the anti-repeat, wherein the first stem loop comprises a first stem and a second stem, and wherein the first stem of the first stem loop comprises a total length of at least 3, 4, 5, 6, 7, 8, 9, 10, or 11 base pairs (bp).
158. The gRNA of any one of embodiments 117-124, wherein the gRNA comprises a first stem loop formed by hybridization of the crRNA repeat and the anti-repeat, wherein the first stem loop comprises a first stem and a second stem, and wherein the first stem of the first stem loop comprises a total length of at most 3, 4, 5, 6, 7, 8, 9, 10, or 11 bp.
159. The gRNA of embodiment 157 or 158, wherein the first stem of the first stem loop comprises a total length of 6 bp.
160. The gRNA of embodiment 157 or 158, wherein the first stem of the first stem loop comprises a total length of 3 bp.
161. The gRNA of any one of embodiments 117-124, wherein the tail of the tracrRNA comprises a total length of at least 1, 2, 3, 4, 5, 6, or 7 nucleotides.
162. The gRNA of any one of embodiments 117-124, wherein the tail of the tracrRNA comprises a total length of at most 1, 2, 3, 4, 5, 6, or 7 nucleotides.
163. The gRNA of embodiment 161 or 162, wherein the tail of the tracrRNA comprises a total length of 3 nucleotides.
164. The gRNA of embodiment 161 or 162, wherein the tail of the tracrRNA comprises a total length of 1 nucleotide.
165. The gRNA of embodiment 157 or 158, wherein the gRNA further comprises a second stem loop most proximal to the tail, wherein the second stem loop comprises a first stem and a second stem.
166. The gRNA of embodiment 165, wherein the first stem of the second stem loop comprises a total length of at least 1, 2, 3, 4, 5, or 6 bp.
167. The gRNA of embodiment 165, wherein the first stem of the second stem loop comprises a total length of at most 1, 2, 3, 4, 5, or 6 bp.
168. The gRNA of embodiment 166 or 167, wherein the first stem of the second stem loop comprises a total length of 5 bp.
169. The gRNA of any one of embodiments 165-168, wherein the first stem of the first stem loop comprises a total length of 6 bp, the tail of the tracrRNA comprises a total length of 3 nucleotides, and the first stem of the second stem loop comprises a total length of 5 bp.
170. The gRNA of any one of embodiments 117-124, wherein the gRNA is a dual guide RNA (dgRNA).
171. The gRNA of embodiment 170, wherein the crRNA repeat of the dgRNA comprises a total length of at least 10, 11, 12, 13, 14, 15, 16, 17, 18, 19, 20, or 21 nucleotides.
172. The gRNA of embodiment 170, wherein the crRNA repeat of the dgRNA comprises a total length of at most 10, 11, 12, 13, 14, 15, 16, 17, 18, 19, 20, or 21 nucleotides.
173. The gRNA of embodiment 171 or 172, wherein the crRNA repeat of the dgRNA comprises a total length of 13 nucleotides.
174. The gRNA of embodiment 171 or 172, wherein the crRNA repeat of the dgRNA comprises a total length of 16 nucleotides.
175. The gRNA of embodiment 171 or 172, wherein the crRNA repeat of the dgRNA comprises a total length of 21 nucleotides.
176. The gRNA of embodiment 170, wherein the tracrRNA of the dgRNA comprises a total length of at least 65, 70, 75, 80, or 85 nucleotides.
177. The gRNA of embodiment 170, wherein the tracrRNA of the dgRNA comprises a total length of at most 65, 70, 75, 80, or 85 nucleotides.
178. The gRNA of embodiment 176 or 177, wherein the tracrRNA of the dgRNA comprises a total length of 74 nucleotides.
179. The gRNA of embodiment 176 or 177, wherein the tracrRNA of the dgRNA comprises a total length of 77 nucleotides.
180. The gRNA of any one of embodiments 117-179, wherein the gRNA comprises a total length of at least 85, 90, 95, 100, 105, 110, 115, 120, 125, 130, or 135 nucleotides.
181. The gRNA of any one of embodiments 117-179, wherein the gRNA comprises a total length of at most 85, 90, 95, 100, 105, 110, 115, 120, 125, 130, or 135 nucleotides.
182. The gRNA of any one of embodiments 117-179, wherein the gRNA comprises a total length of 106 to 135 nucleotides.
183. The gRNA of embodiment 182, wherein the gRNA comprises a total length of 117 to 119 nucleotides.
184. The gRNA of any one of embodiments 117-183, wherein the gRNA is capable of targeting a bound RNA-guided nuclease (RGN) polypeptide to the target sequence.
185. The gRNA of embodiment 184, wherein the RGN polypeptide is capable of recognizing a consensus protospacer adjacent motif (PAM) having the nucleotide sequence set forth as NNNNCC or NNRNCC.
186. The gRNA of embodiment 185, wherein the RGN polypeptide is capable of recognizing a full PAM having the nucleotide sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG.
187. The gRNA of any one of embodiments 184-186, wherein the RGN polypeptide has an amino acid sequence having at least 80% sequence identity to SEQ ID NO: 105; and wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 1 to 5 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 1 to 5 nucleotides.
188. The gRNA of embodiment 187, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 5 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 5 nucleotides.
189. The gRNA of embodiment 187, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 4 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 4 nucleotides.
190. The gRNA of embodiment 187, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 3 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 3 nucleotides.
191. The gRNA of embodiment 187, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 2 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 2 nucleotides.
192. The gRNA of embodiment 187, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 1 nucleotide; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 1 nucleotide.
193. The gRNA of embodiment 187, wherein the spacer has the nucleotide sequence set forth as SEQ ID NO: 7 or 9.
194. The gRNA of any one of embodiments 187-193, wherein the RGN polypeptide has an amino acid sequence having at least 90% sequence identity to SEQ ID NO: 105.
195. The gRNA of any one of embodiments 187-193, wherein the RGN polypeptide has an amino acid sequence having at least 95% sequence identity to SEQ ID NO: 105.
196. The gRNA of any one of embodiments 187-193, wherein the RGN polypeptide has the amino acid sequence set forth as SEQ ID NO: 105.
197. The gRNA of any one of embodiments 184-186, wherein the RGN polypeptide has an amino acid sequence having at least 80% sequence identity to SEQ ID NO: 333.
198. The gRNA of embodiment 197, wherein the RGN polypeptide has an amino acid sequence having at least 90% sequence identity to SEQ ID NO: 333.
199. The gRNA of embodiment 197, wherein the RGN polypeptide has an amino acid sequence having at least 95% sequence identity to SEQ ID NO: 333.
200. The gRNA of embodiment 197, wherein the RGN polypeptide has the amino acid sequence set forth as SEQ ID NO: 333.
201. The gRNA of any one of embodiments 184-200, wherein the gRNA has a nucleotide sequence having at least 80% sequence identity to any one of SEQ ID NOs: 198-200, 202-213, 215-233, 235-241, and 243-259.
202. The gRNA of embodiment 201, wherein the gRNA has a nucleotide sequence having at least 90% sequence identity to any one of SEQ ID NOs: 198-200, 202-213, 215-233, 235-241, and 243-259.
203. The gRNA of embodiment 201, wherein the gRNA has a nucleotide sequence having at least 95% sequence identity to any one of SEQ ID NOs: 198-200, 202-213, 215-233, 235-241, and 243-259.
204. The gRNA of embodiment 201, wherein the gRNA has the nucleotide sequence set forth as any one of SEQ ID NOs: 198-200, 202-213, 215-233, 235-241, and 243-259.
205. The gRNA of embodiment 204, wherein the gRNA has a nucleotide sequence set forth as SEQ ID NO: 204 or 205.
206. The gRNA of embodiment 184 or 185, wherein the RGN polypeptide has an amino acid sequence having at least 80% sequence identity to SEQ ID NO: 327 or 330.
207. The gRNA of embodiment 206, wherein the RGN polypeptide has an amino acid sequence having at least 90% sequence identity to SEQ ID NO: 327 or 330.
208. The gRNA of embodiment 206, wherein the RGN polypeptide has an amino acid sequence having at least 95% sequence identity to SEQ ID NO: 327 or 330.
209. The gRNA of embodiment 206, wherein the RGN polypeptide has an amino acid sequence set forth as SEQ ID NO: 327 or 330.
210. The gRNA of embodiment 186, wherein the RGN polypeptide is capable of recognizing a full PAM having the nucleotide sequence set forth as GGGCCCAG.
211. The gRNA of embodiment 2010, wherein the RGN polypeptide has an amino acid sequence having at least 80% sequence identity to SEQ ID NO: 324.
212. The gRNA of embodiment 211, wherein the RGN polypeptide has an amino acid sequence having at least 90% sequence identity to SEQ ID NO: 324.
213. The gRNA of embodiment 211, wherein the RGN polypeptide has an amino acid sequence having at least 95% sequence identity to SEQ ID NO: 324.
214. The gRNA of embodiment 211, wherein the RGN polypeptide has the amino acid sequence set forth as SEQ ID NO: 324.
215. The gRNA of embodiment 186, wherein the RGN polypeptide is capable of recognizing a full PAM having the nucleotide sequence set forth as CAGGCCAA.
216. The gRNA of embodiment 215, wherein the RGN polypeptide has an amino acid sequence having at least 80% sequence identity to SEQ ID NO: 404.
217. The gRNA of embodiment 216, wherein the RGN polypeptide has an amino acid sequence having at least 90% sequence identity to SEQ ID NO: 404.
218. The gRNA of embodiment 216, wherein the RGN polypeptide has an amino acid sequence having at least 95% sequence identity to SEQ ID NO: 404.
219. The gRNA of embodiment 216, wherein the RGN polypeptide has the amino acid sequence set forth as SEQ ID NO: 404.
220. The gRNA of any one of embodiments 117-219, wherein the gRNA comprises at least one chemical modification.
221. The gRNA of embodiment 220, wherein the at least one chemical modification comprises a bridged nucleic acid (BNA) modification; 2′-O-methyl(2′-O-Me) modification; 2′-O-methoxy-ethyl(2′MOE) modification; 2′-fluoro (2′-F) modification; 2′F-4′Cα-OMe modification; 2′,4′-di-Cα-OMe modification; 2′-O-methyl 3′phosphorothioate (MS) modification; 2′-O-methyl 3′thiophosphonoacetate (MSP) modification; 2′-O-methyl 3′phosphonoacetate (MP) modification phosphorothioate (PS) modification; or a combination thereof.
222. The gRNA of embodiment 221, wherein the at least one chemical modification comprises MS modifications at the 3 terminal nucleotides at the 5′ region and at the 3 terminal nucleotides at the 3′ region of the gRNA.
223. The gRNA of embodiment 222, wherein the crRNA repeat has the nucleotide sequence set forth as any one of SEQ ID NOs: 430, 432-435, 583, 585, and 587.
224. The gRNA of embodiment 222 or 223, wherein the crRNA has the nucleotide sequence set forth as any one of SEQ ID NOs: 459-520.
225. The gRNA of any one of embodiments 222-224, wherein the tracrRNA has the nucleotide sequence set forth as any one of SEQ ID NOs: 431, 437-446, 584, 586, and 588.
226. The gRNA of any one of embodiments 222-225, wherein the gRNA has the nucleotide sequence set forth as any one of SEQ ID NOs: 521-523, 525-536, 538-556, 558-564, and 566-582.
227. The gRNA of embodiment 221, wherein the BNA comprises a 2′,4′ BNA modification.
228. The gRNA of embodiment 227, wherein the 2′,4′ BNA modification is selected from the group consisting of: locked nucleic acid (LNA) modification, BNA^{NC}[N-Me] modification, 2′-O,4′-C-ethylene bridged nucleic acid (2′,4′-ENA) modification, and S-constrained ethyl (cEt) modification.
229. The gRNA of embodiment 228, wherein the 2′,4′ BNA is a LNA modification.
230. The gRNA of embodiment 228, wherein the 2′,4′ BNA is a cEt modification.
231. The gRNA of embodiment 221, wherein the at least one chemical modification comprises a BNA modification, 2′-O-Me modification, PS modification, or a combination thereof.
232. The gRNA of any one of embodiments 117-231, wherein the gRNA further comprises an extension comprising an edit template for reverse transcriptase (RT) editing.
233. A nucleic acid molecule comprising a CRISPR RNA (crRNA) or encoding a crRNA, wherein the crRNA comprises a spacer and a crRNA repeat, wherein the spacer is capable of hybridizing to a target sequence in a T cell receptor alpha chain constant (TRAC) gene, and wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, and 104.
234. The nucleic acid molecule of embodiment 233, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 to 5 nucleotides.
235. The nucleic acid molecule of embodiment 234, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 5 nucleotides.
236. The nucleic acid molecule of embodiment 234, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 4 nucleotides.
237. The nucleic acid molecule of embodiment 234, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 3 nucleotides.
238. The nucleic acid molecule of embodiment 234, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 2 nucleotides.
239. The nucleic acid molecule of embodiment 234, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 nucleotide.
240. The nucleic acid molecule of embodiment 233, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103.
241. The nucleic acid molecule of any one of embodiments 233-240, wherein the crRNA repeat has the nucleotide sequence set forth as SEQ ID NO: 106 or a nucleotide sequence that differs in length and/or sequence from SEQ ID NO: 106 by 1 to 8 nucleotides.
242. The nucleic acid molecule of embodiment 241, wherein the crRNA repeat has a nucleotide sequence that differs in length and/or sequence from SEQ ID NO: 106 by 8 nucleotides.
243. The nucleic acid molecule of embodiment 241, wherein the crRNA repeat has a nucleotide sequence that differs in length and/or sequence from SEQ ID NO: 106 by 7 nucleotides.
244. The nucleic acid molecule of embodiment 241, wherein the crRNA repeat has a nucleotide sequence that differs in length and/or sequence from SEQ ID NO: 106 by 6 nucleotides.
245. The nucleic acid molecule of embodiment 241, wherein the crRNA repeat has a nucleotide sequence that differs in length and/or sequence from SEQ ID NO: 106 by 5 nucleotides.
246. The nucleic acid molecule of embodiment 241, wherein the crRNA repeat has a nucleotide sequence that differs in length and/or sequence from SEQ ID NO: 106 by 4 nucleotides.
247. The nucleic acid molecule of embodiment 241, wherein the crRNA repeat has a nucleotide sequence that differs in length and/or sequence from SEQ ID NO: 106 by 3 nucleotides.
248. The nucleic acid molecule of embodiment 241, wherein the crRNA repeat has a nucleotide sequence that differs in length and/or sequence from SEQ ID NO: 106 by 2 nucleotides.
249. The nucleic acid molecule of embodiment 241, wherein the crRNA repeat has a nucleotide sequence that differs in length and/or sequence from SEQ ID NO: 106 by 1 nucleotide.
250. The nucleic acid molecule of embodiment 241, wherein the crRNA repeat has the nucleotide sequence set forth as any one of SEQ ID NOs: 106, 109-112, 328, 331, and 334.
251. The nucleic acid molecule of any one of embodiments 233-241, wherein the crRNA has a nucleotide sequence having at least 80% sequence identity to SEQ ID NOs: 136-197.
252. The nucleic acid molecule of embodiment 251, wherein the crRNA has a nucleotide sequence having at least 90% sequence identity to SEQ ID NOs: 136-197.
253. The nucleic acid molecule of embodiment 251, wherein the crRNA has a nucleotide sequence having at least 95% sequence identity to SEQ ID NOs: 136-197.
254. The nucleic acid molecule of embodiment 251, wherein the crRNA has the nucleotide sequence set forth as any one of SEQ ID NOs: 136-197.
255. The nucleic acid molecule of any one of embodiments 233-254, wherein the crRNA is capable of binding a trans-activating CRISPR RNA (tracrRNA) to form a guide RNA (gRNA), wherein the tracrRNA comprises an anti-repeat and a tail.
256. The nucleic acid molecule of embodiment 255, wherein the tracrRNA has a nucleotide sequence having at least 80% sequence identity to SEQ ID NO: 107.
257. The nucleic acid molecule of embodiment 256, wherein the tracrRNA has a nucleotide sequence having at least 90% sequence identity to SEQ ID NO: 107.
258. The nucleic acid molecule of embodiment 256, wherein the tracrRNA has a nucleotide sequence having at least 95% sequence identity to SEQ ID NO: 107.
259. The nucleic acid molecule of embodiment 256, wherein the tracrRNA has a nucleotide sequence that differs in length from SEQ ID NO: 107 by 1 to 16 nucleotides.
260. The nucleic acid molecule of embodiment 259, wherein the tracrRNA has a nucleotide sequence that is 8 nucleotides shorter than SEQ ID NO: 107.
261. The nucleic acid molecule of embodiment 259, wherein the tracrRNA has a nucleotide sequence that is 11 nucleotides shorter than SEQ ID NO: 107.
262. The nucleic acid molecule of any one of embodiments 256-261, wherein the tracrRNA has the nucleotide sequence set forth as any one of SEQ ID NOs: 107, 114-123, 329, 332, and 335.
263. The nucleic acid molecule of embodiment 255, wherein the gRNA is a single guide RNA (sgRNA) comprising the crRNA and the tracrRNA linked by a linker, wherein the sgRNA comprises a backbone and the spacer, and wherein the backbone of the sgRNA comprises the crRNA repeat, the linker, and the tracrRNA.
264. The nucleic acid molecule of embodiment 263, wherein the backbone of the sgRNA comprises a total length of 86 to 98 nucleotides.
265. The nucleic acid molecule of embodiment 263, wherein the backbone of the sgRNA comprises a total length of 94 nucleotides.
266. The nucleic acid molecule of embodiment 263, wherein the backbone of the sgRNA has a nucleotide sequence having at least 80% sequence identity to any one of SEQ ID NOs: 124-134.
267. The nucleic acid molecule of embodiment 266, wherein the backbone of the sgRNA has a nucleotide sequence having at least 90% sequence identity to any one of SEQ ID NOs: 124-134.
268. The nucleic acid molecule of embodiment 266, wherein the backbone of the sgRNA has a nucleotide sequence having at least 95% sequence identity to any one of SEQ ID NOs: 124-134.
269. The nucleic acid molecule of embodiment 266, wherein the backbone of the sgRNA has the nucleotide sequence set forth as any one of SEQ ID NOs: 124-134.
270. The nucleic acid molecule of any one of embodiments 255-269, wherein the gRNA comprises a first stem loop comprising a first stem and a second stem formed by hybridization of the crRNA repeat and the anti-repeat, and wherein the first stem of the first stem loop comprises a total length of at least 3, 4, 5, 6, 7, 8, 9, 10, or 11 bp.
271. The nucleic acid molecule of any one of embodiments 255-269, wherein the gRNA comprises a first stem loop comprising a first stem and a second stem formed by hybridization of the crRNA repeat and the anti-repeat, and wherein the first stem of the first stem loop comprises a total length of at most 3, 4, 5, 6, 7, 8, 9, 10, or 11 bp.
272. The nucleic acid molecule of embodiment 270 or 271, wherein the first stem of the first stem loop comprises a total length of 6 bp.
273. The nucleic acid molecule of embodiment 270 or 271, wherein the first stem of the first stem loop comprises a total length of 3 bp.
274. The nucleic acid molecule of any one of embodiments 255-273, wherein the tail of the tracrRNA comprises a total length of at least 1, 2, 3, 4, 5, 6, or 7 nucleotides.
275. The nucleic acid molecule of any one of embodiments 255-273, wherein the tail of the tracrRNA comprises a total length of at most 1, 2, 3, 4, 5, 6, or 7 nucleotides.
276. The nucleic acid molecule of embodiment 274 or 275, wherein the tail of the tracrRNA comprises a total length of 3 nucleotides.
277. The nucleic acid molecule of embodiment 274 or 275, wherein the tail of the tracrRNA comprises a total length of 1 nucleotide.
278. The nucleic acid molecule of any one of embodiments 270-277, wherein the gRNA further comprises a second stem loop most proximal to the tail, wherein the second stem loop comprises a first stem and a second stem.
279. The nucleic acid molecule of embodiment 278, wherein the first stem of the second stem loop comprises a total length of at least 1, 2, 3, 4, 5, or 6 bp.
280. The nucleic acid molecule of embodiment 278, wherein the first stem of the second stem loop comprises a total length of at most 1, 2, 3, 4, 5, or 6 bp.
281. The nucleic acid molecule of embodiment 279 or 280, wherein the first stem of the second stem loop comprises a total length of 5 bp.
282. The nucleic acid molecule of any one of embodiments 278-281, wherein the first stem of the first stem loop comprises a total length of 6 bp, the tail of the tracrRNA comprises a total length of 3 nucleotides, and the first stem of the second stem loop comprises a total length of 5 bp.
283. The nucleic acid molecule of embodiment 255, wherein the gRNA is a dual guide RNA (dgRNA).
284. The nucleic acid molecule of embodiment 283, wherein the crRNA repeat comprises a total length of at least 10, 11, 12, 13, 14, 15, 16, 17, 18, 19, 20, or 21 nucleotides.
285. The nucleic acid molecule of embodiment 283, wherein the crRNA repeat comprises a total length of at most 10, 11, 12, 13, 14, 15, 16, 17, 18, 19, 20, or 21 nucleotides.
286. The nucleic acid molecule of embodiment 284 or 285, wherein the crRNA repeat comprises a total length of 13 nucleotides.
287. The nucleic acid molecule of embodiment 284 or 285, wherein the crRNA repeat comprises a total length of 16 nucleotides.
288. The nucleic acid molecule of embodiment 284 or 285, wherein the crRNA repeat of the dgRNA comprises a total length of 21 nucleotides.
289. The nucleic acid molecule of any one of embodiments 283-288, wherein the tracrRNA comprises a total length of at least 65, 70, 75, 80, or 85 nucleotides.
290. The nucleic acid molecule of any one of embodiments 283-288, wherein the tracrRNA comprises a total length of at most 65, 70, 75, 80, or 85 nucleotides.
291. The nucleic acid molecule of embodiment 289 or 290, wherein the tracrRNA comprises a total length of 74 nucleotides.
292. The nucleic acid molecule of embodiment 289 or 290, wherein the tracrRNA comprises a total length of 77 nucleotides.
293. The nucleic acid molecule of any one of embodiments 255-292, wherein the gRNA comprises a total length of at least 85, 90, 95, 100, 105, 110, 115, 120, 125, 130, or 135 nucleotides.
294. The nucleic acid molecule of any one of embodiments 255-292, wherein the gRNA comprises a total length of at most 85, 90, 95, 100, 105, 110, 115, 120, 125, 130, or 135 nucleotides.
295. The nucleic acid molecule of any one of embodiments 255-292, wherein the gRNA comprises a total length of 106 to 135 nucleotides.
296. The nucleic acid molecule of embodiment 285, wherein the gRNA comprises a total length of 117 to 119 nucleotides.
297. The nucleic acid molecule of any one of embodiments 255-296, wherein the gRNA is capable of targeting a bound RNA-guided nuclease (RGN) polypeptide to a target sequence.
298. The nucleic acid molecule of embodiment 297, wherein the RGN polypeptide is capable of recognizing a consensus protospacer adjacent motif (PAM) having the nucleotide sequence set forth as NNNNCC or NNRNCC.
299. The nucleic acid molecule of embodiment 298, wherein the RGN polypeptide is capable of recognizing a full protospacer adjacent motif (PAM) having the nucleotide sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG.
300. The nucleic acid molecule of any one of embodiments 297-299, wherein the RGN polypeptide comprises an amino acid sequence having at least 80% sequence identity to SEQ ID NO: 105; and wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 1 to 5 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 1 to 5 nucleotides.
301. The nucleic acid molecule of embodiment 300, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 5 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 5 nucleotides.
302. The nucleic acid molecule of embodiment 300, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 4 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 4 nucleotides.
303. The nucleic acid molecule of embodiment 300, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 3 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 3 nucleotides.
304. The nucleic acid molecule of embodiment 300, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 2 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 2 nucleotides.
305. The nucleic acid molecule of embodiment 300, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 1 nucleotide; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 1 nucleotide.
306. The nucleic acid molecule of embodiment 300, wherein the spacer has the nucleotide sequence set forth as SEQ ID NO: 7 or 9.
307. The nucleic acid molecule of any one of embodiments 300-306, wherein the RGN polypeptide has an amino acid sequence having at least 90% sequence identity to SEQ ID NO: 105.
308. The nucleic acid molecule of any one of embodiments 300-306, wherein the RGN polypeptide has an amino acid sequence having at least 95% sequence identity to SEQ ID NO: 105.
309. The nucleic acid molecule of any one of embodiments 300-306, wherein the RGN polypeptide has the amino acid sequence set forth as SEQ ID NO: 105.
310. The nucleic acid molecule of any one of embodiments 297-299, wherein the RGN polypeptide has an amino acid sequence having at least 80% sequence identity to SEQ ID NO: 333.
311. The nucleic acid molecule of embodiment 310, wherein the RGN polypeptide has an amino acid sequence having at least 90% sequence identity to SEQ ID NO: 333.
312. The nucleic acid molecule of embodiment 310, wherein the RGN polypeptide has an amino acid sequence having at least 95% sequence identity to SEQ ID NO: 333.
313. The nucleic acid molecule of embodiment 310, wherein the RGN polypeptide has the amino acid sequence set forth as SEQ ID NO: 333.
314. The nucleic acid molecule of any one of embodiments 297-313, wherein the gRNA has a nucleotide sequence having at least 80% sequence identity to any one of SEQ ID NOs: 198-200, 202-213, 215-233, 235-241, and 243-259.
315. The nucleic acid molecule of any one of embodiments 297-313, wherein the gRNA has a nucleotide sequence having at least 90% sequence identity to any one of SEQ ID NOs: 198-200, 202-213, 215-233, 235-241, and 243-259.
316. The nucleic acid molecule of any one of embodiments 297-313, wherein the gRNA has a nucleotide sequence having at least 95% sequence identity to any one of SEQ ID NOs: 198-200, 202-213, 215-233, 235-241, and 243-259.
317. The nucleic acid molecule of any one of embodiments 297-313, wherein the gRNA has a nucleotide sequence set forth as any one of SEQ ID NOs: 198-200, 202-213, 215-233, 235-241, and 243-259.
318. The nucleic acid molecule of embodiment 317, wherein the gRNA has a nucleotide sequence set forth as SEQ ID NO: 204 or 205.
319. The nucleic acid molecule of embodiment 297 or 298, wherein the RGN polypeptide has an amino acid sequence having at least 80% sequence identity to SEQ ID NO: 327 or 330.
320. The nucleic acid molecule of embodiment 319, wherein the RGN polypeptide has an amino acid sequence having at least 90% sequence identity to SEQ ID NO: 327 or 330.
321. The nucleic acid molecule of embodiment 319, wherein the RGN polypeptide has an amino acid sequence having at least 95% sequence identity to SEQ ID NO: 327 or 330.
322. The nucleic acid molecule of embodiment 319, wherein the RGN polypeptide has an amino acid sequence set forth as SEQ ID NO: 327 or 330.
323. The nucleic acid molecule of embodiment 299, wherein the RGN polypeptide is capable of recognizing a full PAM having the nucleotide sequence set forth as GGGCCCAG.
324. The nucleic acid molecule of embodiment 323, wherein the RGN polypeptide has an amino acid sequence having at least 80% sequence identity to SEQ ID NO: 324.
325. The nucleic acid molecule of embodiment 324, wherein the RGN polypeptide has an amino acid sequence having at least 90% sequence identity to SEQ ID NO: 324.
326. The nucleic acid molecule of embodiment 324, wherein the RGN polypeptide has an amino acid sequence having at least 95% sequence identity to SEQ ID NO: 324.
327. The nucleic acid molecule of embodiment 324, wherein the RGN polypeptide has the amino acid sequence set forth as SEQ ID NO: 324.
328. The nucleic acid molecule of embodiment 299, wherein the RGN polypeptide is capable of recognizing a full PAM having the nucleotide sequence set forth as CAGGCCAA.
329. The nucleic acid molecule of embodiment 328, wherein the RGN polypeptide has an amino acid sequence having at least 80% sequence identity to SEQ ID NO: 404.
330. The nucleic acid molecule of embodiment 329, wherein the RGN polypeptide has an amino acid sequence having at least 90% sequence identity to SEQ ID NO: 404.
331. The nucleic acid molecule of embodiment 329, wherein the RGN polypeptide has an amino acid sequence having at least 95% sequence identity to SEQ ID NO: 404.
332. The nucleic acid molecule of embodiment 329, wherein the RGN polypeptide has the amino acid sequence set forth as SEQ ID NO: 404.
333. The nucleic acid molecule of any one of embodiments 233-332, wherein the gRNA comprises at least one chemical modification.
334. The nucleic acid molecule of embodiment 333, wherein the at least one chemical modification comprises a bridged nucleic acid (BNA) modification; 2′-O-methyl(2′-O-Me) modification; 2′-O-methoxy-ethyl(2′MOE) modification; 2′-fluoro (2′-F) modification; 2′F-4′Cα-OMe modification; 2′,4′-di-Cα-OMe modification; 2′-O-methyl 3′phosphorothioate (MS) modification; 2′-O-methyl 3′thiophosphonoacetate (MSP) modification; 2′-O-methyl 3′phosphonoacetate (MP) modification; phosphorothioate (PS) modification; or a combination thereof.
335. The nucleic acid molecule of embodiment 334, wherein the at least one chemical modification comprises MS modifications at the 3 terminal nucleotides at the 5′ region and at the 3 terminal nucleotides at the 3′ region of the gRNA.
336. The nucleic acid molecule of embodiment 335, wherein the crRNA repeat has the nucleotide sequence set forth as any one of SEQ ID NOs: 430, 432-435, 583, 585, and 587.
337. The nucleic acid molecule of embodiment 335 or 336, wherein the crRNA has the nucleotide sequence set forth as any one of SEQ ID NOs: 459-520.
338. The nucleic acid molecule of any one of embodiments 335-337, wherein the tracrRNA has the nucleotide sequence set forth as any one of SEQ ID NOs: 431, 437-446, 584, 586, and 588.
339. The nucleic acid molecule of any one of embodiments 335-338, wherein the gRNA has the nucleotide sequence set forth as any one of SEQ ID NOs: 521-523, 525-536, 538-556, 558-564, and 566-582.
340. The nucleic acid molecule of embodiment 334, wherein the BNA comprises a 2′,4′BNA modification.
341. The nucleic acid molecule of embodiment 340, wherein the 2′,4′ BNA modification is selected from the group consisting of: locked nucleic acid (LNA) modification, BNA^{NC}[N-Me] modification, 2′-O,4′-C-ethylene bridged nucleic acid (2′,4′-ENA) modification, and S-constrained ethyl (cEt) modification.
342. The nucleic acid molecule of embodiment 341, wherein the 2′,4′ BNA is a LNA modification.
343. The nucleic acid molecule of embodiment 341, wherein the 2′,4′ BNA is a cEt modification.
344. The nucleic acid molecule of embodiment 334, wherein the at least one chemical modification comprises a BNA modification, 2′-O-Me modification, PS modification, or a combination thereof.
345. The nucleic acid molecule of any one of embodiments 255-344, wherein the gRNA further comprises an extension comprising an edit template for reverse transcriptase editing.
346. A vector comprising the nucleic acid molecule of any one of embodiments 233-254, wherein the nucleic acid molecule comprises a polynucleotide encoding the crRNA.
347. The vector of embodiment 346, wherein the nucleic acid molecule further comprises a heterologous promoter operably linked to the polynucleotide encoding the crRNA.
348. The vector of embodiment 347, wherein the heterologous promoter is an RNA polymerase III (pol III) promoter.
349. The vector of any one of embodiments 346-348, wherein the vector further comprises a nucleic acid molecule encoding an RGN polypeptide.
350. The vector of embodiment 349, wherein the crRNA is capable of binding a tracrRNA to form a guide RNA, and wherein the guide RNA is capable of binding to the RGN polypeptide.
351. The vector of embodiment 349 or 350, wherein the vector further comprises a promoter operably linked to the nucleic acid molecule encoding the RGN polypeptide.
352. A vector comprising the nucleic acid molecule of any one of embodiments 255-345, wherein the nucleic acid molecule comprises a polynucleotide encoding the crRNA, and wherein the vector further comprises a polynucleotide encoding the tracrRNA.
353. The vector of embodiment 352, wherein the polynucleotide encoding the crRNA and the polynucleotide encoding the tracrRNA are operably linked to the same promoter and are encoded as a sgRNA.
354. The vector of embodiment 352, wherein the polynucleotide encoding the crRNA and the polynucleotide encoding the tracrRNA are operably linked to separate promoters.
355. The vector of any one of embodiments 352-354, wherein the vector further comprises a nucleic acid molecule encoding an RGN polypeptide.
356. The vector of embodiment 355, wherein the crRNA is capable of binding the tracrRNA to form a guide RNA, and wherein the guide RNA is capable of binding to the RGN polypeptide.
357. The vector of embodiment 355 or 356, wherein the vector further comprises a promoter operably linked to the nucleic acid molecule encoding the RGN polypeptide.
358. A nucleic acid molecule comprising a CRISPR RNA (crRNA) or encoding a crRNA, wherein the crRNA comprises a spacer and a crRNA repeat, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103, or has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 to 5 nucleotides.
359. The nucleic acid molecule of embodiment 358, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 to 5 nucleotides.
360. The nucleic acid molecule of embodiment 359, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 5 nucleotides.
361. The nucleic acid molecule of embodiment 359, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 4 nucleotides.
362. The nucleic acid molecule of embodiment 359, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 3 nucleotides.
363. The nucleic acid molecule of embodiment 359, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 2 nucleotides.
364. The nucleic acid molecule of embodiment 359, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 nucleotide.
365. The nucleic acid molecule of embodiment 358, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103.
366. The nucleic acid molecule of any one of embodiments 358-365, wherein the spacer is capable of hybridizing to a target sequence, and wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, and 104.
367. The nucleic acid molecule of any one of embodiments 358-366, wherein the crRNA repeat has the nucleotide sequence set forth as SEQ ID NO: 106 or a nucleotide sequence that differs in length and/or sequence from SEQ ID NO: 106 by 1 to 8 nucleotides.
368. The nucleic acid molecule of embodiment 367, wherein the crRNA repeat has a nucleotide sequence that differs in length and/or sequence from SEQ ID NO: 106 by 8 nucleotides.
369. The nucleic acid molecule of embodiment 367, wherein the crRNA repeat has a nucleotide sequence that differs in length and/or sequence from SEQ ID NO: 106 by 7 nucleotides.
370. The nucleic acid molecule of embodiment 367, wherein the crRNA repeat has a nucleotide sequence that differs in length and/or sequence from SEQ ID NO: 106 by 6 nucleotides.
371. The nucleic acid molecule of embodiment 367, wherein the crRNA repeat has a nucleotide sequence that differs in length and/or sequence from SEQ ID NO: 106 by 5 nucleotides.
372. The nucleic acid molecule of embodiment 367, wherein the crRNA repeat has a nucleotide sequence that differs in length and/or sequence from SEQ ID NO: 106 by 4 nucleotides.
373. The nucleic acid molecule of embodiment 367, wherein the crRNA repeat has a nucleotide sequence that differs in length and/or sequence from SEQ ID NO: 106 by 3 nucleotides.
374. The nucleic acid molecule of embodiment 367, wherein the crRNA repeat has a nucleotide sequence that differs in length and/or sequence from SEQ ID NO: 106 by 2 nucleotides.
375. The nucleic acid molecule of embodiment 367, wherein the crRNA repeat has a nucleotide sequence that differs in length and/or sequence from SEQ ID NO: 106 by 1 nucleotide.
376. The nucleic acid molecule of embodiment 367, wherein the crRNA repeat has the nucleotide sequence set forth as any one of SEQ ID NOs: 106, 109-112, 328, 331, and 334.
377. The nucleic acid molecule of any one of embodiments 358-367, wherein the crRNA has a nucleotide sequence having at least 80% sequence identity to SEQ ID NOs: 136-197.
378. The nucleic acid molecule of embodiment 377, wherein the crRNA has a nucleotide sequence having at least 90% sequence identity to SEQ ID NOs: 136-197.
379. The nucleic acid molecule of embodiment 377, wherein the crRNA has a nucleotide sequence having at least 95% sequence identity to SEQ ID NOs: 136-197.
380. The nucleic acid molecule of embodiment 377, wherein the crRNA has the nucleotide sequence set forth as any one of SEQ ID NOs: 136-197.
381. The nucleic acid molecule of any one of embodiments 358-80, wherein the crRNA is capable of binding a trans-activating CRISPR RNA (tracrRNA) to form a guide RNA (gRNA), wherein the tracrRNA comprises an anti-repeat and a tail.
382. The nucleic acid molecule of embodiment 381, wherein the tracrRNA has a nucleotide sequence having at least 80% sequence identity to SEQ ID NO: 107.
383. The nucleic acid molecule of embodiment 382, wherein the tracrRNA has a nucleotide sequence having at least 90% sequence identity to SEQ ID NO: 107.
384. The nucleic acid molecule of embodiment 382, wherein the tracrRNA has a nucleotide sequence having at least 95% sequence identity to SEQ ID NO: 107.
385. The nucleic acid molecule of embodiment 382, wherein the tracrRNA has a nucleotide sequence that differs in length from SEQ ID NO: 107 by 1 to 16 nucleotides.
386. The nucleic acid molecule of embodiment 385, wherein the tracrRNA has a nucleotide sequence that is 8 nucleotides shorter than SEQ ID NO: 107.
387. The nucleic acid molecule of embodiment 385, wherein the tracrRNA has a nucleotide sequence that is 11 nucleotides shorter than SEQ ID NO: 107.
388. The nucleic acid molecule of any one of embodiments 382-387, wherein the tracrRNA has the nucleotide sequence set forth as any one of SEQ ID NOs: 107, 114-123, 329, 332, and 335.
389. The nucleic acid molecule of embodiment 381, wherein the gRNA is a single guide RNA (sgRNA) comprising the crRNA and the tracrRNA linked by a linker, wherein the sgRNA comprises a backbone and the spacer, and wherein the backbone of the sgRNA comprises the crRNA repeat, the linker, and the tracrRNA.
390. The nucleic acid molecule of embodiment 389, wherein the backbone of the sgRNA comprises a total length of 86 to 98 nucleotides.
391. The nucleic acid molecule of embodiment 389, wherein the backbone of the sgRNA comprises a total length of 94 nucleotides.
392. The nucleic acid molecule of embodiment 389, wherein the backbone of the sgRNA has a nucleotide sequence having at least 80% sequence identity to any one of SEQ ID NOs: 124-134.
393. The nucleic acid molecule of embodiment 392, wherein the backbone of the sgRNA has a nucleotide sequence having at least 90% sequence identity to any one of SEQ ID NOs: 124-134.
394. The nucleic acid molecule of embodiment 392, wherein the backbone of the sgRNA has a nucleotide sequence having at least 95% sequence identity to any one of SEQ ID NOs: 124-134.
395. The nucleic acid molecule of embodiment 392, wherein the backbone of the sgRNA has the nucleotide sequence set forth as any one of SEQ ID NOs: 124-134.
396. The nucleic acid molecule of any one of embodiments 381-395, wherein the gRNA comprises a first stem loop comprising a first stem and a second stem formed by hybridization of the crRNA repeat and the anti-repeat, and wherein the first stem of the first stem loop comprises a total length of at least 3, 4, 5, 6, 7, 8, 9, 10, or 11 bp.
397. The nucleic acid molecule of any one of embodiments 381-395, wherein the gRNA comprises a first stem loop comprising a first stem and a second stem formed by hybridization of the crRNA repeat and the anti-repeat, and wherein the first stem of the first stem loop comprises a total length of at most 3, 4, 5, 6, 7, 8, 9, 10, or 11 bp.
398. The nucleic acid molecule of embodiment 396 or 397, wherein the first stem of the first stem loop comprises a total length of 6 bp.
399. The nucleic acid molecule of embodiment 396 or 397, wherein the first stem of the first stem loop comprises a total length of 3 bp.
400. The nucleic acid molecule of any one of embodiments 381-399, wherein the tail of the tracrRNA comprises a total length of at least 1, 2, 3, 4, 5, 6, or 7 nucleotides.
401. The nucleic acid molecule of any one of embodiments 381-399, wherein the tail of the tracrRNA comprises a total length of at most 1, 2, 3, 4, 5, 6, or 7 nucleotides.
402. The nucleic acid molecule of embodiment 400 or 401, wherein the tail of the tracrRNA comprises a total length of 3 nucleotides.
403. The nucleic acid molecule of embodiment 400 or 401, wherein the tail of the tracrRNA comprises a total length of 1 nucleotide.
404. The nucleic acid molecule of any one of embodiments 396-403, wherein the gRNA further comprises a second stem loop most proximal to the tail, wherein the second stem loop comprises a first stem and a second stem.
405. The nucleic acid molecule of embodiment 404, wherein the first stem of the second stem loop comprises a total length of at least 1, 2, 3, 4, 5, or 6 bp.
406. The nucleic acid molecule of embodiment 404, wherein the first stem of the second stem loop comprises a total length of at most 1, 2, 3, 4, 5, or 6 bp.
407. The nucleic acid molecule of embodiment 405 or 406, wherein the first stem of the second stem loop comprises a total length of 5 bp.
408. The nucleic acid molecule of any one of embodiments 404-407, wherein the first stem of the first stem loop comprises a total length of 6 bp, the tail of the tracrRNA comprises a total length of 3 nucleotides, and the first stem of the second stem loop comprises a total length of 5 bp.
409. The nucleic acid molecule of embodiment 381, wherein the gRNA is a dual guide RNA (dgRNA).
410. The nucleic acid molecule of embodiment 409, wherein the crRNA repeat comprises a total length of at least 10, 11, 12, 13, 14, 15, 16, 17, 18, 19, 20, or 21 nucleotides.
411. The nucleic acid molecule of embodiment 409, wherein the crRNA repeat comprises a total length of at most 10, 11, 12, 13, 14, 15, 16, 17, 18, 19, 20, or 21 nucleotides.
412. The nucleic acid molecule of embodiment 410 or 411, wherein the crRNA repeat comprises a total length of 13 nucleotides.
413. The nucleic acid molecule of embodiment 410 or 411, wherein the crRNA repeat comprises a total length of 16 nucleotides.
414. The nucleic acid molecule of embodiment 410 or 411, wherein the crRNA repeat of the dgRNA comprises a total length of 21 nucleotides.
415. The nucleic acid molecule of any one of embodiments 409-414, wherein the tracrRNA comprises a total length of at least 65, 70, 75, 80, or 85 nucleotides.
416. The nucleic acid molecule of any one of embodiments 409-414, wherein the tracrRNA comprises a total length of at most 65, 70, 75, 80, or 85 nucleotides.
417. The nucleic acid molecule of embodiment 415 or 416, wherein the tracrRNA comprises a total length of 74 nucleotides.
418. The nucleic acid molecule of embodiment 415 or 416, wherein the tracrRNA comprises a total length of 77 nucleotides.
419. The nucleic acid molecule of any one of embodiments 381-418, wherein the gRNA comprises a total length of at least 85, 90, 95, 100, 105, 110, 115, 120, 125, 130, or 135 nucleotides.
420. The nucleic acid molecule of any one of embodiments 381-418, wherein the gRNA comprises a total length of at most 85, 90, 95, 100, 105, 110, 115, 120, 125, 130, or 135 nucleotides.
421. The nucleic acid molecule of any one of embodiments 381-418, wherein the gRNA comprises a total length of 106 to 135 nucleotides.
422. The nucleic acid molecule of embodiment 421, wherein the gRNA comprises a total length of 117 to 119 nucleotides.
423. The nucleic acid molecule of any one of embodiments 381-422, wherein the gRNA is capable of targeting a bound RNA-guided nuclease (RGN) polypeptide to a target sequence.
424. The nucleic acid molecule of embodiment 423, wherein the RGN polypeptide is capable of recognizing a consensus protospacer adjacent motif (PAM) having the nucleotide sequence set forth as NNNNCC or NNRNCC.
425. The nucleic acid molecule of embodiment 424, wherein the RGN polypeptide is capable of recognizing a full protospacer adjacent motif (PAM) having the nucleotide sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG.
426. The nucleic acid molecule of any one of embodiments 423-425, wherein the RGN polypeptide comprises an amino acid sequence having at least 80% sequence identity to SEQ ID NO: 105; and wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 1 to 5 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 1 to 5 nucleotides.
427. The nucleic acid molecule of embodiment 426, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 5 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 5 nucleotides.
428. The nucleic acid molecule of embodiment 426, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 4 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 4 nucleotides.
429. The nucleic acid molecule of embodiment 426, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 3 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 3 nucleotides.
430. The nucleic acid molecule of embodiment 426, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 2 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 2 nucleotides.
431. The nucleic acid molecule of embodiment 426, wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 1 nucleotide; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 1 nucleotide.
432. The nucleic acid molecule of embodiment 426, wherein the spacer has the nucleotide sequence set forth as SEQ ID NO: 7 or 9.
433. The nucleic acid molecule of any one of embodiments 426-432, wherein the RGN polypeptide has an amino acid sequence having at least 90% sequence identity to SEQ ID NO: 105.
434. The nucleic acid molecule of any one of embodiments 426-432, wherein the RGN polypeptide has an amino acid sequence having at least 95% sequence identity to SEQ ID NO: 105.
435. The nucleic acid molecule of any one of embodiments 426-432, wherein the RGN polypeptide has the amino acid sequence set forth as SEQ ID NO: 105.
436. The nucleic acid molecule of any one of embodiments 423-425, wherein the RGN polypeptide has an amino acid sequence having at least 80% sequence identity to SEQ ID NO: 333.
437. The nucleic acid molecule of embodiment 436, wherein the RGN polypeptide has an amino acid sequence having at least 90% sequence identity to SEQ ID NO: 333.
438. The nucleic acid molecule of embodiment 436, wherein the RGN polypeptide has an amino acid sequence having at least 95% sequence identity to SEQ ID NO: 333.
439. The nucleic acid molecule of embodiment 436, wherein the RGN polypeptide has the amino acid sequence set forth as SEQ ID NO: 333.
440. The nucleic acid molecule of any one of embodiments 423-439, wherein the gRNA has a nucleotide sequence having at least 80% sequence identity to any one of SEQ ID NOs: 198-200, 202-213, 215-233, 235-241, and 243-259.
441. The nucleic acid molecule of embodiment 440, wherein the gRNA has a nucleotide sequence having at least 90% sequence identity to any one of SEQ ID NOs: 198-200, 202-213, 215-233, 235-241, and 243-259.
442. The nucleic acid molecule of embodiment 440, wherein the gRNA has a nucleotide sequence having at least 95% sequence identity to any one of SEQ ID NOs: 198-200, 202-213, 215-233, 235-241, and 243-259.
443. The nucleic acid molecule of embodiment 440, wherein the gRNA has the nucleotide sequence set forth as any one of SEQ ID NOs: 198-200, 202-213, 215-233, 235-241, and 243-259.
444. The nucleic acid molecule of embodiment 443, wherein the gRNA has a nucleotide sequence set forth as SEQ ID NO: 204 or 205.
445. The nucleic acid molecule of embodiment 423 or 424, wherein the RGN polypeptide has an amino acid sequence having at least 80% sequence identity to SEQ ID NO: 327 or 330.
446. The nucleic acid molecule of embodiment 445, wherein the RGN polypeptide has an amino acid sequence having at least 90% sequence identity to SEQ ID NO: 327 or 330.
447. The nucleic acid molecule of embodiment 445, wherein the RGN polypeptide has an amino acid sequence having at least 95% sequence identity to SEQ ID NO: 327 or 330.
448. The nucleic acid molecule of embodiment 445, wherein the RGN polypeptide has an amino acid sequence set forth as SEQ ID NO: 327 or 330.
449. The nucleic acid molecule of embodiment 425, wherein the RGN polypeptide is capable of recognizing a full PAM having the nucleotide sequence set forth as GGGCCCAG.
450. The nucleic acid molecule of embodiment 449, wherein the RGN polypeptide has an amino acid sequence having at least 80% sequence identity to SEQ ID NO: 324.
451. The nucleic acid molecule of embodiment 450, wherein the RGN polypeptide has an amino acid sequence having at least 90% sequence identity to SEQ ID NO: 324.
452. The nucleic acid molecule of embodiment 450, wherein the RGN polypeptide has an amino acid sequence having at least 95% sequence identity to SEQ ID NO: 324.
453. The nucleic acid molecule of embodiment 450, wherein the RGN polypeptide has the amino acid sequence set forth as SEQ ID NO: 324.
454. The nucleic acid molecule of embodiment 425, wherein the RGN polypeptide is capable of recognizing a full PAM having the nucleotide sequence set forth as CAGGCCAA.
455. The nucleic acid molecule of embodiment 454, wherein the RGN polypeptide has an amino acid sequence having at least 80% sequence identity to SEQ ID NO: 404.
456. The nucleic acid molecule of embodiment 455, wherein the RGN polypeptide has an amino acid sequence having at least 90% sequence identity to SEQ ID NO: 404.
457. The nucleic acid molecule of embodiment 455, wherein the RGN polypeptide has an amino acid sequence having at least 95% sequence identity to SEQ ID NO: 404.
458. The nucleic acid molecule of embodiment 455, wherein the RGN polypeptide has the amino acid sequence set forth as SEQ ID NO: 404.
459. The nucleic acid molecule of any one of embodiments 381-458, wherein the gRNA comprises at least one chemical modification.
460. The nucleic acid molecule of embodiment 459, wherein the at least one chemical modification comprises a bridged nucleic acid (BNA) modification; 2′-O-methyl(2′-O-Me) modification; 2′-O-methoxy-ethyl(2′MOE) modification; 2′-fluoro (2′-F) modification; 2′F-4′Cα-OMe modification; 2′,4′-di-Cα-OMe modification; 2′-O-methyl 3′phosphorothioate (MS) modification; 2′-O-methyl 3′thiophosphonoacetate (MSP) modification; 2′-O-methyl 3′phosphonoacetate (MP) modification; phosphorothioate (PS) modification; or a combination thereof.
461. The nucleic acid molecule of embodiment 460, wherein the at least one chemical modification comprises MS modifications at the 3 terminal nucleotides at the 5′ region and at the 3 terminal nucleotides at the 3′ region of the gRNA.
462. The nucleic acid molecule of embodiment 461, wherein the crRNA repeat has the nucleotide sequence set forth as any one of SEQ ID NOs: 430, 432-435, 583, 585, and 587.
463. The nucleic acid molecule of embodiment 461 or 462, wherein the crRNA has the nucleotide sequence set forth as any one of SEQ ID NOs: 459-520.
464. The nucleic acid molecule of any one of embodiments 461-463, wherein the tracrRNA has the nucleotide sequence set forth as any one of SEQ ID NOs: 431, 437-446, 584, 586, and 588.
465. The nucleic acid molecule of any one of embodiments 461-464, wherein the gRNA has the nucleotide sequence set forth as any one of SEQ ID NOs: 521-523, 525-536, 538-556, 558-564, and 566-582.
466. The nucleic acid molecule of embodiment 460, wherein the BNA comprises a 2′,4′ BNA modification.
467. The nucleic acid molecule of embodiment 466, wherein the 2′,4′ BNA modification is selected from the group consisting of: locked nucleic acid (LNA) modification, BNA^{NC}[N-Me] modification, 2′-O,4′-C-ethylene bridged nucleic acid (2′,4′-ENA) modification, and S-constrained ethyl (cEt) modification.
468. The nucleic acid molecule of embodiment 467, wherein the 2′,4′ BNA is a LNA modification.
469. The nucleic acid molecule of embodiment 467, wherein the 2′,4′ BNA is a cEt modification.
470. The nucleic acid molecule of embodiment 460, wherein the at least one chemical modification comprises a BNA modification, 2′-O-Me modification, PS modification, or a combination thereof.
471. The nucleic acid molecule of any one of embodiments 381-470, wherein the gRNA further comprises an extension comprising an edit template for reverse transcriptase editing.
472. A vector comprising the nucleic acid molecule of any one of embodiments 358-380, wherein the nucleic acid molecule comprises a polynucleotide encoding the crRNA.
473. The vector of embodiment 472, wherein the nucleic acid molecule further comprises a heterologous promoter operably linked to the polynucleotide encoding the crRNA.
474. The vector of embodiment 473, wherein the heterologous promoter is an RNA polymerase III (pol III) promoter.
475. The vector of any one of embodiments 472-474, wherein the vector further comprises a nucleic acid molecule encoding an RGN polypeptide.
476. The vector of embodiment 475, wherein the crRNA is capable of binding a tracrRNA to form a guide RNA, and wherein the guide RNA is capable of binding to the RGN polypeptide.
477. The vector of embodiment 475 or 476, wherein the vector further comprises a promoter operably linked to the nucleic acid molecule encoding the RGN polypeptide.
478. A vector comprising the nucleic acid molecule of any one of embodiments 381-471, wherein the nucleic acid molecule comprises a polynucleotide encoding the crRNA, and wherein the vector further comprises a polynucleotide encoding the tracrRNA.
479. The vector of embodiment 478, wherein the polynucleotide encoding the crRNA and the polynucleotide encoding the tracrRNA are operably linked to the same promoter and are encoded as a sgRNA.
480. The vector of embodiment 478, wherein the polynucleotide encoding the crRNA and the polynucleotide encoding the tracrRNA are operably linked to separate promoters.
481. The vector of any one of embodiments 478-480, wherein the vector further comprises a nucleic acid molecule encoding an RGN polypeptide.
482. The vector of embodiment 481, wherein the crRNA is capable of binding the tracrRNA to form a guide RNA, and wherein the guide RNA is capable of binding to the RGN polypeptide.
483. The vector of embodiment 481 or 482, wherein the vector further comprises a promoter operably linked to the nucleic acid molecule encoding the RGN polypeptide.
484. A cell comprising the gRNA of any one of embodiments 1-232, the nucleic acid molecule of any one of embodiments 233-345 and 358-471, or the vector of any one of embodiments 346-357 and 472-483.
485. An RNA-guided nuclease (RGN) system for binding a target sequence within a TRAC gene, wherein the RGN system comprises:
a) one or more guide RNA (gRNA) of any one of embodiments 1-232, or one or more polynucleotides comprising one or more nucleotide sequences encoding the one or more gRNA of any one of embodiments 1-232; and
b) an RGN polypeptide, or a polynucleotide comprising a nucleotide sequence encoding the RGN polypeptide.
486. The RGN system of embodiment 485, wherein the one or more gRNA are capable of forming a complex with the RGN polypeptide to direct the RGN polypeptide to bind to the target sequence.
487. The RGN system of embodiment 485 or 486, wherein the RGN polypeptide is capable of recognizing a consensus protospacer adjacent motif (PAM) having the nucleotide sequence set forth as NNNNCC or NNRNCC.
488. The RGN system of embodiment 487, wherein the RGN polypeptide is capable of recognizing a full PAM having a nucleotide sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG.
489. The RGN system of any one of embodiments 485-488, wherein the RGN polypeptide comprises an amino acid sequence having at least 80% sequence identity to SEQ ID NO: 105.
490. The RGN system of embodiment 489, wherein the RGN polypeptide comprises an amino acid sequence having at least 90% sequence identity to SEQ ID NO: 105.
491. The RGN system of embodiment 489, wherein the RGN polypeptide comprises an amino acid sequence having at least 95% sequence identity to SEQ ID NO: 105.
492. The RGN system of embodiment 489, wherein the RGN polypeptide comprises the amino acid sequence set forth as SEQ ID NO: 105.
493. The RGN system of any one of embodiments 485-488, wherein the RGN polypeptide has an amino acid sequence having at least 80% sequence identity to SEQ ID NO: 333.
494. The RGN system of embodiment 493, wherein the RGN polypeptide has an amino acid sequence having at least 90% sequence identity to SEQ ID NO: 333.
495. The RGN system of embodiment 493, wherein the RGN polypeptide has an amino acid sequence having at least 95% sequence identity to SEQ ID NO: 333.
496. The RGN system of embodiment 493, wherein the RGN polypeptide has the amino acid sequence set forth as SEQ ID NO: 333.
497. The RGN system of any one of embodiments 485-487, wherein the RGN polypeptide has an amino acid sequence having at least 80% sequence identity to SEQ ID NO: 327 or 330.
498. The RGN system of embodiment 497, wherein the RGN polypeptide has an amino acid sequence having at least 90% sequence identity to SEQ ID NO: 327 or 330.
499. The RGN system of embodiment 497, wherein the RGN polypeptide has an amino acid sequence having at least 95% sequence identity to SEQ ID NO: 327 or 330.
500. The RGN system of embodiment 497, wherein the RGN polypeptide has an amino acid sequence set forth as SEQ ID NO: 327 or 330.
501. The RGN system of any one of embodiments 485-500, wherein the polynucleotide comprising a nucleotide sequence encoding the RGN polypeptide is codon optimized for expression in a mammalian cell.
502. The RGN system of any one of embodiments 485-501, wherein at least one of the one or more nucleotide sequences encoding the one or more gRNA and the nucleotide sequence encoding the RGN polypeptide is operably linked to a promoter heterologous to the nucleotide sequence.
503. The RGN system of any one of embodiments 485-502, wherein the one or more nucleotide sequences encoding the one or more gRNAs and the nucleotide sequence encoding the RGN polypeptide are located on one vector.
504. The RGN system of any one of embodiments 485-500, wherein the polynucleotide comprising a nucleotide sequence encoding the RGN polypeptide comprises an mRNA.
505. The RGN system of any one of embodiments 485-504, wherein the RGN polypeptide is nuclease inactive or is a nickase.
506. The RGN system of any one of embodiments 485-505, wherein the RGN polypeptide is fused to a base-editing polypeptide.
507. The RGN system of embodiment 506, wherein the base-editing polypeptide comprises a deaminase.
508. The RGN system of any one of embodiments 485-505, wherein the RGN polypeptide is fused to a reverse transcriptase (RT) editing polypeptide.
509. The RGN system of embodiment 508, wherein the RT editing polypeptide comprises a DNA polymerase.
510. The RGN system of embodiment 509, wherein the DNA polymerase comprises a reverse transcriptase.
511. The RGN system of any one of embodiments 508-510, wherein the gRNA further comprises an extension comprising an edit template for RT editing.
512. The RGN system of any one of embodiments 485-511, wherein the RGN polypeptide comprises one or more nuclear localization signals.
513. A ribonucleoprotein (RNP) complex comprising the one or more gRNA and the RGN polypeptide of the RGN system of any one of embodiments 485-512.
514. A cell comprising the RGN system of any one of embodiments 485-512 or the RNP complex of embodiment 513.
515. The cell of embodiment 514, wherein the cell is a eukaryotic cell.
516. The cell of embodiment 515, wherein the eukaryotic cell is a mammalian cell.
517. The cell of embodiment 516, wherein the mammalian cell is a human cell.
518 The cell of embodiment 516 or 517, wherein the mammalian cell or human cell is a T cell or an induced pluripotent stem cell.
519. A method for binding a target sequence within a TRAC gene, comprising delivering the RGN system of any one of embodiments 485-512 or the RNP complex of embodiment 513 to the target sequence or a cell comprising the target sequence.
520. The method of embodiment 519, wherein cleavage or modification of the target sequence occurs.
521. A method for assembling an RNA-guided nuclease (RGN) ribonucleoprotein complex, the method comprising combining under conditions suitable for formation of the complex:
a) the guide RNA of any one of embodiments 1-232; and
b) an RGN polypeptide that binds the guide RNA.
522. The method of embodiment 521, wherein the RGN polypeptide is capable of recognizing a consensus protospacer adjacent motif (PAM) having the nucleotide sequence set forth as NNNNCC or NNRNCC.
523. The method of embodiment 521 or 522, wherein the complex directs cleavage of the target sequence.
524. The method of embodiment 523, wherein the cleavage generates a double-stranded break.
525. The method of embodiment 523, wherein the cleavage generates a single-stranded break.
526. A method for binding a target sequence within a TRAC gene, the method comprising:
a) combining under conditions suitable for formation of a ribonucleoprotein (RNP) complex:
   i) the guide RNA of any one of embodiments 1-232; and
   ii) an RGN polypeptide that binds the guide RNA;
thereby assembling an RNP complex; and
b) contacting the target sequence or a cell comprising the target sequence with the assembled RNP complex.
527. The method of embodiment 526, wherein the guide RNA hybridizes to the target sequence, thereby directing binding of the RNP complex to the target sequence.
528. The method of embodiment 526 or 527, wherein the RGN polypeptide is capable of recognizing a consensus protospacer adjacent motif (PAM) having the nucleotide sequence set forth as NNNNCC or NNRNCC.
529. The method of embodiment 528, wherein the RGN polypeptide is capable of recognizing a full protospacer adjacent motif (PAM) having the nucleotide sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG.
530. The method of any one of embodiments 526-529, wherein the RGN polypeptide comprises an amino acid sequence having at least 80% sequence identity to SEQ ID NO: 105.
531. The method of embodiment 530, wherein the RGN polypeptide comprises an amino acid sequence having at least 90% sequence identity to SEQ ID NO: 105.
532. The method of embodiment 530, wherein the RGN polypeptide comprises an amino acid sequence having at least 95% sequence identity to SEQ ID NO: 105.
533. The method of embodiment 530, wherein the RGN polypeptide comprises the amino acid sequence set forth as SEQ ID NO: 105.
534. The method of any one of embodiments 526-529, wherein the RGN polypeptide has an amino acid sequence having at least 80% sequence identity to SEQ ID NO: 333.
535. The method of embodiment 534, wherein the RGN polypeptide has an amino acid sequence having at least 90% sequence identity to SEQ ID NO: 333.
536. The method of embodiment 534, wherein the RGN polypeptide has an amino acid sequence having at least 95% sequence identity to SEQ ID NO: 333.
537. The method of embodiment 534, wherein the RGN polypeptide has an amino acid sequence set forth as SEQ ID NO: 333.
538. The method of any one of embodiments 526-528, wherein the RGN polypeptide has an amino acid sequence having at least 80% sequence identity to SEQ ID NO: 327 or 330.
539. The method of embodiment 538, wherein the RGN polypeptide has an amino acid sequence having at least 90% sequence identity to SEQ ID NO: 327 or 330.
540. The method of embodiment 538, wherein the RGN polypeptide has an amino acid sequence having at least 95% sequence identity to SEQ ID NO: 327 or 330.
541. The method of embodiment 538, wherein the RGN polypeptide has an amino acid sequence set forth as SEQ ID NO: 327 or 330.
542. The method of any one of embodiments 526-541, wherein the method is performed in vitro or ex vivo.
543. The method of any one of embodiments 526-542, wherein the RGN polypeptide is capable of cleaving the target sequence, thereby allowing for the cleaving and/or modifying of the target sequence.
544. The method of embodiment 543, wherein the cleaving generates a single-stranded break.
545. The method of embodiment 543, wherein the cleaving generates a double-stranded break.
546. The method of embodiment 543, wherein the cleaving results in insertion of a heterologous sequence within the target sequence.
547. The method of any one of embodiments 526-542, wherein the RGN polypeptide is nuclease inactive or is a nickase.
548. The method of embodiment 547, wherein the RGN polypeptide is fused to a base-editing polypeptide.
549. The method of embodiment 548, wherein the base-editing polypeptide comprises a deaminase.
550. The method of any one of embodiments 526-542, wherein the RGN is fused to a reverse transcriptase (RT) editing polypeptide.
551. The method of embodiment 550, wherein the RT editing polypeptide comprises a DNA polymerase.
552. The method of embodiment 551, wherein the DNA polymerase comprises a reverse transcriptase.
553. The method of any one of embodiments 550-552, wherein the gRNA further comprises an extension comprising an edit template for RT editing.
554. The method of any one of embodiments 526-553, wherein the target sequence is within a cell.
555. The method of embodiment 554, wherein the cell is a eukaryotic cell.
556. The method of embodiment 555, wherein the eukaryotic cell is a mammalian cell.
557. The method of embodiment 556, wherein the mammalian cell is a human cell.
558. The method of embodiment 556 or 557, wherein the mammalian cell or human cell is a T cell or an induced pluripotent stem cell.
559. The method of any one of embodiments 526-558, further comprising selecting a cell comprising a modified target sequence.
560. A cell comprising a modified target sequence obtained according to the method of embodiment 559.
561. The cell of embodiment 560, wherein the cell is a eukaryotic cell.
562 The cell of embodiment 561, wherein the eukaryotic cell is a mammalian cell.
563. The cell of embodiment 562, wherein the mammalian cell is a human cell.
564. The cell of embodiment 562 or 563, wherein the mammalian cell or human cell is a T cell or an induced pluripotent stem cell.
565. A method for producing a genetically modified cell comprising insertions and/or deletions within a T cell receptor alpha chain constant (TRAC) gene, wherein the method comprises introducing into a cell the RGN system of any one of embodiments 485-512 or an RNP complex of embodiment 513.
566. The method of embodiment 565, wherein the genetically modified cell has lower levels of TRAC protein compared to a cell that has not been genetically modified.
567. The method of embodiment 565 or 566, wherein the cell is a mammalian cell.
568 The method of embodiment 567, wherein the mammalian cell is a human cell.
569. The method of embodiment 567 or 568, wherein the mammalian cell or human cell is a T cell or an induced pluripotent stem cell.
570. A genetically modified cell comprising insertions and/or deletions within a TRAC gene produced according to the method of any one of embodiments 565-569.
571. A method for modulating expression of a T cell receptor alpha chain (TRAC) gene in a population of cells, comprising delivering the RGN system of any one of embodiments 485-512 or the RNP complex of embodiment 513 to the population of cells, wherein the population of cells comprises the target sequence, and wherein TRAC gene expression is modulated as compared to TRAC gene expression in a control population of cells.
572. The method of embodiment 571, wherein cleavage or modification of the target sequence occurs.
573. The method of embodiment 572, wherein cleavage or modification of the target sequence is detected by sequencing.
574. The method of any one of embodiments 571-573, wherein TRAC gene expression is measured by quantitative PCR, microarray, RNA-seq, flow cytometry, immunoblot, enzyme-linked immunosorbent assay (ELISA), protein immunoprecipitation, immunostaining, high performance liquid chromatography (HPLC), liquid chromatography-mass spectrometry (LC/MS), mass spectrometry, or a combination thereof.
575. The method of any one of embodiments 571-574, wherein TRAC gene expression is decreased.
576. The method of embodiment 575, wherein the decrease in TRAC gene expression comprises decrease in TRAC mRNA and/or TRAC protein level.
577. The method of embodiment 576, wherein the decrease in TRAC protein level is measured by flow cytometry for detection of CD3+ cells.
578. The method of embodiment 577, wherein a decrease in CD3+ cells as compared to a level of CD3+ cells in the control population of cells is indicative of the decrease in TRAC protein level.
579. The method of embodiment 578, wherein the decrease in CD3+ cells is 30% to 100%.
580. The method of embodiment 578, wherein the decrease in CD3+ cells is 50% to 100%.
581. The method of any one of embodiments 572-580, wherein cleavage or modification of the target sequence occurs at a rate of 40% to 100%.
582. The method of any one of embodiments 572-580, wherein cleavage or modification of the target sequence occurs at a rate of 80% to 100%.
583. The method of any one of embodiments 571-582, wherein the control population of cells has not been subjected to the delivering.
584. The method of any one of embodiments 571-583, wherein the population of cells comprises T cells.

The following examples are offered by way of illustration and not by way of limitation.

### EXAMPLES

### Example 1. Establishing Conditions for Gene Editing of a T Cell Receptor Alpha Chain Constant (TRAC) Gene

Conditions were established to edit the TRAC gene using an APG07433.1 RGN (SEQ ID NO: 105) or an APG08290.1 RGN (SEQ ID NO: 333) and guide RNAs disclosed herein. RGN expression cassettes were produced and introduced into vectors for mammalian expression. The APG07433.1 and APG08290.1 RGNs were codon-optimized for human expression (APG07433.1, SEQ ID NO: 108; APG08290.1, SEQ ID NO: 428), and operably fused at the 5′ end to an SV40 nuclear localization sequence (NLS; SEQ ID NO: 411) and to 3×FLAG tag (SEQ ID NO: 425), and operably fused at the 3′ end to nucleoplasmin NLS sequences (SEQ ID NO: 412). Guide RNA (gRNA) expression constructs each encoding a single gRNA under the control of a human RNA polymerase III U6 promoter (SEQ ID NO: 413) were produced and introduced into the pTwist High Copy Amp vector. Guides were synthesized with phosphorothioated 2′-O-methyl modifications to the 5′ terminal 3 nt and 3′ terminal 3 nt of each guide. Spacer and target sequences for each guide are included in the Sequence Listing and sequence descriptions are in Table 10.

The constructs described above were introduced into mammalian cells. One day prior to transfection, 1×10⁵ HEK293T cells (Sigma) were plated in 24-well dishes in Dulbecco's modified Eagle medium (DMEM) plus 10% (vol/vol) fetal bovine serum (Gibco) and 1% Penicillin-Streptomycin (Gibco). The next day when the cells were at 50-60% confluency, 500 ng of an RGN expression plasmid plus 500 ng of a single gRNA expression plasmid were co-transfected using 1.5 μL of Lipofectamine 3000 (Thermo Scientific) per well, following the manufacturer's instructions. After 48 hours of growth, total genomic DNA was harvested using a genomic DNA isolation kit (Machery-Nagel) according to the manufacturer's instructions.

The total genomic DNA was then analyzed to determine the rate of editing for each TRAC target. First, oligonucleotides were produced to be used for PCR amplification and subsequent analysis of the amplified TRAC target site. Oligonucleotide sequences used are listed in Table 4.

All PCR reactions were performed using 10 μL of 2× Master Mix Phusion High-Fidelity DNA polymerase (Thermo Scientific) in a 20 μL reaction including 0.5 μM of each primer. Large genomic regions encompassing each target gene were first amplified using PCR #1 primers, using a program of: 98° C., 1 min; 30 cycles of [98° C., 10 sec; 62° C., 15 sec; 72° C., 5 min]; 72° C., 5 min; 12° C., forever. One μL of this PCR reaction was then further amplified using primers specific for each guide (PCR #2 primers), using a program of: 98° C., 1 min; 35 cycles of [98° C., 10 sec; 67° C., 15 sec; 72° C., 30 sec]; 72° C., 5 min; 12° C., forever. Primers for PCR #2 include Nextera Read 1 and Read 2 Transposase Adapter overhang sequences for Illumina sequencing.

Table 2 lists TRAC guide RNAs used in experiments described in the Examples, along with sequence identifiers for the guide RNAs and their target sequences.

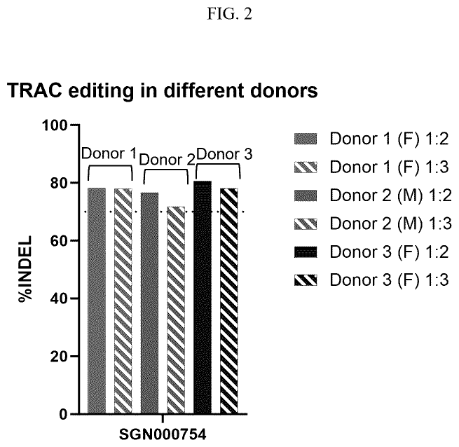

Consistent editing with TRAC guide RNAs were obtained at higher doses of ribonucleoprotein (RNP) complex of guide RNA and APG07433.1 RGN (FIG. 1). FIG. 2 shows that a TRAC guide RNA has >70% editing at TRAC in cells from different donors in association with the APG07433.1 RGN.

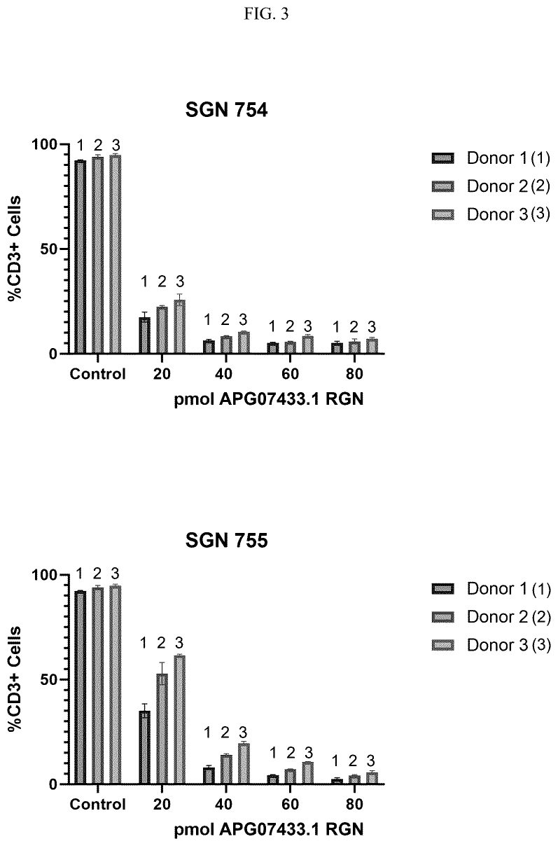

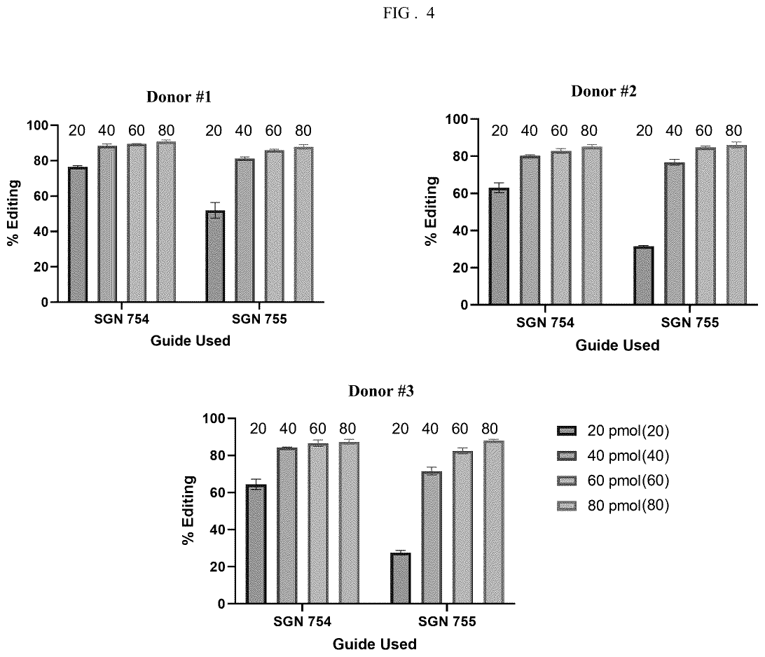

Consistently high TRAC editing was obtained using APG07433.1 RGN in cells from different donors and across a range of RNP complex doses, as measured by a decrease in CD3+ cells by flow cytometry (FIG. 3) and by sequencing of the TRAC target (FIG. 4).

### Example 2. A Screen to Identify APG07433.1 Guide RNA Backbones Effective for Targeting the TRAC Gene

Shortened backbone variants of the native APG07433.1 backbone (native backbone length of 110 nucleotides (nt)) were tested to see which were most effective in editing of the TRAC gene. Guide RNAs with two different spacers (1880 and 1881) were tested with the indicated backbone variants and spacer lengths, and compared to guide RNA with native backbone and 25 nt spacer (‘Full Length’). TRAC editing was measured by knockdown of the CD3 surface marker in cells.

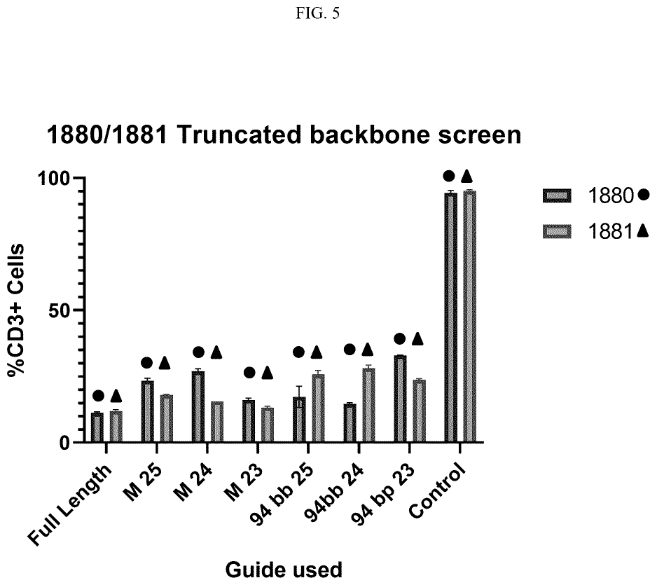

The highest editing for the ‘1880’ spacer TRAC guide RNA was observed with a 24 nt spacer and a 94 nt backbone (118 nt total length of guide, SGN3156 guide RNA SEQ ID NO: 204), and the highest editing for the ‘1881’ spacer TRAC guide RNA was observed with a 23 nt spacer and the M backbone (117 nt total length of guide, SGN6286 guide RNA SEQ ID NO: 205) (FIG. 5). The M backbone has: a deletion of 10 nt in the first stem of stem loop 1 formed by hybridization of the crRNA repeat and anti-repeat; a deletion of 2 nt in stem loop 3 most proximal to the tail of the guide RNA; and a deletion of 4 nt from the tail of the guide RNA; as compared to the native APG07433.1 backbone. The 94bb has a deletion of 16 nt in the first stem of stem loop 1, as compared to the native APG07433.1 backbone.

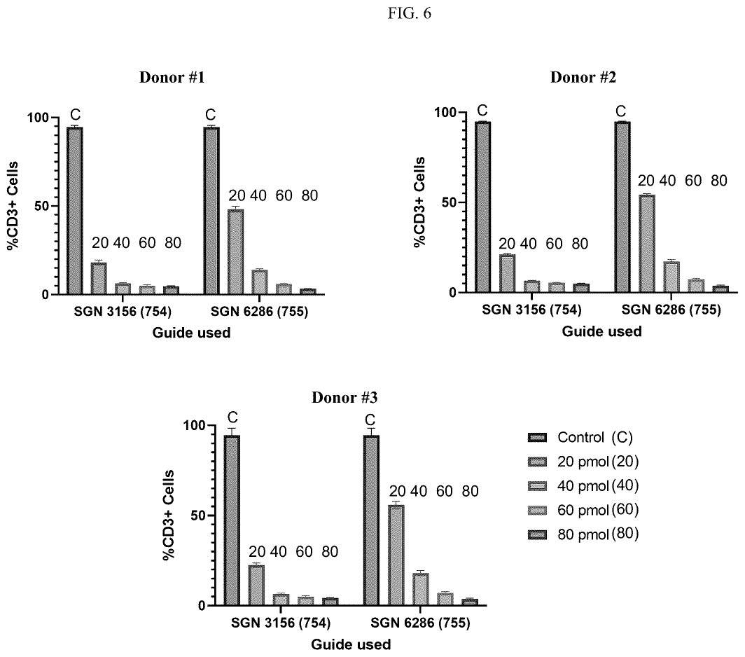

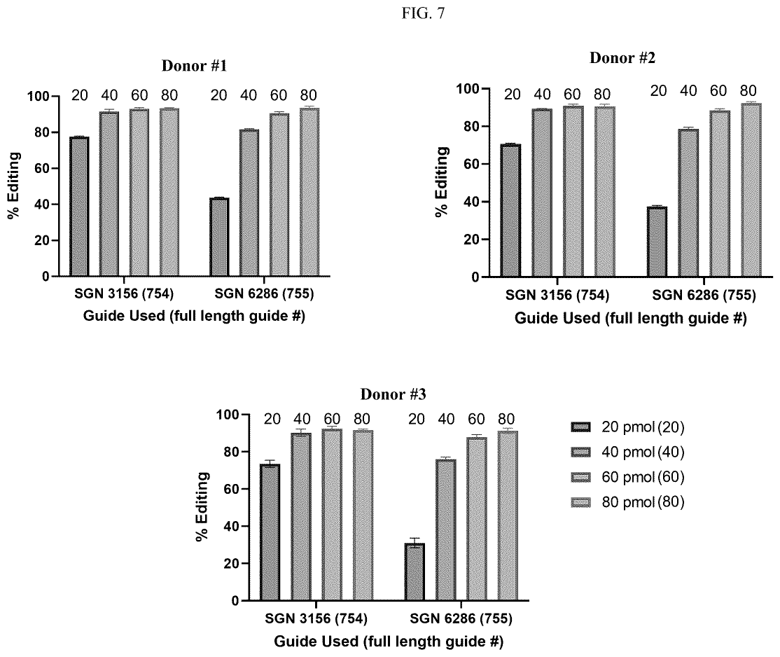

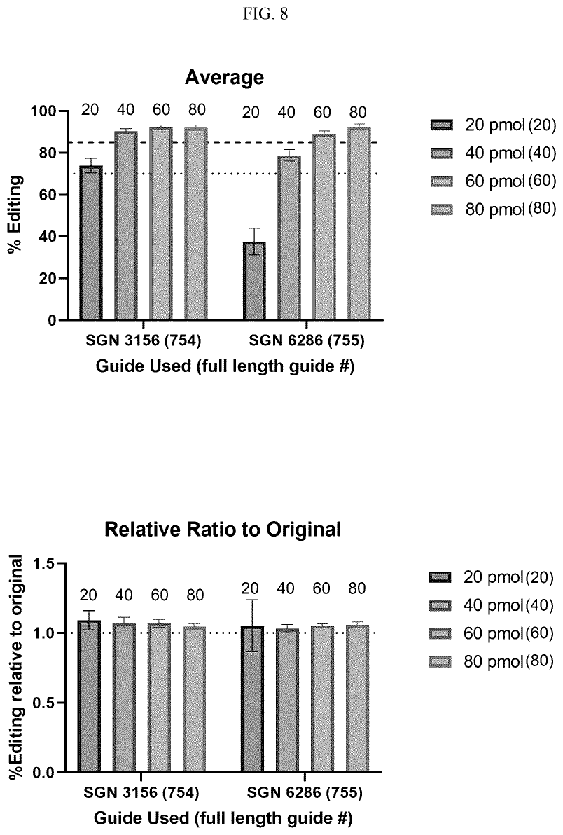

The SGN3156 and SGN6286 truncated guide RNAs (shortened in spacer and backbone) were effective at editing 2 TRAC target sites across a dose range of RNP complex of guide RNA and APG07433.1 RGN and across multiple donors (FIGS. 6, 7). All 3 donors showed over 95% knockdown with both guides on average at highest dose.

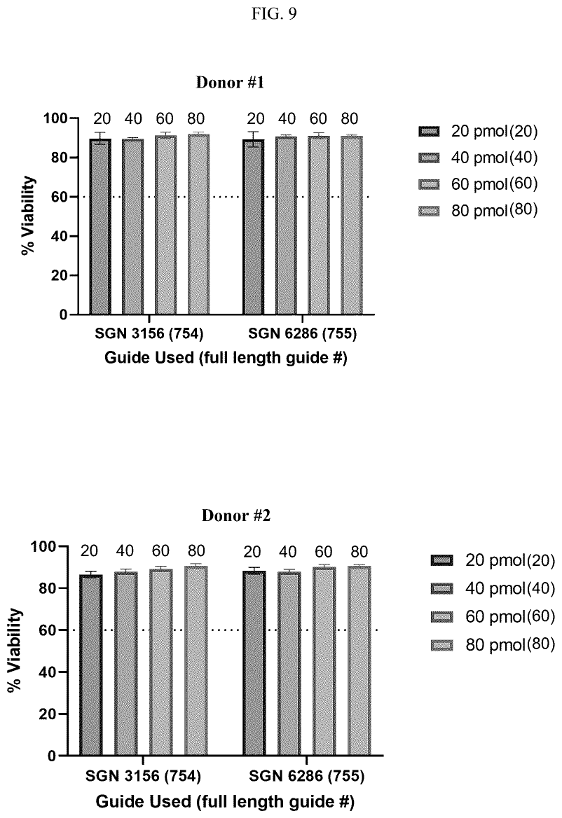

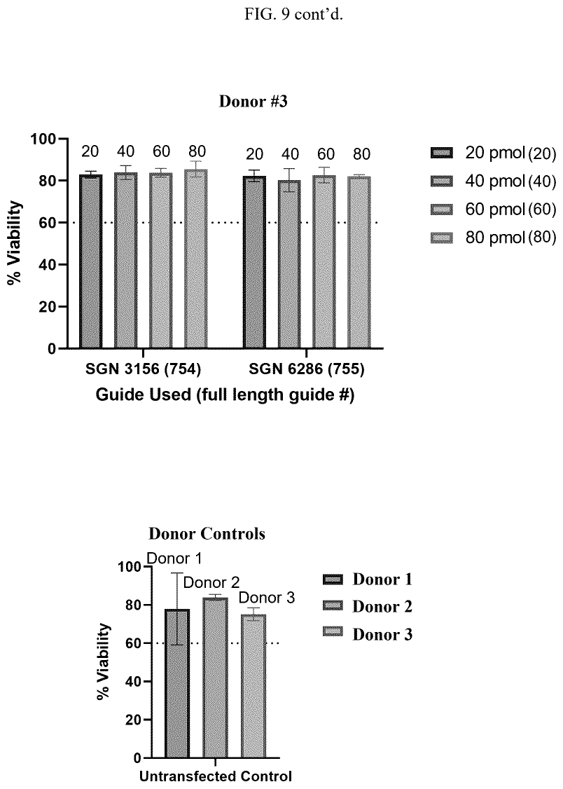

The SGN3156 and SGN6286 truncated TRAC guide RNAs showed equal or slightly improved editing as compared to the original guide RNA with native backbone and 25 nt spacer (FIG. 8). Cell viability was at or above 80% for most samples, across multiple donors, and across a dose range of RNP complex for these truncated TRAC guide RNA and APG07433.1 RGN (FIG. 9).

### Example 3. Screens to Identify Guide RNAs Effective in Targeting the TRAC Gene for Editing

Guide RNAs were screened for their effectiveness in cutting target sequences in the TRAC gene in association with the APG07433.1 RGN or the APG08290.1 RGN as described in Example 1. The RGN was delivered as RNP, mRNA, or plasmid. The tested guide RNAs, their target sequences, and PAM sequences are listed in Table 2. Table 2 also indicates which TRAC target sequences could also be targeted by S. pyogenes Cas9 (SpyCas9) and/or LPG10145 RGN due to the PAM sequences. Two days after lipofection, the genomic DNA (gDNA) was extracted from the cells, and next generation sequencing (NGS) was performed on an amplified fragment of the TRAC gene. Table 4 shows the primer sequences used for amplifications. This initial screen demonstrated that not all guides show robust editing at the TRAC locus (Table 3). Table 3 shows gene editing as percent insertions and deletions (indels) using TRAC guide RNAs with APG07433.1 RGN or APG08290.1 RGN. It was determined that multiple guide RNAs showed >50% editing at TRAC by plasmid delivery. Robust editing was obtained with guides having spacer sequences of SEQ ID NOs: 7 and 9, and with the APG07433.1 RGN (Table 5). Table 5 shows gene editing data for lead TRAC guide RNAs as a function of ribonucleoprotein (RNP) dose response. The guide RNAs from the screening that were most effective in targeting TRAC for gene editing are listed in Table 6.

TABLE 2
List of TRAC guide RNAs screened and/or used in gene editing experiments.

| | | | | | | Targetable |
|---|---|---|---|---|---|---|
| | Guide | | Target | | Target | by SpyCas9 |
| | RNA SEQ | | SEQ | PAM | Length | and/or |
| Guide name | ID NO: | Target Sequence | ID NO: | Sequence | (in nt) | LPG10145* |
| SGN000753 | 198 | TCTTGTCCCACAGATATCC | 2 | CTGACC | 25 | |
| | | AGAACC | | CT | | |
| SGN000754 | 199 | TGCCGTGTACCAGCTGAGA | 4 | AAATCC | 25 | |
| | | GACTCT | | AG | | |
| SGN001755 | 200 | TGATCCTCTTGTCCCACAG | 6 | AGAACC | 25 | |
| | | ATATCC | | CT | | |
| SGN001154 | 20 | TGCCGTGTACCAGCTGAGA | 4 | AAATCC | 25 | |
| | | GACTCT | | AG | | |
| SGN001880 | 202 | TGCCGTGTACCAGCTGAGA | 4 | AAATCC | 25 | |
| | | GACTCT | | AG | | |
| SGN001881 | 203 | TGATCCTCTTGTCCCACAG | 6 | AGAACC | 25 | |
| | | ATATCC | | CT | | |
| SGN003156 | 204 | GCCGTGTACCAGCTGAGAG | 8 | AAATCC | 24 | |
| | | ACTCT | | AG | | |
| SGN006286 | 205 | ATCCTCTTGTCCCACAGAT | 10 | AGAACC | 23 | |
| | | ATCC | | CT | | |
| SGN000908 | 206 | CCTAGAGCCCAAGAGAACC | 12 | AGATCC | 25 | |
| | | CACTGA | | AT | | |
| SGN000909 | 207 | TTTAGGTTCGTATCTGTAAA | 14 | GAGGCC | 25 | |
| | | ACCAA | | AC | | |
| SGN000910 | 208 | CAGGGCCTCGATAATGAGA | 16 | CCCCCC | 25 | |
| | | TAATTT | | AC | | |
| SGN000911 | 209 | CTCAACATTCCAGGGCTCA | 18 | CCTCCC | 25 | |
| | | AGCGAT | | AT | | |
| SGN000912 | 210 | AAGAGTATAAACACGTACT | 20 | TGTTCC | 25 | |
| | | TTCATA | | AA | | |
| SGN000914 | 211 | TGTCTCAAATAATAAAAAA | 22 | AACTCC | 25 | |
| | | AATAAG | | AG | | |
| SGN000915 | 212 | ATCATCCTGAGGACAGCCA | 24 | TTTGCC | 25 | |
| | | AGAGGT | | TT | | |
| SGN000916 | 213 | GACCTCAGGTGATCCACCC | 26 | GCCTCC | 25 | |
| | | GCTTCA | | CA | | |
| SGN000919 | 214 | GTAGGTGCTCAGAATTTAT | 28 | AATACC | 25 | |
| | | CATCAC | | TC | | |
| SGN000921 | 215 | ACTCATTAACCCACCAATC | 30 | TGTGCC | 25 | |
| | | ACTGAT | | GG | | |
| SGN000922 | 216 | TTCCTTGCTTCAGGAATGG | 32 | TCTGCC | 25 | |
| | | CCAGGT | | CA | | |
| SGN000923 | 217 | TCTTTTATTTAATAGGATCT | 34 | AAAACC | 25 | |
| | | TCTTC | | CC | | |
| SGN000924 | 218 | CATTCCAGATCTGCAAGAT | 36 | ACAGCC | 25 | |
| | | TGTAAG | | TG | | |
| SGN000925 | 219 | CCCAGAAGGGCTCAGAAAT | 38 | AGAGCC | 25 | |
| | | AAAATA | | AA | | |
| SGN000926 | 220 | AGAGCCATCACCATCACAT | 40 | CAGTCC | 25 | |
| | | CCCTAA | | TG | | |
| SGN000927 | 221 | GGCCAAAATCCAGCTGCCT | 42 | ATCCCC | 25 | |
| | | TCCTTG | | TC | | |
| SGN000928 | 222 | GCTTCTACTCTTGGCTTACA | 44 | CTCTCC | 25 | |
| | | ACCCA | | GT | | |
| SGN000929 | 223 | ATCTGGAGGGAACTTACAG | 46 | AGCACC | 25 | |
| | | CATATG | | TG | | |
| SGN002930 | 224 | GAACAACTCAAATGGAAAT | 48 | GGGCCC | 25 | SpyCas9 |
| | | GAATAT | | AG | | |
| SGN000931 | 225 | TCCTGTTCCATCACCATCAA | 50 | AACTCC | 25 | |
| | | AAAAA | | AG | | |
| SGN000932 | 226 | TAAAATGATTTGGAAGAGC | 52 | TGTGCC | 25 | |
| | | AGAGAC | | TC | | |
| SGN000933 | 227 | CCACCCAGCCTGCTCTGCC | 54 | AAAACC | 25 | |
| | | TTGGGG | | GT | | |
| SGN000934 | 228 | AAGACATTACACTCATATT | 56 | CAATCC | 25 | |
| | | ACACCC | | TG | | |
| SGN000935 | 229 | TGTTGGTTACCTCCCTGCCA | 58 | CTGCCC | 25 | |
| | | CCACC | | AG | | |
| SGN000936 | 230 | CGGCACTATCATTGACTCA | 60 | CAGGCC | 25 | LPG10145 |
| | | GCTGCT | | AA | | |
| SGN000937 | 231 | CCCTGGCTGTGCACATTCC | 62 | CTCCCC | 25 | |
| | | CTCCTG | | AG | | |
| SGN000938 | 232 | GACATCATTGACCAGAGCT | 64 | AGAACC | 25 | |
| | | CTGGGC | | TG | | |
| SGN000939 | 233 | CAGAAGTCAGGCTGGACAC | 66 | AGACCC | 25 | |
| | | TGAGGA | | AG | | |
| SGN000940 | 234 | GGAGGCCTGCAGCTCCTGC | 68 | TGTCCC | 25 | |
| | | ACCTCC | | TT | | |
| SGN000942 | 235 | GCAGCCTCCCCTGGCTGTG | 70 | CCCTCC | 25 | |
| | | CACATT | | TG | | |
| SGN000943 | 236 | GCTACCACCTCTGTGCCCC | 72 | AATGCC | 25 | |
| | | CCCGGC | | AC | | |
| SGN000944 | 23 | AACCCCACTCCCAGCTTCA | 74 | CTCACC | 25 | |
| | | AGGCCC | | TC | | |
| SGN000947 | 238 | CAGGCCAAAATCCAGCTGC | 76 | TGATCC | 25 | |
| | | CTTCCT | | CC | | |
| SGN000948 | 239 | TCCACTTCAACACCTGGTG | 78 | TGTGCC | 25 | |
| | | CATTCA | | GG | | |
| SGN000949 | 240 | GGCAGGACCGATACCTCGG | 80 | GACACC | 25 | |
| | | CTCCTG | | AT | | |
| SGN000950 | 241 | TGGTGCATCTTGGCTCACT | 82 | TCCGCC | 25 | |
| | | GCAACC | | TC | | |
| SGN000951 | 242 | ACCAGCCTGGGCAACACAG | 84 | CCCGCC | 25 | |
| | | GGAGAC | | TC | | |
| SGN001065 | 243 | GTGCAAACGCCTTCAACAA | 86 | TATTCC | 25 | |
| | | CAGCAT | | AG | | |
| SGN001066 | 244 | ATCCAGTGACAAGTCTGTC | 88 | TTCACC | 25 | |
| | | TGCCTA | | GA | | |
| SGN001268 | 245 | TTTGAAAGTTTAGGTTCGT | 90 | AAAACC | 25 | |
| | | ATCTGT | | AA | | |
| SGN001269 | 246 | TGTCTTACCTGTTTCAAAGC | 92 | TCGACC | 25 | |
| | | TTTTC | | AG | | |
| SGN001794 | 247 | TGTACCAGCTGAGAGACTC | 94 | AAATCC | 20 | |
| | | T | | AG | | |
| SGN001856 | 248 | TCCCACAGATATCCAGAAC | 96 | CCTGCC | 25 | |
| | | CCTGAC | | GT | | |
| SGN001857 | 249 | GATCCTCTTGTCCCACAGA | 98 | GAACCC | 25 | |
| | | TATCCA | | TG | | |
| SGN003197 | 250 | TGCCGTGTACCAGCTGAGA | 4 | AAATCC | 25 | |
| | | GACTCT | | AG | | |
| SGN006283 | 251 | GCCGTGTACCAGCTGAGAG | 8 | AAATCC | 24 | |
| | | ACTCT | | AG | | |
| SGN006284 | 252 | CCGTGTACCAGCTGAGAGA | 100 | AAATCC | 23 | |
| | | CTCT | | AG | | |
| SGN006285 | 253 | GATCCTCTTGTCCCACAGA | 102 | AGAACC | 24 | |
| | | TATCC | | CT | | |
| SGN003261 | 254 | TGATCCTCTTGTCCCACAG | 6 | AGAACC | 25 | |
| | | ATATCC | | CT | | |
| SGN003146 | 255 | TGCCGTGTACCAGCTGAGA | 4 | AAATCC | 25 | |
| | | GACTCT | | AG | | |
| SGN003166 | 256 | CCGTGTACCAGCTGAGAGA | 100 | AAATCC | 23 | |
| | | CTCT | | AG | | |
| SGN003210 | 257 | TGATCCTCTTGTCCCACAG | 6 | AGAACC | 25 | |
| | | ATATCC | | CT | | |
| SGN003220 | 258 | GATCCTCTTGTCCCACAGA | 102 | AGAACC | 24 | |
| | | TATCC | | CT | | |
| SGN003230 | 259 | ATCCTCTTGTCCCACAGAT | 104 | AGAACC | 23 | |
| | | ATCC | | CT | | |

*If the TRAC target sequence can be targeted by S. pyogenes Cas9 (SpyCas9) and/or LPG10145
RGN, the respective polypeptide(s) is indicated.

TABLE 3
Gene editing of TRAC gene with indicated guide RNA.

| | | RGN | Average |
|---|---|---|---|
| Guide | RGN | delivery | Percent |
| Name | system | method | Indels |
| SGN000753 | APG08290.1 | RNP | 2.59 |
| SGN000753 | APG08290.1 | Plasmid | 15.54 |
| SGN000753 | APG08290.1 | mRNA | 11.52 |
| SGN000754 | APG08290.1 | RNP | 12.50 |
| SGN000754 | APG08290.1 | Plasmid | 25.77 |
| SGN000754 | APG08290.1 | mRNA | 63.64 |
| SGN000755 | APG08290.1 | RNP | 41.70 |
| SGN000755 | APG08290.1 | Plasmid | 30.63 |
| SGN000755 | APG08290.1 | mRNA | 63.34 |
| SGN000908 | APG08290.1 | Plasmid | 33.31 |
| SGN000909 | APG08290.1 | Plasmid | 33.70 |
| SGN000910 | APG08290.1 | Plasmid | 25.23 |
| SGN000911 | APG08290.1 | Plasmid | 1.53 |
| SGN000912 | APG08290.1 | Plasmid | 16.77 |
| SGN000914 | APG08290.1 | Plasmid | 14.41 |
| SGN000915 | APG08290.1 | Plasmid | 3.23 |
| SGN000916 | APG08290.1 | Plasmid | 4.66 |
| SGN000919 | APG08290.1 | Plasmid | 0 |
| SGN000921 | APG08290.1 | Plasmid | 8.71 |
| SGN000922 | APG08290.1 | Plasmid | 0.39 |
| SGN000923 | APG08290.1 | Plasmid | 1.02 |
| SGN000924 | APG08290.1 | Plasmid | 17.81 |
| SGN000925 | APG08290.1 | Plasmid | 19.68 |
| SGN000926 | APG08290.1 | Plasmid | 32.17 |
| SGN000927 | APG08290.1 | Plasmid | 16.54 |
| SGN000928 | APG08290.1 | Plasmid | 44.47 |
| SGN000929 | APG08290.1 | Plasmid | 20.82 |
| SGN000930 | APG08290.1 | Plasmid | 28.36 |
| SGN000931 | APG08290.1 | Plasmid | 4.37 |
| SGN000932 | APG08290.1 | Plasmid | 7.47 |
| SGN000933 | APG08290.1 | Plasmid | 17.28 |
| SGN000934 | APG08290.1 | Plasmid | 19.37 |
| SGN000935 | APG08290.1 | Plasmid | 15.29 |
| SGN000936 | APG08290.1 | Plasmid | 32.06 |
| SGN000937 | APG08290.1 | Plasmid | 22.38 |
| SGN000938 | APG08290.1 | Plasmid | 11.80 |
| SGN000939 | APG08290.1 | Plasmid | 33.17 |
| SGN000940 | APG08290.1 | Plasmid | 0 |
| SGN000942 | APG08290.1 | Plasmid | 7.54 |
| SGN000943 | APG08290.1 | Plasmid | 8.65 |
| SGN000944 | APG08290.1 | Plasmid | 10.94 |
| SGN000947 | APG08290.1 | Plasmid | 23.47 |
| SGN000948 | APG08290.1 | Plasmid | 20.26 |
| SGN000949 | APG08290.1 | Plasmid | 16.05 |
| SGN000950 | APG08290.1 | Plasmid | 100 |
| SGN000951 | APG08290.1 | Plasmid | 0 |
| SGN001065 | APG08290.1 | Plasmid | 19.35 |
| SGN001066 | APG08290.1 | Plasmid | 16.07 |
| SGN001154 | APG07433.1 | RNP | 0 |
| SGN001154 | APG07433.1 | mRNA | 0.03 |
| SGN001268 | APG07433.1 | Plasmid | 17.98 |
| SGN001269 | APG07433.1 | Plasmid | 1.11 |
| SGN001794 | APG07433.1 | Plasmid | 35.31 |
| SGN001856 | APG07433.1 | RNP | 1.71 |
| SGN001856 | APG07433.1 | mRNA | 6.32 |
| SGN001857 | APG07433.1 | RNP | 65.61 |
| SGN001857 | APG07433.1 | mRNA | 69.77 |
| SGN001880 | APG07433.1 | RNP | 52.32 |
| SGN001880 | APG07433.1 | mRNA | 66.31 |
| SGN001881 | APG07433.1 | RNP | 63.26 |
| SGN001881 | APG07433.1 | mRNA | 66.12 |

TABLE 4
Primer sets for TRAC gene amplifications

| | Left primer | Right primer |
|---|---|---|
| Primer Set | SEQ ID NO: | SEQ ID NO: |
| A | 260 | 280 |
| B | 261 | 281 |
| C | 262 | 282 |
| D | 263 | 283 |
| E | 264 | 284 |
| F | 265 | 285 |
| G | 266 | 286 |
| H | 267 | 287 |
| I | 268 | 288 |
| J | 269 | 289 |
| K | 270 | 290 |
| L | 271 | 291 |
| M | 272 | 292 |
| N | 273 | 293 |
| TRAC_ex1 | 274 | 294 |
| 713/714 | | |
| TRAC1-2 | 275 | 295 |
| TRAC1-3 | 276 | 296 |
| TRAC1-4 | 277 | 297 |
| TRAC1-6 | 278 | 298 |
| TRAC1-10 | 279 | 299 |

TABLE 5
Lead guide RNA dose response data in multiple donors.

| | | RNP | |
|---|---|---|---|
| Guide | | Concentration | Percent |
| Name | Cell Line | (pmol) | Indels |
| SGN003156 | Tcell-001 | 20 | 77.72 |
| SGN003156 | Tcell-001 | 20 | 77.84 |
| SGN003156 | Tcell-001 | 20 | 76.98 |
| SGN003156 | Tcell-001 | 40 | 90.34 |
| SGN003156 | Tcell-001 | 40 | 91.77 |
| SGN003156 | Tcell-001 | 40 | 92.67 |
| SGN003156 | Tcell-001 | 60 | 92.48 |
| SGN003156 | Tcell-001 | 60 | 93.17 |
| SGN003156 | Tcell-001 | 60 | 93.72 |
| SGN003156 | Tcell-001 | 80 | 93.43 |
| SGN003156 | Tcell-001 | 80 | 93.65 |
| SGN003156 | Tcell-001 | 80 | 92.98 |
| SGN006286 | Tcell-001 | 20 | 43.86 |
| SGN006286 | Tcell-001 | 20 | 43.96 |
| SGN006286 | Tcell-001 | 20 | 43.46 |
| SGN006286 | Tcell-001 | 40 | 81.97 |
| SGN006286 | Tcell-001 | 40 | 81.17 |
| SGN006286 | Tcell-001 | 40 | 81.74 |
| SGN006286 | Tcell-001 | 60 | 90.38 |
| SGN006286 | Tcell-001 | 60 | 90.08 |
| SGN006286 | Tcell-001 | 60 | 91.53 |
| SGN006286 | Tcell-001 | 80 | 92.93 |
| SGN006286 | Tcell-001 | 80 | 93.53 |
| SGN006286 | Tcell-001 | 80 | 94.65 |
| SGN003156 | Tcell-002 | 20 | 70.28 |
| SGN003156 | Tcell-002 | 20 | 71.11 |
| SGN003156 | Tcell-002 | 20 | 70.45 |
| SGN003156 | Tcell-002 | 40 | 89.40 |
| SGN003156 | Tcell-002 | 40 | 89.30 |
| SGN003156 | Tcell-002 | 40 | 89.43 |
| SGN003156 | Tcell-002 | 60 | 90.01 |
| SGN003156 | Tcell-002 | 60 | 91.23 |
| SGN003156 | Tcell-002 | 60 | 91.60 |
| SGN003156 | Tcell-002 | 80 | 91.37 |
| SGN003156 | Tcell-002 | 80 | 89.48 |
| SGN003156 | Tcell-002 | 80 | 91.34 |
| SGN006286 | Tcell-002 | 20 | 37.85 |
| SGN006286 | Tcell-002 | 20 | 36.91 |
| SGN006286 | Tcell-002 | 20 | 37.72 |
| SGN006286 | Tcell-002 | 40 | 78.18 |
| SGN006286 | Tcell-002 | 40 | 78.14 |
| SGN006286 | Tcell-002 | 40 | 79.70 |
| SGN006286 | Tcell-002 | 60 | 87.87 |
| SGN006286 | Tcell-002 | 60 | 88.35 |
| SGN006286 | Tcell-002 | 60 | 89.49 |
| SGN006286 | Tcell-002 | 80 | 91.83 |
| SGN006286 | Tcell-002 | 80 | 92.48 |
| SGN006286 | Tcell-002 | 80 | 93.16 |
| SGN003156 | Tcell-003 | 20 | 75.65 |
| SGN003156 | Tcell-003 | 20 | 71.84 |
| SGN003156 | Tcell-003 | 20 | 72.99 |
| SGN003156 | Tcell-003 | 40 | 87.90 |
| SGN003156 | Tcell-003 | 40 | 91.50 |
| SGN003156 | Tcell-003 | 40 | 91.16 |
| SGN003156 | Tcell-003 | 60 | 92.32 |
| SGN003156 | Tcell-003 | 60 | 91.30 |
| SGN003156 | Tcell-003 | 60 | 93.76 |
| SGN003156 | Tcell-003 | 80 | 91.81 |
| SGN003156 | Tcell-003 | 80 | 91.10 |
| SGN003156 | Tcell-003 | 80 | 92.19 |
| SGN006286 | Tcell-003 | 20 | 34.01 |
| SGN006286 | Tcell-003 | 20 | 29.21 |
| SGN006286 | Tcell-003 | 20 | 29.81 |
| SGN006286 | Tcell-003 | 40 | 75.82 |
| SGN006286 | Tcell-003 | 40 | 75.14 |
| SGN006286 | Tcell-003 | 40 | 77.21 |
| SGN006286 | Tcell-003 | 60 | 86.74 |
| SGN006286 | Tcell-003 | 60 | 87.84 |
| SGN006286 | Tcell-003 | 60 | 89.25 |
| SGN006286 | Tcell-003 | 80 | 90.46 |
| SGN006286 | Tcell-003 | 80 | 90.50 |
| SGN006286 | Tcell-003 | 80 | 92.92 |
| SGN006286\hat{ } | Tcell-001 | 0 | 0.14 |
| SGN006286 | Tcell-002 | 0 | 0 |
| SGN006286 | Tcell-003 | 0 | 0 |

\hat{ }Unedited controls are italicized

TABLE 6
Lead TRAC guide RNAs that yield the best gene
editing with APG07433.1 RGN from the screens.

| | Guide | spacer | Target |
|---|---|---|---|
| | RNA SEQ | SEQ ID | SEQ ID |
| Guide Name | ID NO: | NO: | NO: |
| SGN003156 | 204 | 7 | 8 |
| SGN006286 | 205 | 9 | 10 |

### Example 4. No Bona Fide Off-Target Gene Editing was Seen for the Lead TRAC Guide RNAs

Bioinformatics was used to identify potential off-target sequences. The criteria for potential off-target site selection included no mismatches in the PAM sequence and 5 or less mismatches in the spacer (which includes RNA and DNA bulges). Table 7 shows predicted off-target sites for some TRAC guide RNAs and their spacer lengths. Manipulation of spacer length can alter the predicted off-target sites.

TABLE 7
Bioinformatic identification of potential off-target sites.

| Original Guide | | # | Shortened | Spacer | # |
|---|---|---|---|---|---|
| RNA Name | Spacer (nt) | POTS* | Guide | (bp) | POTS |
| SGN000754 | 25 | 4 | SGN003156 | 24 | 1 |
| SGN000755 | 25 | 4 | SGN006286 | 23 | 7 |

*POTS - Predicted Off Target Sites

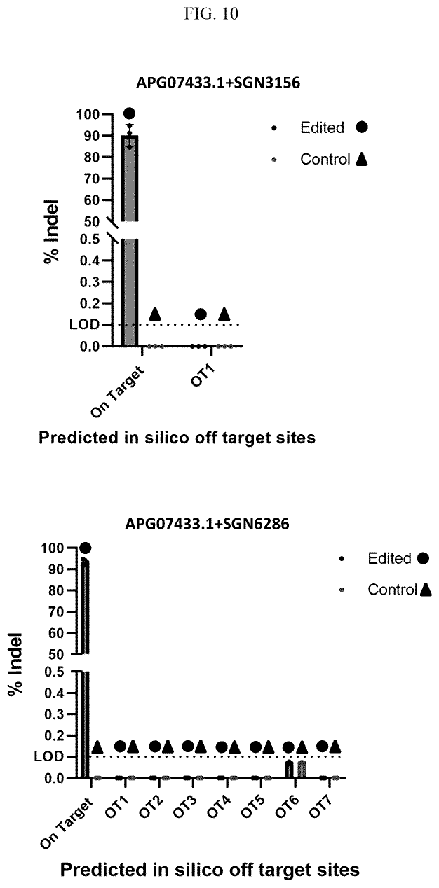

Amplicon sequencing (Amp-Seq) was used to confirm bona fide off-target sites at 0.1% limit of detection. Table 8 shows gene editing rates for off-target sites for the lead TRAC guide RNAs. The SGN3156 and SGN6286 truncated TRAC guide RNAs had no bona fide off-target gene editing (FIG. 10). Table 9 lists the primers used in generating amplicons for Amp-Seq.

TABLE 8
Gene editing of off-target sequences for lead TRAC guide RNAs.

| | Off-Target | | Control | Reads | Reads | Percent |
|---|---|---|---|---|---|---|
| Guide | Number | Cell Line | Type | In | Aligned | Indels |
| SGN003156 | 3156-OT1 | Cell line-001 | | 180629 | 179771 | 0 |
| | | Cell line-002 | | 178124 | 177582 | 0 |
| | | Cell line-003 | | 158209 | 157794 | 0 |
| | | Cell line-001 | Unedited Control | 179382 | 178893 | 0 |
| | | Cell line-002 | Unedited Control | 122428 | 122107 | 0 |
| | | Cell line-003 | Unedited Control | 125438 | 125146 | 0 |
| SGN006286 | 6286-OT1 | Cell line-001 | | 165312 | 164958 | 0 |
| | | Cell line-002 | | 141628 | 141319 | 0 |
| | | Cell line-003 | | 152891 | 152575 | 0 |
| | | Cell line-001 | Unedited Control | 131680 | 131352 | 0 |
| | | Cell line-002 | Unedited Control | 121138 | 120873 | 0 |
| | | Cell line-003 | Unedited Control | 124221 | 123986 | 0 |
| | 6286-OT2 | Cell line-001 | | 185022 | 184750 | 0 |
| | | Cell line-002 | | 132275 | 132103 | 0 |
| | | Cell line-003 | | 167494 | 167313 | 0 |
| | | Cell line-001 | Unedited Control | 195458 | 195268 | 0 |
| | | Cell line-002 | Unedited Control | 170452 | 170236 | 0 |
| | | Cell line-003 | Unedited Control | 195562 | 195375 | 0 |
| | 6286-OT3 | Cell line-001 | | 159519 | 159273 | 0 |
| | | Cell line-002 | | 166558 | 166336 | 0 |
| | | Cell line-003 | | 151063 | 150876 | 0 |
| | | Cell line-001 | Unedited Control | 158957 | 158727 | 0 |
| | | Cell line-002 | Unedited Control | 127702 | 127490 | 0 |
| | | Cell line-003 | Unedited Control | 161789 | 161572 | 0 |
| | 6286-OT4 | Cell line-001 | | 147865 | 147137 | 0 |
| | | Cell line-002 | | 132968 | 132597 | 0 |
| | | Cell line-003 | | 152265 | 151859 | 0 |
| | | Cell line-001 | Unedited Control | 158917 | 158187 | 0 |
| | | Cell line-002 | Unedited Control | 145360 | 144676 | 0 |
| | | Cell line-003 | Unedited Control | 134660 | 134093 | 0 |
| | 6286-OT5 | Cell line-001 | | 228521 | 228292 | 0 |
| | | Cell line-002 | | 159923 | 159751 | 0 |
| | | Cell line-003 | | 149501 | 149306 | 0 |
| | | Cell line-001 | Unedited Control | 171870 | 171678 | 0 |
| | | Cell line-002 | Unedited Control | 159279 | 159100 | 0 |
| | | Cell line-003 | Unedited Control | 138036 | 137851 | 0 |
| | 6286-OT6 | Cell line-001 | | 200749 | 200137 | 0.069687819 |
| | | Cell line-002 | | 168209 | 167738 | 0.069323463 |
| | | Cell line-003 | | 221454 | 220696 | 0.075978799 |
| | | Cell line-001 | Unedited Control | 202006 | 201481 | 0.069685363 |
| | | Cell line-002 | Unedited Control | 189059 | 188481 | 0.072944155 |
| | | Cell line-003 | Unedited Control | 199317 | 198920 | 0.074704232 |
| | 6286-OT7 | Cell line-001 | | 164025 | 163238 | 0 |
| | | Cell line-002 | | 117164 | 116676 | 0 |
| | | Cell line-003 | | 211755 | 211171 | 0 |
| | | Cell line-001 | Unedited Control | 179538 | 178857 | 0 |
| | | Cell line-002 | Unedited Control | 185875 | 185379 | 0 |
| | | Cell line-003 | Unedited Control | 185820 | 184578 | 0 |

TABLE 9
Primer sequences for amplification of the off-target sites.

| | Off- | | Left | Right |
|---|---|---|---|---|
| | Target | | Primer | Primer |
| Off-Target | SEQ | | SEQ ID | SEQ ID |
| Number | ID NO: | Chromosome | NO: | NO: |
| 3156-OT1 | 300 | 17 | 308 | 316 |
| 6286-OT1 | 301 | 16 | 309 | 317 |
| 6286-OT2 | 302 | 8 | 310 | 318 |
| 6286-OT3 | 303 | 22 | 311 | 319 |
| 6286-OT4 | 304 | 8 | 312 | 320 |
| 6286-OT5 | 305 | 21 | 313 | 321 |
| 6286-OT6 | 306 | 12 | 314 | 322 |
| 6286-OT7 | 307 | 12 | 315 | 323 |

TABLE 10
Description of Sequence Identifiers of the application

| SEQ ID NO | Description |
|---|---|
| 1 | SGN000753 spacer |
| 2 | SGN000753 target |
| 3 | SGN000754 |
| | SGN001154 |
| | SGN001880 |
| | SGN003197 |
| | SGN003146 |
| | spacer |
| 4 | SGN000754 |
| | SGN001154 |
| | SGN001880 |
| | SGN003197 |
| | SGN003146 |
| | target |
| 5 | SGN000755 |
| | SGN001881 |
| | SGN003261 |
| | SGN003210 |
| | spacer |
| 6 | SGN000755 |
| | SGN001881 |
| | SGN003261 |
| | SGN003210 |
| | target |
| 7 | SGN003156 |
| | SGN006283 |
| | spacer |
| 8 | SGN003156 |
| | SGN006283 |
| | target |
| 9 | SGN006286 spacer |
| 10 | SGN006286 target |
| 11 | SGN000908 spacer |
| 12 | SGN000908 target |
| 13 | SGN000909 spacer |
| 14 | SGN000909 target |
| 15 | SGN000910 spacer |
| 16 | SGN000910 target |
| 17 | SGN000911 spacer |
| 18 | SGN000911 target |
| 19 | SGN000912 spacer |
| 20 | SGN000912 target |
| 21 | SGN000914 spacer |
| 22 | SGN000914 target |
| 23 | SGN000915 spacer |
| 24 | SGN000915 target |
| 25 | SGN000916 spacer |
| 26 | SGN000916 target |
| 27 | SGN000919 spacer |
| 28 | SGN000919 target |
| 29 | SGN000921 spacer |
| 30 | SGN000921 target |
| 31 | SGN000922 spacer |
| 32 | SGN000922 target |
| 33 | SGN000923 spacer |
| 34 | SGN000923 target |
| 35 | SGN000924 spacer |
| 36 | SGN000924 target |
| 37 | SGN000925 spacer |
| 38 | SGN000925 target |
| 39 | SGN000926 spacer |
| 40 | SGN000926 target |
| 41 | SGN000927 spacer |
| 42 | SGN000927 target |
| 43 | SGN000928 spacer |
| 44 | SGN000928 target |
| 45 | SGN000929 spacer |
| 46 | SGN000929 target |
| 47 | SGN000930 spacer |
| 48 | SGN000930 target |
| 49 | SGN000931 spacer |
| 50 | SGN000931 target |
| 51 | SGN000932 spacer |
| 52 | SGN000932 target |
| 53 | SGN000933 spacer |
| 54 | SGN000933 target |
| 55 | SGN000934 spacer |
| 56 | SGN000934 target |
| 57 | SGN000935 spacer |
| 58 | SGN000935 target |
| 59 | SGN000936 spacer |
| 60 | SGN000936 target |
| 61 | SGN000937 spacer |
| 62 | SGN000937 target |
| 63 | SGN000938 spacer |
| 64 | SGN000938 target |
| 65 | SGN000939 spacer |
| 66 | SGN000939 target |
| 67 | SGN000940 spacer |
| 68 | SGN000940 target |
| 69 | SGN000942 spacer |
| 70 | SGN000942 target |
| 71 | SGN000943 spacer |
| 72 | SGN000943 target |
| 73 | SGN000944 spacer |
| 74 | SGN000944 target |
| 75 | SGN000947 spacer |
| 76 | SGN000947 target |
| 77 | SGN000948 spacer |
| 78 | SGN000948 target |
| 79 | SGN000949 spacer |
| 80 | SGN000949 target |
| 81 | SGN000950 spacer |
| 82 | SGN000950 target |
| 83 | SGN000951 spacer |
| 84 | SGN000951 target |
| 85 | SGN001065 spacer |
| 86 | SGN001065 target |
| 87 | SGN001066 spacer |
| 88 | SGN001066 target |
| 89 | SGN001268 spacer |
| 90 | SGN001268 target |
| 91 | SGN001269 spacer |
| 92 | SGN001269 target |
| 93 | SGN001794 spacer |
| 94 | SGN001794 target |
| 95 | SGN001856 spacer |
| 96 | SGN001856 target |
| 97 | SGN001857 spacer |
| 98 | SGN001857 target |
| 99 | SGN006284 |
| | SGN003166 |
| | spacer |
| 100 | SGN006284 |
| | SGN003166 |
| | target |
| 101 | SGN006285 |
| | SGN003220 |
| | spacer |
| 102 | SGN006285 |
| | SGN003220 |
| | target |
| 103 | SGN003230 spacer |
| 104 | SGN003230 target |
| 105 | APG07433.1 RGN |
| 106 | APG07433.1 crRNA |
| | repeat |
| 107 | APG07433.1 tracrRNA |
| 108 | APG07433.1 coding |
| | sequence, mammalian |
| | codon optimized |
| 109 | APG07433.1 crRNA |
| | repeat truncated by 5 nt |
| | at 3′ end |
| 110 | APG07433.1 crRNA |
| | repeat truncated by 6 nt |
| | at 3′ end |
| 111 | APG07433.1 crRNA |
| | repeat truncated by 7 nt |
| | at 3′ end |
| 112 | APG07433.1 crRNA |
| | repeat truncated by 8 nt |
| | at 3′ end |
| 113 | APG07433.1 truncated |
| | crRNA repeat in |
| | SGN001154 |
| 114 | APG07433.1 tracrRNA |
| | truncated by 5 nt at 5′ |
| | end, -4 tail, -2SL3 |
| 115 | APG07433.1 tracrRNA |
| | truncated by 6 nt at 5′ |
| | end (98 bb) |
| 116 | APG07433.1 tracrRNA |
| | 98bb_-6 tail |
| 117 | APG07433.1 tracrRNA |
| | 98bb_-6 tail_-2SL3 |
| 118 | APG07433.1 tracrRNA |
| | truncated by 7 nt at 5′ |
| | end (96 bb) |
| 119 | APG07433.1 tracrRNA |
| | 96bb_-6 tail |
| 120 | APG07433.1 tracrRNA |
| | 96bb_-6 tail_-2SL3 |
| 121 | APG07433.1 tracrRNA |
| | truncated by 8 nt at 5′ |
| | end (94 bb) |
| 122 | APG07433.1 tracrRNA |
| | 94bb_-6 tail |
| 123 | APG07433.1 tracrRNA |
| | 94bb_-6tail-2SL3 |
| 124 | APG07433.1 M |
| | backbone |
| 125 | APG07433.1 98 bb |
| | backbone |
| 126 | APG07433.1 98bb_-6 |
| | tail backbone |
| 127 | APG07433.1 98bb_-6 |
| | tail_-2SL3 backbone |
| 128 | APG07433.1 96 bb |
| | backbone |
| 129 | APG07433.1 96bb_-6 |
| | tail backbone |
| 130 | APG07433.1 96bb_-6 |
| | tail_-2SL3 backbone |
| 131 | APG07433.1 94 bb |
| | backbone |
| 132 | APG07433.1 94bb_-6 |
| | tail backbone |
| 133 | APG07433.1 94bb_- |
| | 6tail-2SL3 backbone |
| 134 | SGN001268, |
| | SGN001269, |
| | SGN001794, |
| | SGN001856, |
| | SGN001857, |
| | SGN001880, |
| | SGN001881, and |
| | Full-length |
| | APG07433.1 backbone |
| 135 | SGN001154 backbone |
| 136 | SGN000753 crRNA |
| 137 | SGN000754 crRNA |
| 138 | SGN000755 crRNA |
| 139 | SGN001154 crRNA |
| 140 | SGN001880 crRNA |
| 141 | SGN001881 crRNA |
| 142 | SGN003156 crRNA |
| 143 | SGN006286 crRNA |
| 144 | SGN000908 crRNA |
| 145 | SGN000909 crRNA |
| 146 | SGN000910 crRNA |
| 147 | SGN000911 crRNA |
| 148 | SGN000912 crRNA |
| 149 | SGN000914 crRNA |
| 150 | SGN000915 crRNA |
| 151 | SGN000916 crRNA |
| 152 | SGN000919 crRNA |
| 153 | SGN000921 crRNA |
| 154 | SGN000922 crRNA |
| 155 | SGN000923 crRNA |
| 156 | SGN000924 crRNA |
| 157 | SGN000925 crRNA |
| 158 | SGN000926 crRNA |
| 159 | SGN000927 crRNA |
| 160 | SGN000928 crRNA |
| 161 | SGN000929 crRNA |
| 162 | SGN000930 crRNA |
| 163 | SGN000931 crRNA |
| 164 | SGN000932 crRNA |
| 165 | SGN000933 crRNA |
| 166 | SGN000934 crRNA |
| 167 | SGN000935 crRNA |
| 168 | SGN000936 crRNA |
| 169 | SGN000937 crRNA |
| 170 | SGN000938 crRNA |
| 171 | SGN000939 crRNA |
| 172 | SGN000940 crRNA |
| 173 | SGN000942 crRNA |
| 174 | SGN000943 crRNA |
| 175 | SGN000944 crRNA |
| 176 | SGN000947 crRNA |
| 177 | SGN000948 crRNA |
| 178 | SGN000949 crRNA |
| 179 | SGN000950 crRNA |
| 180 | SGN000951 crRNA |
| 181 | SGN001065 crRNA |
| 182 | SGN001066 crRNA |
| 183 | SGN001268 crRNA |
| 184 | SGN001269 crRNA |
| 185 | SGN001794 crRNA |
| 186 | SGN001856 crRNA |
| 187 | SGN001857 crRNA |
| 188 | SGN003197 crRNA |
| 189 | SGN006283 crRNA |
| 190 | SGN006284 crRNA |
| 191 | SGN006285 crRNA |
| 192 | SGN003261 crRNA |
| 193 | SGN003146 crRNA |
| 194 | SGN003166 crRNA |
| 195 | SGN003210 crRNA |
| 196 | SGN003220 crRNA |
| 197 | SGN003230 crRNA |
| 198 | SGN000753 guide |
| | RNA |
| 199 | SGN000754 guide |
| | RNA |
| 200 | SGN000755 guide |
| | RNA |
| 201 | SGN001154 guide |
| | RNA |
| 202 | SGN001880 guide |
| | RNA |
| 203 | SGN001881 guide |
| | RNA |
| 204 | SGN003156 guide |
| | RNA |
| 205 | SGN006286 guide |
| | RNA |
| 206 | SGN000908 guide |
| | RNA |
| 207 | SGN000909 guide |
| | RNA |
| 208 | SGN000910 guide |
| | RNA |
| 209 | SGN000911 guide |
| | RNA |
| 210 | SGN000912 guide |
| | RNA |
| 211 | SGN000914 guide |
| | RNA |
| 212 | SGN000915 guide |
| | RNA |
| 213 | SGN000916 guide |
| | RNA |
| 214 | SGN000919 guide |
| | RNA |
| 215 | SGN000921 guide |
| | RNA |
| 216 | SGN000922 guide |
| | RNA |
| 217 | SGN000923 guide |
| | RNA |
| 218 | SGN000924 guide |
| | RNA |
| 219 | SGN000925 guide |
| | RNA |
| 220 | SGN000926 guide |
| | RNA |
| 221 | SGN000927 guide |
| | RNA |
| 222 | SGN000928 guide |
| | RNA |
| 223 | SGN000929 guide |
| | RNA |
| 224 | SGN000930 guide |
| | RNA |
| 225 | SGN000931 guide |
| | RNA |
| 226 | SGN000932 guide |
| | RNA |
| 227 | SGN000933 guide |
| | RNA |
| 228 | SGN000934 guide |
| | RNA |
| 229 | SGN000935 guide |
| | RNA |
| 230 | SGN000936 guide |
| | RNA |
| 231 | SGN000937 guide |
| | RNA |
| 232 | SGN000938 guide |
| | RNA |
| 233 | SGN000939 guide |
| | RNA |
| 234 | SGN000940 guide |
| | RNA |
| 235 | SGN000942 guide |
| | RNA |
| 236 | SGN000943 guide |
| | RNA |
| 237 | SGN000944 guide |
| | RNA |
| 238 | SGN000947 guide |
| | RNA |
| 239 | SGN000948 guide |
| | RNA |
| 240 | SGN000949 guide |
| | RNA |
| 241 | SGN000950 guide |
| | RNA |
| 242 | SGN000951 guide |
| | RNA |
| 243 | SGN001065 guide |
| | RNA |
| 244 | SGN001066 guide |
| | RNA |
| 245 | SGN001268 guide |
| | RNA |
| 246 | SGN001269 guide |
| | RNA |
| 247 | SGN001794 guide |
| | RNA |
| 248 | SGN001856 guide |
| | RNA |
| 249 | SGN001857 guide |
| | RNA |
| 250 | SGN003197 guide |
| | RNA |
| 251 | SGN006283 guide |
| | RNA |
| 252 | SGN006284 guide |
| | RNA |
| 253 | SGN006285 guide |
| | RNA |
| 254 | SGN003261 guide |
| | RNA |
| 255 | SGN003146 guide |
| | RNA |
| 256 | SGN003166 guide |
| | RNA |
| 257 | SGN003210 guide |
| | RNA |
| 258 | SGN003220 guide |
| | RNA |
| 259 | SGN003230 guide |
| | RNA |
| 260 | A left primer |
| 261 | B left primer |
| 262 | C left primer |
| 263 | D left primer |
| 264 | E left primer |
| 265 | F left primer |
| 266 | G left primer |
| 267 | H left primer |
| 268 | I left primer |
| 269 | J left primer |
| 270 | K left primer |
| 271 | L left primer |
| 272 | M left primer |
| 273 | N left primer |
| 274 | TRAC_ex1 713/714 |
| | left primer |
| 275 | TRAC1-2 left primer |
| 276 | TRAC1-3 left primer |
| 277 | TRAC1-4 left primer |
| 278 | TRAC1-6 left primer |
| 279 | TRAC1-10 left primer |
| 280 | A right primer |
| 281 | B right primer |
| 282 | C right primer |
| 283 | D right primer |
| 284 | E right primer |
| 285 | F right primer |
| 286 | G right primer |
| 287 | H right primer |
| 288 | I right primer |
| 289 | J right primer |
| 290 | K right primer |
| 291 | L right primer |
| 292 | M right primer |
| 293 | N right primer |
| 294 | TRAC_ex1 713/714 |
| | right primer |
| 295 | TRAC1-2 right primer |
| 296 | TRAC1-3 right primer |
| 297 | TRAC1-4 right primer |
| 298 | TRAC1-6 right primer |
| 299 | TRAC1-10 right primer |
| 300 | 3156-OT1 off-target |
| | sequence |
| 301 | 6286-OT1 off-target |
| | sequence |
| 302 | 6286-OT2 off-target |
| | sequence |
| 303 | 6286-OT3 off-target |
| | sequence |
| 304 | 6286-OT4 off-target |
| | sequence |
| 305 | 6286-OT5 off-target |
| | sequence |
| 306 | 6286-OT6 off-target |
| | sequence |
| 307 | 6286-OT7 off-target |
| | sequence |
| 308 | 3156-OT1 left primer |
| | sequence |
| 309 | 6286-OT1 left primer |
| | sequence |
| 310 | 6286-OT2 left primer |
| | sequence |
| 311 | 6286-OT3 left primer |
| | sequence |
| 312 | 6286-OT4 left primer |
| | sequence |
| 313 | 6286-OT5 left primer |
| | sequence |
| 314 | 6286-OT6 left primer |
| | sequence |
| 315 | 6286-OT7 left primer |
| | sequence |
| 316 | 3156-OT1 right |
| | primer sequence |
| 317 | 6286-OT1 right |
| | primer sequence |
| 318 | 6286-OT2 right |
| | primer sequence |
| 319 | 6286-OT3 right |
| | primer sequence |
| 320 | 6286-OT4 right |
| | primer sequence |
| 321 | 6286-OT5 right |
| | primer sequence |
| 322 | 6286-OT6 right |
| | primer sequence |
| 323 | 6286-OT7 right |
| | primer sequence |
| 324 | Streptococcus |
| | pyogenes Cas9 |
| 325 | Streptococcus |
| | pyogenes Cas9 D10A |
| | nickase |
| 326 | Neisseria |
| | meningitidis Cas9 |
| | (Nme2Cas9) |
| 327 | APG05083.1 RGN |
| 328 | APG05083.1 crRNA |
| | repeat |
| 329 | APG05083.1 |
| | tracrRNA |
| 330 | APG07513.1 RGN |
| 331 | APG07513.1 crRNA |
| | repeat |
| 332 | APG07513.1 |
| | tracrRNA |
| 333 | APG08290.1 RGN |
| 334 | APG08290.1 crRNA |
| | repeat sequence |
| 335 | APG08290.1 |
| | tracrRNA |
| 336 | APG05459.1 RGN |
| 337 | APG05459.1 crRNA |
| | repeat |
| 338 | APG05459.1 |
| | tracrRNA |
| 339 | APG01688.1 RGN |
| 340 | APG01688.1 crRNA |
| | repeat |
| 341 | APG01688.1 |
| | tracrRNA |
| 342 | APG03128 RGN |
| 343 | APG03128 crRNA |
| | repeat |
| 344 | APG03128 tracrRNA |
| 345 | APG05733.1 RGN |
| 346 | APG05733.1 crRNA |
| | repeat |
| 347 | APG05733.1 |
| | tracrRNA |
| 348 | APG01658.1 RGN |
| 349 | APG01658.1 crRNA |
| | repeat |
| 350 | APG01658.1 |
| | tracrRNA |
| 351 | APG06498.1 RGN |
| 352 | APG06498.1 crRNA |
| | repeat |
| 353 | APG06498.1 |
| | tracrRNA |
| 354 | APG06877.1 RGN |
| 355 | APG06877.1 crRNA |
| | repeat |
| 356 | APG06877.1 |
| | tracrRNA |
| 357 | APG09053.1 RGN |
| 358 | APG09053.1 crRNA |
| | repeat |
| 359 | APG09053.1 |
| | tracrRNA |
| 360 | APG04293.1 RGN |
| 361 | APG04293.1 crRNA |
| | repeat |
| 362 | APG04293.1 |
| | tracrRNA |
| 363 | APG06646.1 RGN |
| 364 | APG06646.1 crRNA |
| | repeat |
| 365 | APG06646.1 |
| | tracrRNA |
| 366 | APG02874 RGN |
| 367 | APG02874 crRNA |
| | repeat |
| 368 | APG02874 tracrRNA |
| 369 | APG03031 RGN |
| 370 | APG03031 crRNA |
| | repeat |
| 371 | APG03031 tracrRNA |
| 372 | APG09208 RGN |
| 373 | APG09208 crRNA |
| | repeat |
| 374 | APG09208 tracrRNA |
| 375 | APG09344 RGN |
| 376 | APG09344 crRNA |
| | repeat |
| 377 | APG09344 tracrRNA |
| 378 | APG07991 RGN |
| 379 | APG07991 crRNA |
| | repeat |
| 380 | APG07991 tracrRNA |
| 381 | APG01868 RGN |
| 382 | APG01868 crRNA |
| | repeat |
| 383 | APG01868 tracrRNA |
| 384 | APG02998 RGN |
| 385 | APG02998 crRNA |
| | repeat |
| 386 | APG02998 tracrRNA |
| 387 | APG07433.1 deletion |
| | variant RGN |
| 388 | APG08290.1 deletion |
| | variant RGN |
| 389 | LPG10134 RGN |
| 390 | LPG10134 crRNA |
| | repeat |
| 391 | LPG10134 tracrRNA |
| 392 | LPG10136 RGN |
| 393 | LPG10136 crRNA |
| | repeat |
| 394 | LPG10136 tracrRNA |
| 395 | LPG10138 RGN |
| 396 | LPG10138 crRNA |
| | repeat |
| 397 | LPG10138 tracrRNA |
| 398 | LPG10139 RGN |
| 399 | LPG10139 crRNA |
| | repeat |
| 400 | LPG10139 tracrRNA |
| 401 | LPG10141 RGN |
| 402 | LPG10141 crRNA |
| | repeat |
| 403 | LPG10141 tracrRNA |
| 404 | LPG10145 RGN |
| 405 | LPG10145 crRNA |
| | repeat |
| 406 | LPG10145 tracrRNA |
| 407 | SpyCas9 crRNA |
| | repeat |
| 408 | SpyCas9 tracrRNA |
| 409 | SpyCas9 sgRNA |
| 410 | Conserved amino |
| | acid motif in a |
| | meganuclease |
| 411 | SV40 nuclear |
| | localization signal |
| 412 | nucleoplasmin |
| | nuclear localization |
| | signal |
| 413 | Human U6 promoter |
| 414 | NC_041760 promoter |
| 415 | NW_004848155 |
| | promoter |
| 416 | LR738627 promoter |
| 417 | NC_044556 promoter |
| 418 | XM_030845548 |
| | promoter |
| 419 | Mini NC_041760 |
| | promoter |
| 420 | Mini |
| | NW_004848155 |
| | promoter |
| 421 | Mini LR738627 |
| | promoter |
| 422 | Mini NC_044556 |
| | promoter |
| 423 | Mini XM_030845548 |
| | promoter |
| 424 | CSB/ERCC6, a |
| | member of a |
| | nucleotide excision |
| | repair (NER) |
| | pathway |
| 425 | 3xFLAG tag |
| 426 | Homo sapiens T cell |
| | receptor alpha chain |
| | constant (UniProt ID |
| | P01848) |
| 427 | Homo sapiens TRAC |
| | gene |
| 428 | mammalian codon |
| | optimized |
| | APG08290.1 RGN |
| | coding sequence |
| 429 | mammalian codon |
| | optimized |
| | APG05083.1 RGN |
| | coding sequence |
| 430 | MS modified |
| | APG07433.1 crRNA |
| | repeat |
| 431 | MS modified |
| | APG07433.1 |
| | tracrRNA |
| 432 | MS modified |
| | APG07433.1 crRNA |
| | repeat truncated by 5 |
| | nt at 3′ end |
| 433 | MS modified |
| | APG07433.1 crRNA |
| | repeat truncated by 6 |
| | nt at 3′ end |
| 434 | MS modified |
| | APG07433.1 crRNA |
| | repeat truncated by 7 |
| | nt at 3′ end |
| 435 | MS modified |
| | APG07433.1 crRNA |
| | repeat truncated by 8 |
| | nt at 3′ end |
| 436 | MS modified |
| | APG07433.1 |
| | truncated crRNA |
| | repeat in SGN001154 |
| 437 | MS modified |
| | APG07433.1 |
| | tracrRNA truncated |
| | by 5 nt at 5′ end, -4 |
| | tail, -2SL3 |
| 438 | MS modified |
| | APG07433.1 |
| | tracrRNA truncated |
| | by 6 nt at 5′ end (98 |
| | bb) |
| 439 | MS modified |
| | APG07433.1 |
| | tracrRNA 98bb_-6 |
| | tail |
| 440 | MS modified |
| | APG07433.1 |
| | tracrRNA 98bb_-6 |
| | tail_-2SL3 |
| 441 | MS modified |
| | APG07433.1 |
| | tracrRNA truncated |
| | by 7 nt at 5′ end (96 |
| | bb) |
| 442 | MS modified |
| | APG07433.1 |
| | tracrRNA 96bb_-6 |
| | tail |
| 443 | MS modified |
| | APG07433.1 |
| | tracrRNA 96bb_-6 |
| | tail_-2SL3 |
| 444 | MS modified |
| | APG07433.1 |
| | tracrRNA truncated |
| | by 8 nt at 5′ end (94 |
| | bb) |
| 445 | MS modified |
| | APG07433.1 |
| | tracrRNA 94bb_-6 |
| | tail |
| 446 | MS modified |
| | APG07433.1 |
| | tracrRNA 94bb_-6 |
| | tail-2SL3 |
| 447 | MS modified |
| | APG07433.1 M |
| | backbone |
| 448 | MS modified |
| | APG07433.1 98 bb |
| | backbone |
| 449 | MS modified |
| | APG07433.1 98bb_-6 |
| | tail backbone |
| 450 | MS modified |
| | APG07433.1 98bb_-6 |
| | tail_-2SL3 |
| | backbone |
| 451 | MS modified |
| | APG07433.1 96 bb |
| | backbone |
| 452 | MS modified |
| | APG07433.1 96bb_-6 |
| | tail backbone |
| 453 | MS modified |
| | APG07433.1 96bb_-6 |
| | tail_-2SL3 |
| | backbone |
| 454 | MS modified |
| | APG07433.1 94 bb |
| | backbone |
| 455 | MS modified |
| | APG07433.1 94bb_-6 |
| | tail backbone |
| 456 | MS modified |
| | APG07433.1 94bb_-6 |
| | tail-2SL3 backbone |
| 457 | SGN001268, |
| | SGN001269, |
| | SGN001794, |
| | SGN001856, |
| | SGN001857, |
| | SGN001880, |
| | SGN001881, and |
| | Full-length |
| | APG07433.1 |
| | backbone, MS |
| | modified |
| 458 | MS modified |
| | SGN001154 |
| | backbone |
| 459 | MS modified |
| | SGN000753 crRNA |
| 460 | MS modified |
| | SGN000754 crRNA |
| 461 | MS modified |
| | SGN000755 crRNA |
| 462 | MS modified |
| | SGN001154 crRNA |
| 463 | MS modified |
| | SGN001880 crRNA |
| 464 | MS modified |
| | SGN001881 crRNA |
| 465 | MS modified |
| | SGN003156 crRNA |
| 466 | MS modified |
| | SGN006286 crRNA |
| 467 | MS modified |
| | SGN000908 crRNA |
| 468 | MS modified |
| | SGN000909 crRNA |
| 469 | MS modified |
| | SGN000910 crRNA |
| 470 | MS modified |
| | SGN000911 crRNA |
| 471 | MS modified |
| | SGN000912 crRNA |
| 472 | MS modified |
| | SGN000914 crRNA |
| 473 | MS modified |
| | SGN000915 crRNA |
| 474 | MS modified |
| | SGN000916 crRNA |
| 475 | MS modified |
| | SGN000919 crRNA |
| 476 | MS modified |
| | SGN000921 crRNA |
| 477 | MS modified |
| | SGN000922 crRNA |
| 478 | MS modified |
| | SGN000923 crRNA |
| 479 | MS modified |
| | SGN000924 crRNA |
| 480 | MS modified |
| | SGN000925 crRNA |
| 481 | MS modified |
| | SGN000926 crRNA |
| 482 | MS modified |
| | SGN000927 crRNA |
| 483 | MS modified |
| | SGN000928 crRNA |
| 484 | MS modified |
| | SGN000929 crRNA |
| 485 | MS modified |
| | SGN000930 crRNA |
| 486 | MS modified |
| | SGN000931 crRNA |
| 487 | MS modified |
| | SGN000932 crRNA |
| 488 | MS modified |
| | SGN000933 crRNA |
| 489 | MS modified |
| | SGN000934 crRNA |
| 490 | MS modified |
| | SGN000935 crRNA |
| 491 | MS modified |
| | SGN000936 crRNA |
| 492 | MS modified |
| | SGN000937 crRNA |
| 493 | MS modified |
| | SGN000938 crRNA |
| 494 | MS modified |
| | SGN000939 crRNA |
| 495 | MS modified |
| | SGN000940 crRNA |
| 496 | MS modified |
| | SGN000942 crRNA |
| 497 | MS modified |
| | SGN000943 crRNA |
| 498 | MS modified |
| | SGN000944 crRNA |
| 499 | MS modified |
| | SGN000947 crRNA |
| 500 | MS modified |
| | SGN000948 crRNA |
| 501 | MS modified |
| | SGN000949 crRNA |
| 502 | MS modified |
| | SGN000950 crRNA |
| 503 | MS modified |
| | SGN000951 crRNA |
| 504 | MS modified |
| | SGN001065 crRNA |
| 505 | MS modified |
| | SGN001066 crRNA |
| 506 | MS modified |
| | SGN001268 crRNA |
| 507 | MS modified |
| | SGN001269 crRNA |
| 508 | MS modified |
| | SGN001794 crRNA |
| 509 | MS modified |
| | SGN001856 crRNA |
| 510 | MS modified |
| | SGN001857 crRNA |
| 511 | MS modified |
| | SGN003197 crRNA |
| 512 | MS modified |
| | SGN006283 crRNA |
| 513 | MS modified |
| | SGN006284 crRNA |
| 514 | MS modified |
| | SGN006285 crRNA |
| 515 | MS modified |
| | SGN003261 crRNA |
| 516 | MS modified |
| | SGN003146 crRNA |
| 517 | MS modified |
| | SGN003166 crRNA |
| 518 | MS modified |
| | SGN003210 crRNA |
| 519 | MS modified |
| | SGN003220 crRNA |
| 520 | MS modified |
| | SGN003230 crRNA |
| 521 | MS modified |
| | SGN000753 guide |
| | RNA |
| 522 | MS modified |
| | SGN000754 guide |
| | RNA |
| 523 | MS modified |
| | SGN000755 guide |
| | RNA |
| 524 | MS modified |
| | SGN001154 guide |
| | RNA |
| 525 | MS modified |
| | SGN001880 guide |
| | RNA |
| 526 | MS modified |
| | SGN001881 guide |
| | RNA |
| 527 | MS modified |
| | SGN003156 guide |
| | RNA |
| 528 | MS modified |
| | SGN006286 guide |
| | RNA |
| 529 | MS modified |
| | SGN000908 guide |
| | RNA |
| 530 | MS modified |
| | SGN000909 guide |
| | RNA |
| 531 | MS modified |
| | SGN000910 guide |
| | RNA |
| 532 | MS modified |
| | SGN000911 guide |
| | RNA |
| 533 | MS modified |
| | SGN000912 guide |
| | RNA |
| 534 | MS modified |
| | SGN000914 guide |
| | RNA |
| 535 | MS modified |
| | SGN000915 guide |
| | RNA |
| 536 | MS modified |
| | SGN000916 guide |
| | RNA |
| 537 | MS modified |
| | SGN000919 guide |
| | RNA |
| 538 | MS modified |
| | SGN000921 guide |
| | RNA |
| 539 | MS modified |
| | SGN000922 guide |
| | RNA |
| 540 | MS modified |
| | SGN000923 guide |
| | RNA |
| 541 | MS modified |
| | SGN000924 guide |
| | RNA |
| 542 | MS modified |
| | SGN000925 guide |
| | RNA |
| 543 | MS modified |
| | SGN000926 guide |
| | RNA |
| 544 | MS modified |
| | SGN000927 guide |
| | RNA |
| 545 | MS modified |
| | SGN000928 guide |
| | RNA |
| 546 | MS modified |
| | SGN000929 guide |
| | RNA |
| 547 | MS modified |
| | SGN000930 guide |
| | RNA |
| 548 | MS modified |
| | SGN000931 guide |
| | RNA |
| 549 | MS modified |
| | SGN000932 guide |
| | RNA |
| 550 | MS modified |
| | SGN000933 guide |
| | RNA |
| 551 | MS modified |
| | SGN000934 guide |
| | RNA |
| 552 | MS modified |
| | SGN000935 guide |
| | RNA |
| 553 | MS modified |
| | SGN000936 guide |
| | RNA |
| 554 | MS modified |
| | SGN000937 guide |
| | RNA |
| 555 | MS modified |
| | SGN000938 guide |
| | RNA |
| 556 | MS modified |
| | SGN000939 guide |
| | RNA |
| 557 | MS modified |
| | SGN000940 guide |
| | RNA |
| 558 | MS modified |
| | SGN000942 guide |
| | RNA |
| 559 | MS modified |
| | SGN000943 guide |
| | RNA |
| 560 | MS modified |
| | SGN000944 guide |
| | RNA |
| 561 | MS modified |
| | SGN000947 guide |
| | RNA |
| 562 | MS modified |
| | SGN000948 guide |
| | RNA |
| 563 | MS modified |
| | SGN000949 guide |
| | RNA |
| 564 | MS modified |
| | SGN000950 guide |
| | RNA |
| 565 | MS modified |
| | SGN000951 guide |
| | RNA |
| 566 | MS modified |
| | SGN001065 guide |
| | RNA |
| 567 | MS modified |
| | SGN001066 guide |
| | RNA |
| 568 | MS modified |
| | SGN001268 guide |
| | RNA |
| 569 | MS modified |
| | SGN001269 guide |
| | RNA |
| 570 | MS modified |
| | SGN001794 guide |
| | RNA |
| 571 | MS modified |
| | SGN001856 guide |
| | RNA |
| 572 | MS modified |
| | SGN001857 guide |
| | RNA |
| 573 | MS modified |
| | SGN003197 guide |
| | RNA |
| 574 | MS modified |
| | SGN006283 guide |
| | RNA |
| 575 | MS modified |
| | SGN006284 guide |
| | RNA |
| 576 | MS modified |
| | SGN006285 guide |
| | RNA |
| 577 | MS modified |
| | SGN003261 guide |
| | RNA |
| 578 | MS modified |
| | SGN003146 guide |
| | RNA |
| 579 | MS modified |
| | SGN003166 guide |
| | RNA |
| 580 | MS modified |
| | SGN003210 guide |
| | RNA |
| 581 | MS modified |
| | SGN003220 guide |
| | RNA |
| 582 | MS modified |
| | SGN003230 guide |
| | RNA |
| 583 | MS modified |
| | APG05083.1 crRNA |
| | repeat |
| 584 | MS modified |
| | APG05083.1 |
| | tracrRNA |
| 585 | MS modified |
| | APG08290.1 crRNA |
| | repeat sequence |
| 586 | MS modified |
| | APG08290.1 |
| | tracrRNA |
| 587 | MS modified |
| | APG07513.1 crRNA |
| | repeat |
| 588 | MS modified |
| | APG07513.1 |
| | tracrRNA |

That which is claimed:

1. A guide RNA (gRNA) comprising a CRISPR RNA (crRNA) and a trans-activating CRISPR RNA (tracrRNA), wherein the crRNA comprises
(i) a crRNA repeat; and
(ii) a spacer,
wherein the tracrRNA comprises:
(iii) an anti-repeat; and
(iv) a tail,
wherein the spacer is capable of hybridizing to a target sequence in a T cell receptor alpha chain constant (TRAC) gene, wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, and 104.

2. The gRNA of claim 1, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 to 5 nucleotides.

3. The gRNA of claim 1, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103.

4. The gRNA of any one of claims 1-3, wherein the crRNA repeat has the nucleotide sequence set forth as SEQ ID NO: 106 or a nucleotide sequence that differs in length and/or sequence from SEQ ID NO: 106 by 1 to 8 nucleotides.

5. The gRNA of claim 4, wherein the crRNA repeat has the nucleotide sequence set forth as any one of SEQ ID NOs: 106, 109-112, 328, 331, and 334.

6. The gRNA of any one of claims 1-5, wherein the crRNA has the nucleotide sequence set forth as any one of SEQ ID NOs: 136-197.

7. The gRNA of any one of claims 1-6, wherein the tracrRNA has a nucleotide sequence having at least 80% sequence identity, at least 90% sequence identity, or at least 95% sequence identity to SEQ ID NO: 107.

8. The gRNA of any one of claims 1-6, wherein the tracrRNA has a nucleotide sequence that differs in length from SEQ ID NO: 107 by 1 to 16 nucleotides.

9. The gRNA of claim 8, wherein the tracrRNA has a nucleotide sequence that is 8 nucleotides shorter than SEQ ID NO: 107.

10. The gRNA of claim 8, wherein the tracrRNA has a nucleotide sequence that is 11 nucleotides shorter than SEQ ID NO: 107.

11. The gRNA of any one of claims 7-10, wherein the tracrRNA has the nucleotide sequence set forth as any one of SEQ ID NOs: 107, 114-123, 329, 332, and 335.

12. The gRNA of any one of claims 1-3, wherein the gRNA is a single guide RNA (sgRNA) comprising the crRNA and the tracrRNA linked by a linker, wherein the sgRNA comprises a backbone and the spacer, and wherein the backbone of the sgRNA comprises the crRNA repeat, the linker, and the tracrRNA.

13. The gRNA of claim 12, wherein the linker has a nucleotide sequence set forth as AAAG, GAAA, ACUU, or CAAAGG.

14. The gRNA of claim 13, wherein the linker has a nucleotide sequence set forth as AAAG.

15. The gRNA of any one of claims 12-14, wherein the backbone of the sgRNA comprises a total length of at least 85, 90, 95, 100, 105, 110, 115, or 120 nucleotides.

16. The gRNA of any one of claims 12-14, wherein the backbone of the sgRNA comprises a total length of at most 85, 90, 95, 100, 105, 110, 115, or 120 nucleotides.

17. The gRNA of any one of claims 12-14, wherein the backbone of the sgRNA comprises a total length of 86 to 98 nucleotides.

18. The gRNA of any one of claims 12-14, wherein the backbone of the sgRNA comprises a total length of 94 nucleotides.

19. The gRNA of any one of claims 12-14, wherein the backbone of the sgRNA has a nucleotide sequence having at least 80% sequence identity, at least 90% sequence identity, at least 95% sequence identity, or 100% sequence identity to any one of SEQ ID NOs: 124-134.

20. The gRNA of any one of claims 1-3, wherein the gRNA comprises a first stem loop formed by hybridization of the crRNA repeat and the anti-repeat, wherein the first stem loop comprises a first stem and a second stem, and wherein the first stem of the first stem loop comprises a total length of at least 3, 4, 5, 6, 7, 8, 9, 10, or 11 base pairs (bp).

21. The gRNA of any one of claims 1-3, wherein the gRNA comprises a first stem loop formed by hybridization of the crRNA repeat and the anti-repeat, wherein the first stem loop comprises a first stem and a second stem, and wherein the first stem of the first stem loop comprises a total length of at most 3, 4, 5, 6, 7, 8, 9, 10, or 11 bp.

22. The gRNA of claim 20 or 21, wherein the first stem of the first stem loop comprises a total length of 6 bp.

23. The gRNA of claim 20 or 21, wherein the first stem of the first stem loop comprises a total length of 3 bp.

24. The gRNA of any one of claims 1-3, wherein the tail of the tracrRNA comprises a total length of at least 1, 2, 3, 4, 5, 6, or 7 nucleotides.

25. The gRNA of any one of claims 1-3, wherein the tail of the tracrRNA comprises a total length of at most 1, 2, 3, 4, 5, 6, or 7 nucleotides.

26. The gRNA of claim 24 or 25, wherein the tail of the tracrRNA comprises a total length of 3 nucleotides.

27. The gRNA of claim 24 or 25, wherein the tail of the tracrRNA comprises a total length of 1 nucleotide.

28. The gRNA of claim 20 or 21, wherein the gRNA further comprises a second stem loop most proximal to the tail, wherein the second stem loop comprises a first stem and a second stem.

29. The gRNA of claim 28, wherein the first stem of the second stem loop comprises a total length of at least 1, 2, 3, 4, 5, or 6 bp.

30. The gRNA of claim 28, wherein the first stem of the second stem loop comprises a total length of at most 1, 2, 3, 4, 5, or 6 bp.

31. The gRNA of claim 29 or 30, wherein the first stem of the second stem loop comprises a total length of 5 bp.

32. The gRNA of any one of claims 28-31, wherein the first stem of the first stem loop comprises a total length of 6 bp, the tail of the tracrRNA comprises a total length of 3 nucleotides, and the first stem of the second stem loop comprises a total length of 5 bp.

33. The gRNA of any one of claims 1-3, wherein the gRNA is a dual guide RNA (dgRNA).

34. The gRNA of claim 33, wherein the crRNA repeat of the dgRNA comprises a total length of at least 10, 11, 12, 13, 14, 15, 16, 17, 18, 19, 20, or 21 nucleotides.

35. The gRNA of claim 33, wherein the crRNA repeat of the dgRNA comprises a total length of at most 10, 11, 12, 13, 14, 15, 16, 17, 18, 19, 20, or 21 nucleotides.

36. The gRNA of claim 34 or 35, wherein the crRNA repeat of the dgRNA comprises a total length of 13 nucleotides.

37. The gRNA of claim 34 or 35, wherein the crRNA repeat of the dgRNA comprises a total length of 16 nucleotides.

38. The gRNA of claim 34 or 35, wherein the crRNA repeat of the dgRNA comprises a total length of 21 nucleotides.

39. The gRNA of claim 33, wherein the tracrRNA of the dgRNA comprises a total length of at least 65, 70, 75, 80, or 85 nucleotides.

40. The gRNA of claim 33, wherein the tracrRNA of the dgRNA comprises a total length of at most 65, 70, 75, 80, or 85 nucleotides.

41. The gRNA of claim 39 or 40, wherein the tracrRNA of the dgRNA comprises a total length of 74 nucleotides.

42. The gRNA of claim 39 or 40, wherein the tracrRNA of the dgRNA comprises a total length of 77 nucleotides.

43. The gRNA of any one of claims 1-42, wherein the gRNA comprises a total length of at least 85, 90, 95, 100, 105, 110, 115, 120, 125, 130, or 135 nucleotides.

44. The gRNA of any one of claims 1-42, wherein the gRNA comprises a total length of at most 85, 90, 95, 100, 105, 110, 115, 120, 125, 130, or 135 nucleotides.

45. The gRNA of any one of claims 1-42, wherein the gRNA comprises a total length of 106 to 135 nucleotides.

46. The gRNA of claim 45, wherein the gRNA comprises a total length of 117 to 119 nucleotides.

47. The gRNA of any one of claims 1-46, wherein the gRNA is capable of targeting a bound RNA-guided nuclease (RGN) polypeptide to the target sequence.

48. The gRNA of claim 47, wherein the RGN polypeptide is capable of recognizing a consensus protospacer adjacent motif (PAM) having the nucleotide sequence set forth as NNNNCC or NNRNCC.

49. The gRNA of claim 48, wherein the RGN polypeptide is capable of recognizing a full PAM having the nucleotide sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG.

50. The gRNA of any one of claims 47-49, wherein the RGN polypeptide has an amino acid sequence having at least 80% sequence identity to SEQ ID NO: 105; and wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 1 to 5 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 1 to 5 nucleotides.

51. The gRNA of claim 50, wherein the spacer has the nucleotide sequence set forth as SEQ ID NO: 7 or 9.

52. The gRNA of claim 50 or 51, wherein the RGN polypeptide has the amino acid sequence set forth as SEQ ID NO: 105.

53. The gRNA of any one of claims 47-49, wherein the RGN polypeptide has an amino acid sequence having at least 80% sequence identity, at least 90% sequence identity, at least 95% sequence identity, or 100% sequence identity to SEQ ID NO: 333.

54. The gRNA of claim 53, wherein the RGN polypeptide has an amino acid sequence set forth as SEQ ID NO: 333.

55. The gRNA of any one of claims 47-54, wherein the gRNA has a nucleotide sequence set forth as any one of SEQ ID NOs: 198-200, 202-213, 215-233, 235-241, and 243-259.

56. The gRNA of claim 55, wherein the gRNA has a nucleotide sequence set forth as SEQ ID NO: 204 or 205.

57. The gRNA of claim 47 or 48, wherein the RGN polypeptide has an amino acid sequence having at least 80% sequence identity, at least 90% sequence identity, at least 95% sequence identity, or 100% sequence identity to SEQ ID NO: 327 or 330.

58. The gRNA of claim 57, wherein the RGN polypeptide has an amino acid sequence set forth as SEQ ID NO: 327 or 330.

59. The gRNA of any one of claims 1-58, wherein the gRNA comprises at least one chemical modification.

60. The gRNA of claim 59, wherein the at least one chemical modification comprises a bridged nucleic acid (BNA) modification; 2′-O-methyl(2′-O-Me) modification; 2′-O-methoxy-ethyl(2′MOE) modification; 2′-fluoro (2′-F) modification; 2′F-4′Cα-OMe modification; 2′,4′-di-Cα-OMe modification; 2′-O-methyl 3′phosphorothioate (MS) modification; 2′-O-methyl 3′thiophosphonoacetate (MSP) modification; 2′-O-methyl 3′phosphonoacetate (MP) modification; phosphorothioate (PS) modification; or a combination thereof.

61. The gRNA of claim 60, wherein the at least one chemical modification comprises MS modifications at the 3 terminal nucleotides at the 5′ region and at the 3 terminal nucleotides at the 3′ region of the gRNA.

62. The gRNA of claim 61, wherein the crRNA repeat has the nucleotide sequence set forth as any one of SEQ ID NOs: 430, 432-435, 583, 585, and 587.

63. The gRNA of claim 61 or 62, wherein the crRNA has the nucleotide sequence set forth as any one of SEQ ID NOs: 459-520.

64. The gRNA of any one of claims 61-63, wherein the tracrRNA has the nucleotide sequence set forth as any one of SEQ ID NOs: 431, 437-446, 584, 586, and 588.

65. The gRNA of any one of claims 61-64, wherein the gRNA has the nucleotide sequence set forth as any one of SEQ ID NOs: 521-523, 525-536, 538-556, 558-564, and 566-582.

66. The gRNA of claim 60, wherein the BNA comprises a 2′,4′ BNA modification.

67. The gRNA of claim 66, wherein the 2′,4′ BNA modification is selected from the group consisting of: locked nucleic acid (LNA) modification, BNA^{NC}[N-Me] modification, 2′-O,4′-C-ethylene bridged nucleic acid (2′,4′-ENA) modification, and S-constrained ethyl (cEt) modification.

68. The gRNA of claim 67, wherein the 2′,4′ BNA is a LNA modification.

69. The gRNA of claim 67, wherein the 2′,4′ BNA is a cEt modification.

70. The gRNA of claim 60, wherein the at least one chemical modification comprises a BNA modification, 2′-O-Me modification, PS modification, or a combination thereof.

71. The gRNA of any one of claims 1-70, wherein the gRNA further comprises an extension comprising an edit template for reverse transcriptase (RT) editing.

72. A guide RNA (gRNA) comprising a CRISPR RNA (crRNA) and a trans-activating CRISPR RNA (tracrRNA), wherein the crRNA comprises
(i) a crRNA repeat; and
(ii) a spacer,
wherein the tracrRNA comprises:
(iii) an anti-repeat; and
(iv) a tail,
wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103, or has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 to 5 nucleotides.

73. The gRNA of claim 72, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103.

74. The gRNA of claim 72 or 73, wherein the spacer is capable of hybridizing to a target sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, and 104.

75. A nucleic acid molecule comprising a CRISPR RNA (crRNA) or encoding a crRNA, wherein the crRNA comprises a spacer and a crRNA repeat, wherein the spacer is capable of hybridizing to a target sequence in a T cell receptor alpha chain constant (TRAC) gene, and wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, and 104.

76. The nucleic acid molecule of claim 75, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 to 5 nucleotides.

77. The nucleic acid molecule of claim 75, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103.

78. The nucleic acid molecule of any one of claims 75-77, wherein the crRNA repeat has the nucleotide sequence set forth as SEQ ID NO: 106 or a nucleotide sequence that differs in length and/or sequence from SEQ ID NO: 106 by 1 to 8 nucleotides.

79. The nucleic acid molecule of claim 78, wherein the crRNA repeat has the nucleotide sequence set forth as any one of SEQ ID NOs: 106, 109-112, 328, 331, and 334.

80. The nucleic acid molecule of any one of claims 75-79, wherein the crRNA has the nucleotide sequence set forth as any one of SEQ ID NOs: 136-197.

81. The nucleic acid molecule of any one of claims 75-80, wherein the crRNA is capable of binding a trans-activating CRISPR RNA (tracrRNA) to form a guide RNA (gRNA), wherein the tracrRNA comprises an anti-repeat and a tail.

82. The nucleic acid molecule of claim 81, wherein the tracrRNA has a nucleotide sequence having at least 80% sequence identity, at least 90% sequence identity, or at least 95% sequence identity to SEQ ID NO: 107.

83. The nucleic acid molecule of claim 81, wherein the tracrRNA has a nucleotide sequence that differs in length from SEQ ID NO: 107 by 1 to 16 nucleotides.

84. The nucleic acid molecule of claim 83, wherein the tracrRNA has a nucleotide sequence that is 8 nucleotides shorter than SEQ ID NO: 107.

85. The nucleic acid molecule of claim 83, wherein the tracrRNA has a nucleotide sequence that is 11 nucleotides shorter than SEQ ID NO: 107.

86. The nucleic acid molecule of claim 82 or 83, wherein the tracrRNA has the nucleotide sequence set forth as any one of SEQ ID NOs: 107, 114-123, 329, 332, and 335.

87. The nucleic acid molecule of claim 81, wherein the gRNA is a single guide RNA (sgRNA) comprising the crRNA and the tracrRNA linked by a linker, wherein the sgRNA comprises a backbone and the spacer, and wherein the backbone of the sgRNA comprises the crRNA repeat, the linker, and the tracrRNA.

88. The nucleic acid molecule of claim 87, wherein the backbone of the sgRNA comprises a total length of 86 to 98 nucleotides.

89. The nucleic acid molecule of claim 87, wherein the backbone of the sgRNA comprises a total length of 94 nucleotides.

90. The nucleic acid molecule of any one of claims 87-89, wherein the backbone of the gRNA has a nucleotide sequence having at least 80% sequence identity, at least 90% sequence identity, at least 95% sequence identity, or 100% sequence identity to any one of SEQ ID NOs: 124-134.

91. The nucleic acid molecule of any one of claims 81-90, wherein the gRNA comprises a first stem loop comprising a first stem and a second stem formed by hybridization of the crRNA repeat and the anti-repeat, and wherein the first stem of the first stem loop comprises a total length of at least 3, 4, 5, 6, 7, 8, 9, 10, or 11 bp.

92. The nucleic acid molecule of any one of claims 81-90, wherein the gRNA comprises a first stem loop comprising a first stem and a second stem formed by hybridization of the crRNA repeat and the anti-repeat, and wherein the first stem of the first stem loop comprises a total length of at most 3, 4, 5, 6, 7, 8, 9, 10, or 11 bp.

93. The nucleic acid molecule of claim 91 or 92, wherein the first stem of the first stem loop comprises a total length of 6 bp.

94. The nucleic acid molecule of claim 91 or 92, wherein the first stem of the first stem loop comprises a total length of 3 bp.

95. The nucleic acid molecule of any one of claims 81-94, wherein the tail of the tracrRNA comprises a total length of at least 1, 2, 3, 4, 5, 6, or 7 nucleotides.

96. The nucleic acid molecule of any one of claims 81-94, wherein the tail of the tracrRNA comprises a total length of at most 1, 2, 3, 4, 5, 6, or 7 nucleotides.

97. The nucleic acid molecule of claim 95 or 96, wherein the tail of the tracrRNA comprises a total length of 3 nucleotides.

98. The nucleic acid molecule of claim 95 or 96, wherein the tail of the tracrRNA comprises a total length of 1 nucleotide.

99. The nucleic acid molecule of any one of claims 91-98, wherein the gRNA further comprises a second stem loop most proximal to the tail, wherein the second stem loop comprises a first stem and a second stem.

100. The nucleic acid molecule of claim 99, wherein the first stem of the second stem loop comprises a total length of at least 1, 2, 3, 4, 5, or 6 bp.

101. The nucleic acid molecule of claim 99, wherein the first stem of the second stem loop comprises a total length of at most 1, 2, 3, 4, 5, or 6 bp.

102. The nucleic acid molecule of claim 100 or 101, wherein the first stem of the second stem loop comprises a total length of 5 bp.

103. The nucleic acid molecule of any one of claims 99-102, wherein the first stem of the first stem loop comprises a total length of 6 bp, the tail of the tracrRNA comprises a total length of 3 nucleotides, and the first stem of the second stem loop comprises a total length of 5 bp.

104. The nucleic acid molecule of claim 81, wherein the gRNA is a dual guide RNA (dgRNA).

105. The nucleic acid molecule of claim 104, wherein the crRNA repeat comprises a total length of at least 10, 11, 12, 13, 14, 15, 16, 17, 18, 19, 20, or 21 nucleotides.

106. The nucleic acid molecule of claim 104, wherein the crRNA repeat comprises a total length of at most 10, 11, 12, 13, 14, 15, 16, 17, 18, 19, 20, or 21 nucleotides.

107. The nucleic acid molecule of claim 105 or 106, wherein the crRNA repeat comprises a total length of 13 nucleotides.

108. The nucleic acid molecule of claim 105 or 106, wherein the crRNA repeat comprises a total length of 16 nucleotides.

109. The nucleic acid molecule of claim 105 or 106, wherein the crRNA repeat of the dgRNA comprises a total length of 21 nucleotides.

110. The nucleic acid molecule of any one of claims 104-109, wherein the tracrRNA comprises a total length of at least 65, 70, 75, 80, or 85 nucleotides.

111. The nucleic acid molecule of any one of claims 104-109, wherein the tracrRNA comprises a total length of at most 65, 70, 75, 80, or 85 nucleotides.

112. The nucleic acid molecule of claim 110 or 111, wherein the tracrRNA comprises a total length of 74 nucleotides.

113. The nucleic acid molecule of claim 110 or 111, wherein the tracrRNA comprises a total length of 77 nucleotides.

114. The nucleic acid molecule of any one of claims 81-113, wherein the gRNA comprises a total length of at least 85, 90, 95, 100, 105, 110, 115, 120, 125, 130, or 135 nucleotides.

115. The nucleic acid molecule of any one of claims 81-113, wherein the gRNA comprises a total length of at most 85, 90, 95, 100, 105, 110, 115, 120, 125, 130, or 135 nucleotides.

116. The nucleic acid molecule of any one of claims 81-113, wherein the gRNA comprises a total length of 106 to 135 nucleotides.

117. The nucleic acid molecule of claim 116, wherein the gRNA comprises a total length of 117 to 119 nucleotides.

118. The nucleic acid molecule of any one of claims 81-117, wherein the gRNA is capable of targeting a bound RNA-guided nuclease (RGN) polypeptide to a target sequence.

119. The nucleic acid molecule of claim 118, wherein the RGN polypeptide is capable of recognizing a consensus protospacer adjacent motif (PAM) having the nucleotide sequence set forth as NNNNCC or NNRNCC.

120. The nucleic acid molecule of claim 119, wherein the RGN polypeptide is capable of recognizing a full protospacer adjacent motif (PAM) having the nucleotide sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG.

121. The nucleic acid molecule of any one of claims 118-120, wherein the RGN polypeptide comprises an amino acid sequence having at least 80% sequence identity to SEQ ID NO: 105; and wherein the target sequence and the spacer are selected from the group consisting of:
a) a target sequence having the nucleotide sequence set forth as SEQ ID NO: 8 and a spacer having the nucleotide sequence set forth as SEQ ID NO: 7 or a nucleotide sequence that differs in length and/or sequence from SEQ ID NO: 7 by 1 to 5 nucleotides; and
b) a target sequence having the nucleotide sequence set forth as SEQ ID NO: 10 and a spacer having the nucleotide sequence set forth as SEQ ID NO: 9 or a nucleotide sequence that differs in length and/or sequence from SEQ ID NO: 9 by 1 to 5 nucleotides.

122. The nucleic acid molecule of claim 121, wherein the spacer has the nucleotide sequence set forth as SEQ ID NO: 7 or 9.

123. The nucleic acid molecule of claim 121 or 122, wherein the RGN polypeptide comprises an amino acid sequence set forth as SEQ ID NO: 105.

124. The nucleic acid molecule of any one of claims 118-120, wherein the RGN polypeptide has an amino acid sequence having at least 80% sequence identity, at least 90% sequence identity, at least 95% sequence identity, or 100% sequence identity to SEQ ID NO: 333.

125. The nucleic acid molecule of claim 124, wherein the RGN polypeptide has an amino acid sequence set forth as SEQ ID NO: 333.

126. The nucleic acid molecule of any one of claims 81-125, wherein the gRNA has a nucleotide sequence set forth as any one of SEQ ID NOs: 198-200, 202-213, 215-233, 235-241, and 243-259.

127. The nucleic acid molecule of claim 126, wherein the gRNA has a nucleotide sequence set forth as SEQ ID NO: 204 or 205.

128. The nucleic acid molecule of claim 118 or 119, wherein the RGN polypeptide has an amino acid sequence having at least 80% sequence identity, at least 90% sequence identity, at least 95% sequence identity, or 100% sequence identity to SEQ ID NO: 327 or 330.

129. The nucleic acid molecule of claim 128, wherein the RGN polypeptide has an amino acid sequence set forth as SEQ ID NO: 327 or 330.

130. The nucleic acid molecule of any one of claims 81-125, wherein the gRNA comprises at least one chemical modification.

131. The nucleic acid molecule of claim 130, wherein the at least one chemical modification comprises a bridged nucleic acid (BNA) modification; 2′-O-methyl(2′-O-Me) modification; 2′-O-methoxy-ethyl(2′MOE) modification; 2′-fluoro (2′-F) modification; 2′F-4′Cα-OMe modification; 2′,4′-di-Cα-OMe modification; 2′-O-methyl 3′phosphorothioate (MS) modification; 2′-O-methyl 3′thiophosphonoacetate (MSP) modification; 2′-O-methyl 3′phosphonoacetate (MP) modification; phosphorothioate (PS) modification; or a combination thereof.

132. The nucleic acid molecule of claim 131, wherein the at least one chemical modification comprises MS modifications at the 3 terminal nucleotides at the 5′ region and at the 3 terminal nucleotides at the 3′ region of the gRNA.

133. The nucleic acid molecule of claim 132, wherein the crRNA repeat has the nucleotide sequence set forth as any one of SEQ ID NOs: 430, 432-435, 583, 585, and 587.

134. The nucleic acid molecule of claim 132 or 133, wherein the crRNA has the nucleotide sequence set forth as any one of SEQ ID NOs: 459-520.

135. The nucleic acid molecule of any one of claims 132-134, wherein the tracrRNA has the nucleotide sequence set forth as any one of SEQ ID NOs: 431, 437-446, 584, 586, and 588.

136. The nucleic acid molecule of any one of claims 132-135, wherein the gRNA has the nucleotide sequence set forth as any one of SEQ ID NOs: 521-523, 525-536, 538-556, 558-564, and 566-582.

137. The nucleic acid molecule of claim 131, wherein the BNA comprises a 2′,4′ BNA modification.

138. The nucleic acid molecule of claim 137, wherein the 2′,4′ BNA modification is selected from the group consisting of: locked nucleic acid (LNA) modification, BNA^{NC}[N-Me] modification, 2′-O,4′-C-ethylene bridged nucleic acid (2′,4′-ENA) modification, and S-constrained ethyl (cEt) modification.

139. The nucleic acid molecule of claim 138, wherein the 2′,4′ BNA is a LNA modification.

140. The nucleic acid molecule of claim 138, wherein the 2′,4′ BNA is a cEt modification.

141. The nucleic acid molecule of claim 131, wherein the at least one chemical modification comprises a BNA modification, 2′-O-Me modification, PS modification, or a combination thereof.

142. The nucleic acid molecule of any one of claims 81-141, wherein the gRNA further comprises an extension comprising an edit template for reverse transcriptase editing.

143. A nucleic acid molecule comprising a CRISPR RNA (crRNA) or encoding a crRNA, wherein the crRNA comprises a spacer and a crRNA repeat, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103, or has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103 by 1 to 5 nucleotides.

144. The nucleic acid molecule of claim 143, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, and 103.

145. The nucleic acid molecule of claim 143 or 144, wherein the spacer is capable of hybridizing to a target sequence, and wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, and 104.

146. A vector comprising the nucleic acid molecule of any one of claims 75-80, wherein the nucleic acid molecule comprises a polynucleotide encoding the crRNA.

147. The vector of claim 146, wherein the nucleic acid molecule further comprises a heterologous promoter operably linked to the polynucleotide encoding the crRNA.

148. The vector of claim 147, wherein the heterologous promoter is an RNA polymerase III (pol III) promoter.

149. The vector of any one of claims 146-148, wherein the vector further comprises a nucleic acid molecule encoding an RGN polypeptide.

150. The vector of claim 149, wherein the crRNA is capable of binding a tracrRNA to form a guide RNA, and wherein the guide RNA is capable of binding to the RGN polypeptide.

151. The vector of claim 149 or 150, wherein the vector further comprises a promoter operably linked to the nucleic acid molecule encoding the RGN polypeptide.

152. A vector comprising the nucleic acid molecule of any one of claims 81-142, wherein the nucleic acid molecule comprises a polynucleotide encoding the crRNA, and wherein the vector further comprises a polynucleotide encoding the tracrRNA.

153. The vector of claim 152, wherein the polynucleotide encoding the crRNA and the polynucleotide encoding the tracrRNA are operably linked to the same promoter and are encoded as a sgRNA.

154. The vector of claim 152, wherein the polynucleotide encoding the crRNA and the polynucleotide encoding the tracrRNA are operably linked to separate promoters.

155. The vector of any one of claims 152-154, wherein the vector further comprises a nucleic acid molecule encoding an RGN polypeptide.

156. The vector of claim 155, wherein the crRNA is capable of binding the tracrRNA to form a guide RNA, and wherein the guide RNA is capable of binding to the RGN polypeptide.

157. The vector of claim 155 or 156, wherein the vector further comprises a promoter operably linked to the nucleic acid molecule encoding the RGN polypeptide.

158. A cell comprising the gRNA of any one of claims 1-74, the nucleic acid molecule of any one of claims 75-145, or the vector of any one of claims 146-157.

159. An RNA-guided nuclease (RGN) system for binding a target sequence within a TRAC gene, wherein the RGN system comprises:
a) one or more guide RNA (gRNA) of any one of claims 1-74, or one or more polynucleotides comprising one or more nucleotide sequences encoding the one or more gRNA of any one of claims 1-74; and
b) an RGN polypeptide, or a polynucleotide comprising a nucleotide sequence encoding the RGN polypeptide.

160. The RGN system of claim 159, wherein the one or more gRNA is capable of forming a complex with the RGN polypeptide to direct the RGN polypeptide to bind to the target sequence.

161. The RGN system of claim 159 or 160, wherein the RGN polypeptide is capable of recognizing a consensus protospacer adjacent motif (PAM) having the nucleotide sequence set forth as NNNNCC or NNRNCC.

162. The RGN system of claim 161, wherein the RGN polypeptide is capable of recognizing a full PAM having a nucleotide sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG.

163. The RGN system of any one of claims 159-162, wherein the RGN polypeptide comprises an amino acid sequence set forth as SEQ ID NO: 105.

164. The RGN system of any one of claims 159-162, wherein the RGN polypeptide has an amino acid sequence set forth as SEQ ID NO: 333.

165. The RGN system of any one of claims 159-161, wherein the RGN polypeptide has an amino acid sequence set forth as SEQ ID NO: 327 or 330.

166. The RGN system of any one of claims 159-165, wherein the polynucleotide comprising a nucleotide sequence encoding the RGN polypeptide is codon optimized for expression in a mammalian cell.

167. The RGN system of any one of claims 159-166, wherein at least one of the one or more nucleotide sequences encoding the one or more gRNA and the nucleotide sequence encoding the RGN polypeptide is operably linked to a promoter heterologous to the nucleotide sequence.

168. The RGN system of any one of claims 159-167, wherein the one or more nucleotide sequences encoding the one or more gRNAs and the nucleotide sequence encoding the RGN polypeptide are located on one vector.

169. The RGN system of any one of claims 159-165, wherein the polynucleotide comprising a nucleotide sequence encoding the RGN polypeptide comprises an mRNA.

170. The RGN system of any one of claims 159-169, wherein the RGN polypeptide is nuclease inactive or is a nickase.

171. The RGN system of any one of claims 159-170, wherein the RGN polypeptide is fused to a base-editing polypeptide.

172. The RGN system of claim 171, wherein the base-editing polypeptide comprises a deaminase.

173. The RGN system of any one of claims 159-170, wherein the RGN polypeptide is fused to a reverse transcriptase (RT) editing polypeptide.

174. The RGN system of claim 173, wherein the RT editing polypeptide comprises a DNA polymerase.

175. The RGN system of claim 174, wherein the DNA polymerase comprises a reverse transcriptase.

176. The RGN system of any one of claims 173-175, wherein the gRNA further comprises an extension comprising an edit template for RT editing.

177. The RGN system of any one of claims 159-176, wherein the RGN polypeptide comprises one or more nuclear localization signals.

178. A ribonucleoprotein (RNP) complex comprising the one or more gRNA and the RGN polypeptide of the RGN system of any one of claims 159-177.

179. A cell comprising the RGN system of any one of claims 159-177 or the RNP complex of claim 178.

180. The cell of claim 179, wherein the cell is a eukaryotic cell.

181. The cell of claim 180, wherein the eukaryotic cell is a mammalian cell.

182. The cell of claim 181, wherein the mammalian cell is a human cell.

183. The cell of claim 181 or 182, wherein the mammalian cell or human cell is a T cell or an induced pluripotent stem cell.

184. A method for binding a target sequence within a TRAC gene, comprising delivering the RGN system of any one of claims 159-177 or the RNP complex of claim 178 to the target sequence or a cell comprising the target sequence.

185. The method of claim 184, wherein cleavage or modification of the target sequence occurs.

186. A method for assembling an RNA-guided nuclease (RGN) ribonucleoprotein complex, the method comprising combining under conditions suitable for formation of the complex:
a) the guide RNA of any one of claims 1-74; and
b) an RGN polypeptide that binds the guide RNA.

187. The method of claim 186, wherein the RGN polypeptide is capable of recognizing a consensus protospacer adjacent motif (PAM) having the nucleotide sequence set forth as NNNNCC or NNRNCC.

188. The method of claim 186 or 187, wherein the complex directs cleavage of the target sequence.

189. The method of claim 188, wherein the cleavage generates a double-stranded break.

190. The method of claim 188, wherein the cleavage generates a single-stranded break.

191. A method for binding a target sequence within a TRAC gene, the method comprising:
a) combining under conditions suitable for formation of a ribonucleoprotein (RNP) complex:
i) the guide RNA of any one of claims 1-74; and
ii) an RGN polypeptide that binds the guide RNA;
thereby assembling an RNP complex; and
b) contacting the target sequence or a cell comprising the target sequence with the assembled RNP complex.

192. The method of claim 191, wherein the guide RNA hybridizes to the target sequence, thereby directing binding of the RNP complex to the target sequence.

193. The method of claim 191 or 192, wherein the RGN polypeptide is capable of recognizing a consensus protospacer adjacent motif (PAM) having the nucleotide sequence set forth as NNNNCC or NNRNCC.

194. The method of claim 193, wherein the RGN polypeptide is capable of recognizing a full protospacer adjacent motif (PAM) having the nucleotide sequence set forth as any one of AAATCCAG, CTGACCCT, AGAACCCT, AGATCCAT, GAGGCCAC, CCCCCCAC, CCTCCCAT, TGTTCCAA, AACTCCAG, TTTGCCTT, GCCTCCCA, AATACCTC, TGTGCCGG, TCTGCCCA, AAAACCCC, ACAGCCTG, AGAGCCAA, CAGTCCTG, ATCCCCTC, CTCTCCGT, AGCACCTG, GGGCCCAG, TGTGCCTC, AAAACCGT, CAATCCTG, CTGCCCAG, CAGGCCAA, CTCCCCAG, AGAACCTG, AGACCCAG, TGTCCCTT, CCCTCCTG, AATGCCAC, CTCACCTC, TGATCCCC, GACACCAT, TCCGCCTC, CCCGCCTC, TATTCCAG, TTCACCGA, AAAACCAA, TCGACCAG, CCTGCCGT, and GAACCCTG.

195. The method of any one of claims 191-194, wherein the RGN polypeptide comprises an amino acid sequence set forth as SEQ ID NO: 105.

196. The method of any one of claims 191-194, wherein the RGN polypeptide has an amino acid sequence set forth as SEQ ID NO: 333.

197. The method of any one of claims 191-193, wherein the RGN polypeptide has an amino acid sequence set forth as SEQ ID NO: 327 or 330.

198. The method of any one of claims 191-197, wherein the method is performed in vitro or ex vivo.

199. The method of any one of claims 191-198, wherein the RGN polypeptide is capable of cleaving the target sequence, thereby allowing for the cleaving and/or modifying of the target sequence.

200. The method of claim 199, wherein the cleaving generates a single-stranded break.

201. The method of claim 199, wherein the cleaving generates a double-stranded break.

202. The method of claim 199, wherein the cleaving results in insertion of a heterologous sequence within the target sequence.

203. The method of any one of claims 191-198, wherein the RGN polypeptide is nuclease inactive or is a nickase.

204. The method of claim 203, wherein the RGN polypeptide is fused to a base-editing polypeptide.

205. The method of claim 204, wherein the base-editing polypeptide comprises a deaminase.

206. The method of any one of claims 191-198, wherein the RGN is fused to a reverse transcriptase (RT) editing polypeptide.

207. The method of claim 206, wherein the RT editing polypeptide comprises a DNA polymerase.

208. The method of claim 207, wherein the DNA polymerase comprises a reverse transcriptase.

209. The method of any one of claims 206-208, wherein the gRNA further comprises an extension comprising an edit template for RT editing.

210. A method for modulating expression of a T cell receptor alpha chain (TRAC) gene in a population of cells, comprising delivering the RGN system of any one of claims 159-177 or the RNP complex of claim 178 to the population of cells, wherein the population of cells comprises the target sequence, and wherein TRAC gene expression is modulated as compared to TRAC gene expression in a control population of cells.

211. The method of claim 210, wherein cleavage or modification of the target sequence occurs.

212. The method of claim 211, wherein cleavage or modification of the target sequence is detected by sequencing.

213. The method of any one of claims 210-212, wherein TRAC gene expression is measured by quantitative PCR, microarray, RNA-seq, flow cytometry, immunoblot, enzyme-linked immunosorbent assay (ELISA), protein immunoprecipitation, immunostaining, high performance liquid chromatography (HPLC), liquid chromatography-mass spectrometry (LC/MS), mass spectrometry, or a combination thereof.

214. The method of any one of claims 210-213, wherein TRAC gene expression is decreased.

215. The method of claim 214, wherein the decrease in TRAC gene expression comprises decrease in TRAC mRNA and/or TRAC protein level.

216. The method of claim 215, wherein the decrease in TRAC protein level is measured by flow cytometry for detection of CD3+ cells.

217. The method of claim 216, wherein a decrease in CD3+ cells as compared to a level of CD3+ cells in the control population of cells is indicative of the decrease in TRAC protein level.

218. The method of claim 217, wherein the decrease in CD3+ cells is 30% to 100%.

219. The method of claim 217, wherein the decrease in CD3+ cells is 50% to 100%.

220. The method of any one of claims 211-219, wherein cleavage or modification of the target sequence occurs at a rate of 40% to 100%.

221. The method of any one of claims 211-219, wherein cleavage or modification of the target sequence occurs at a rate of 80% to 100%.

222. The method of any one of claims 210-221, wherein the control population of cells has not been subjected to the delivering.

223. The method of any one of claims 210-222, wherein the population of cells comprises T cells.


SEQUENCE LISTING (588 sequences)

SEQ ID NO: 1 - RNA, 25 nt, synthetic construct
  source 1..25: mol_type = other RNA
tcttgtcccacagatatccagaacc

SEQ ID NO: 2 - DNA, 25 bp, Homo sapiens
  source 1..25: mol_type = genomic DNA
tcttgtcccacagatatccagaacc

SEQ ID NO: 3 - RNA, 25 nt, synthetic construct
  source 1..25: mol_type = other RNA
tgccgtgtaccagctgagagactct

SEQ ID NO: 4 - DNA, 25 bp, Homo sapiens
  source 1..25: mol_type = genomic DNA
tgccgtgtaccagctgagagactct

SEQ ID NO: 5 - RNA, 25 nt, synthetic construct
  source 1..25: mol_type = other RNA
tgatcctcttgtcccacagatatcc

SEQ ID NO: 6 - DNA, 25 bp, Homo sapiens
  source 1..25: mol_type = genomic DNA
tgatcctcttgtcccacagatatcc

SEQ ID NO: 7 - RNA, 24 nt, synthetic construct
  source 1..24: mol_type = other RNA
gccgtgtaccagctgagagactct

SEQ ID NO: 8 - DNA, 24 bp, Homo sapiens
  source 1..24: mol_type = genomic DNA
gccgtgtaccagctgagagactct

SEQ ID NO: 9 - RNA, 23 nt, synthetic construct
  source 1..23: mol_type = other RNA
atcctcttgtcccacagatatcc

SEQ ID NO: 10 - DNA, 23 bp, Homo sapiens
  source 1..23: mol_type = genomic DNA
atcctcttgtcccacagatatcc

SEQ ID NO: 11 - RNA, 25 nt, synthetic construct
  source 1..25: mol_type = other RNA
cctagagcccaagagaacccactga

SEQ ID NO: 12 - DNA, 25 bp, Homo sapiens
  source 1..25: mol_type = genomic DNA
cctagagcccaagagaacccactga

SEQ ID NO: 13 - RNA, 25 nt, synthetic construct
  source 1..25: mol_type = other RNA
tttaggttcgtatctgtaaaaccaa

SEQ ID NO: 14 - DNA, 25 bp, Homo sapiens
  source 1..25: mol_type = genomic DNA
tttaggttcgtatctgtaaaaccaa

SEQ ID NO: 15 - RNA, 25 nt, synthetic construct
  source 1..25: mol_type = other RNA
cagggcctcgataatgagataattt

SEQ ID NO: 16 - DNA, 25 bp, Homo sapiens
  source 1..25: mol_type = genomic DNA
cagggcctcgataatgagataattt

SEQ ID NO: 17 - RNA, 25 nt, synthetic construct
  source 1..25: mol_type = other RNA
ctcaacattccagggctcaagcgat

SEQ ID NO: 18 - DNA, 25 bp, Homo sapiens
  source 1..25: mol_type = genomic DNA
ctcaacattccagggctcaagcgat

SEQ ID NO: 19 - RNA, 25 nt, synthetic construct
  source 1..25: mol_type = other RNA
aagagtataaacacgtactttcata

SEQ ID NO: 20 - DNA, 25 bp, Homo sapiens
  source 1..25: mol_type = genomic DNA
aagagtataaacacgtactttcata

SEQ ID NO: 21 - RNA, 25 nt, synthetic construct
  source 1..25: mol_type = other RNA
tgtctcaaataataaaaaaaataag

SEQ ID NO: 22 - DNA, 25 bp, Homo sapiens
  source 1..25: mol_type = genomic DNA
tgtctcaaataataaaaaaaataag

SEQ ID NO: 23 - RNA, 25 nt, synthetic construct
  source 1..25: mol_type = other RNA
atcatcctgaggacagccaagaggt

SEQ ID NO: 24 - DNA, 25 bp, Homo sapiens
  source 1..25: mol_type = genomic DNA
atcatcctgaggacagccaagaggt

SEQ ID NO: 25 - RNA, 25 nt, synthetic construct
  source 1..25: mol_type = other RNA
gacctcaggtgatccacccgcttca

SEQ ID NO: 26 - DNA, 25 bp, Homo sapiens
  source 1..25: mol_type = genomic DNA
gacctcaggtgatccacccgcttca

SEQ ID NO: 27 - RNA, 25 nt, synthetic construct
  source 1..25: mol_type = other RNA
gtaggtgctcagaatttatcatcac

SEQ ID NO: 28 - DNA, 25 bp, Homo sapiens
  source 1..25: mol_type = genomic DNA
gtaggtgctcagaatttatcatcac

SEQ ID NO: 29 - RNA, 25 nt, synthetic construct
  source 1..25: mol_type = other RNA
actcattaacccaccaatcactgat

SEQ ID NO: 30 - DNA, 25 bp, Homo sapiens
  source 1..25: mol_type = genomic DNA
actcattaacccaccaatcactgat

SEQ ID NO: 31 - RNA, 25 nt, synthetic construct
  source 1..25: mol_type = other RNA
ttccttgcttcaggaatggccaggt

SEQ ID NO: 32 - DNA, 25 bp, Homo sapiens
  source 1..25: mol_type = genomic DNA
ttccttgcttcaggaatggccaggt

SEQ ID NO: 33 - RNA, 25 nt, synthetic construct
  source 1..25: mol_type = other RNA
tcttttatttaataggatcttcttc

SEQ ID NO: 34 - DNA, 25 bp, Homo sapiens
  source 1..25: mol_type = genomic DNA
tcttttatttaataggatcttcttc

SEQ ID NO: 35 - RNA, 25 nt, synthetic construct
  source 1..25: mol_type = other RNA
cattccagatctgcaagattgtaag

SEQ ID NO: 36 - DNA, 25 bp, Homo sapiens
  source 1..25: mol_type = genomic DNA
cattccagatctgcaagattgtaag

SEQ ID NO: 37 - RNA, 25 nt, synthetic construct
  source 1..25: mol_type = other RNA
cccagaagggctcagaaataaaata

SEQ ID NO: 38 - DNA, 25 bp, Homo sapiens
  source 1..25: mol_type = genomic DNA
cccagaagggctcagaaataaaata

SEQ ID NO: 39 - RNA, 25 nt, synthetic construct
  source 1..25: mol_type = other RNA
agagccatcaccatcacatccctaa

SEQ ID NO: 40 - DNA, 25 bp, Homo sapiens
  source 1..25: mol_type = genomic DNA
agagccatcaccatcacatccctaa

SEQ ID NO: 41 - RNA, 25 nt, synthetic construct
  source 1..25: mol_type = other RNA
ggccaaaatccagctgccttccttg

SEQ ID NO: 42 - DNA, 25 bp, Homo sapiens
  source 1..25: mol_type = genomic DNA
ggccaaaatccagctgccttccttg

SEQ ID NO: 43 - RNA, 25 nt, synthetic construct
  source 1..25: mol_type = other RNA
gcttctactcttggcttacaaccca

SEQ ID NO: 44 - DNA, 25 bp, Homo sapiens
  source 1..25: mol_type = genomic DNA
gcttctactcttggcttacaaccca

SEQ ID NO: 45 - RNA, 25 nt, synthetic construct
  source 1..25: mol_type = other RNA
atctggagggaacttacagcatatg

SEQ ID NO: 46 - DNA, 25 bp, Homo sapiens
  source 1..25: mol_type = genomic DNA
atctggagggaacttacagcatatg

SEQ ID NO: 47 - RNA, 25 nt, synthetic construct
  source 1..25: mol_type = other RNA
gaacaactcaaatggaaatgaatat

SEQ ID NO: 48 - DNA, 25 bp, Homo sapiens
  source 1..25: mol_type = genomic DNA
gaacaactcaaatggaaatgaatat

SEQ ID NO: 49 - RNA, 25 nt, synthetic construct
  source 1..25: mol_type = other RNA
tcctgttccatcaccatcaaaaaaa

SEQ ID NO: 50 - DNA, 25 bp, Homo sapiens
  source 1..25: mol_type = genomic DNA
tcctgttccatcaccatcaaaaaaa

SEQ ID NO: 51 - RNA, 25 nt, synthetic construct
  source 1..25: mol_type = other RNA
taaaatgatttggaagagcagagac

SEQ ID NO: 52 - DNA, 25 bp, Homo sapiens
  source 1..25: mol_type = genomic DNA
taaaatgatttggaagagcagagac

SEQ ID NO: 53 - RNA, 25 nt, synthetic construct
  source 1..25: mol_type = other RNA
ccacccagcctgctctgccttgggg

SEQ ID NO: 54 - DNA, 25 bp, Homo sapiens
  source 1..25: mol_type = genomic DNA
ccacccagcctgctctgccttgggg

SEQ ID NO: 55 - RNA, 25 nt, synthetic construct
  source 1..25: mol_type = other RNA
aagacattacactcatattacaccc

SEQ ID NO: 56 - DNA, 25 bp, Homo sapiens
  source 1..25: mol_type = genomic DNA
aagacattacactcatattacaccc

SEQ ID NO: 57 - RNA, 25 nt, synthetic construct
  source 1..25: mol_type = other RNA
tgttggttacctccctgccaccacc

SEQ ID NO: 58 - DNA, 25 bp, Homo sapiens
  source 1..25: mol_type = genomic DNA
tgttggttacctccctgccaccacc

SEQ ID NO: 59 - RNA, 25 nt, synthetic construct
  source 1..25: mol_type = other RNA
cggcactatcattgactcagctgct

SEQ ID NO: 60 - DNA, 25 bp, Homo sapiens
  source 1..25: mol_type = genomic DNA
cggcactatcattgactcagctgct

SEQ ID NO: 61 - RNA, 25 nt, synthetic construct
  source 1..25: mol_type = other RNA
ccctggctgtgcacattccctcctg

SEQ ID NO: 62 - DNA, 25 bp, Homo sapiens
  source 1..25: mol_type = genomic DNA
ccctggctgtgcacattccctcctg

SEQ ID NO: 63 - RNA, 25 nt, synthetic construct
  source 1..25: mol_type = other RNA
gacatcattgaccagagctctgggc

SEQ ID NO: 64 - DNA, 25 bp, Homo sapiens
  source 1..25: mol_type = genomic DNA
gacatcattgaccagagctctgggc

SEQ ID NO: 65 - RNA, 25 nt, synthetic construct
  source 1..25: mol_type = other RNA
cagaagtcaggctggacactgagga

SEQ ID NO: 66 - DNA, 25 bp, Homo sapiens
  source 1..25: mol_type = genomic DNA
cagaagtcaggctggacactgagga

SEQ ID NO: 67 - RNA, 25 nt, synthetic construct
  source 1..25: mol_type = other RNA
ggaggcctgcagctcctgcacctcc

SEQ ID NO: 68 - DNA, 25 bp, Homo sapiens
  source 1..25: mol_type = genomic DNA
ggaggcctgcagctcctgcacctcc

SEQ ID NO: 69 - RNA, 25 nt, synthetic construct
  source 1..25: mol_type = other RNA
gcagcctcccctggctgtgcacatt

SEQ ID NO: 70 - DNA, 25 bp, Homo sapiens
  source 1..25: mol_type = genomic DNA
gcagcctcccctggctgtgcacatt

SEQ ID NO: 71 - RNA, 25 nt, synthetic construct
  source 1..25: mol_type = other RNA
gctaccacctctgtgcccccccggc

SEQ ID NO: 72 - DNA, 25 bp, Homo sapiens
  source 1..25: mol_type = genomic DNA
gctaccacctctgtgcccccccggc

SEQ ID NO: 73 - RNA, 25 nt, synthetic construct
  source 1..25: mol_type = other RNA
aaccccactcccagcttcaaggccc

SEQ ID NO: 74 - DNA, 25 bp, Homo sapiens
  source 1..25: mol_type = genomic DNA
aaccccactcccagcttcaaggccc

SEQ ID NO: 75 - RNA, 25 nt, synthetic construct
  source 1..25: mol_type = other RNA
caggccaaaatccagctgccttcct

SEQ ID NO: 76 - DNA, 25 bp, Homo sapiens
  source 1..25: mol_type = genomic DNA
caggccaaaatccagctgccttcct

SEQ ID NO: 77 - RNA, 25 nt, synthetic construct
  source 1..25: mol_type = other RNA
tccacttcaacacctggtgcattca

SEQ ID NO: 78 - DNA, 25 bp, Homo sapiens
  source 1..25: mol_type = genomic DNA
tccacttcaacacctggtgcattca

SEQ ID NO: 79 - RNA, 25 nt, synthetic construct
  source 1..25: mol_type = other RNA
ggcaggaccgatacctcggctcctg

SEQ ID NO: 80 - DNA, 25 bp, Homo sapiens
  source 1..25: mol_type = genomic DNA
ggcaggaccgatacctcggctcctg

SEQ ID NO: 81 - RNA, 25 nt, synthetic construct
  source 1..25: mol_type = other RNA
tggtgcatcttggctcactgcaacc

SEQ ID NO: 82 - DNA, 25 bp, Homo sapiens
  source 1..25: mol_type = genomic DNA
tggtgcatcttggctcactgcaacc

SEQ ID NO: 83 - RNA, 25 nt, synthetic construct
  source 1..25: mol_type = other RNA
accagcctgggcaacacagggagac

SEQ ID NO: 84 - DNA, 25 bp, Homo sapiens
  source 1..25: mol_type = genomic DNA
accagcctgggcaacacagggagac

SEQ ID NO: 85 - RNA, 25 nt, synthetic construct
  source 1..25: mol_type = other RNA
gtgcaaacgccttcaacaacagcat

SEQ ID NO: 86 - DNA, 25 bp, Homo sapiens
  source 1..25: mol_type = genomic DNA
gtgcaaacgccttcaacaacagcat

SEQ ID NO: 87 - RNA, 25 nt, synthetic construct
  source 1..25: mol_type = other RNA
atccagtgacaagtctgtctgccta

SEQ ID NO: 88 - DNA, 25 bp, Homo sapiens
  source 1..25: mol_type = genomic DNA
atccagtgacaagtctgtctgccta

SEQ ID NO: 89 - RNA, 25 nt, synthetic construct
  source 1..25: mol_type = other RNA
tttgaaagtttaggttcgtatctgt

SEQ ID NO: 90 - DNA, 25 bp, Homo sapiens
  source 1..25: mol_type = genomic DNA
tttgaaagtttaggttcgtatctgt

SEQ ID NO: 91 - RNA, 25 nt, synthetic construct
  source 1..25: mol_type = other RNA
tgtcttacctgtttcaaagcttttc

SEQ ID NO: 92 - DNA, 25 bp, Homo sapiens
  source 1..25: mol_type = genomic DNA
tgtcttacctgtttcaaagcttttc

SEQ ID NO: 93 - RNA, 20 nt, synthetic construct
  source 1..20: mol_type = other RNA
tgtaccagctgagagactct

SEQ ID NO: 94 - DNA, 20 bp, Homo sapiens
  source 1..20: mol_type = genomic DNA
tgtaccagctgagagactct

SEQ ID NO: 95 - RNA, 25 nt, synthetic construct
  source 1..25: mol_type = other RNA
tcccacagatatccagaaccctgac

SEQ ID NO: 96 - DNA, 25 bp, Homo sapiens
  source 1..25: mol_type = genomic DNA
tcccacagatatccagaaccctgac

SEQ ID NO: 97 - RNA, 25 nt, synthetic construct
  source 1..25: mol_type = other RNA
gatcctcttgtcccacagatatcca

SEQ ID NO: 98 - DNA, 25 bp, Homo sapiens
  source 1..25: mol_type = genomic DNA
gatcctcttgtcccacagatatcca

SEQ ID NO: 99 - RNA, 23 nt, synthetic construct
  source 1..23: mol_type = other RNA
ccgtgtaccagctgagagactct

SEQ ID NO: 100 - DNA, 23 bp, Homo sapiens
  source 1..23: mol_type = genomic DNA
ccgtgtaccagctgagagactct

SEQ ID NO: 101 - RNA, 24 nt, synthetic construct
  source 1..24: mol_type = other RNA
gatcctcttgtcccacagatatcc

SEQ ID NO: 102 - DNA, 24 bp, Homo sapiens
  source 1..24: mol_type = genomic DNA
gatcctcttgtcccacagatatcc

SEQ ID NO: 103 - RNA, 24 nt, synthetic construct
  source 1..24: mol_type = other RNA
gatcctcttgtcccacagatatcc

SEQ ID NO: 104 - DNA, 24 bp, Homo sapiens
  source 1..24: mol_type = genomic DNA
gatcctcttgtcccacagatatcc

SEQ ID NO: 105 - protein, 1071 aa, Bacillus sp.
MRELDYRIGLDIGTNSIGWGVIELSWNKDRERYEKVRIVDQGVRMFDRAEMPKTGASLAEPRRIARSSRRRLNRKSQRKKNIRNLLVQHGVITQEELDSLYPLSKKSMDIWGIRLDGLDRLLNHFEWARLLIHLAQRRGFKSNRKSELKDTETGKVLSSIQLNEKRLSLYRTVGEMWMKDPDFSKYDRKRNSPNEYVFSVSRAELEKEIVTLFAAQRRFQSPYASKDLQETYLQIWTHQLPFASGNAILNKVGYCSLLKGKERRIPKATYTFQYFSALDQVNRTRLGPDFQPFTKEQREIILNNMFQRTDYYKKKTIPEVTYYDIRKWLELDETIQFKGLNYDPNEELKKIEKKPFINLKAFYEINKVVANYSERTNETFSTLDYDGIGYALTVYKTDKDIRSYLKSSHNLPKRCYDDQLIEELLSLSYTKFGHLSLKAINHVLSIMQKGNTYKEAVDQLGYDTSGLKKEKRSKFLPPISDEITNPIVKRALTQARKVVNAIIRRHGSPHSVHIELARELSKNHDERTKIVSAQDENYKKNKGAISILSEHGILNPTGYDIVRYKLWKEQGERCAYSLKEIPADTFFNELKKERNGAPILEVDHILPYSQSFIDSYHNKVLVYSDENRKKGNRIPYTYFLETNKDWEAFERYVRSNKFFSKKKREYLLKRAYLPRESELIKERHLNDTRYASTFLKNFIEQNLQFKEAEDNPRKRRVQTVNGVITAHFRKRWGLEKDRQETYLHHAMDAIIVACTDHHMVTRVTEYYQIKESNKSVKKPYFPMPWEGFRDELLSHLASQPIAKKISEELKAGYQSLDYIFVSRMPKRSITGAAHKQTIMRKGGIDKKGKTIIIERLHLKDIKFDENGDFKMVGKEQDMATYEAIKQRYLEHGKNSKKAFETPLYKPSKKGTGNLIKRVKVEGQAKSFVREVNGGVAQNGDLVRVDLFEKDDKYYMVPIYVPDTVCSELPKKVVASSKGYEQWLTLDNSFTFKFSLYPYDLVRLVKGDEDRFLYFGTLDIDSDRLNFKDVNKPSKKNEYRYSLKTIEDLEKYEVGVLGDLRLVRKETRRNFH

SEQ ID NO: 106 - RNA, 21 nt, Bacillus sp.
  source 1..21: mol_type = genomic RNA
gtcatagttccattaaagcca

SEQ ID NO: 107 - RNA, 85 nt, Bacillus sp.
  source 1..85: mol_type = genomic RNA
tggctttgatgtttctatgataagggtttcgacccgtggcgtcggggatcgcctgcccattgaaatgggcttctccccatttatt

SEQ ID NO: 108 - DNA, 3213 bp, synthetic construct
  source 1..3213: mol_type = other DNA
atgagagagctggactacagaattggcctggacatcggcaccaacagcatcggatggggcgtgatcgagctgtcctggaacaaagaccgggagagatacgagaaggtcagaatcgtggatcaaggcgtgagaatgttcgacagagccgagatgcccaagacaggcgccagcttagctgaacccagaagaatcgccagatccagcagacggagactgaatcgcaagtcccagagaaagaaaaacatccggaacctgctggtgcaacacggcgtgatcacacaggaggaactggatagcctgtaccccctgagcaaaaagagcatggacatctggggcattcggctcgacggcctggacagactcctcaatcatttcgagtgggccagactgctgatccacctggctcagagacggggctttaagtccaacagaaagagtgaactgaaagatacagagacaggcaaggtgctgagcagcatccaactgaacgagaaacggctgagcttgtatagaaccgtgggcgagatgtggatgaaggaccccgacttctctaaatacgataggaagagaaatagccccaacgaatacgtgttcagcgtgtctagagccgagctggaaaaggaaatcgtgaccctgttcgccgcccagcggagattccagagcccttacgccagcaaagatctgcaggagacatatctgcagatctggacccaccaactgcctttcgccagcggcaatgccatcctgaacaaggtcggatactgctccctgttgaaaggcaaagaaagaaggattcccaaggctacatacaccttccaatacttctctgctctggaccaggtgaatcggaccagactgggacctgatttccagcccttcaccaaggagcaacgggaaattatcttgaacaacatgttccagaggacagattactacaagaagaaaaccatccccgaggtgacctactatgacatacggaagtggctggaattggacgagacaattcagttcaagggcctgaactacgaccctaacgaggaactgaagaagatcgagaagaagccttttatcaatctgaaggccttctacgagatcaacaaggtggtggccaactacagcgaaagaaccaacgagaccttctccaccctggactacgacggcatcggctacgccctgaccgtgtacaaaaccgacaaggatatccgcagctacctgaagagcagtcacaacctacctaagagatgctacgacgaccaactgatcgaggaactgctgagcctgagctacacaaagttcggccacctgtccctgaaagccatcaaccacgtgctgtctatcatgcagaagggcaatacctacaaggaagccgtggaccaactgggctacgacaccagcggccttaagaaggagaagaggtccaagttcctgccacctatttctgatgaaatcacgaatccaatcgtgaaaagggccctgacccaggccagaaaagtggtgaacgccataattagaagacacggatctcctcactccgtgcacatcgagctggccagagagctgagcaagaaccacgacgagcggacaaagatcgtcagcgcccaggatgaaaactacaagaaaaacaagggcgctatcagcatcctgtctgagcacggcatcctgaaccctacaggctacgacatcgtgagatacaaactgtggaaggagcagggcgaacggtgcgcctacagcctgaaggaaatccctgccgatacatttttcaacgagctgaagaaggaacgcaacggcgcccctatccttgaagtggaccacatcctgccctacagccagtccttcatcgactcctaccacaacaaggtcctggtgtacagcgacgaaaaccggaaaaagggcaacagaatcccttatacctacttcctggaaaccaacaaggattgggaggcctttgagcggtacgtgcggagcaacaaattcttctccaagaaaaagcgagagtaccttctgaagcgggcttatctgcctagagaatctgagctgatcaaagaacgccacctgaacgacaccagatacgcctctaccttcctgaagaacttcatcgagcagaacctgcagttcaaggaagccgaggacaaccccagaaaaagacgggtgcaaaccgtgaacggcgttatcaccgcccacttcagaaagcggtggggcctggagaaggaccggcaggagacatacctccatcacgctatggacgccatcatcgtggcttgtacagaccaccacatggtcaccagagtgaccgagtactatcagatcaaggaaagcaacaagagcgtgaagaagccctattttcctatgccttgggaaggcttccgggacgagctgctgagccacttggcttctcagcctatcgccaagaaaatcagcgaggaactgaaggccggctaccagagcctggactacatcttcgtgtccagaatgcctaagagaagcattacaggcgctgctcataagcagaccatcatgcggaagggaggaattgacaagaagggcaaaacaatcatcatcgaacggctgcacctgaaggatatcaagttcgacgagaacggagatttcaagatggtgggcaaggaacaggacatggccacatacgaagctattaaacagagatacctggagcacggcaagaatagcaagaaggccttcgagacccctctgtacaagcccagcaaaaagggcacaggtaacctgatcaagcgggtgaaggtggaaggacaggccaagagctttgtgagggaagtgaacggcggagtggcccaaaatggcgatctggttagagttgatttgtttgagaaggatgataagtactacatggtccccatctacgtgccagacaccgtgtgtagcgagctgcccaaaaaggtggtcgccagctctaagggctatgagcagtggctgacactggataacagcttcacctttaagttcagcctgtacccttatgatctggtgcggctggtcaagggagatgaggatcggttcctgtactttggcaccctggacatcgacagcgacagacttaacttcaaggacgtgaacaagccaagcaagaagaacgagtaccggtacagcttgaaaaccatcgaggacttggagaagtacgaggtgggcgtgctgggcgatctaagactggtccggaaggaaactcgaagaaacttccac

SEQ ID NO: 109 - RNA, 16 nt, synthetic construct
  source 1..16: mol_type = other RNA
gtcatagttccattaa

SEQ ID NO: 110 - RNA, 15 nt, synthetic construct
  source 1..15: mol_type = other RNA
gtcatagttccatta

SEQ ID NO: 111 - RNA, 14 nt, synthetic construct
  source 1..14: mol_type = other RNA
gtcatagttccatt

SEQ ID NO: 112 - RNA, 13 nt, synthetic construct
  source 1..13: mol_type = other RNA
gtcatagttccat

SEQ ID NO: 113 - RNA, 11 nt, synthetic construct
  source 1..11: mol_type = other RNA
gtcatagccat

SEQ ID NO: 114 - RNA, 74 nt, synthetic construct
  source 1..74: mol_type = other RNA
ttgatgtttctatgataagggtttcgacccgtggcgtcggggatcgcctgcccttgaaagggcttctccccatt

SEQ ID NO: 115 - RNA, 79 nt, synthetic construct
  source 1..79: mol_type = other RNA
tgatgtttctatgataagggtttcgacccgtggcgtcggggatcgcctgcccattgaaatgggcttctccccatttatt

SEQ ID NO: 116 - RNA, 73 nt, synthetic construct
  source 1..73: mol_type = other RNA
tgatgtttctatgataagggtttcgacccgtggcgtcggggatcgcctgcccattgaaatgggcttctcccca

SEQ ID NO: 117 - RNA, 71 nt, synthetic construct
  source 1..71: mol_type = other RNA
tgatgtttctatgataagggtttcgacccgtggcgtcggggatcgcctgcccttgaaagggcttctcccca

SEQ ID NO: 118 - RNA, 78 nt, synthetic construct
  source 1..78: mol_type = other RNA
gatgtttctatgataagggtttcgacccgtggcgtcggggatcgcctgcccattgaaatgggcttctccccatttatt

SEQ ID NO: 119 - RNA, 72 nt, synthetic construct
  source 1..72: mol_type = other RNA
gatgtttctatgataagggtttcgacccgtggcgtcggggatcgcctgcccattgaaatgggcttctcccca

SEQ ID NO: 120 - RNA, 70 nt, synthetic construct
  source 1..70: mol_type = other RNA
gatgtttctatgataagggtttcgacccgtggcgtcggggatcgcctgcccttgaaagggcttctcccca

SEQ ID NO: 121 - RNA, 77 nt, synthetic construct
  source 1..77: mol_type = other RNA
atgtttctatgataagggtttcgacccgtggcgtcggggatcgcctgcccattgaaatgggcttctccccatttatt

SEQ ID NO: 122 - RNA, 71 nt, synthetic construct
  source 1..71: mol_type = other RNA
atgtttctatgataagggtttcgacccgtggcgtcggggatcgcctgcccattgaaatgggcttctcccca

SEQ ID NO: 123 - RNA, 69 nt, synthetic construct
  source 1..69: mol_type = other RNA
atgtttctatgataagggtttcgacccgtggcgtcggggatcgcctgcccttgaaagggcttctcccca

SEQ ID NO: 124 - RNA, 94 nt, synthetic construct
  source 1..94: mol_type = other RNA
gtcatagttccattaaaaagttgatgtttctatgataagggtttcgacccgtggcgtcggggatcgcctgcccttgaaagggcttctccccatt

SEQ ID NO: 125 - RNA, 98 nt, synthetic construct
  source 1..98: mol_type = other RNA
gtcatagttccattaaaagtgatgtttctatgataagggtttcgacccgtggcgtcggggatcgcctgcccattgaaatgggcttctccccatttatt

SEQ ID NO: 126 - RNA, 92 nt, synthetic construct
  source 1..92: mol_type = other RNA
gtcatagttccattaaaagtgatgtttctatgataagggtttcgacccgtggcgtcggggatcgcctgcccattgaaatgggcttctcccca

SEQ ID NO: 127 - RNA, 90 nt, synthetic construct
  source 1..90: mol_type = other RNA
gtcatagttccattaaaagtgatgtttctatgataagggtttcgacccgtggcgtcggggatcgcctgcccttgaaagggcttctcccca

SEQ ID NO: 128 - RNA, 96 nt, synthetic construct
  source 1..96: mol_type = other RNA
gtcatagttccattaaaggatgtttctatgataagggtttcgacccgtggcgtcggggatcgcctgcccattgaaatgggcttctccccatttatt

SEQ ID NO: 129 - RNA, 90 nt, synthetic construct
  source 1..90: mol_type = other RNA
gtcatagttccattaaaggatgtttctatgataagggtttcgacccgtggcgtcggggatcgcctgcccattgaaatgggcttctcccca

SEQ ID NO: 130 - RNA, 88 nt, synthetic construct
  source 1..88: mol_type = other RNA
gtcatagttccattaaaggatgtttctatgataagggtttcgacccgtggcgtcggggatcgcctgcccttgaaagggcttctcccca

SEQ ID NO: 131 - RNA, 94 nt, synthetic construct
  source 1..94: mol_type = other RNA
gtcatagttccataaagatgtttctatgataagggtttcgacccgtggcgtcggggatcgcctgcccattgaaatgggcttctccccatttatt

SEQ ID NO: 132 - RNA, 88 nt, synthetic construct
  source 1..88: mol_type = other RNA
gtcatagttccataaagatgtttctatgataagggtttcgacccgtggcgtcggggatcgcctgcccattgaaatgggcttctcccca

SEQ ID NO: 133 - RNA, 86 nt, synthetic construct
  source 1..86: mol_type = other RNA
gtcatagttccataaagatgtttctatgataagggtttcgacccgtggcgtcggggatcgcctgcccttgaaagggcttctcccca

SEQ ID NO: 134 - RNA, 110 nt, synthetic construct
  source 1..110: mol_type = other RNA
gtcatagttccattaaagccaaaagtggctttgatgtttctatgataagggtttcgacccgtggcgtcggggatcgcctgcccattgaaatgggcttctccccatttatt

SEQ ID NO: 135 - RNA, 90 nt, synthetic construct
  source 1..90: mol_type = other RNA
gtcatagccataaagatgtctatgataagggtttcgacccgtggcgtcggggatcgcctgcccattgaaatgggcttctccccatttatt

SEQ ID NO: 136 - RNA, 46 nt, synthetic construct
  source 1..46: mol_type = other RNA
tcttgtcccacagatatccagaaccgtcatagttccatgaaagcca

SEQ ID NO: 137 - RNA, 46 nt, synthetic construct
  source 1..46: mol_type = other RNA
tgccgtgtaccagctgagagactctgtcatagttccatgaaagcca

SEQ ID NO: 138 - RNA, 46 nt, synthetic construct
  source 1..46: mol_type = other RNA
tgatcctcttgtcccacagatatccgtcatagttccatgaaagcca

SEQ ID NO: 139 - RNA, 36 nt, synthetic construct
  source 1..36: mol_type = other RNA
tgccgtgtaccagctgagagactctgtcatagccat

SEQ ID NO: 140 - RNA, 46 nt, synthetic construct
  source 1..46: mol_type = other RNA
tgccgtgtaccagctgagagactctgtcatagttccattaaagcca

SEQ ID NO: 141 - RNA, 46 nt, synthetic construct
  source 1..46: mol_type = other RNA
tgatcctcttgtcccacagatatccgtcatagttccattaaagcca

SEQ ID NO: 142 - RNA, 37 nt, synthetic construct
  source 1..37: mol_type = other RNA
gccgtgtaccagctgagagactctgtcatagttccat

SEQ ID NO: 143 - RNA, 39 nt, synthetic construct
  source 1..39: mol_type = other RNA
atcctcttgtcccacagatatccgtcatagttccattaa

SEQ ID NO: 144 - RNA, 46 nt, synthetic construct
  source 1..46: mol_type = other RNA
cctagagcccaagagaacccactgagtcatagttccatgaaagcca

SEQ ID NO: 145 - RNA, 46 nt, synthetic construct
  source 1..46: mol_type = other RNA
tttaggttcgtatctgtaaaaccaagtcatagttccatgaaagcca

SEQ ID NO: 146 - RNA, 46 nt, synthetic construct
  source 1..46: mol_type = other RNA
cagggcctcgataatgagataatttgtcatagttccatgaaagcca

SEQ ID NO: 147 - RNA, 46 nt, synthetic construct
  source 1..46: mol_type = other RNA
ctcaacattccagggctcaagcgatgtcatagttccatgaaagcca

SEQ ID NO: 148 - RNA, 46 nt, synthetic construct
  source 1..46: mol_type = other RNA
aagagtataaacacgtactttcatagtcatagttccatgaaagcca

SEQ ID NO: 149 - RNA, 46 nt, synthetic construct
  source 1..46: mol_type = other RNA
tgtctcaaataataaaaaaaataaggtcatagttccatgaaagcca

SEQ ID NO: 150 - RNA, 46 nt, synthetic construct
  source 1..46: mol_type = other RNA
atcatcctgaggacagccaagaggtgtcatagttccatgaaagcca

SEQ ID NO: 151 - RNA, 46 nt, synthetic construct
  source 1..46: mol_type = other RNA
gacctcaggtgatccacccgcttcagtcatagttccatgaaagcca

SEQ ID NO: 152 - RNA, 46 nt, synthetic construct
  source 1..46: mol_type = other RNA
gtaggtgctcagaatttatcatcacgtcatagttccatgaaagcca

SEQ ID NO: 153 - RNA, 46 nt, synthetic construct
  source 1..46: mol_type = other RNA
actcattaacccaccaatcactgatgtcatagttccatgaaagcca

SEQ ID NO: 154 - RNA, 46 nt, synthetic construct
  source 1..46: mol_type = other RNA
ttccttgcttcaggaatggccaggtgtcatagttccatgaaagcca

SEQ ID NO: 155 - RNA, 46 nt, synthetic construct
  source 1..46: mol_type = other RNA
tcttttatttaataggatcttcttcgtcatagttccatgaaagcca

SEQ ID NO: 156 - RNA, 46 nt, synthetic construct
  source 1..46: mol_type = other RNA
cattccagatctgcaagattgtaaggtcatagttccatgaaagcca

SEQ ID NO: 157 - RNA, 46 nt, synthetic construct
  source 1..46: mol_type = other RNA
cccagaagggctcagaaataaaatagtcatagttccatgaaagcca

SEQ ID NO: 158 - RNA, 46 nt, synthetic construct
  source 1..46: mol_type = other RNA
agagccatcaccatcacatccctaagtcatagttccatgaaagcca

SEQ ID NO: 159 - RNA, 46 nt, synthetic construct
  source 1..46: mol_type = other RNA
ggccaaaatccagctgccttccttggtcatagttccatgaaagcca

SEQ ID NO: 160 - RNA, 46 nt, synthetic construct
  source 1..46: mol_type = other RNA
gcttctactcttggcttacaacccagtcatagttccatgaaagcca

SEQ ID NO: 161 - RNA, 46 nt, synthetic construct
  source 1..46: mol_type = other RNA
atctggagggaacttacagcatatggtcatagttccatgaaagcca

SEQ ID NO: 162 - RNA, 46 nt, synthetic construct
  source 1..46: mol_type = other RNA
gaacaactcaaatggaaatgaatatgtcatagttccatgaaagcca

SEQ ID NO: 163 - RNA, 46 nt, synthetic construct
  source 1..46: mol_type = other RNA
tcctgttccatcaccatcaaaaaaagtcatagttccatgaaagcca

SEQ ID NO: 164 - RNA, 46 nt, synthetic construct
  source 1..46: mol_type = other RNA
taaaatgatttggaagagcagagacgtcatagttccatgaaagcca

SEQ ID NO: 165 - RNA, 46 nt, synthetic construct
  source 1..46: mol_type = other RNA
ccacccagcctgctctgccttgggggtcatagttccatgaaagcca

SEQ ID NO: 166 - RNA, 46 nt, synthetic construct
  source 1..46: mol_type = other RNA
aagacattacactcatattacacccgtcatagttccatgaaagcca

SEQ ID NO: 167 - RNA, 46 nt, synthetic construct
  source 1..46: mol_type = other RNA
tgttggttacctccctgccaccaccgtcatagttccatgaaagcca

SEQ ID NO: 168 - RNA, 46 nt, synthetic construct
  source 1..46: mol_type = other RNA
cggcactatcattgactcagctgctgtcatagttccatgaaagcca

SEQ ID NO: 169 - RNA, 46 nt, synthetic construct
  source 1..46: mol_type = other RNA
ccctggctgtgcacattccctcctggtcatagttccatgaaagcca

SEQ ID NO: 170 - RNA, 46 nt, synthetic construct
  source 1..46: mol_type = other RNA
gacatcattgaccagagctctgggcgtcatagttccatgaaagcca

SEQ ID NO: 171 - RNA, 46 nt, synthetic construct
  source 1..46: mol_type = other RNA
cagaagtcaggctggacactgaggagtcatagttccatgaaagcca

SEQ ID NO: 172 - RNA, 46 nt, synthetic construct
  source 1..46: mol_type = other RNA
ggaggcctgcagctcctgcacctccgtcatagttccatgaaagcca

SEQ ID NO: 173 - RNA, 46 nt, synthetic construct
  source 1..46: mol_type = other RNA
gcagcctcccctggctgtgcacattgtcatagttccatgaaagcca

SEQ ID NO: 174 - RNA, 46 nt, synthetic construct
  source 1..46: mol_type = other RNA
gctaccacctctgtgcccccccggcgtcatagttccatgaaagcca

SEQ ID NO: 175 - RNA, 46 nt, synthetic construct
  source 1..46: mol_type = other RNA
aaccccactcccagcttcaaggcccgtcatagttccatgaaagcca

SEQ ID NO: 176 - RNA, 46 nt, synthetic construct
  source 1..46: mol_type = other RNA
caggccaaaatccagctgccttcctgtcatagttccatgaaagcca

SEQ ID NO: 177 - RNA, 46 nt, synthetic construct
  source 1..46: mol_type = other RNA
tccacttcaacacctggtgcattcagtcatagttccatgaaagcca

SEQ ID NO: 178 - RNA, 46 nt, synthetic construct
  source 1..46: mol_type = other RNA
ggcaggaccgatacctcggctcctggtcatagttccatgaaagcca

SEQ ID NO: 179 - RNA, 46 nt, synthetic construct
  source 1..46: mol_type = other RNA
tggtgcatcttggctcactgcaaccgtcatagttccatgaaagcca

SEQ ID NO: 180 - RNA, 46 nt, synthetic construct
  source 1..46: mol_type = other RNA
accagcctgggcaacacagggagacgtcatagttccatgaaagcca

SEQ ID NO: 181 - RNA, 46 nt, synthetic construct
  source 1..46: mol_type = other RNA
gtgcaaacgccttcaacaacagcatgtcatagttccatgaaagcca

SEQ ID NO: 182 - RNA, 46 nt, synthetic construct
  source 1..46: mol_type = other RNA
atccagtgacaagtctgtctgcctagtcatagttccatgaaagcca

SEQ ID NO: 183 - RNA, 46 nt, synthetic construct
  source 1..46: mol_type = other RNA
tttgaaagtttaggttcgtatctgtgtcatagttccattaaagcca

SEQ ID NO: 184 - RNA, 46 nt, synthetic construct
  source 1..46: mol_type = other RNA
tgtcttacctgtttcaaagcttttcgtcatagttccattaaagcca

SEQ ID NO: 185 - RNA, 41 nt, synthetic construct
  source 1..41: mol_type = other RNA
tgtaccagctgagagactctgtcatagttccattaaagcca

SEQ ID NO: 186 - RNA, 46 nt, synthetic construct
  source 1..46: mol_type = other RNA
tcccacagatatccagaaccctgacgtcatagttccattaaagcca

SEQ ID NO: 187 - RNA, 46 nt, synthetic construct
  source 1..46: mol_type = other RNA
gatcctcttgtcccacagatatccagtcatagttccattaaagcca

SEQ ID NO: 188 - RNA, 41 nt, synthetic construct
  source 1..41: mol_type = other RNA
tgccgtgtaccagctgagagactctgtcatagttccattaa

SEQ ID NO: 189 - RNA, 40 nt, synthetic construct
  source 1..40: mol_type = other RNA
gccgtgtaccagctgagagactctgtcatagttccattaa

SEQ ID NO: 190 - RNA, 39 nt, synthetic construct
  source 1..39: mol_type = other RNA
ccgtgtaccagctgagagactctgtcatagttccattaa

SEQ ID NO: 191 - RNA, 40 nt, synthetic construct
  source 1..40: mol_type = other RNA
gatcctcttgtcccacagatatccgtcatagttccattaa

SEQ ID NO: 192 - RNA, 41 nt, synthetic construct
  source 1..41: mol_type = other RNA
tgatcctcttgtcccacagatatccgtcatagttccattaa

SEQ ID NO: 193 - RNA, 38 nt, synthetic construct
  source 1..38: mol_type = other RNA
tgccgtgtaccagctgagagactctgtcatagttccat

SEQ ID NO: 194 - RNA, 36 nt, synthetic construct
  source 1..36: mol_type = other RNA
ccgtgtaccagctgagagactctgtcatagttccat

SEQ ID NO: 195 - RNA, 38 nt, synthetic construct
  source 1..38: mol_type = other RNA
tgatcctcttgtcccacagatatccgtcatagttccat

SEQ ID NO: 196 - RNA, 37 nt, synthetic construct
  source 1..37: mol_type = other RNA
gatcctcttgtcccacagatatccgtcatagttccat

SEQ ID NO: 197 - RNA, 36 nt, synthetic construct
  source 1..36: mol_type = other RNA
atcctcttgtcccacagatatccgtcatagttccat

SEQ ID NO: 198 - RNA, 135 nt, synthetic construct
  source 1..135: mol_type = other RNA
tcttgtcccacagatatccagaaccgtcatagttccatgaaagccaaaagtggctttgatgtttctatgataagggtttcggcccgtggcgtcggggatcgcctgcccattccgatgggcttctccccatttatt

SEQ ID NO: 199 - RNA, 135 nt, synthetic construct
  source 1..135: mol_type = other RNA
tgccgtgtaccagctgagagactctgtcatagttccatgaaagccaaaagtggctttgatgtttctatgataagggtttcggcccgtggcgtcggggatcgcctgcccattccgatgggcttctccccatttatt

SEQ ID NO: 200 - RNA, 135 nt, synthetic construct
  source 1..135: mol_type = other RNA
tgatcctcttgtcccacagatatccgtcatagttccatgaaagccaaaagtggctttgatgtttctatgataagggtttcggcccgtggcgtcggggatcgcctgcccattccgatgggcttctccccatttatt

SEQ ID NO: 201 - RNA, 115 nt, synthetic construct
  source 1..115: mol_type = other RNA
tgccgtgtaccagctgagagactctgtcatagccataaagatgtctatgataagggtttcgacccgtggcgtcggggatcgcctgcccattgaaatgggcttctccccatttatt

SEQ ID NO: 202 - RNA, 135 nt, synthetic construct
  source 1..135: mol_type = other RNA
tgccgtgtaccagctgagagactctgtcatagttccattaaagccaaaagtggctttgatgtttctatgataagggtttcgacccgtggcgtcggggatcgcctgcccattgaaatgggcttctccccatttatt

SEQ ID NO: 203 - RNA, 135 nt, synthetic construct
  source 1..135: mol_type = other RNA
tgatcctcttgtcccacagatatccgtcatagttccattaaagccaaaagtggctttgatgtttctatgataagggtttcgacccgtggcgtcggggatcgcctgcccattgaaatgggcttctccccatttatt

SEQ ID NO: 204 - RNA, 118 nt, synthetic construct
  source 1..118: mol_type = other RNA
gccgtgtaccagctgagagactctgtcatagttccataaagatgtttctatgataagggtttcgacccgtggcgtcggggatcgcctgcccattgaaatgggcttctccccatttatt

SEQ ID NO: 205 - RNA, 117 nt, synthetic construct
  source 1..117: mol_type = other RNA
atcctcttgtcccacagatatccgtcatagttccattaaaaagttgatgtttctatgataagggtttcgacccgtggcgtcggggatcgcctgcccttgaaagggcttctccccatt

SEQ ID NO: 206 - RNA, 135 nt, synthetic construct
  source 1..135: mol_type = other RNA
cctagagcccaagagaacccactgagtcatagttccatgaaagccaaaagtggctttgatgtttctatgataagggtttcggcccgtggcgtcggggatcgcctgcccattccgatgggcttctccccatttatt

SEQ ID NO: 207 - RNA, 135 nt, synthetic construct
  source 1..135: mol_type = other RNA
tttaggttcgtatctgtaaaaccaagtcatagttccatgaaagccaaaagtggctttgatgtttctatgataagggtttcggcccgtggcgtcggggatcgcctgcccattccgatgggcttctccccatttatt

SEQ ID NO: 208 - RNA, 135 nt, synthetic construct
  source 1..135: mol_type = other RNA
cagggcctcgataatgagataatttgtcatagttccatgaaagccaaaagtggctttgatgtttctatgataagggtttcggcccgtggcgtcggggatcgcctgcccattccgatgggcttctccccatttatt

SEQ ID NO: 209 - RNA, 135 nt, synthetic construct
  source 1..135: mol_type = other RNA
ctcaacattccagggctcaagcgatgtcatagttccatgaaagccaaaagtggctttgatgtttctatgataagggtttcggcccgtggcgtcggggatcgcctgcccattccgatgggcttctccccatttatt

SEQ ID NO: 210 - RNA, 135 nt, synthetic construct
  source 1..135: mol_type = other RNA
aagagtataaacacgtactttcatagtcatagttccatgaaagccaaaagtggctttgatgtttctatgataagggtttcggcccgtggcgtcggggatcgcctgcccattccgatgggcttctccccatttatt

SEQ ID NO: 211 - RNA, 135 nt, synthetic construct
  source 1..135: mol_type = other RNA
tgtctcaaataataaaaaaaataaggtcatagttccatgaaagccaaaagtggctttgatgtttctatgataagggtttcggcccgtggcgtcggggatcgcctgcccattccgatgggcttctccccatttatt

SEQ ID NO: 212 - RNA, 135 nt, synthetic construct
  source 1..135: mol_type = other RNA
atcatcctgaggacagccaagaggtgtcatagttccatgaaagccaaaagtggctttgatgtttctatgataagggtttcggcccgtggcgtcggggatcgcctgcccattccgatgggcttctccccatttatt

SEQ ID NO: 213 - RNA, 135 nt, synthetic construct
  source 1..135: mol_type = other RNA
gacctcaggtgatccacccgcttcagtcatagttccatgaaagccaaaagtggctttgatgtttctatgataagggtttcggcccgtggcgtcggggatcgcctgcccattccgatgggcttctccccatttatt

SEQ ID NO: 214 - RNA, 135 nt, synthetic construct
  source 1..135: mol_type = other RNA
gtaggtgctcagaatttatcatcacgtcatagttccatgaaagccaaaagtggctttgatgtttctatgataagggtttcggcccgtggcgtcggggatcgcctgcccattccgatgggcttctccccatttatt

SEQ ID NO: 215 - RNA, 135 nt, synthetic construct
  source 1..135: mol_type = other RNA
actcattaacccaccaatcactgatgtcatagttccatgaaagccaaaagtggctttgatgtttctatgataagggtttcggcccgtggcgtcggggatcgcctgcccattccgatgggcttctccccatttatt

SEQ ID NO: 216 - RNA, 135 nt, synthetic construct
  source 1..135: mol_type = other RNA
ttccttgcttcaggaatggccaggtgtcatagttccatgaaagccaaaagtggctttgatgtttctatgataagggtttcggcccgtggcgtcggggatcgcctgcccattccgatgggcttctccccatttatt

SEQ ID NO: 217 - RNA, 135 nt, synthetic construct
  source 1..135: mol_type = other RNA
tcttttatttaataggatcttcttcgtcatagttccatgaaagccaaaagtggctttgatgtttctatgataagggtttcggcccgtggcgtcggggatcgcctgcccattccgatgggcttctccccatttatt

SEQ ID NO: 218 - RNA, 135 nt, synthetic construct
  source 1..135: mol_type = other RNA
cattccagatctgcaagattgtaaggtcatagttccatgaaagccaaaagtggctttgatgtttctatgataagggtttcggcccgtggcgtcggggatcgcctgcccattccgatgggcttctccccatttatt

SEQ ID NO: 219 - RNA, 135 nt, synthetic construct
  source 1..135: mol_type = other RNA
cccagaagggctcagaaataaaatagtcatagttccatgaaagccaaaagtggctttgatgtttctatgataagggtttcggcccgtggcgtcggggatcgcctgcccattccgatgggcttctccccatttatt

SEQ ID NO: 220 - RNA, 135 nt, synthetic construct
  source 1..135: mol_type = other RNA
agagccatcaccatcacatccctaagtcatagttccatgaaagccaaaagtggctttgatgtttctatgataagggtttcggcccgtggcgtcggggatcgcctgcccattccgatgggcttctccccatttatt

SEQ ID NO: 221 - RNA, 135 nt, synthetic construct
  source 1..135: mol_type = other RNA
ggccaaaatccagctgccttccttggtcatagttccatgaaagccaaaagtggctttgatgtttctatgataagggtttcggcccgtggcgtcggggatcgcctgcccattccgatgggcttctccccatttatt

SEQ ID NO: 222 - RNA, 135 nt, synthetic construct
  source 1..135: mol_type = other RNA
gcttctactcttggcttacaacccagtcatagttccatgaaagccaaaagtggctttgatgtttctatgataagggtttcggcccgtggcgtcggggatcgcctgcccattccgatgggcttctccccatttatt

SEQ ID NO: 223 - RNA, 135 nt, synthetic construct
  source 1..135: mol_type = other RNA
atctggagggaacttacagcatatggtcatagttccatgaaagccaaaagtggctttgatgtttctatgataagggtttcggcccgtggcgtcggggatcgcctgcccattccgatgggcttctccccatttatt

SEQ ID NO: 224 - RNA, 135 nt, synthetic construct
  source 1..135: mol_type = other RNA
gaacaactcaaatggaaatgaatatgtcatagttccatgaaagccaaaagtggctttgatgtttctatgataagggtttcggcccgtggcgtcggggatcgcctgcccattccgatgggcttctccccatttatt

SEQ ID NO: 225 - RNA, 135 nt, synthetic construct
  source 1..135: mol_type = other RNA
tcctgttccatcaccatcaaaaaaagtcatagttccatgaaagccaaaagtggctttgatgtttctatgataagggtttcggcccgtggcgtcggggatcgcctgcccattccgatgggcttctccccatttatt

SEQ ID NO: 226 - RNA, 135 nt, synthetic construct
  source 1..135: mol_type = other RNA
taaaatgatttggaagagcagagacgtcatagttccatgaaagccaaaagtggctttgatgtttctatgataagggtttcggcccgtggcgtcggggatcgcctgcccattccgatgggcttctccccatttatt

SEQ ID NO: 227 - RNA, 135 nt, synthetic construct
  source 1..135: mol_type = other RNA
ccacccagcctgctctgccttgggggtcatagttccatgaaagccaaaagtggctttgatgtttctatgataagggtttcggcccgtggcgtcggggatcgcctgcccattccgatgggcttctccccatttatt

SEQ ID NO: 228 - RNA, 135 nt, synthetic construct
  source 1..135: mol_type = other RNA
aagacattacactcatattacacccgtcatagttccatgaaagccaaaagtggctttgatgtttctatgataagggtttcggcccgtggcgtcggggatcgcctgcccattccgatgggcttctccccatttatt

SEQ ID NO: 229 - RNA, 135 nt, synthetic construct
  source 1..135: mol_type = other RNA
tgttggttacctccctgccaccaccgtcatagttccatgaaagccaaaagtggctttgatgtttctatgataagggtttcggcccgtggcgtcggggatcgcctgcccattccgatgggcttctccccatttatt

SEQ ID NO: 230 - RNA, 135 nt, synthetic construct
  source 1..135: mol_type = other RNA
cggcactatcattgactcagctgctgtcatagttccatgaaagccaaaagtggctttgatgtttctatgataagggtttcggcccgtggcgtcggggatcgcctgcccattccgatgggcttctccccatttatt

SEQ ID NO: 231 - RNA, 135 nt, synthetic construct
  source 1..135: mol_type = other RNA
ccctggctgtgcacattccctcctggtcatagttccatgaaagccaaaagtggctttgatgtttctatgataagggtttcggcccgtggcgtcggggatcgcctgcccattccgatgggcttctccccatttatt

SEQ ID NO: 232 - RNA, 135 nt, synthetic construct
  source 1..135: mol_type = other RNA
gacatcattgaccagagctctgggcgtcatagttccatgaaagccaaaagtggctttgatgtttctatgataagggtttcggcccgtggcgtcggggatcgcctgcccattccgatgggcttctccccatttatt

SEQ ID NO: 233 - RNA, 135 nt, synthetic construct
  source 1..135: mol_type = other RNA
cagaagtcaggctggacactgaggagtcatagttccatgaaagccaaaagtggctttgatgtttctatgataagggtttcggcccgtggcgtcggggatcgcctgcccattccgatgggcttctccccatttatt

SEQ ID NO: 234 - RNA, 135 nt, synthetic construct
  source 1..135: mol_type = other RNA
ggaggcctgcagctcctgcacctccgtcatagttccatgaaagccaaaagtggctttgatgtttctatgataagggtttcggcccgtggcgtcggggatcgcctgcccattccgatgggcttctccccatttatt

SEQ ID NO: 235 - RNA, 135 nt, synthetic construct
  source 1..135: mol_type = other RNA
gcagcctcccctggctgtgcacattgtcatagttccatgaaagccaaaagtggctttgatgtttctatgataagggtttcggcccgtggcgtcggggatcgcctgcccattccgatgggcttctccccatttatt

SEQ ID NO: 236 - RNA, 135 nt, synthetic construct
  source 1..135: mol_type = other RNA
gctaccacctctgtgcccccccggcgtcatagttccatgaaagccaaaagtggctttgatgtttctatgataagggtttcggcccgtggcgtcggggatcgcctgcccattccgatgggcttctccccatttatt

SEQ ID NO: 237 - RNA, 135 nt, synthetic construct
  source 1..135: mol_type = other RNA
aaccccactcccagcttcaaggcccgtcatagttccatgaaagccaaaagtggctttgatgtttctatgataagggtttcggcccgtggcgtcggggatcgcctgcccattccgatgggcttctccccatttatt

SEQ ID NO: 238 - RNA, 135 nt, synthetic construct
  source 1..135: mol_type = other RNA
caggccaaaatccagctgccttcctgtcatagttccatgaaagccaaaagtggctttgatgtttctatgataagggtttcggcccgtggcgtcggggatcgcctgcccattccgatgggcttctccccatttatt

SEQ ID NO: 239 - RNA, 135 nt, synthetic construct
  source 1..135: mol_type = other RNA
tccacttcaacacctggtgcattcagtcatagttccatgaaagccaaaagtggctttgatgtttctatgataagggtttcggcccgtggcgtcggggatcgcctgcccattccgatgggcttctccccatttatt

SEQ ID NO: 240 - RNA, 135 nt, synthetic construct
  source 1..135: mol_type = other RNA
ggcaggaccgatacctcggctcctggtcatagttccatgaaagccaaaagtggctttgatgtttctatgataagggtttcggcccgtggcgtcggggatcgcctgcccattccgatgggcttctccccatttatt

SEQ ID NO: 241 - RNA, 135 nt, synthetic construct
  source 1..135: mol_type = other RNA
tggtgcatcttggctcactgcaaccgtcatagttccatgaaagccaaaagtggctttgatgtttctatgataagggtttcggcccgtggcgtcggggatcgcctgcccattccgatgggcttctccccatttatt

SEQ ID NO: 242 - RNA, 135 nt, synthetic construct
  source 1..135: mol_type = other RNA
accagcctgggcaacacagggagacgtcatagttccatgaaagccaaaagtggctttgatgtttctatgataagggtttcggcccgtggcgtcggggatcgcctgcccattccgatgggcttctccccatttatt

SEQ ID NO: 243 - RNA, 135 nt, synthetic construct
  source 1..135: mol_type = other RNA
gtgcaaacgccttcaacaacagcatgtcatagttccatgaaagccaaaagtggctttgatgtttctatgataagggtttcggcccgtggcgtcggggatcgcctgcccattccgatgggcttctccccatttatt

SEQ ID NO: 244 - RNA, 135 nt, synthetic construct
  source 1..135: mol_type = other RNA
atccagtgacaagtctgtctgcctagtcatagttccatgaaagccaaaagtggctttgatgtttctatgataagggtttcggcccgtggcgtcggggatcgcctgcccattccgatgggcttctccccatttatt

SEQ ID NO: 245 - RNA, 135 nt, synthetic construct
  source 1..135: mol_type = other RNA
tttgaaagtttaggttcgtatctgtgtcatagttccattaaagccaaaagtggctttgatgtttctatgataagggtttcgacccgtggcgtcggggatcgcctgcccattgaaatgggcttctccccatttatt

SEQ ID NO: 246 - RNA, 135 nt, synthetic construct
  source 1..135: mol_type = other RNA
tgtcttacctgtttcaaagcttttcgtcatagttccattaaagccaaaagtggctttgatgtttctatgataagggtttcgacccgtggcgtcggggatcgcctgcccattgaaatgggcttctccccatttatt

SEQ ID NO: 247 - RNA, 130 nt, synthetic construct
  source 1..130: mol_type = other RNA
tgtaccagctgagagactctgtcatagttccattaaagccaaaagtggctttgatgtttctatgataagggtttcgacccgtggcgtcggggatcgcctgcccattgaaatgggcttctccccatttatt

SEQ ID NO: 248 - RNA, 135 nt, synthetic construct
  source 1..135: mol_type = other RNA
tcccacagatatccagaaccctgacgtcatagttccattaaagccaaaagtggctttgatgtttctatgataagggtttcgacccgtggcgtcggggatcgcctgcccattgaaatgggcttctccccatttatt

SEQ ID NO: 249 - RNA, 135 nt, synthetic construct
  source 1..135: mol_type = other RNA
gatcctcttgtcccacagatatccagtcatagttccattaaagccaaaagtggctttgatgtttctatgataagggtttcgacccgtggcgtcggggatcgcctgcccattgaaatgggcttctccccatttatt

SEQ ID NO: 250 - RNA, 119 nt, synthetic construct
  source 1..119: mol_type = other RNA
tgccgtgtaccagctgagagactctgtcatagttccattaaaaagttgatgtttctatgataagggtttcgacccgtggcgtcggggatcgcctgcccttgaaagggcttctccccatt

SEQ ID NO: 251 - RNA, 118 nt, synthetic construct
  source 1..118: mol_type = other RNA
gccgtgtaccagctgagagactctgtcatagttccattaaaaagttgatgtttctatgataagggtttcgacccgtggcgtcggggatcgcctgcccttgaaagggcttctccccatt

SEQ ID NO: 252 - RNA, 117 nt, synthetic construct
  source 1..117: mol_type = other RNA
ccgtgtaccagctgagagactctgtcatagttccattaaaaagttgatgtttctatgataagggtttcgacccgtggcgtcggggatcgcctgcccttgaaagggcttctccccatt

SEQ ID NO: 253 - RNA, 118 nt, synthetic construct
  source 1..118: mol_type = other RNA
gatcctcttgtcccacagatatccgtcatagttccattaaaaagttgatgtttctatgataagggtttcgacccgtggcgtcggggatcgcctgcccttgaaagggcttctccccatt

SEQ ID NO: 254 - RNA, 119 nt, synthetic construct
  source 1..119: mol_type = other RNA
tgatcctcttgtcccacagatatccgtcatagttccattaaaaagttgatgtttctatgataagggtttcgacccgtggcgtcggggatcgcctgcccttgaaagggcttctccccatt

SEQ ID NO: 255 - RNA, 119 nt, synthetic construct
  source 1..119: mol_type = other RNA
tgccgtgtaccagctgagagactctgtcatagttccataaagatgtttctatgataagggtttcgacccgtggcgtcggggatcgcctgcccattgaaatgggcttctccccatttatt

SEQ ID NO: 256 - RNA, 117 nt, synthetic construct
  source 1..117: mol_type = other RNA
ccgtgtaccagctgagagactctgtcatagttccataaagatgtttctatgataagggtttcgacccgtggcgtcggggatcgcctgcccattgaaatgggcttctccccatttatt

SEQ ID NO: 257 - RNA, 119 nt, synthetic construct
  source 1..119: mol_type = other RNA
tgatcctcttgtcccacagatatccgtcatagttccataaagatgtttctatgataagggtttcgacccgtggcgtcggggatcgcctgcccattgaaatgggcttctccccatttatt

SEQ ID NO: 258 - RNA, 118 nt, synthetic construct
  source 1..118: mol_type = other RNA
gatcctcttgtcccacagatatccgtcatagttccataaagatgtttctatgataagggtttcgacccgtggcgtcggggatcgcctgcccattgaaatgggcttctccccatttatt

SEQ ID NO: 259 - RNA, 117 nt, synthetic construct
  source 1..117: mol_type = other RNA
atcctcttgtcccacagatatccgtcatagttccataaagatgtttctatgataagggtttcgacccgtggcgtcggggatcgcctgcccattgaaatgggcttctccccatttatt

SEQ ID NO: 260 - DNA, 56 bp, synthetic construct
  source 1..56: mol_type = other DNA
tcgtcggcagcgtcagatgtgtataagagacagcagtattctaaggacgccagaaa

SEQ ID NO: 261 - DNA, 53 bp, synthetic construct
  source 1..53: mol_type = other DNA
tcgtcggcagcgtcagatgtgtataagagacagaccaactggatcctacccga

SEQ ID NO: 262 - DNA, 53 bp, synthetic construct
  source 1..53: mol_type = other DNA
tcgtcggcagcgtcagatgtgtataagagacagccagcctaagttggggagac

SEQ ID NO: 263 - DNA, 53 bp, synthetic construct
  source 1..53: mol_type = other DNA
tcgtcggcagcgtcagatgtgtataagagacagccgtgtaccagctgagagac

SEQ ID NO: 264 - DNA, 53 bp, synthetic construct
  source 1..53: mol_type = other DNA
tcgtcggcagcgtcagatgtgtataagagacagctgcacacctgtagtcccag

SEQ ID NO: 265 - DNA, 53 bp, synthetic construct
  source 1..53: mol_type = other DNA
tcgtcggcagcgtcagatgtgtataagagacagcttgtccatcactggcatct

SEQ ID NO: 266 - DNA, 53 bp, synthetic construct
  source 1..53: mol_type = other DNA
tcgtcggcagcgtcagatgtgtataagagacaggcatcttggctcactgcaac

SEQ ID NO: 267 - DNA, 53 bp, synthetic construct
  source 1..53: mol_type = other DNA
tcgtcggcagcgtcagatgtgtataagagacagggggtctctcctgaatccct

SEQ ID NO: 268 - DNA, 53 bp, synthetic construct
  source 1..53: mol_type = other DNA
tcgtcggcagcgtcagatgtgtataagagacagtcactgcctgacattcacgg

SEQ ID NO: 269 - DNA, 53 bp, synthetic construct
  source 1..53: mol_type = other DNA
tcgtcggcagcgtcagatgtgtataagagacagtcatttcagccgcctcagtt

SEQ ID NO: 270 - DNA, 53 bp, synthetic construct
  source 1..53: mol_type = other DNA
tcgtcggcagcgtcagatgtgtataagagacagtccagcctggaagacaaagc

SEQ ID NO: 271 - DNA, 53 bp, synthetic construct
  source 1..53: mol_type = other DNA
tcgtcggcagcgtcagatgtgtataagagacagtccattgccaccaaaaccct

SEQ ID NO: 272 - DNA, 53 bp, synthetic construct
  source 1..53: mol_type = other DNA
tcgtcggcagcgtcagatgtgtataagagacagtgctgacaactgcatcctgt

SEQ ID NO: 273 - DNA, 53 bp, synthetic construct
  source 1..53: mol_type = other DNA
tcgtcggcagcgtcagatgtgtataagagacagtgggacatgcaagcccataa

SEQ ID NO: 274 - DNA, 53 bp, synthetic construct
  source 1..53: mol_type = other DNA
tcgtcggcagcgtcagatgtgtataagagacaggggcaaagagggaaatgaga

SEQ ID NO: 275 - DNA, 53 bp, synthetic construct
  source 1..53: mol_type = other DNA
tcgtcggcagcgtcagatgtgtataagagacagaatgaagccaggcaagagca

SEQ ID NO: 276 - DNA, 53 bp, synthetic construct
  source 1..53: mol_type = other DNA
tcgtcggcagcgtcagatgtgtataagagacaggacagccaagaggttttgcc

SEQ ID NO: 277 - DNA, 53 bp, synthetic construct
  source 1..53: mol_type = other DNA
tcgtcggcagcgtcagatgtgtataagagacagactcactaaggggcccatct

SEQ ID NO: 278 - DNA, 53 bp, synthetic construct
  source 1..53: mol_type = other DNA
tcgtcggcagcgtcagatgtgtataagagacagacccggcctgcttttcttaa

SEQ ID NO: 279 - DNA, 53 bp, synthetic construct
  source 1..53: mol_type = other DNA
tcgtcggcagcgtcagatgtgtataagagacagcagtctcacgcagtcactca

SEQ ID NO: 280 - DNA, 52 bp, synthetic construct
  source 1..52: mol_type = other DNA
gtctcgtgggctcggagatgtgtataagagacaggcactttgggaggctgaa

SEQ ID NO: 281 - DNA, 54 bp, synthetic construct
  source 1..54: mol_type = other DNA
gtctcgtgggctcggagatgtgtataagagacagaggacatacaacacgcccag

SEQ ID NO: 282 - DNA, 54 bp, synthetic construct
  source 1..54: mol_type = other DNA
gtctcgtgggctcggagatgtgtataagagacagatgccagtgatggacaaggg

SEQ ID NO: 283 - DNA, 54 bp, synthetic construct
  source 1..54: mol_type = other DNA
gtctcgtgggctcggagatgtgtataagagacagagggttttggtggcaatgga

SEQ ID NO: 284 - DNA, 54 bp, synthetic construct
  source 1..54: mol_type = other DNA
gtctcgtgggctcggagatgtgtataagagacagggtggtagcagctttcacct

SEQ ID NO: 285 - DNA, 56 bp, synthetic construct
  source 1..56: mol_type = other DNA
gtctcgtgggctcggagatgtgtataagagacaggacctcatgtctagcacagttt

SEQ ID NO: 286 - DNA, 54 bp, synthetic construct
  source 1..54: mol_type = other DNA
gtctcgtgggctcggagatgtgtataagagacagggcctccatcatctctctgc

SEQ ID NO: 287 - DNA, 54 bp, synthetic construct
  source 1..54: mol_type = other DNA
gtctcgtgggctcggagatgtgtataagagacagccgctaccacaggaaggatg

SEQ ID NO: 288 - DNA, 54 bp, synthetic construct
  source 1..54: mol_type = other DNA
gtctcgtgggctcggagatgtgtataagagacagacccagtcaaacagaggcac

SEQ ID NO: 289 - DNA, 54 bp, synthetic construct
  source 1..54: mol_type = other DNA
gtctcgtgggctcggagatgtgtataagagacagggattacaggtgtgagcccc

SEQ ID NO: 290 - DNA, 54 bp, synthetic construct
  source 1..54: mol_type = other DNA
gtctcgtgggctcggagatgtgtataagagacaggcagtgtttggcagctcttc

SEQ ID NO: 291 - DNA, 54 bp, synthetic construct
  source 1..54: mol_type = other DNA
gtctcgtgggctcggagatgtgtataagagacagcacctggtgcattcatgtgc

SEQ ID NO: 292 - DNA, 54 bp, synthetic construct
  source 1..54: mol_type = other DNA
gtctcgtgggctcggagatgtgtataagagacagggctctgtttggggctatgt

SEQ ID NO: 293 - DNA, 54 bp, synthetic construct
  source 1..54: mol_type = other DNA
gtctcgtgggctcggagatgtgtataagagacagaactgaggcggctgaaatga

SEQ ID NO: 294 - DNA, 54 bp, synthetic construct
  source 1..54: mol_type = other DNA
gtctcgtgggctcggagatgtgtataagagacaggaacctggccattcctgaag

SEQ ID NO: 295 - DNA, 54 bp, synthetic construct
  source 1..54: mol_type = other DNA
gtctcgtgggctcggagatgtgtataagagacagctgtgcaaacccaggctaga

SEQ ID NO: 296 - DNA, 54 bp, synthetic construct
  source 1..54: mol_type = other DNA
gtctcgtgggctcggagatgtgtataagagacagctgtcccttgcagcttctgt

SEQ ID NO: 297 - DNA, 54 bp, synthetic construct
  source 1..54: mol_type = other DNA
gtctcgtgggctcggagatgtgtataagagacagcaggaggaggattcggaacc

SEQ ID NO: 298 - DNA, 54 bp, synthetic construct
  source 1..54: mol_type = other DNA
gtctcgtgggctcggagatgtgtataagagacagggcagcgaggcatacatagt

SEQ ID NO: 299 - DNA, 54 bp, synthetic construct
  source 1..54: mol_type = other DNA
gtctcgtgggctcggagatgtgtataagagacagaactcatgcctgctgctctt

SEQ ID NO: 300 - DNA, 23 bp, Homo sapiens
  source 1..23: mol_type = genomic DNA
ccagtgtgacagctgagagactt

SEQ ID NO: 301 - DNA, 23 bp, Homo sapiens
  source 1..23: mol_type = genomic DNA
ctcctcctgccccacagataccc

SEQ ID NO: 302 - DNA, 23 bp, Homo sapiens
  source 1..23: mol_type = genomic DNA
ctcctcttgtcccacagaggctc

SEQ ID NO: 303 - DNA, 23 bp, Homo sapiens
  source 1..23: mol_type = genomic DNA
atcctcttttcccactgaaatta

SEQ ID NO: 304 - DNA, 23 bp, Homo sapiens
  source 1..23: mol_type = genomic DNA
atgactcttgtcccacaggtact

SEQ ID NO: 305 - DNA, 23 bp, Homo sapiens
  source 1..23: mol_type = genomic DNA
agcctcttgtcccacagctaaat

SEQ ID NO: 306 - DNA, 23 bp, Homo sapiens
  source 1..23: mol_type = genomic DNA
ctcctcttgtcccacagggatct

SEQ ID NO: 307 - DNA, 23 bp, Homo sapiens
  source 1..23: mol_type = genomic DNA
tccctcttgtcccacagctacca

SEQ ID NO: 308 - DNA, 20 bp, synthetic construct
  source 1..20: mol_type = other DNA
tgctgacattctgcctggag

SEQ ID NO: 309 - DNA, 20 bp, synthetic construct
  source 1..20: mol_type = other DNA
ttctcaggttctgcacatgc

SEQ ID NO: 310 - DNA, 20 bp, synthetic construct
  source 1..20: mol_type = other DNA
ctggagcctgcaggtgaaat

SEQ ID NO: 311 - DNA, 20 bp, synthetic construct
  source 1..20: mol_type = other DNA
cctgtccacacctgatgtcc

SEQ ID NO: 312 - DNA, 20 bp, synthetic construct
  source 1..20: mol_type = other DNA
aagtcaggcctggaggatct

SEQ ID NO: 313 - DNA, 20 bp, synthetic construct
  source 1..20: mol_type = other DNA
atcctttgtaccgcagccat

SEQ ID NO: 314 - DNA, 20 bp, synthetic construct
  source 1..20: mol_type = other DNA
cgagagagaatttggaggcc

SEQ ID NO: 315 - DNA, 20 bp, synthetic construct
  source 1..20: mol_type = other DNA
acaggcacctatcaccatgc

SEQ ID NO: 316 - DNA, 20 bp, synthetic construct
  source 1..20: mol_type = other DNA
ggcaccagcagttgaacaag

SEQ ID NO: 317 - DNA, 20 bp, synthetic construct
  source 1..20: mol_type = other DNA
agacccatctatctgctgct

SEQ ID NO: 318 - DNA, 20 bp, synthetic construct
  source 1..20: mol_type = other DNA
tcctctacagtggggagcag

SEQ ID NO: 319 - DNA, 20 bp, synthetic construct
  source 1..20: mol_type = other DNA
ggtctgagcatgcttcctgt

SEQ ID NO: 320 - DNA, 20 bp, synthetic construct
  source 1..20: mol_type = other DNA
cccaagtagccgagattgca

SEQ ID NO: 321 - DNA, 20 bp, synthetic construct
  source 1..20: mol_type = other DNA
tgagaaagctcagtggaccc

SEQ ID NO: 322 - DNA, 20 bp, synthetic construct
  source 1..20: mol_type = other DNA
ggcacaactgtcttcgagac

SEQ ID NO: 323 - DNA, 20 bp, synthetic construct
  source 1..20: mol_type = other DNA
cccaaagcacaacttcctgc

SEQ ID NO: 324 - protein, 1368 aa, Streptococcus pyogenes
MDKKYSIGLDIGTNSVGWAVITDDYKVPSKKLKGLGNTDRHGIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLADSTDKVDLRLIYLALAHMIKFRGHFLIEGDLNPDNSDVDKLFIQLVQTYNQLFEENPINASRVDAKAILSARLSKSRRLENLIAQLPGEKKNGLFGNLIALSLGLTPNFKSNFDLAEDAKLQLSKDTYDDDLDNLLAQIGDQYADLFLAAKNLSDATLLSDILRVNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPYQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSDILKEYPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKVGFIKRQLVETRQITKHVAQILDSRMNTKYDENDKLIREVRVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKDPIDFLEAKGYKEVRKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 325 - protein, 1368 aa, Streptococcus pyogenes
MDKKYSIGLAIGTNSVGWAVITDDYKVPSKKLKGLGNTDRHGIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLADSTDKVDLRLIYLALAHMIKFRGHFLIEGDLNPDNSDVDKLFIQLVQTYNQLFEENPINASRVDAKAILSARLSKSRRLENLIAQLPGEKKNGLFGNLIALSLGLTPNFKSNFDLAEDAKLQLSKDTYDDDLDNLLAQIGDQYADLFLAAKNLSDATLLSDILRVNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPYQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSDILKEYPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKVGFIKRQLVETRQITKHVAQILDSRMNTKYDENDKLIREVRVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKDPIDFLEAKGYKEVRKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 326 - protein, 1082 aa, Neisseria meningitidis
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 327 - protein, 1070 aa, Bacillus sp.
MRELDYRIGLDIGTNSIGWGIIELSWNKDREQYEKARIVDKGVRMFDKAEIPKTGASLAEPRRIARSSRRRLNRKSQRKKDIRNLLVQHEIISQKELASLYPLTKSSMDIWDIRLDGLDRLLDRFEWTRLLIHLAQRRGFKSNRKSELKDVETGKVLSSIQANEKRLSLYRTVGEMWMKNEDFSKYDKRRNSSNEYVFSVSRADLEKEIVTLFEAQRKFQSSYASADLQKTYLQIWAHQLPFASGNAIVNKVGYCSLLKGKEKRVPKATYTFQYFSTLDQINRTRLGPNFQPFTKEQRDVILDEMFNRTDYYKKKTIPEVTYYDIRKWLALDETIQFKGLTYDPNEELKKIELKSFINLKPFYEIKKVVTNYAKKTNEAFSTLDYDTFAYALTVYKTDKDIRSYLKKSNNLSKCCYDDQLIEELLTLSYTKFGHLSFKAINHVLPIMQEGRTYQEAIHQLGYDATNLKKENRSMFLPLFPDEITNPIVKRALTQARKVVNAIIRRYGSPNSVHIELARELSKSHDERTKIMKAHDENYKKNKGAISILIENGILNPTGYDIVRYKLWKEQGERCAYSLKQIPANTFFNEMKKERSGSPVLEIDHILPYSQSFIDSYHNKVLVYGDENQKKGNRIPYTYFLEGNKDWESFESYVRLNSFFSKKKRGYLLKKAYLPRESNMIKERHLNDTRYASSYLKNFIEKNLKFKEVEGSTRKKHVQTVNGIITAHLRKRWGLEKDRQETYLHHAMDAIIVACTDHHMVTKVTEYYQIKESNKSIRKPYFPLPWVGFREEILSHLARQPIARKISEELKIGYQSFDYILVSRMPKRSVTGAAHEQTIMKKGGIDKKGKTIIIKRVYLKDIKFDENGDFKMVGKEQDLATYEAIKQRYIEYGKESKKAFETPLYKPSKKGKGNLIKKIKVEVQTKSFVREVNGGVAQNGDLVRVDLFEKDNRYYMIPIYVMDTVHSELPNKAVTSSKGYEQWLTIDNSFTFKFSLYPYDLVRLVKGNEDRFLYFSTLDINSDRLNFKDVNKPSKQAENRYSLKTIENLEKYEVGVLGDLRFVRQEIRKNF

SEQ ID NO: 328 - RNA, 19 nt, Bacillus sp.
  source 1..19: mol_type = genomic RNA
gtcatagttccattattgc

SEQ ID NO: 329 - RNA, 84 nt, Bacillus sp.
  source 1..84: mol_type = genomic RNA
gctttgatgtttctatgataagggcttaggcccgtggcgttggggatcgcctgcccattttaatgggcttctccccatctattt

SEQ ID NO: 330 - protein, 1071 aa, Bacillus sp.
MRELDYRIGLDIGTNSIGWGVIELSWNKDREQYEKTRIVDKGVRMFDKAEIPKTGASLAEPRRIARSSRRRLNRKSQRKKDIRNLLVQHEIISQKELTSLYPLSKSSMDIWDIRLDGLDRLLDRFEWARLLIHLAQRRGFKSNRKSELKDVETGKVLSSIQVNEKRLSLYRTVGEMWMKNADCSKYGKRRNSPNEYVFSVSRADLEKEIVTLFEAQRKFHSSYASVDLQKTYIQIWAHQLPFASGNAIVNKVGYCSLLKGKEKRVPKATYTFQYFNTLDQINRTRLGPNFQPFTKEQRDIILDKMFQRTDYYKKKTIPEVTYYDIRKWLALDETIQFKGLTYDPNEELKKIEMKPFINLKPFYEIKKVVTNYAKKTNEVFSALDYDTVAYALTVYKTDKDIRSYLKRSNNLSKRCYDDQLIEELLTLSYTKFGHLSFKAINHVLPIMQEGRTYQEAIHQLGYDTTNLKKENRSMFLPIIPDEITNPIVKRALTQARKVVNAIIRRYGSPNSVHIELARELSKSHDERKKIMTAHDENYKKNKGAVSILIDNGILNPTGYDIVRYKLWKEQGERCAYSLKKIPANTFFNELKKERSGPPVLEVDHILPYSQSFIDSYHNKVLVYGDENQKKGNRIPYTFFSEEDKEWESFESYVRSNSFFSKKKRGYLLKKAYLPRESNLIKERHLNDTRYASSYLKNFIEKNLKFKEAVGITRKKYVQTVNGVITAHLRKRWGLEKDRQETYLHHAMDAIIVACTDHHMVTKVTEYYQIKEGNKSIKKPYFPLPWMGFREEILSHLESQPIARKISEELKIGYQSPDYILVSRMPKRSVTGSAHDQTVMKKGDIDKKGKTIIIKRVHLKDIKFDENGDFKMVGKEQDLATYEAIKQRYLEYRKESKKAFETPLYKPSKKGKGNLIKKIKVEVQTKSFVREINGGVAQNGDLVRVDLFEKDNRYYMVPIYVVDTVRSELPNKAVTSSKGYEQWLSIDNSFTFKFSLYPYDLVRLVKGDEDRFLYFSTLDINSDRLNFKDVNKPSKQAEYRYSLKTIENLEKYEIGVLGDLRLVRQETRKIFK

SEQ ID NO: 331 - RNA, 27 nt, Bacillus sp.
  source 1..27: mol_type = genomic RNA
gtcatagttccattaaagccattgctg

SEQ ID NO: 332 - RNA, 93 nt, Bacillus sp.
  source 1..93: mol_type = genomic RNA
acagcaatggctttgatgtttctatgataagggcttcggcccgtggcgttggggatcgcctgcccattttaatgggcttctccccatctattt

SEQ ID NO: 333 - protein, 1072 aa, Bacillus sp.
MSELDYRIGLDIGTNSIGWGVIELFWNKDRERYEKVRIVDKGVRMFDKAEIPNKGASLAEPRRIARSSRRRLNRKSQRKKEIRNLLVQHGMITQEELDLLYPLSKKSIDIWDIRLDGLDRLLNHLEWARLLIHLAQRRGFKSNRKSELKDAETGKVLSSIQVNEKRLFLYRTVGEMWIKDAEFSKYDRRRNSPNEYVFSVSRADLEKEIVTLFEAQRKFQSSYASKNLQETYLQIWAHQLPFASGNAILNKVGYCSLLKGKERRIPKATYTFQYFSALDQVNRTRLGPDFQPFTQEQKEIILDKMFQRTDYYKKKTIPEVSYYDIRKWLELDETIQFKGLNYDPNEELKKIEKKPFINLKAFYEIKKVVANYAERTNEAFSTLDYDAIAYALTVYKTDKDIRSYLKKSNNLSKRCYDDQLIEELFTLSYTKFGHLSFKAINHVLPIMQEGRTYQEAIHQLGYDTTNLKKENRSMFLPLIPDEITNPIVKRAITQARKVVNAIIRRYGSPNSVHIELARELSKSHDERKKIMTAHDENYKKNKGAISILIENGILNPTGYDIVRYKLWKEQGERCAYSLKEIPPDTFFNELKKERNGSPILEVDHILPYSQSFIDSYHNKVLVYSDENRNKGNRIPYTYFLETNKDWEAFERYVRSNKLFSKKKREYLLKKTYLPRESELIKERHLNDTRYASTFLKNFIEQNLQFKEVEVNLRKKRVQTVNGVITAHLRKRWGLEKNRQETYLHHAMDAIIVACTDHHMVTRITEYYQIKESNKSVKKPYFPMPWEGFRDELLSHLASQPIAKKISEELKAGYQSSDYIFVSRMPKRSVTGAAHDQTIRRKGGIDKKGKTIIIKRVRLKDIKFDENGDFKMVGKEQDLATYEAIKQRYLEHRKNSKKAFETPLYKPSKKGTGNLIKRVKIEGQTKAFVREVNGGVAQNSDLVRVDLFEKDDKYYMVPIYVPDTVCSELPKKVVKSGKGYEQWLTLDNSFTFKSSLYPYDLVRLVKGNEDRFLYFGTLDIDSDRLNFKDVNKPSKQNEYRYSLKTIENLEKYEVGVLGDLRLVKQETRRIFNR

SEQ ID NO: 334 - RNA, 21 nt, Bacillus sp.
  source 1..21: mol_type = genomic RNA
gtcatagttccatgaaagcca

SEQ ID NO: 335 - RNA, 85 nt, Bacillus sp.
  source 1..85: mol_type = genomic RNA
tggctttgatgtttctatgataagggtttcggcccgtggcgtcggggatcgcctgcccattccgatgggcttctccccatttatt

SEQ ID NO: 336 - protein, 1337 aa, Enterococcus sp.
MKKDYVIGLDIGTNSVGWAVMTEDYQLVKKKMPIYGNTEKKKIKKNFWGVRLFEEGHTAEDRRLKRTARRRISRRRNRLRYLQAFFEEAMTALDENFFARLQESFLVPEDKKWHRHPIFAKLEDEVAYHETYPTIYHLRKKLADSSEQADLRLIYLALAHIVKYRGHFLIEGKLSTENISVKEQFQQFMIIYNQTFVNGESRLVSAPLPESVLIEEELTEKASRTKKSEKVLQQFPQEKANGLFGQFLKLMVGNKADFKKVFGLEEEAKITYASESYEEDLEGILAKVGDEYSDVFLAAKNVYDAVELSTILADSDKKSHAKLSSSMIVRFTEHQEDLKKFKRFIRENCPDEYDNLFKNEQKDGYAGYIAHAGKVSQLKFYQYVKKIIQDIAGAEYFLEKIAQENFLRKQRTFDNGVIPHQIHLAELQAIIHRQAAYYPFLKENQEKIEQLVTFRIPYYVGPLSKGDASTFAWLKRQSEEPIRPWNLQETVDLDQSATAFIERMTNFDTYLPSEKVLPKHSLLYEKFMVFNELTKISYTDDRGIKANFSGKEKEKIFDYLFKTRRKVKKKDIIQFYRNEYNTEIVTLSGLEEDQFNASFSTYQDLLKCGLTRAELDHPDNAEKLEDIIKILTIFEDRQRIRTQLSTFKGQFSAEVLKKLERKHYTGWGRLSKKLINGIYDKESGKTILDYLIKDDGVSKHYNRNFMQLINDSQLSFKNAIQKAQSSEHEETLSETVNELAGSPAIKKGIYQSLKIVDELVAIMGYAPKRIVVEMARENQTTSTGKRRSIQRLKIVEKAMAEIGSNLLKEQPTTNEQLRDTRLFLYYMQNGKDMYTGDELSLHRLSHYDIDHIIPQSFMKDDSLDNLVLVGSTENRGKSDDVPSKEVVKDMKAYWEKLYAAGLISQRKFQRLTKGEQGGLTLEDKAHFIQRQLVETRQITKNVAGILDQRYNANSKEKKVQIITLKASLTSQFRSIFGLYKVREVNDYHHGQDAYLNCVVATTLLKVYPNLAPEFVYGEYPKFQAFKENKATAKTIIYTNLMRFFTEDEPRFMKDGEILWSNSYLKNIKKELNYHQMNIVKKVEVQKGGFSKESIKPKGPSNKLIPVKNGLDPQKYGGFDSPVVAYTVLFTHEKGKKPLIKQEILGITIMEKTRFEQNPILFLEEKGFLRPRVLMKLPKYTLYEFPEGRRRLLASAKEAQKGNQMVLPEHLLTLLYHAKQCLLPNQSESLAYVEQHQPEFQEILERVVDFAEVHTLAKSKVQQIVKLFEANQTADVKEIAASFIQLMQFNAMGAPSTFKFFQKDIERARYTSIKEIFDATIIYQSTTGLYETRRKVVD

SEQ ID NO: 337 - RNA, 16 nt, Enterococcus sp.
  source 1..16: mol_type = genomic RNA
gttttagagtcatgtt

SEQ ID NO: 338 - RNA, 74 nt, Enterococcus sp.
  source 1..74: mol_type = genomic RNA
aacatagcaagttaaaataaggttttaaccgtaatcaactgtaaagtggcgctgtttcggcgctttttttgttt

SEQ ID NO: 339 - protein, 1456 aa, Empedobacter sp.
MMIKNILGLDLGTNSIGWALIKQDFENKHGEILGMGSRIIPMSQDILGDFGKGNSISQTADRTKYRSVRRLRERFLLRRERLHRVLHLLNFLPQHYASQIDFEKKFGKFKSETEPKLAWENWGGKFSFLFQNSFNEMLEDFKAAGQGLKIPYDWTIYYLRKKALSQKIEKEELAWILLNFNQKRGYYQLRGEEEEENPNKLVEFYSLKIVDVVADEPQKGKSDIWYSLILENGWVYRRASKIPLFDWKDKTRDFIVTTDLNDDRSVKTDKEGNEKRSFRAPSENDWTLVKKKTEQEIDQSHKTVGTYIYETLLLNPKQKIKGKLVRTIERKFYKDELKQILEKQKEFHQELKNDDLYNDCIRELYRNNEAHQLTLSKKDFVHLLMDDLIFYQRPLRSQKSSISNCTLEFRKYKDENGIEHTQYLKAIPKSNPYYQEFRLWQWMYNLNIYRKDDEANVTKEFLNTNKDFESLFEFLNNRKEIEQKPLIKFLLEQKDINKKLLNAEAEKYRWNYVEDKKYPCNETKTMISSRLDKVENISDDFLTRDIEQKIWHIIYSVNDKIEYEKALKSFATRNDLDENSFIEAFKKFSPFKSEYGSFSEKAIKKLLPLMRLGKYWYEDEIVKHSDIYFKNIENLLGDFSNRDKKISEEDKEKWNKSINLKLQEELKDFQTAEIDLFQGLRLHIAQYLVYGRHSEASMIGKWNSAEDLEEFLKDFKQHSLRNPIVEQVITETLRVVKDIWLKYGNGAKDFFNEIHIELGREMKLPADDRKKLTNQISENENTNFRIKALLAEMMNDSSVENVRPFSPMQQEILKIYEDDVLKSDIEIEDDILKISKTAQPSPSDLKRYKLWLEQKYKSPYTGQIIPLNKLFTPEYEIEHIIPQSRYFDDSFSNKVICESAVNKLKDNYIGLEFIKQFGGTIIELGFGKSIKVFETKEYEDFVKKHYANNQGKRNKLLMEDIPEKMIERQMNDTRYISKYISGVLSNIVRVEDGSDEGVNSKNIVPGNGKITTQLKQDWGLNDVWNDLILPRFERMNQLTNSKVFTAWNENYQKFLPTVPIEYSKGFSKKRIDHRHHALDALVIACATKDHVNLLNNQSAKSDTKRYDLKKKSMKFEKVVYNDAKTGEKIEREVPKQFLKPWENFTLDVKHNLETIIVSFKQNLRVINKATNYYEKYVEKDGTKNKERVEQTGTNWAIRKPMHKDTVSGKVDLPWVKVPKGKILTATRKSLDSSFDLKSIGSITDTGIQKILKNYLAFKDGNPELAFSPEGIDDLNKNIEKYNDGKPHQPINKVRVFELGSKFQVGQSGNKKDKYVEAAKGTNLFFAVYEDEKGKRNYETIPLNEVIERQKQGLSVVDLKGTNDFYLCPNDFVYIPSGDELENINNVDFKDIKKEINERIYKVVSFTGNRLSCIPYMVATTIVNKLEFTQLNKIEFTKEKEICIKLNVDRLGNISKA

SEQ ID NO: 340 - RNA, 18 nt, Empedobacter sp.
  source 1..18: mol_type = genomic RNA
gttgtgaattgctttcaa

SEQ ID NO: 341 - RNA, 75 nt, Empedobacter sp.
  source 1..75: mol_type = genomic RNA
ttgaaaagcaattcacaataaggattattccgttgtgaaaacattcaaggcggggcaactcgtcttttttctttt

SEQ ID NO: 342 - protein, 1090 aa, Rhizobium sp.
MNNLLYSFDIGTNSIGWCVFALDEVGDPCRIVDLGARIYADGRDPQTKTSLAVARREARAMSRRRDRSLRRRKATLRTMIEYGLMPAHKVEQETLLRKTGDREGGDEGFNPYALRARALGEKLPPFYIGRALFHLGQRRGFKSNRKTDRKDNDKGKIALGIDELRAAMHRSGSPTLGAWLAMRRADGHPVRLRAGSEVFDAEGYAFYPERSLLEDEFRQIWTAQAVHHPQLLTSERRAHLFRVMFYQRPLKKPLVGRCSFNPAEARLSRAHPLFQEFRLYKEVNDLEVVLPDQSHRKLTLDERNALVAKLKSSRKASFSVLRRTLKLTPDLAFNKESEARKDLLGDEINSALADAKMFGVRWGGFPRARQWEIITHLKEEENPARLSDWLKSEFGLDDERVVAIANIALPEGYGRLGETALASMLEEMKTAVIPESEAAKRCGYDHSNLAKEQDEGLDILPEYQEILERHIPPGTGDPDDIYDIRKGRITNPTVHIGLNQLRRVVNALIKRHGKPRHIVVELARDLQLSEKQKADVNRRIAKNTREAEGRSQKLIEMGQLDTGYNRLLLKLWEELNQDKPEDRVCIYSGKPIGIAMLFSGEVDIDHILPWSKTLDDSQANKLLCLKSANRQKRNRTPSDVPEWRDCYEEVLARAARLPKNKRWRFAADAMQQFEAEGGFLARQLTDTQYLSRMAFEYLSALFPSEEADKWGELRQRKRVHVVPGRLTELLRRNWGLNTLLPDHNLGEMAQEKNRKDHRHHAIDAAVIGVTSRSLLQRMSGAAARLDDVAFDDLVRTVVKDNPPWPGFREELLGCINRVTVSHKPDHGTVSRAAYAQGKGQTAGKLHNDTAYGITGLQDMKGSPLVVRRKPFMALEVKDIASIRDTELQSALYDAIGSLTEKKALQEALVRFRDRHPQFKGIRRVRVLEALSVIPIVDNNGKAYKGYKGDANYRYEVWETLDGRWHTEVVSMFDAHQPGWQSPFHRQHPAARRVLKLQQNDMVAYEHPADGYTIARVVKFSNDKRIYFASHRESGSLKARDADKGDPFTYFAKANNGLRDIKCRQVRIDAAGRVFDPGPQDREARQSKGRA

SEQ ID NO: 343 - RNA, 31 nt, Rhizobium sp.
  source 1..31: mol_type = genomic RNA
ggttgcggctggaccgcgttttctgatctgc

SEQ ID NO: 344 - RNA, 79 nt, Rhizobium sp.
  source 1..79: mol_type = genomic RNA
gcaaatcagaaaacgcggtctggccgttaacaagcctcgaccagcaccagataaggcggtccctccgggggccgttttt

SEQ ID NO: 345 - protein, 1071 aa, Bacillus sp.
MRELDYRIGLDIGTNSIGWGVIELSWNKDREQYEKTRIVDKGVRMFDKAEIPKTGASLAEPRRIARSSRRRLNRKSQRKKDIRNLLVQHEIISQKELTSLYPLSKSSMDIWDIRLDGLDRLLDRFEWARLLIHLAQRRGFKSNRKSELKDVETGKVLSSIQVNEKRLSLYRTVGEMWMKNADCSKYGKRRNSPNEYVFSVSRADLEKEIVTLFEAQRKFHSSYASVDLQKTYIQIWAHQLPFASGNAIVNKVGYCSLLKGKEKRVPKATYTFQYFNTLDQINRTRLGPNFQPFTKEQRDIILDKMFQRTDYYKKKTIPEVTYYDIRKWLALDETIQFKGLTYDPNEELKKIEMKPFINLKPFYEIKKVVTNYAKKTNEVFSALDYDTVAYALTVYKTDKDIRSYLKRSNNLSKRCYDDQLIEELLTLSYTKFGHLSFKAINHVLPIMQEGRTYQEAIHQLGYDTTNLKKENRSMFLPIIPDEITNPIVKRALTQARKVVNAIIRRYGSPNSVHIELARELSKSHDERKKIMTAHDENYKKNKGAISILIENGILNPTGYDIVRYKLWKEQGERCAYSLKKIPANTFFNELKKERSGPPVLEVDHILPYSQSFIDSYHNKVLVYGDENQKKGNRIPYTFFSEEDKEWESFESYVRSNSFFSKKKRGYLLKKAYLPRESNLIKERHLNDTRYASSYLKNFIEKNLKFKEAVGITRKKYVQTVNGVITAHLRKRWGLEKDRQETYLHHAMDAIIVACTDHHMVTKVTEYYQIKEGNKSIKKPYFPLPWMGFREEILSHLESQPIARKISEELKIGYQSSDYILVSRMPKRSVTGSAHDQTVMKKGGIDKKGKTIIIKRVHLKDIKFDENGDFKMVGKEQDLATYEAIKQRYLEYRKKSKKAFETPLYKPSKKGKGNLIKKIKVEVQTKSFVREINGGVAQNGDLVRVDLFEKDNRYYMVPIYVVDTVRSELPNKAVTSSKGYEQWLSIDNSFTFKFSLYPYDLVRLVKGDEDRFLYFSTLDINSDRLNFKDVNKPSKQAEYRYSLKTIENLEKYEIGVLGDLRLVRQETRKIFK

SEQ ID NO: 346 - RNA, 34 nt, Bacillus sp.
  source 1..34: mol_type = genomic RNA
gtcatagttccattaaagccattgctgttttatg

SEQ ID NO: 347 - RNA, 148 nt, Bacillus sp.
  source 1..148: mol_type = genomic RNA
catgatacagcaatggctttgatgtttctatgataagggcttcggcccgtggcgttggggatcgcctgcccattttaatgggcttctccccatctatttaatgagaaatttacaaccttggctattcttaaatagctaaggttttttt

SEQ ID NO: 348 - protein, 1054 aa, Staphylococcus sp.
MKEKYILGLDLGITSVGYGIINFETKKIIDAGVRLFPEANVDNNEGRRSKRGSRRLKRRRIHRLERVKLLLTEYDLINKEQIPTSNNPYQIRVKGLSEILSKDELAIALLHLAKRRGIHNINVSSEDEDASNELSTKEQINRNNKLLKDKYVCEVQLQRLKEGQIRGEKNRFKTTDILKEIDQLLKVQKDYHNLDIDFINQYKEIVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPQFTEFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVKRTFIQSINVINKVIEKYGIPEDIIIELARENNSDDRKKFINNLQKKNEATRKRINEIIGQTGNQNAKRIVEKIRLHDQQEGKCLYSLESIALMDLLNNPQNYEVDHIIPRSVAFDNSIHNKVLVKQIENSKKGNRTPYQYLNSSDAKLSYNQFKQHILNLSKSKDRISKKKKDYLLEERDINKFEVQKEFINRNLVDTRYATRELTSYLKAYFSANNMDVKVKTINGSFTNHLRKVWRFDKYRNHGYKHHAEDALIIANADFLFKENKKLQNANKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 349 - RNA, 28 nt, Staphylococcus sp.
  source 1..28: mol_type = genomic RNA
gttttagtactctgtaattttaggtatg

SEQ ID NO: 350 - RNA, 85 nt, Staphylococcus sp.
  source 1..85: mol_type = genomic RNA
tcatacctaaaattacagaatctactgaaacaagactatatgtcgtgtttatcccactaatttattagtgggatttttttgtttt

SEQ ID NO: 351 - protein, 1076 aa, Bacillus sp.
MSELDYRIGLDIGTNSIGWGVIELFWNKDRERYEKVRIVDKGVRMFDKAEIPKTGASLAEPRRIARSSRRRLNRKSQRKKEIRNLLVQHGVITQEELDLLYPLSKKSMDIWDIRLDGLDRLLNHLEWTRLLIHLAQRRGFKSNRKSELKDAETGKVLSSIQVNEKRLSLYRTVGEMWIKDAEFSKYDRRRNSPNEYVFSVSRADLEKEIVTLFEAQRKFQSSYSSKNLQETYLQIWAHQLPFASGNAILNKVGYCSLLKGKERRIPKATYTFQYFSALDQVNRTRLGPDFQPFTQEQKEIILDKMFQRTDYYKKKTIPEVSYYDIRKWLELDETIQFKGLNYDPNEELKKIEKKPFINLKAFYEIKKVVANYAERTNEAFSTLDYDAIAYALTVYKTDKDIRSYLKKSNNLSKRCYDDQLIEELFTLSYTKFGHLSFKAINRVLPIMQEGRTYQEAIQQLGYDTTNLKKENRSMFLPLIPDEITNPIVKRALTQARKVVNAIIRRYGSPNSVHIELARELSKSHDERKKIMTAHDENYKKNKGAISILIENGILNPTGYDIVRYKLWKEQGERCAYSLKEIPPDTFFNELKKERNGSSILEVDHILPYSQSFIDSYHNKVLVYSDENRNKGNRIPYTYFLETNKDWEAFERYVRSNKLFSKKKREYLLKKAYLPRESELIKERHLNDTRYASTFLKNFIEQNLQFKEVEVNLRKKRVQTVNGVITAHLRKRWGLEKNRQETYLHHAMDAIIVACTDHHMVTRITEYYQIKESNKSVKKPYFPMPWEGFRDELLSHLASQPIAKKISEELKAGYQSSDYIFVSRMPKRSVTGAAHNQMIRRKGGIDKKGKTIIIKRVRLKDIKFDENGDFKMVGKEQDLATYEAIKQRYLDHGKNLKKAFETPLYKPSKKGTGNLIKRVKIEGQTKAFVREVNGGVAQNSDLVRVDLFEKDDKYYMVPIYVPDTVCSELPKKVVKSGKGYEQWLTLDNSFTFKFSLYPYDLVRLVKGDEDRFLYFGTLDIDSDRLNFKDVNKPSKQNEYRYSLKTIEDLEKYEVGVLGDLRLVRKETRRNFHEIKIN

SEQ ID NO: 352 - RNA, 20 nt, Bacillus sp.
  source 1..20: mol_type = genomic RNA
gtcatagttccattaaagcc

SEQ ID NO: 353 - RNA, 84 nt, Bacillus sp.
  source 1..84: mol_type = genomic RNA
ggctttgatgtttctatgataagggtttcggcccgtggcgtcggggatcgcctgcccattccgatgggcttctccccatttatt

SEQ ID NO: 354 - protein, 1071 aa, Bacillus sp.
MRELDYRIGLDIGTNSIGWGVIELSWNKDREQYEKTRIVDKGVRMFDKAEIPKTGASLAEPRRIARSSRRRLNRKSQRKKDIRNLLVQHEIISQKELTSLYPLSKSSMDIWDIRLDGLDRLLDRFEWARLLIHLAQRRGFKSNRKSELKDVETGKVLSSIQVNEKRLSLYRTVGEMWMKNADFSKYGKRRNSPNEYVFSVSRADLEKEIVTLFEAQRKFHSSYASVDLQKTYIQIWAHQLPFASGNAIVNKVGYCSLLKGKEKRVPKATYTFQYFNTLDQINRTRLGPNFQPFTKEQRDIILDKMFQRTDYYKKKTIPEVTYYDIRKWLALDETIQFKGLTYDPNEELKKIEMKPFINLKPFYEIKKVVTNYAKKTNEVFSALDYDTVAYALTVYKTDKDIRSYLKRSNNLSKRCYDDQLIEELLTLSYTKFGHLSFKAINHVLPIMQEGRTYQEAIHQLGYDTTNLKKENRSMFLPIIPDEITNPIVKRALTQARKVVNAIIRRYGSPNSVHIELARELSKSHDERKKIMTAHDENYKKNKGAISILIENGILNPTGYDIVRYKLWKEQGERCAYSLKKISANTFFNELKKERSGPPVLEVDHILPYSQSFIDSYHNKVLVYGDENQKKGNRIPYTFFSEEDKEWESFESYVRSNSFFSKKKRGYLLKKAYLPRESNLIKERHLNDTRYASSYLKNFIEKNLKFKEAVGITRKKYVQTVNGVITAHLRKRWGLEKDRQETYLHHAMDAIIVACTDHHMVTKVTEYYQIKEGNKSIKKPYFPLPWMGFREEILSHLESQPIARKISEELKIGYQSSDYILVSRMPKRSVTGSAHDQTVMKKGGIDKKGKTIIIKRVHLKDIKFDENGDFKMVGKEQDLATYEAIKQRYLEYRKESKKAFETPLYKPSKKGKGNLIKKIKVEVQTKSFVREINGGVAQNGDLVRVDLFEKDNRYYMVPIYVVDTVRSELPNKAVTSSKGYEQWLSIDNSFTFKFSLYPYDLVRLVKGDEDRFLYFSTLDINSDRLNFKDVNKPSKQAEYRYSLKTIENLEKYEIGVLGDLRLVRQETRKIFK

SEQ ID NO: 355 - RNA, 24 nt, Bacillus sp.
  source 1..24: mol_type = genomic RNA
gtcatagttccattaaagccattg

SEQ ID NO: 356 - RNA, 138 nt, Bacillus sp.
  source 1..138: mol_type = genomic RNA
caatggctttgatgtttctatgataagggcttcggcccgtggcgttggggatcgcctgcccattttaatgggcttctccccatctatttaatgagaattttacaaccttggctattcttaaatagctaaggttttttt

SEQ ID NO: 357 - protein, 1091 aa, Rhizobium sp.
MDIETLFAFDIGTNSIGFCVFALDENGEPYRIIDIGTRIYADGRDPQSKASLAVARREARAMSRRRDRYVNRRKAVLRTLTEYGLMPAEASARQALIAETSDRNGAAGEASANPYALRAMALDEKLPLYWIGRVLFHLNQRRGFKSNRKTDRKDNEKGVIALGIGELRAAMHKTKARTYGEWLAARREDGHVTRLRAGSDAFEGDGYAFYPERSLLEAEFREIWARQSSFYPDVLTEARGDHLFQVMFYQRPLKKPRVGKCAFNPLEERLAKAHPLFQEFRLYKEVNELELVLPDHSHKKLDIEQRNALVILLRANREVTFKKLRTALKLAPDIVFNKESESREKLRGDEVHSVLADKKRFGPQWGALSRERQWQIIQTLKDEENPEKLFGWLKEEFGFEGEKADAIANAPLPEGYGRLGETALSSMLDEMKAAVIPEAEAAKNCGYDHSKLGEDREEGEAFLPPYQEILSRQIPPGTNDPADIYDIRMGRFTNPTVHIGLNQLRRVVNALIARHGKPQFVSLELARDLQLSEKQKAEANRAIAKNTREAAARSKKLGEMGQLDTGYNRLLLKLWEELNPSKPEDRVCIYSGKPIGIDMLFSAEVDVDHILPWSKTLDDSQANKLLCLKSANRQKRNRAPADVPEWRDRYEEILARAARLAPNKRRRFAPNAMAEFEAEGGFLARQLTDTQYLSRMAREYLSCLFPSAEIDRRGERKKKIRVIVSPGRLTEMLRRNWGLNNILPDHNLGEMTQAKNRKDHRHHAIDAAVVGVTTRSLLQRIATAAGRLDEADFENLVRKMVSENPPWPTFREELQAAVNGIVVSHKPDHGTVSRKGYAEGKGRTAGRLHNDTAYGFGTDASGNPVAVRRKLFTSLEAKDIPMIRDPELQAELYAAIDGLDDRRALQDALLSFRRTHPKFKGIRRVRMAETLSFIPIRDSQGNVYKGYKGDANYRYDVWETLDGKWHADVVTMFNAHQPDWRSPVHQEHPTARRVLRLQQNDMVAYEHPNDGYTIARVVKFNTAGIVYFASHRESGSLKARDADKQDPFKYFSKSAAGLKDIQCRQIRIDAAGRVFDPGPQDRASKSTRKTN

SEQ ID NO: 358 - RNA, 29 nt, Rhizobium sp.
  source 1..29: mol_type = genomic RNA
gttgcggctggaccgcgttttctgatctg

SEQ ID NO: 359 - RNA, 78 nt, Rhizobium sp.
  source 1..78: mol_type = genomic RNA
cagatcagaaaacgcggtctggccgttaacaagctagaagcaccaaataaggccgttccttcgggagcggctttttct

SEQ ID NO: 360 - protein, 1401 aa, Myroides sp.
MKRILGIDLGTTSIGWAYVHEAENENEISSIEKLGVRVNPLTVDEQINFEKGKPITTNATRTLFRSARRNLQRFKLRRKHLIEILKHNNWISKQTLLTENGNHTTFETISLRDKAATQKVTLEELARVLLMINKKRGYKSSRKVNNTEEGQLIDGMEVAKKLYHEQLTPGQLSLQLIKQGVKRLPDYYRSDLNHELDLIWAFQQKFYPHILTSEFKELLKGKGLRATSNQFWTNYNFNTADNKGTREEKKLQNYQWRSDALIKQLDKEIVAYVISEINGQINNSSGYLGAISDRSKELYFNQLTVGQYLHRQLIANPHTRLKSQVFYRQDYLDEFEKIWETQSKFYPKELTTVLKNEIRDTVIFYQRKLKSQKGLISFCEFEQEQKIINGKTKTIGHRVIPKSSPLFQEFKIWQQLHNVVLRNKKTNVITPLAEEQKSHLFEELNLKGKLSSTQILKLIEDKPKDWELNYSELEGNNTNKALYNAYLDILDLEGYDIRDELKIKLNKDDIELSDLDVNASEIKEMIFSIFKHLGINTEILEFDATLKDKAFEKQASYQLWHLLYSYEEDNSPTGLDRLHNLLQKKFNFTLDQAKLVGNVLFQDDYGNLSSKAIKNIFPYITDNNYSTACELAGYKHSKHSLTKEENEKRELKSRLTILTKNSLRNPVVEKILNQMINVINTLIESENDKLVAQGKEPNFQFDEIRIELARELKKNAKEREELTKSMTQGKTNHEKIIKILQKEDGIKNPTRNDITRFKLYSELKNNGYKDLYTNEYIERKDIFSKEYDIEHIIPQSKLFDDSFSNKTLVRRNVNLKKGNQTAYDFILSEYGQEKANEFETRITNLYSLGKDEGISKSKYKKLLMQESEIGKGFIERDLRETQYIAKKAKSLLLEITRNVVSTSGGITDRLREDWGLVNVMKELNLKKFRDAGLTEFVEMKDGNKKEVIIDWTKRNDHRHHAMDALTIAFTKHNHIQYLNHLNARKNTKDELHTNIIAIEKKETTIITDERGNKKRIFKEPIPNFRNIAKSHLEAILVSHKAKNKVVTKNINKISGKKIGQQTLTPRGQLHKETVYGKIRQYVSKEEKVGPKFTKEVIELVSNPTYRDLLLKRLQENNNDPKKAFGGKNALSKSPIYINLEKNIIVPEVVKLVWLEDDYTIRKEISPELKIDKVVDEGVKRILQNRLALYNGDAKIAFSDLDNNPIWLSKDKGITIKRVTLSGVKNATALHTKKDHLGQPILDHKGQEIPVDFISTGNNHHVAIYEDKNGKLQESVVSHFEAVERVNQQLPIIDKTFNQHLGWKFLFTMKQNELFLFPSDDFNPNEIDLFDTRNYAVISKHLFRAQKLSTKDYSFRHHLETNVEDIKDLKTITWRREGLNGIQNIVKIRTNHLGEIVHIGEY

SEQ ID NO: 361 - RNA, 21 nt, Myroides sp.
  source 1..21: mol_type = genomic RNA
gttgtgaattgctttcaaaca

SEQ ID NO: 362 - RNA, 105 nt, Myroides sp.
  source 1..105: mol_type = genomic RNA
tgaaagcaattcacaataaggattattccgttgtgaaaacatctagtcctcgactaatttcggggaataaagccttaactctgttaaggctttttttagtttttt

SEQ ID NO: 363 - protein, 1092 aa, Bacillus sp.
MRYSIGLDIGTTSIGNAVINKDLQRFEHLGVRIFDAAENPKDGSSLSAPRRLARSSRRRLRRRKHRVERTKQLLINKGLLTKTQVKNLYNSKNINLDIWDIRVSGIDRKLFNNEFARVLIHFSKNRGFKSNRKSELKEDDNGAILSAVKENRELMDEKGYRTIAEMLVSDEKYEGTKRNKGGDYSHVVARSDIENEICLLFQKQREYGHPFATEENEEAFLSIWSSQRPFSTKDDIVKKIGNCTLEPKEKRAPKSTYTFERFRALDKLNRLRILSTTAPSRPLTNEERKSILSSLFSKKEVKYKELRKLLKLTDDQRFNEIYYSPDETIEKTENRTFLSLESQYKIKKIIEKTESKNMQSSYHPIDYDTIGYALTVFKDDKDIQHYLQNSYIDSKGKAIPNMANREYNLELIEELLGLSFAKFGHLSLKALNNILPYMEEGEPYHIACEMASYQFSQRLSKEKHRLLPPIPVDEIPNPVVVRALTQVRKVLNSIIKKYGPPSDIYIELAREMSKPFKERKSLEREFNENRQINEKAKAHISELYRIPNDPRPHDILKFKLWNEQNGICPYSLKPISIEYLFNIGYAEVDHIIPYSRSFDDSNGNKVLVLTRENQNKLNRTPYEWFGHEENRWEDFVSFIRTMKVGKKKKNMLLKKNFDEEQEEQILSRNLNDTRYITRYIKSFIEDNLEFRTEENKEQYVHTVNGAYTSLMRKRWGLNKDRRGNDLHHAVDAAIIAVSLPFKNKVNAYFKRQETGLSKLLNNKKDIFPEPWRNFIKELEARMIQDPEKMKRALESLELETYGEIFLNKLKPIFVSRMPKHSIKGQIHEETIRRVRGFTEEGFLVTVKKTRLDQIPFDKNGDFPMYGKETDIKTYMAIKQRYLEYGQDKQKAFAVPLRKPSKNPKNAPIVRSVKIEGKANRVVMLDDKAAADNASIVRTEVFRHKKTGEYYLTPVYVADILSNKIPDRLITIKKSYSDWDRITDEHEYLFSLYNNDLVKIILPKEKETKKYTGGNHLWQEGFFYFKGVDSSNAGIKIINHLNSFEARIGTKRLIAFEKYQVNPLGEINKVHGEKRPGELLNKEEIKENRKNIS

SEQ ID NO: 364 - RNA, 23 nt, Bacillus sp.
  source 1..23: mol_type = genomic RNA
gccataattcctctgtaaaactt

SEQ ID NO: 365 - RNA, 112 nt, Bacillus sp.
  source 1..112: mol_type = genomic RNA
aaggtttatagagttattatggtaaggcaatatgccgtggcgttggggatcgcctatgtccggttttaccggatctccctaaaggtgactaactttggttagtcaccttttt

SEQ ID NO: 366 - protein, 1075 aa, Bacillus sp.
MDYRIGLDIGTNSVGWGVIRMQYNDLLERYEKQGIVDAGVRMFDKAEIPKTGASLAAPRREARSSRKRLRRRSQRKKAIRYLIILHELLSEDQLQQLYPLQKESIDIWDIRLESLDRVLNQTEWARLLIHLGQRRGFKSNRKSEYKEEETGKVLVNIQENQKALQQYRTVAEMWMKDAKFSQFDKKRNAPGEYLFSVSRYDLQNEIQTIFESQRNLGSPYATTDLEEEYISVWSKQLPFASGEDILKKVGYCSLERKERRIPKATYTFQYFILLDGLNKIRVGADARQLTTEERSILIEKILTRNDYFSKKSLPDLTYGDLRKWLALDSSLLFKDLTYDPNEKVSKVEKKVFANLKSLYELRKVVETSSNGQSFERIDYDTFGYALTVYKTDEDIRRYLKKPSNLAHKVYEEDLIERLLSLSYEKFGHLSLKAIQTILPYMEDGMMYTEAVQAAGYDITGLQSAKRTLLLPTIPADITNPVVRRALTQTRKVVNAIIKRYGSPLTIHIELARELSKDHQERQQILKNQQANYERNKGAISVLVENGILNPTGYDIVRYKLWKEQGERCAYSLKVIPPSVFFGELKRERNHIPTLEVDHILPYSQSFMDNYHNKVLVYSDENQKKGNRIPSQYLMAVSPEKWTKFEEYVSSNKGFSKQKRQYLLKKDYSSRERDIVKERHLVDTRYATRFMKNFIEKTLLFKESRSSSLTKRVQTVNGKITAHFRKRWGLEKERTETYLHHALDAIVVACTDQHMVTRVTEYYQERERNVRQPYFPWPWEGFRDELLNYLNRQPDSLEISQSIKQNLFLRDYLMVSRMPKHSVTGMSHKQTIRSKACVDEKGKVKTTKRVLLQDIKFDSNGDFKMIGKEQDMATYNAIKERFLSSGGNVKSAFTEPLHKPSKKGKGNVIRKVTVEDQTKSFVRDVNGGVAENGSLVRIDLFEKEGMYYVVPIYVGDTVLKQLPIKISASNRGYYKWVELDENYRFKFSLYAHDLIRIVTKDEDRFFYFSYFDNDGNRIKFLNINSPTGKNENRYGVRSIKLIEKYNVSTLGDISLVKSEERQLFQGMKKKVLNIIN

SEQ ID NO: 367 - RNA, 17 nt, Bacillus sp.
  source 1..17: mol_type = genomic RNA
gtcatagttccattaaa

SEQ ID NO: 368 - RNA, 83 nt, Bacillus sp.
  source 1..83: mol_type = genomic RNA
tttgatgttcctatgataagggccttgagcccgtggcgttggggatcgcctgcccgtttatacgggcttctccctacttactt

SEQ ID NO: 369 - protein, 1404 aa, Chryseobacterium sp.
MKKTLGIDLGTNSIGWVIRNADLLDNQIEDFGVITFEKGVASEKGNEFPKVQKRTESRGKRRNYQSEKYRKFALLAFLIQKNMCPLTMDEFCNWKDYRKGKKREYPQSEEFLNWIRFDFNGDGLPDFHLFGKEKDDSYYVFRAFAADENHKNVFDNNPHILGRVLYQMVQRRGFKGRDEEEAKTMLVGSDKNGTKGRNDIEEYIEKYKTLGAALYYHQKDFGGRIKQRYNLRKDNENELKEICKIHQLSQEDYEKLWKAIIWQRPLRTQKGLVGNCIYEKNKKRVAVSHPLYEEYRTWVFINNLNIIPPFGEEKQNYISEKIYPLFYKSKPDFELSDIDKQLKKDGAKRTSKHNDKTKVLSVKLLKQFQDVFGEDWKQKLQWDINGDRNSQPEKKEKSLYTFEDLWHILLTFDGQENLKKFALEKLNLDDEKAIKFSKIKLQQGYATLSISAIKKILPYLQRGFLYSHAVYMANLYKMLGETTISETLTNHFSDEILKILENDDFNRKNSAIINSLISEMLNDDNRYYIADNRNLDKTEEQIVLKSITDNYGLETWGNFDEEKKMEIIDFVSSKYLEFLKKTYTEKGNLFIQPERIHDKIFNFLKETYHVSEDKIKYLWHPSEQENYAKADEYFEYSIGSKKYYVKEESTQNFLSRNPNAEFDGRQLKLLGNPEPLSKGFKNPMALKSLHKLKNLVNYLLQTGKIDEGTRVVIEIARELNDKNKRKAIENWQKDREKENEAFRKEIEVYKEQFPNINLIDENTLIRKIRLWHEQNKICLYTGKTIPFSELIIGNKYDFEHTIPASISFDNELKNLTISDSSYNRLYKGKKFPTQLSNYDSEQTINGESCNSILRNIEFIFGERIVEYKEIKGKTEKIVKWKKIDELEKQFDEWKKKASYASTKEIKDNCIVKYHTIKMDLDYLKSKLATFTITEYKAGWRNSQIRDTQIITKYALPYFKTLFKRVSVEKGGVTDIFKKVYKVQSRNSKKDRSVHSHHAQDAAILTLIPNAFHRERIIKAYENEIDNRTGKTYHEQPLDWENFSEKYILELQDKVLINNLTDNRTITQTYKTVRKRGKVVYDIDENGNSLKRVSKGDTIRGQLHGETFYGAIKQPVRDEDNKILFDENKKMILKDEIYLAVRKPLVYKKDANSPGFKTLDEVEKVIVDKGLFKMIKKQVEESDFKTALIDGVYMLNESGEKVNKIRRIRCFENGLKYTTAIKVHEHSFVSHKEYKQSTLATNGENTYCLFYKNDKGKAMKILSIVDLAELKLKNIQNLYNEPEFSSFEVGKGKNKYKIPLYSVLKSGDKVLFYKENIYELKDLEQKELSDRMFKMYQFEKDGRIKFRHHLAAGIDTELKKENPENSSVNFEEKQVFLRLSQGQWNFAIDGKDFEISLDGKINWKF

SEQ ID NO: 370 - RNA, 15 nt, Chryseobacterium sp.
  source 1..15: mol_type = genomic RNA
gttgtgaattgcttt

SEQ ID NO: 371 - RNA, 68 nt, Chryseobacterium sp.
  source 1..68: mol_type = genomic RNA
aaagcaattcacaataaggcttaaaagccgaagaaaatctttagtcctgcgtactccgtgggattttt

SEQ ID NO: 372 - protein, 1092 aa, Bacillus sp.
MRYSIGLDIGTTSIGSAVINNDLQRFEHLGVRIFDAAENPKDGSSLSAPRRLARSSRKRLRRRKHRVERTKQILINKGLLTKTQVKNLYNSKNVNLDVWDIRVSGLDRKLFNDEFARVLIHFSKNRGFKSNRKSELKEDDNGAILSAVKENKELMEEKGYRTIAEMLVLDERYEGTKRNKDGDYSHVVSRSDIENEIRLLFQKQREYGHPFAIEENEEAFLAIWSSQRPFATKDDIVKKIGTCTLEPREKRAPKSTYTFERFRALDKLNRLRILSTTTPSRPLTNEERDSILNSLFSKKEVKYKELRKMLKLTDDQRFNEIYYSPDETIEKTENRVFLSSESQYKIKKIIEKTESKNMLSTYCPIDYDTIGYALTVFKDDKDIQHYLQNRYVDSKGKAISNMANREYNLELIEELLGLSFSKFGHLSLKALNNILPYMEEGEPYHIACEMAGYQFSQRLTKEKHRLLPPIPVDEIPNPVVVRALTQVRKVLNSIIKKYGPPSDIYIELAREMGKPFKERKSLEREFNENRQVNEKAKAHISELYRVPNDPRPHDILKFKLWKEQNGICPYSFKPISVDYLFNIGYTEVDHIIPYSRSFDDSNGNKVLVLTNENRNKLNRTPYEWFGHDENRWGDFVSFVRTMKVGKKKKDMLLKKNFDEEQEEQILSRNLNDTRYITRYIKSFIEDNLEFRTEENKKQYVHTVNGGYTSLMRKRWGLNKDRRGNDLHHAVDAAIIAVSLPFKHKVNTYFKRQEIGLSNLSNNKKDIFPEPWRNFIKELEARMIQDPEKMKTALESLELETYGEIFLNKLKPIFVSRMPKHSIKGQIHEETIRRVRGLTEEGFLVTVKKTRLDQIPFDKNGDFPMYGKETDIKTYMAIKQRYLEYGQDKQKAFAIPLRKPSKNPENAPIVRSVKIEGKANRVVMLDDKAAADNASIARTEVFRHKETGEYYVTPVYVADIVSNKIPDRLITLKKPYSEWDRITDEYEYLFSLYNNDLIKIVLPKEKETKKYTGGNHLWQEGFFYFKGVDSSNASIKIIDHLNSFEVRIGIRRLIAFEKYQVNPLGEINKVHGEKRPGELLNEEEIKENRKNIS

SEQ ID NO: 373 - RNA, 23 nt, Bacillus sp.
  source 1..23: mol_type = genomic RNA
gccataattcctctgtaaaactt

SEQ ID NO: 374 - RNA, 112 nt, Bacillus sp.
  source 1..112: mol_type = genomic RNA
aaggtttatagagttattatggtaaggcaatatgccgtggcgttggggatcgcctatgtccggttttaccggatctccctagaggtgactaactttggttagtcgccttttt

SEQ ID NO: 375 - protein, 1488 aa, Weissella sp.
MAKQKYTVGLDIGTNSVGWAVINKDFKLVSGKKHINDNGKQKRSRTNLWGVRLFDAADTAADRRLKRGMRRRIARRKERLNYLRGIFQADILAFDDSFFIRMDESFLQDDDKQATAFSYRDQSGAMQTRVVTEKEAVKYPLFNGKSGSGETYTDESAYYNQYPTIYHLRQRLIDNPAQADLRLVYLAMHHILKYRGHFVNQGQTFDLGNINIAGNLTSALDKLDEASSFKFGLADVDADQANAILKNKQWSASKKAYELNTLYEIIQDTVYRRENETQAVAYDSLTVKQQEKWLAEKQKQVKAFLTGIVGNTISPKDIFANKDYDRKQNEDFPEKIKYSNENFEEQIGELEKYLDETEVAAIVAGKDVYESLVLSNILTKSTLSGSMIEKYNVHGTQLKSLKSFSRKVSAEFYDKLFNVVKDEKGKPQIGVYAQYIDGVGNPAKRLAREDFYDALKKVFESEFKGLTFPAGEKGIDFTKTDLSADRMAFIREMNEAINLENYLPKQRQADNGAIPYQVHEHELIRIIENQKQYYPFLGDKVQVTYEDESGQTQVRSEYKIQVLFKFRIPYYVGTLAKNSGWVNEDGKKLVARNVAAKNSWVVRNSDERVTPWNFEQVINKEASSINFIERMTNFDTYLPNEKVLPKNSLLYQEFIVNNELISSGYYLNGRKEYFLPEQRQRIITRLFKRYRKVSAKQMLDFLKNEYQIKLDDDPKKLFGLDTFVKVPSYNGSLSTVVDLTNKMGISEDLIANYPERFEEIIKWQTIFEDKKILKKTIREVNHNKWSGMLTAEQVNKLSKRHYTGWGRLSQKLLDGVKTSQGRTIIESLREGICDNFMRLIEDEKIAEFIANAQVAGAKDGVLAYDLVDQLPGSPAIKKGIWQSLRVLQELEHYLGRDAIGKVVIEMSRDDESSRRTKARKQQLEEFYKKFKESTGSDVTAELKRELSEKDAKEIDDEKVFLYFLQNGKSMYSGDQLYLSRLADYQVDHIVPQTYIKDDSLDNKVLVTQRDNQNKGGDTPSHTVVTRMNSYWDMLAKNGQVSPRKLANLKKGTITLKQREGFINRQLVENRQIIKHVANILTNYYTGTDTLVLTPKSGLTSQLRSGLIYELNPAFDEAKALDRGKAYQVERYTTVKLHDRFVKNRKLNDYHHAHDAYLNAFVAQYVYQEHPEWKNAWVYGKYPRNGQADFGKWATQRKQKSLQLLSSMANDVWNLEDPDTMEKTILNRDETFEQMRQTLCYRNINIVKKLETQTGKFGDESVYKKGNKADHYSLGLKRKYPPQKYGGTKGAISAVTALVKDNSGRVIPVSISASDYDSYQNASDKLAWLLMRKKNIAEILVASLPKYTKYELPSGALRLLASYQEAQSAVELPMLNLGLMDDEAKVLNLYDQLAQFIADNKLFTDKKIALLKGEMRVAFEGLEEFKDKQKVIDELLGVTNGSNQGLQALNSIGLGTSNQRLKSGNTMTNGVTLINESVTGLYSTRTTYN

SEQ ID NO: 376 - RNA, 22 nt, Weissella sp.
  source 1..22: mol_type = genomic RNA
gttttagtgtcatgttgaatag

SEQ ID NO: 377 - RNA, 70 nt, Weissella sp.
  source 1..70: mol_type = genomic RNA
ctattcaacatggcaagttaaaataagctttatgcgtattcaaacttttgttaaggcgacatcgcctttt

SEQ ID NO: 378 - protein, 1337 aa, Enterococcus sp.
MKKDYVIGLDIGTNSVGWAVMTEDYQLVKKKMPIYGNTEKKKIKKNFWGVRLFEEGHTAEDRRLKRTARRRISRRRNRLRYLQAFFEEAMTALDENFFARLQESFLVPEDKKWHRHPIFAKLEDEVAYHETYPTIYHLRKKLADSSEQADLRLIYLALAHIVKYRGHFLIEGKLSTENISVKEQFQQFMIIYNQTFVNGESRLVSAPLPESVLIEEELTEKASRTKKSEKVLQQFPQEKANGLFGQFLKLMVGNKADFKKVFGLEEEAKITYASESYEEDLEGILAKVGDEYSDVFLAAKNVYDAVELSTILADSDKKSHAKLSSSMIVRFTEHQEDLKNFKRFIRENCPDEYDNLFKNEQKDGYAGYIAHAGKVSQLKFYQYVKKIIQDIAGAEYFLEKIAQENFLRKQRTFDNGVIPHQIHLAELQAIIHRQAAYYPFLKENQKKIEQLVTFRIPYYVGPLSKGDASTFAWLKRQSEEPIRPWNLQETVDLDQSATAFIERMTNFDTYLPSEKVLPKHSLLYEKFMVFNELTKISYTDDRGIKANFSGKEKEKIFDYLFKTRRKVKKKDIIQFYRNEYNTEIVTLSGLEEDQFNASFSTYQDLLKCGLTRAELDHPDNAEKLEDIIKILTIFEDRQRIRTQLSTFKGQFSEEVLKKLERKHYTGWGRLSKKLINGIYDKESGKTILDYLIKDDGVSKHYNRNFMQLINDSQLSFKNAIQKAQSSEHEETLSETVNELAGSPAIKKGIYQSLKIVDELVAIMGYAPKRIVVEMARENQTTSTGKRRSIQRLKIVEKAMAEIGSNLLKEQPTTNEQLRDTRLFLYYMQNGKDMYTGDELSLHRLSHYDIDHIIPQSFMKDDSLDNLVLVGSTENRGKSDDVPSKEVVKKMKAYWEKLYAAGLISQRKFQRLTKGEQGGLTLEDKAHFIQRQLVETRQITKNVAGILDQRYNAKSKEKKVQIITLKASLTSQFRSIFGLYKVREVNDYHHGQDAYLNCVVATTLLKVYPNLAPEFVYGEYPKFQAFKENKATAKAIIYTNLLRFFTEDEPRFTKDGEILWSNSYLKTIKKELNYHQMNIVKKVEVQKGGFSKESIKPKGPSNKLIPVKNGLDPQKYGGFDSPVVAYTVLFTHEKGKKPLIKQEILGITIMEKTRFEQNPILFLEEKGFLRPRVLMKLPKYTLYEFPEGRRRLLASAKEAQKGNQMVLPEHLLTLLYHAKQCLLPNQSESLAYVEQHQPEFQEILERVVDFAEVHTLAKSKVQQIVKLFEANQTADVKEIAASFIQLMQFNAMGAPSTFKFFQKDIERARYTSIKEIFDATIIYQSTTGLYETRRKVVD

SEQ ID NO: 379 - RNA, 17 nt, Enterococcus sp.
  source 1..17: mol_type = genomic RNA
gttttagagtcatgttg

SEQ ID NO: 380 - RNA, 68 nt, Enterococcus sp.
  source 1..68: mol_type = genomic RNA
caacatagcaagttaaaataaggttttaaccgtaatcaactgtaaagtggcgctgtttcggcgctttt

SEQ ID NO: 381 - protein, 1340 aa, Enterococcus sp.
MKKEYTIGLDIGTNSVGWSVLTDDYRLVSKKMKVAGNTEKSSTKKNFWGVRLFDEGQTAEARRSKRTARRRLARRRQRILELQKIFAPEILKIDEHFFARLNESFLVPDEKKQSRHPVFATIKQEKSYHQTYPTIYHLRQALADSSEKADIRLVYLAMAHLLKYRGHFLIEGELNTENSSVTETFRQFLSTYNQQFSEAGDKQTEKLDEAVDCSFVFTEKMSKTKKAETLLKYFPHEKSNGYLSQFIKLMVGNQGNFKNVFGLEEEAKLQFSKETYEEDLQELLEKIGDDYIDLFVQAKNVYDAVLLSEILSDSTKNTRAKLSAGMIRRYDAHKEDLVLLKRFVKENLPKKYRAFFGDNSVNGYAGYIEGHATQEDFYKFVKKELTGIRGSEVFLTKIEQENFLRKQRTFDNGVIPHQIHLTELRAIIANQKKHYPFLKEEQEKLESLLTFKIPYYVGPLAKKQENSPFAWLIRKSEEKIKPWNLPEIVDMEDSAVRFIERMINTDMYMPHNKVLPKNSLLYQKFSIYNELTKVRYQDERGQMNYFSSIEKKEIFHELFEKNRKVTKKDLQEFLYLKYDIKHAELSGIEKAFNASYTTYHDFLTMSENKREMKQWLEDPELASMFEEIIKTLTVFEDREMIKTRLSHHEATLGKHIIKKLTKKHYTGWGRLSKELIQGIRDKQSNKTILDYLINDDDFPHHRNRNFMQLINDDSLSFKKEIKKAQMITDTENLEEIVKELTGSPAIKKGILQSLKIVDEIVGIMGYEPANIVVEMARENQTTGRGLKSSRPRLKALEESLKDFGSQLLKEYPTDNSSLQKDRLYLYYLQNGRDMYTGAPLDIHRLSDYDIDHIIPRSFTTDNSIDNKVLVSSKENRLKKDDVPSEKVVKKMRSFWYDLYSSKLISKRKLDNLTKIKLTEEDKAGFIKRQLVETRQITKHVAGILHHRFNKAEDTNEPIRKVRIITLKSALVSQFRNRFGIYKVREINEYHHAHDAYLNGVIALALLKKYPQLAPEFVYGEYLKFNAHKANKATVKKEFYSNIMKFFESDTPVCDENGEIFWDKSKSIAQVKKVINHHHMNIVKKTEIQKGGFSKETVEPKKDSSKLLPRKNNWDPAKYGGLGSPNVAYTVAFTYEKGKARKRTNALEGITIMEREAFEQSPVLFLKNKGYEQAEIEMKLPKYALFELENGRKRMVASNKEAQKANSFLLPEHLVTLLYHAKQYDEISHKESFDYVNEHHKEFSEVFARVLEFAGKYTLAEKNIEKLEKIYKENQTDDLAKLASSFVNLMQFNAMGAPADFKFFDVTIPRKRYTSLTEIWQSTIIHQSITGLYETRIRMGK

SEQ ID NO: 382 - RNA, 15 nt, Enterococcus sp.
  source 1..15: mol_type = genomic RNA
gttttagagctatgc

SEQ ID NO: 383 - RNA, 70 nt, Enterococcus sp.
  source 1..70: mol_type = genomic RNA
gcatagcaagttaaaataaggctttgtccgtcatcagcttttttaaagcagcgctgttctcggcgctttt

SEQ ID NO: 384 - protein, 1327 aa, Enterococcus sp.
MKKNYTIGLDIGTASVGWAVLTEDYDLVKRKMKISGNTQKKAVKKNFWGVRLFEQGETAEGRRLKRTTRRRIARRRQRIQYLRTIFDEAMNQVDANFFARLDESFSITDEKENERHPIFGNVAEEVAYHEQFRTIYRLREVLANSSEQADLRLIYLALAHIIKFRGHFLIEGELNTENSSVSGTFEQFIKVYNETFNVEKVLELTVDLDEIAKQKVSRMKRAELILSLFPEEKSTGDFAQFIKMIVGNQGNVKKTFSLNEDAKIQFSKEEYEENLETLLAEIGEDFRGVFDAAKSVYDAISLANILKVTDAATRAKLSSSMVARFKEHKEDLKALKQFVRDNLPDEYDDLFKNKKVAGYAGYIDGDATQEEFYKYLKKALAKTTGAEYFLAKIEQEDFLRKQRTFDNGVIPYQLHLEELKAIIRNQKPYYPFLDEEKISQLMTFRIPYYVGPLAQGQGNFAWLTRKESGKITPWNLNEKVDIEKSATDFVERMTNNDSYLPMEKVLPKHSLLYEKFTVYNELTKVRYMDDNGRVQNFSSKEKQQIVNDLFKQQRKVKKEILEAFIKNEYGIENPKVEGIEKAFNASLGTYHDLVKLGIRPALFEQPEYEQQFEQIVKILTVFEDRKMRRKQLEQFSDLLTEDEQKQLERKHYKGWGRLSAKLIHGIVDQKTQKTILDYLIDDDDLPRNRNRNFMQLINDENLSFKEEIEKIAFDNDKSTEEIVQELAGSPAIKKGILQSLKIVEEIIEIMGELPTNIVVEMARENQTTAQGNRASQARMKYLEESIKKLGSSILEDEPISKDANLLRNDRLFLYYLQNGRDMYTGDELDINNLSSYDIDHIIPQSFVKDDSIDNRVLTTQRINRGKSNTVPAESVVKDMRATWERLYASNLISKKKLNNLMKAVNGGLTEEDKAYFIQRQLVETRQITKNVAQILHQKYNNEQSSEKPVRVVTLKSALASQFRKDFSLYKIRELNDYHHAQDAYLNGVIAQALLKVYPKLEPEFVYGEYQKVSIRALNKATAKKETYSNIMKFFTNDEVLTNKETGEVLWNRQRDIKTIKKVMNYHQMNIVKKVEIQTGRFTKESIQPKGPSKKLIARKNNWAPVNYGGFDSPTVAYSVIITHEKGKAAKVVQQLVGIKILERQAFEQNEVAFLEGKGFIHPKVQLKLPKYSLYQFADGRRRLLASADEAQKGNQMVLPTELIELLYHAKHVNDSSGESLTYLENHRTKFAELLEVILQFTNRYVGAEDRQKTVRDLYEKNQDADMKELAPSFVQLLQLNKQGAPASFKFFGETIERSRYKNMTELNKATFINQSITGLYETQRRLV

SEQ ID NO: 385 - RNA, 15 nt, Enterococcus sp.
  source 1..15: mol_type = genomic RNA
gttttagagctatgc

SEQ ID NO: 386 - RNA, 66 nt, Enterococcus sp.
  source 1..66: mol_type = genomic RNA
gcatagcaagttaaaataaggcttagtccgtaaacaaactttggcactgtattcggtgcttttttt

SEQ ID NO: 387 - protein, 1063 aa, synthetic construct
MRELDYRIGLDIGTNSIGWGVIELSWNKDRERYEKVRIVDQGVRMFDRAEMPKTGASLAEPRRIARSSRRRLNRKSQRKKNIRNLLVQHGVITQEELDSLYPLSKKSMDIWGIRLDGLDRLLNHFEWARLLIHLAQRRGFKSNRKSELKDTETGKVLSSIQLNEKRLSLYRTVGEMWMKDPDFSKYDRKRNSPNEYVFSVSRAELEKEIVTLFAAQRRFQSPYASKDLQETYLQIWTHQLPFASGNAILNKVGYCSLLKGKERRIPKATYTFQYFSALDQVNRTRLGPDFQPFTKEQREIILNNMFQRTDYYKKKTIPEVTYYDIRKWLELDETIQFKGLNYDPNEELKKIEKKPFINLKAFYEINKVVANYSERTNETFSTLDYDGIGYALTVYKTDKDIRSYLKSSHNLPKRCYDDQLIEELLSLSYTKFGHLSLKAINHVLSIMQKGNTYKEAVDQLGYDTSGLKKEKRSKFLPPISDEITNPIVKRALTQARKVVNAIIRRHGSPHSVHIELARELSKNHDERTKIVSAQDENYKKNKGAISILSEHGILNPTGYDIVRYKLWKEQGERCAYSLKEIPADTFFNEPILEVDHILPYSQSFIDSYHNKVLVYSDENRKKGNRIPYTYFLETNKDWEAFERYVRSNKFFSKKKREYLLKRAYLPRESELIKERHLNDTRYASTFLKNFIEQNLQFKEAEDNPRKRRVQTVNGVITAHFRKRWGLEKDRQETYLHHAMDAIIVACTDHHMVTRVTEYYQIKESNKSVKKPYFPMPWEGFRDELLSHLASQPIAKKISEELKAGYQSLDYIFVSRMPKRSITGAAHKQTIMRKGGIDKKGKTIIIERLHLKDIKFDENGDFKMVGKEQDMATYEAIKQRYLEHGKNSKKAFETPLYKPSKKGTGNLIKRVKVEGQAKSFVREVNGGVAQNGDLVRVDLFEKDDKYYMVPIYVPDTVCSELPKKVVASSKGYEQWLTLDNSFTFKFSLYPYDLVRLVKGDEDRFLYFGTLDIDSDRLNFKDVNKPSKKNEYRYSLKTIEDLEKYEVGVLGDLRLVRKETRRNFH

SEQ ID NO: 388 - protein, 1064 aa, synthetic construct
MSELDYRIGLDIGTNSIGWGVIELFWNKDRERYEKVRIVDKGVRMFDKAEIPNKGASLAEPRRIARSSRRRLNRKSQRKKEIRNLLVQHGMITQEELDLLYPLSKKSIDIWDIRLDGLDRLLNHLEWARLLIHLAQRRGFKSNRKSELKDAETGKVLSSIQVNEKRLFLYRTVGEMWIKDAEFSKYDRRRNSPNEYVFSVSRADLEKEIVTLFEAQRKFQSSYASKNLQETYLQIWAHQLPFASGNAILNKVGYCSLLKGKERRIPKATYTFQYFSALDQVNRTRLGPDFQPFTQEQKEIILDKMFQRTDYYKKKTIPEVSYYDIRKWLELDETIQFKGLNYDPNEELKKIEKKPFINLKAFYEIKKVVANYAERTNEAFSTLDYDAIAYALTVYKTDKDIRSYLKKSNNLSKRCYDDQLIEELFTLSYTKFGHLSFKAINHVLPIMQEGRTYQEAIHQLGYDTTNLKKENRSMFLPLIPDEITNPIVKRAITQARKVVNAIIRRYGSPNSVHIELARELSKSHDERKKIMTAHDENYKKNKGAISILIENGILNPTGYDIVRYKLWKEQGERCAYSLKEIPPDTFFNEPILEVDHILPYSQSFIDSYHNKVLVYSDENRNKGNRIPYTYFLETNKDWEAFERYVRSNKLFSKKKREYLLKKTYLPRESELIKERHLNDTRYASTFLKNFIEQNLQFKEVEVNLRKKRVQTVNGVITAHLRKRWGLEKNRQETYLHHAMDAIIVACTDHHMVTRITEYYQIKESNKSVKKPYFPMPWEGFRDELLSHLASQPIAKKISEELKAGYQSSDYIFVSRMPKRSVTGAAHDQTIRRKGGIDKKGKTIIIKRVRLKDIKFDENGDFKMVGKEQDLATYEAIKQRYLEHRKNSKKAFETPLYKPSKKGTGNLIKRVKIEGQTKAFVREVNGGVAQNSDLVRVDLFEKDDKYYMVPIYVPDTVCSELPKKVVKSGKGYEQWLTLDNSFTFKSSLYPYDLVRLVKGNEDRFLYFGTLDIDSDRLNFKDVNKPSKQNEYRYSLKTIENLEKYEVGVLGDLRLVKQETRRIFNR

SEQ ID NO: 389 - protein, 1055 aa, Brevundimonas vesicularis
MTTRLGLDVGTNSIGWALIDKDSARIIDLGSRIFSDGRDPKSGASLAVDRREARAARRRRDRYLGRRSAFLETLIQHGLMPERADDAKLVAAKDPYALRARALDEPLEPHEIGRALFHLNQRRGFKSNRKAERGRKENEDGKIESGAKALDTAMTVAKARTLGEFLHTQNAKRVRMGGDNQTYDFYPQRRHVETEFAVLWAAQKAHHPALLTDAAEEALHRILFFQRDLKDQEVGLCTFAGWNDVPENERRLPKTHPLFQERRLYEEVNHLKIVSPGAKDRDLTLAERDAVLLKLRDTRKSSFSALGKLIKLGEGERFNKESETRKDLLGDEVRAELADKKRFGARWTFFDIAEQLKIIDRIQNEADPEVLLDWLKAEYGLDDASAVAVSKARLPEGYGRFGETATRRLIDALKAQVVTYDKAALAAGFHHSDRRTGEVWERLPYYGQILTTEIAPGKAEHGDDNERRFGKITNPTVHIELRQLEKLVNAVITVHGRPDEIVVELARELKLNEEDRKKHDARIRRDTAAAIARGEKLEAEGIPDTGANRMAMRMWEDLNPGNPLDRRCPYCAEVIGIRALFSGEADIDHIIPYSQSLDDSAGNKVVAHRHCNRAKGNKTPYERWGHDEARWEIISAQVARMHRSKQWRFGPEARERLEKEGGFIARQLTDTQYLSRATAKYLSSLYTPDEGRRVYALPGRMTAMLRRLWGLNSLLPDHNFVENEHSNAPKNRLDHRHHTIDGAVAAVTDASLMQRIATVAARAEENELDRLFEDLAPPWPTFREELKERLARVTVSHKPDHGRKGRPDRTRDVTAGRLHNDTAYGFTGQVAADGKTPIVVHRVPFASLKPADILDPTRIPDTALREALFEATRDLTGKSYDQAMARFAKQHPVFKGIRRVRVREALNVIPIRSKEGRPYKAYKGDSNARYDVWRLPNGKWVTDIVSMFDAHSPEASDRRPHPAAKKVLSLRQNDMLAIERDGGEAQIVRVVKFSRSGLILAGQNEAGPLKARDAAPNETDPFRYISASASSLKRDNARQIRIDPLGRVFDPGPRD

SEQ ID NO: 390 - RNA, 25 nt, Brevundimonas vesicularis
  source 1..25: mol_type = genomic RNA
gttgccgctggccttcgatttctga

SEQ ID NO: 391 - RNA, 80 nt, Brevundimonas vesicularis
  source 1..80: mol_type = genomic RNA
tgtcagaaatcgaaggctggctgttaacaagcagctagactgcaccaaataagggcggggctacggccccgtcttttttt

SEQ ID NO: 392 - protein, 1079 aa, Bacillus endophyticus
MKDLDYRIGLDIGTNSIGWSIIKLQFNDTTQRYEKSGIIDLGVRMFDKAEIPKTGASLSEPRRLARSSRRRLKRKSERKQIIRRLLVDFDILSQEELDHLFPLPKDSVDIWDIRLEGLERVLNRFEWARLLLHLAQRRGFKSNRKSQRNETETGKMLASIYDNKNRLSAYRTVGEMWMKDKKFAQYDKRRNSPDEYIFNVSRDDLEKEIMTLFEAQRKFLSEYASEQLQEAYLKVWSHQLPFASGNDILKKVGHCSLEKNERRIPKATYTFQYFAALDKINNLRLGDYVQPLRQEQQKLLLKKMFERIDFVKKKSIPIIKYSDLRKWLKLDESICFKGLTYNPNEKLSKVEKKEFINLKPYYEMKKVISKHYEKHENIYEVADFDTFGYALTIYKNDQDIRSYLRNSNNIGKKVYDEALIEDLLNLSYTKFGHLSFKALQNLIPIMEEGKSFVDATKELSYDTTGLKKTKKSRLLPPIPDDITNPIVKRSLTQSRKVVNSIIKTYGPPLSIHIELARELSKDHEERRKISKTQDENYRRNKGAIEVLLENGIINPKGYDIVRYKLWKEQRECCAYSLKKIPADVFFSELRRERGSSPILEVDHILPYSQSFMDSYHNKVLVYSDENRKKGDLIPYDYIKGEEQRWKDFESYVQTNNLFSKKKREYLLKKEYTSRESDMVKERHLNDTRYATRFFKNFMEQRLLFKEAKVSMKKRVQTVNGIITSHFRSRWGLEKVREDTYLHHALDAVVVACTDHHMVTRVTEYYKHKESSLKSRKPFFPWPWKGFRDELLTRLTSQPVPDKISEALQLGTSLPDYRIVSRMPKRSITGAAHKETIMMKGGIDEKTGKTIIVKRVALKDIKFDKNGDFEMVGKEQDMATYTAIKERYLQHKKNAEKAFEKPIYKPSKKGRGNPIKRVKVVFESKAFVREVNGGVANNEKLVRVDLFEKDGKYSMVPIYVMDTVTEKLPDKIVTIGKGHHQWRRLDESYTFLFSLHPYDLVRVRSEEADTFFYFGSIDINSNRIHFKHVNHPTKPNELRYALGKIDLLEKYEIGLLGDIKQVKKETRNTFHQPKKQLTME

SEQ ID NO: 393 - RNA, 14 nt, Bacillus endophyticus
  source 1..14: mol_type = genomic RNA
gtcatagttccact

SEQ ID NO: 394 - RNA, 89 nt, Bacillus endophyticus
  source 1..89: mol_type = genomic RNA
acagcaatggctttgatgtttctatgataagggtttcgacccgtggcgttggggatcgcctgcccatttcgatgggcgtctccccattt

SEQ ID NO: 395 - protein, 1081 aa, Bacillus megaterium
MKNLDYRIGLDIGTNSVGWSVIELNFNQTTNRYEKVGIIDFNVRMFNKAEIPKTGASLAEPRRQFRSTRRQLKRKSIRKQKIRKLLIAQDVISQQELDNLYPLSKGSVDIWDIRVEGLDRYLNKIEWSRLLLHLAQRRGFKSNRKSDNQETEQGKVLPIIQENKQLLCSYRTVGEMWMKDEKFATSDKRKNTNDNYLFNVSREDLKQEIQLLFQQQRQFGSLYASEQTEKEYLDIWEHQLPFASGHDILKKVGYCSLEPKERRIPKATYTFQYFTVIDKLNNLRLGSHFEPLTKDQRDILLQKIMERTDFINKKTIPSIRYSDIRKWLALDDSIQFKGLIYEPNEKTQKIEKKEFVNLKPYYEIKKVILLHSEKTGETYTNMDIDTIGYALTVYKTDKDIRAYFRNSHNLSQKVFDDALIDELLNLSYTKFGHLSIKALRRLLPYLEEGLSYAEAVNKVGYDTTNLKGKKKERLLPKIPDDITNPIVKRSLTQARKVVNAIIRKYGSPLTVHIELARELSKNTNERKKLSKAQTKNYERNQDAIAILVENGILNPTGYDIVRYKLWKEQDERCMYSLRKIPAEQFFAELRRERGSTPLLDVDHILPYSQSFLDGYDNKVLVFSDENRKKGSRIPFYYLQENPKKWLDFEAYVQSNEKLSKKKRGYLVKKVYTARESDDVKERHLNDTRYATRFLKNFIEQFLVFKEAQIPMKKRVQTLNGRITSHFRSRWGFEKLREDTYLHHAVDAVVVACTDQHMVNKVTDYYKSKEQSLKSNAPYFPWPWERFRDEMLTRLHAQPVPDQIKQAITGYSPLSDYRLVSRMSKHSVTGAAHKETIMMNGGENNKTGKTIIVKRMLLKEIKFDNNGDFKMVGKEQDPATYQAIKQRYLSYDKDAEKAFEKPLYKPSKKGHSNLIKRVKVEIQEKTFVREVNGGVAENGDLVRIDLFKKEDTYYMIPIYVLDTSSPELPDHIVTSGKGYKLWKKLDESYTFQFSLTPYDLIRVKINEKDQFLYFSTIDISNNRIICKHVNKPSVPKECTYSLTKIEIIEKCKVGILSDVSFVKNEKRRSFGHPAKKVALQTR

SEQ ID NO: 396 - RNA, 25 nt, Bacillus megaterium
  source 1..25: mol_type = genomic RNA
gtcatagtcccatcatatccattgc

SEQ ID NO: 397 - RNA, 102 nt, Bacillus megaterium
  source 1..102: mol_type = genomic RNA
acagcaatggatatgatgtttctatgataagggctctctaagagagacctgtggcgttggggatcgcctgcccgtttcgacgggtgtctccccatttcattt

SEQ ID NO: 398 - protein, 1081 aa, Bacillus megaterium
MRNLDYRIGLDIGTNSVGWSVIELNFNQTTNRYEKVGIIDFNVRMFNKAEIPKTGASLAEPRRQFRSTRRQLKRKSIRKQKIRQLLVTHDVISQQELDNLYPLSKGSIDIWDIRVEGLDRYLNKIEWSRLLLHLAQRRGFKSNRKSDNQETEQGKILPIIQENKQLLCSYRTVGEMWMKDEKFATSDKRKNTNDNYLFNVSREDLKREIQLLFQQQRQFGSLYASEQTEKEYLDIWEHQLPFASGHDVLKKVGYCSLEPKERRVPKATYTFQYFTVIDKLNNLRLGSHFEPLTKEQRDILLQKIMERTDFINKKTTPSIRYSDIRKWLTLDDSIQFKGLIYEPDEKTQKIEKKEFVNLKSYYEIKKVILLHSEKTGETYTNMDLDTIGYALTVYKTDKDIRAYLRNPHNLSQKVFDDVLIDELLNLSYTKFGHLSIKALHRLLPYLEEGLSYAEAVNKVGYDTTNLKGKKKERLLPKIPDDITNPVVKRSLTQARKVVNAIIRKYGSPLSVHIELARELSKNASERKKLSKAQTKNYERNQGAIAILVENGILNPTGYDIVRYKLWKEQDERCMYSLRKIPSEQFFAELRRERGSTPLLDVDHILPYSQSFLDGYDNKVLVFSDENRKKGDRIPFDYLQENPKKWLDFEAYVQSNEKLSKKKRGYLVKKVYTARESDDVKERHLNDTRYATRFLKNFIEQFLFFKEAQIPMKKRVQTLNGRITSHFRSRWGFKKIREDTYLHHAVDAVVVACTDQHMVNKVTDYYKSKEQSLKSNAPYFPWPWEGFRDEVLTRLHAQPVPDQIKQAITSYSPLPNYRLVSRMSKHSVTGAAHKETIMMNGGENNKTGKTIIVKRMLLREIKFDDNGDFKMVGKEQDPATYQAIKQRYLSYDKDAKKAFDKPLYKPSKKGHSNPIKRVKVEVQEKTFVREVNGGVAENGDLVRIDLFKKEDTYYMIPIYVLDTSSPELPDHIVTSGKGYKLWKKLDESYTFQFSLTPYDLIRVKINEKDQFLYFSTIDISNNRIICKHVNKPSVPKECTYSLTKIEVIEKCKVGILSDVSFVKNETRRSFGQPTKKVALQTR

SEQ ID NO: 399 - RNA, 24 nt, Bacillus megaterium
  source 1..24: mol_type = genomic RNA
gtcatagtcccatcatatccattg

SEQ ID NO: 400 - RNA, 102 nt, Bacillus megaterium; identical to SEQ ID NO: 397
  source 1..102: mol_type = genomic RNA

SEQ ID NO: 401 - protein, 1103 aa, Bosea thiooxidans
MDWRFGGDLGTNSIGWAAAELVLVDGVWTPKRVVAAGSRIFSDGREPKSGASLAGARRDARAMRRRRDRFKQRQAALLKYLTLAGFFPSDAEERRKLAEVDPYELRARALDEALPHPHLGRALFHLNQRRGFKSNRKADRGRNDDDSGKIRIGVRELHAQMDAAGARTLGEFLHGRRAGADQNAIPLVRTRIVAHLAEDGKASEAYNFYPDRRMLEDEFEAVWEAQIPHHPDALTPERKAQFHEIIFHQRPLKEPKIGYCTLRPPEPRLPKAHPLFQRRRLLEEVNALKIVLPGAEARALEKPERDSLLSKLAAKKTVAYASLRKALKLDPDARFNKESDNRKDMKGDEVAAEMSHAKRFGKAWLGMSPETQWQVVDRLRQLESDEQVADFEAWLRERHGLSHEQAQAVTGARLPEGYGRFGETATRALIEALKEKVIVYSEAVTEAGLGHHSDFRDGQVWQDEKGNPALPYYGEILERHILPGTADPADDDVVSRIGRLTNPTVHIGLNQLRRVLNTLIRRYGPPLEIAIELARELKLDEERKREINTRNRENREAAEKRSKKLNELGQADKGGNRALLKLWEELNRDNVLDRRCVYSGRKISMEMLFSGAVEVDHILPFSATLDDSGGNRILCLREVNRIKRKRTPWEAQADLRAHFGPDAEWEAIAARAARLPKEKRWRFEPDAIHRFDAQGGFLARHLTDTQHLSRLAREYLSALYPERGEGSSHVWVSPGRLTEMLRRAWSLNHHLPDHNLAGHASQPKNRKDHRHHAIDAIVVAVTDRSLLNRIAREAGRHGHEEASRVVADIPEPWDGFSDEVGRVLRSIVVSHRPDHGTASKAGLARQKDATAARLHNETAYGLTGEKDARGLDIVVTRKPLSSFRKPADLDAIRDEDLKRKLKDWTAGKEGGAFEQAMRAFGHLERGPRSYPGLRRIRVVEPLSVIPIKDREGRAYKAYKGDSNHRFDMWELTNGKWKDEVVSMFEAHQRDWVSPIRAANPTARKVLSLQQNDVIAIEQEDGRKLMRVVKFSAGTLVLALPQEAGSLKTRDADKDDPFRYVYGSPSSLLRWKARQVRIDEIGRVLDPGFPARKPRAGKTRGEEA

SEQ ID NO: 402 - RNA, 25 nt, Bosea thiooxidans
  source 1..25: mol_type = genomic RNA
gttgccgctggaccgcgatctctga

SEQ ID NO: 403 - RNA, 77 nt, Bosea thiooxidans
  source 1..77: mol_type = genomic RNA
tcagagatcgcggtctggctgttaacaagcagtcaagtctgcaccagataagggcgacgctccggcgtcgccttttt

SEQ ID NO: 404 - protein, 1130 aa, unidentified
MSKVAKNMTRVNLGFDIGIASVGWSVLDNQTGKILETGVSIFPSGSASRNEERRSFRQSRRLIRRKKARICDMDHLLKKNGFPFPGNTGANPYEIRVKGLTEKLSREELAIALHHLVKRRGISYDLKDVEDESAGGTNYQESIAVNQRLLKKETPAEIQLARLTECGKVRGQVKSLGEDNTATTLLNVFPNAAYQEEMVKLLKKQQEFYGEIDDPFMETAIGILSRKREYFIGPGSEKSRTDYGIYRTDGTTLKNLFEILVGKDKIFPDQYRAAGNSYTAQLYNLLNDLNNLEVDATEDGKLTTAHKEQIIEELTTTTGNVNMLKLIAKVAGTSPAGIKKYRVDREGKPEFHSLAIYRRLRKKLGEAGFEINEWPPEFFDDYGPIVTLNTESGELRKWLAEEGSRKYDFLTEPVIEAILANKSAFDSVGKNKWHRFSLKTMQLLIPELLHTFKEQMTILAEMGLLHENKKDYGDQNKVDVKYLTENLYNPVVRKSVKQAMDIFNALFEKYANIDYVVIEMPRDDAEDELEQKKQFQKFQLKNEKEKDASLKEFQELAGVSDLQLEAQLRKRKKLRQKIRMWYQQRGKCPYSGKTIAAVDLFHQDNQFEIDHIIPLSVSFDDGQNNKVLCYSEMNQEKGKQTPYAFMSRGGGQGFSALQAYVKSNNRLENAKKRNLLFTEDINDLEVRKRFIARNLVDTRYASRIVLNELQQFVRSKELDTRVTVIRGKLTSKLRDRWRLNKSRETHHHHAVDAAVIAVSPMLKMWEKNAEIIPLKVDENTVDLKSGEIITDQEYAAQMYELPYARFLEQMPELHKKIKFHHQVDKKMNRKVSDATLYSTRKAKVGTDKKEQEYVIGKIKDIYQFDQYKKFKKLYDGDKSKFLMQRLDPQTFAKLEKIMEDYPAKIDATQPNGTIKLVDISPFELYRREHGPVTKYAKKNNGPAIKSLKFYDSIVGSSVKITPKNAKGKEVILKSLKPWRTDVYYNHEKEQYEIMGIKYADLKFKGDNYGITKARYQEIKEEEGVSEESEFLFSLYRGDRIQVSNGEDKIDLLFLSRSNPAKKGYVELKPIDRNQLNGKEVVSVYGAASGGRLKKQFVKKNHTLHKVNTDILGNPFYIKKESDQPKNILDL

SEQ ID NO: 405 - RNA, 24 nt, unidentified
  source 1..24: mol_type = unassigned RNA
gtttttgtactctcaaggaatcag

SEQ ID NO: 406 - RNA, 85 nt, unidentified
  source 1..85: mol_type = unassigned RNA
taccattactgattccttgagaatctacaaaaataaggctttatgccgaaattcccactcttaacggagtgggttttttattttt

SEQ ID NO: 407 - RNA, 12 nt, Streptococcus pyogenes
  source 1..12: mol_type = genomic RNA
gttttagagcta

SEQ ID NO: 408 - RNA, 65 nt, Streptococcus pyogenes
  source 1..65: mol_type = genomic RNA
tagcaagttaaaataaggctagtccgttatcaacttgaaaaagtggcaccgagtcggtgcttttt

SEQ ID NO: 409 - RNA, 81 nt, synthetic construct
  source 1..81: mol_type = other RNA
gttttagagctagaaatagcaagttaaaataaggctagtccgttatcaacttgaaaaagtggcaccgagtcggtgcttttt

SEQ ID NO: 410 - protein, 9 aa, synthetic construct
LAGLIDADG

SEQ ID NO: 411 - protein, 7 aa, synthetic construct
PKKKRKV

SEQ ID NO: 412 - protein, 16 aa, synthetic construct
KRPAATKKAGQAKKKK

SEQ ID NO: 413 - DNA, 318 bp, synthetic construct
  source 1..318: mol_type = other DNA
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 414 - DNA, 300 bp, synthetic construct
  source 1..300: mol_type = other DNA
agctccacggcaagagaattcaaagccgcgggcctgggttccacgcggggccccttacccaaggtgccccgggcgctcatttgcatgtcccacccaacaggtaaacctgacaggtcagtcgcggccgggtacggcctggcggtcagagcaccaaacgtacgagccttgtgatgcggttccattgcatgaaattctcctaaaggccccaagatgaacgggaaagcgcgcggttcgctcaccgtaactaaaacaggtgagagactcccgtgccttataaggcctgtggacggaggcagttgc

SEQ ID NO: 415 - DNA, 300 bp, synthetic construct
  source 1..300: mol_type = other DNA
tgttctccgaacagtacttgtaatatacaggttccccgatccaccaccgtctgcgggtggcggtacagcctctcccagtgtgctttgcgctcatttgcatagagcacggcaccgaacgcagccactgtcccccgcgtccgctgtcccccgtgcaggccaaacccgggcgcggccccgccgacggtgggtacagagagtggtcggctcggcaccggctgctgcgcggcaccggcactcaccctgtccttatggagccctaactccatggctataaatatctaaggggaggaagggtagatc

SEQ ID NO: 416 - DNA, 300 bp, synthetic construct
  source 1..300: mol_type = other DNA
agcgctccgcggagacttctgggacgcggcggctccgactccgccccgcttccggcttatttgcatacagcaattcctagcaggccctgtgctgaatttagtcggaaaactaccatgttcagtcgaaaaagcaaatacttttgtcagatatggccaaaaacttcacttgacttagccgtgtttcatgtaaagcattaaaaggatggagtgattgttcaaatttcataagaagaattcaccttcagtttaaggtggttcgctttctgcacttcaaataccgcggtggacagaccctgtttc

SEQ ID NO: 417 - DNA, 300 bp, synthetic construct
  source 1..300: mol_type = other DNA
gcctgagtcgcgccgccgcctcccaaagacttctgggaaggcggtgcggctcaggctccgccccgcttccggggatatttgcatacgagcattcccagtaattcccagcagccactgtagctatatttggtagaataacgagcactttctgaactccagttaataactgcgttagttgcgttacacattggactaaaacaaatagaagttgaatctctagagcagtggagataagtcgccgtatgtgtacagaaattgcttccgggggctataaatagctggtagtggggctagaacgtc

SEQ ID NO: 418 - DNA, 300 bp, synthetic construct
  source 1..300: mol_type = other DNA
cgcccgcgcagcgcagccgcttcccagagacttctgggacggcagcggctgcggctccgcccctcttccagtataatttgcatgcgaccatggattcccagcagccacctgagtcatatttggtggaacaaaaaccactttctcaatttcagtgaatgaccccatttggttaaggtattgttgcacaaatatcataaaagaagggacgaatggctgaaccggtttttaatggagttcgcccttagcgtaaaagagtttattctatgccctctaaatagttctgggatcaaccgtactact

SEQ ID NO: 419 - DNA, 111 bp, synthetic construct
  source 1..111: mol_type = other DNA
ccccttacccaaggtgccccgggcgctcatttgcatgtcccaccgctcaccgtaactaaaacaggtgagagactcccgtgccttataaggcctgtggacggaggcagttgc

SEQ ID NO: 420 - DNA, 111 bp, synthetic construct
  source 1..111: mol_type = other DNA
acagcctctcccagtgtgctttgcgctcatttgcatagagcacgactcaccctgtccttatggagccctaactccatggctataaatatctaaggggaggaagggtagatc

SEQ ID NO: 421 - DNA, 111 bp, synthetic construct
  source 1..111: mol_type = other DNA
ggctccgactccgccccgcttccggcttatttgcatacagcaatattcaccttcagtttaaggtggttcgctttctgcacttcaaataccgcggtggacagaccctgtttc

SEQ ID NO: 422 - DNA, 111 bp, synthetic construct
  source 1..111: mol_type = other DNA
gctcaggctccgccccgcttccggggatatttgcatacgagcatagtcgccgtatgtgtacagaaattgcttccgggggctataaatagctggtagtggggctagaacgtc

SEQ ID NO: 423 - DNA, 111 bp, synthetic construct
  source 1..111: mol_type = other DNA
gctgcggctccgcccctcttccagtataatttgcatgcgaccatgttcgcccttagcgtaaaagagtttattctatgccctctaaatagttctgggatcaaccgtactact

SEQ ID NO: 424 - protein, 1493 aa, synthetic construct
MPNEGIPHSSQTQEQDCLQSQPVSNNEEMAIKQESGGDGEVEEYLSFRSVGDGLSTSAVGCASAAPRRGPALLHIDRHQIQAVEPSAQALELQGLGVDVYDQDVLEQGVLQQVDNAIHEASRASQLVDVEKEYRSVLDDLTSCTTSLRQINKIIEQLSPQAATSRDINRKLDSVKRQKYNKEQQLKKITAKQKHLQAILGGAEVKIELDHASLEEDAEPGPSSLGSMLMPVQETAWEELIRTGQMTPFGTQIPQKQEKKPRKIMLNEASGFEKYLADQAKLSFERKKQGCNKRAARKAPAPVTPPAPVQNKNKPNKKARVLSKKEERLKKHIKKLQKRALQFQGKVGLPKARRPWESDMRPEAEGDSEGEESEYFPTEEEEEEEDDEVEGAEADLSGDGTDYELKPLPKGGKRQKKVPVQEIDDDFFPSSGEEAEAASVGEGGGGGRKVGRYRDDGDEDYYKQRLRRWNKLRLQDKEKRLKLEDDSEESDAEFDEGFKVPGFLFKKLFKYQQTGVRWLWELHCQQAGGILGDEMGLGKTIQIIAFLAGLSYSKIRTRGSNYRFEGLGPTVIVCPTTVMHQWVKEFHTWWPPFRVAILHETGSYTHKKEKLIRDVAHCHGILITSYSYIRLMQDDISRYDWHYVILDEGHKIRNPNAAVTLACKQFRTPHRIILSGSPMQNNLRELWSLFDFIFPGKLGTLPVFMEQFSVPITMGGYSNASPVQVKTAYKCACVLRDTINPYLLRRMKSDVKMSLSLPDKNEQVLFCRLTDEQHKVYQNFVDSKEVYRILNGEMQIFSGLIALRKICNHPDLFSGGPKNLKGLPDDELEEDQFGYWKRSGKMIVVESLLKIWHKQGQRVLLFSQSRQMLDILEVFLRAQKYTYLKMDGTTTIASRQPLITRYNEDTSIFVFLLTTRVGGLGVNLTGANRVVIYDPDWNPSTDTQARERAWRIGQKKQVTVYRLLTAGTIEEKIYHRQIFKQFLTNRVLKDPKQRRFFKSNDLYELFTLTSPDASQSTETSAIFAGTGSDVQTPKCHLKRRIQPAFGADHDVPKRKKFPASNISVNDATSSEEKSEAKGAEVNAVTSNRSDPLKDDPHMSSNVTSNDRLGEETNAVSGPEELSVISGNGECSNSSGTGKTSMPSGDESIDEKLGLSYKRERPSQAQTEAFWENKQMENNFYKHKSKTKHHSVAEEETLEKHLRPKQKPKNSKHCRDAKFEGTRIPHLVKKRRYQKQDSENKSEAKEQSNDDYVLEKLFKKSVGVHSVMKHDAIMDGASPDYVLVEAEANRVAQDALKALRLSRQRCLGAVSGVPTWTGHRGISGAPAGKKSRFGKKRNSNFSVQHPSSTSPTEKCQDGIMKKEGKDNVPEHFSGRAEDADSSSGPLASSSLLAKMRARNHLILPERLESESGHLQEASALLPTTEHDDLLVEMRNFIAFQAHTDGQASTREILQEFESKLSASQSCVFRELLRNLCTFHRTSGGEGIWKLKPEYC

SEQ ID NO: 425 - protein, 22 aa, synthetic construct
DYKDHDGDYKDHDIDYKDDDDK

SEQ ID NO: 426 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 427 - DNA, 4627 bp, Homo sapiens
  source 1..4627: mol_type = genomic DNA
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttc

SEQ ID NO: 428 - DNA, 3216 bp, synthetic construct
  source 1..3216: mol_type = other DNA
atgagcgagctggattatagaatcggcctggatatcggcaccaattctatcggatggggagtgatcgagctgttctggaacaaggatcgggaacggtacgagaaggtccgcatcgtggacaaaggggtcagaatgtttgacaaggccgagatccccaacaagggcgccagcctggccgagcctcggagaatcgcccggagcagcagaagacgcctgaacaggaagtcccaaaggaagaaggagatccggaacctgctggtgcaacacggcatgatcacccaagaggaactggacctcctgtaccctctgagcaaaaagtccatcgatatctgggacatcagactagacggcctggacagactgctgaaccacttagagtgggctagactgctgattcacctggcccagcggcggggcttcaagagcaaccgcaaaagcgagctgaaggacgctgaaacaggcaaggtgttgtctagcatccaggtgaacgagaagcggctgttcctgtacagaaccgtgggggaaatgtggatcaaggacgcagagttcagcaagtacgacaggcgtagaaactccccaaacgaatacgtgttcagcgtgtcaagagccgacctggaaaaggaaatcgtgacactgtttgaggctcagagaaagttccagagcagctatgcctctaaaaatctgcaggagacctacctgcagatctgggcccaccaactgcctttcgccagcggcaatgccatcctgaacaaagtgggctactgcagcctgctgaaagggaaggaaaggcggattcccaaggccacatacaccttccagtacttctccgccctggatcaggttaaccggacccggctcggccctgacttccaacctttcacccaggaacaaaaggagatcatcctggataagatgttccagagaacagactactacaagaagaagaccatccccgaggtttcttattatgatatcagaaagtggctggaactcgacgagacaatccagtttaagggactgaattacgaccccaacgaagagctgaagaagatcgaaaaaaaacctttcatcaacctgaaggccttctacgagatcaagaaggtggttgctaactacgccgaaagaacaaatgaagccttttctaccctggactacgacgctatcgcctatgccctgacagtctacaagaccgacaaggacatccggtcctacctgaagaaatccaacaacctgtccaagcggtgctacgatgatcaactgatcgaggaactgtttaccctgagctacaccaaattcggccacctgtctttcaaggccattaaccacgtgctgcctatcatgcaggagggcagaacctaccaggaggccatacaccaactgggctacgacaccaccaacctgaagaaagagaatagaagcatgttcctgcctctgatccctgacgagatcacaaaccccattgtgaagcgggccatcacccaggccagaaaggtggtgaacgccatcatcagaagatacggctctcctaacagtgtgcacatcgaactggccagagagctgagcaagagccacgatgagcggaaaaagatcatgaccgcccacgacgagaactacaagaaaaataagggcgctatttctatcctgatcgagaacggcattctgaaccctaccggctacgacatcgtgagatacaagctgtggaaggaacaaggcgagagatgcgcctacagcctgaaagaaattccacctgatacttttttcaacgagctgaagaaagaaagaaacggcagccccattctggaagtggaccacatcctgccttacagccagtccttcatcgacagctaccacaacaaagtgctggtgtactctgacgagaacagaaacaagggcaacagaatcccttacacctacttcctggaaacaaacaaggactgggaggcctttgaaagatacgtgcggagtaacaaactgttttctaagaaaaagagagaatatctgctgaagaagacttacctgcctagagagtctgaactaatcaaggaacggcacctgaacgacactcggtatgcttctacattcctgaagaatttcatcgagcagaatctgcagttcaaagaggtcgaggtcaatctgagaaaaaagagagtgcaaaccgtgaatggcgtgatcaccgcccacctcagaaagcgttggggcctggagaagaaccggcaggagacgtacctgcaccacgctatggacgccatcatcgtggcttgtacagaccaccacatggtgacccggataacagagtactaccagatcaaggaatccaacaagagcgtgaagaaaccttacttccccatgccttgggagggctttagagatgagctgttgtctcacctcgctagccagcctatcgcaaagaagatcagcgaggaactgaaggccggctaccagagcagcgactacatcttcgtgtccagaatgcccaagagaagcgtgaccggcgccgcccatgatcagaccatcagaagaaagggcggcatcgacaagaagggcaagacaatcatcattaagcgggtgcggctgaaagatatcaagttcgacgaaaacggcgatttcaagatggtgggcaaagagcaggacctggcaacctacgaggccatcaagcagagatacctggagcacagaaagaacagtaagaaggccttcgagacccctctgtacaaacctagcaagaagggaacaggaaacctgattaagcgggtgaaaatcgagggacagactaaggccttcgtgcgggaagtgaacggaggcgtggcccaaaatagcgacctggtcagagtggacctgttcgagaaggacgacaagtactatatggtgcctatctacgtgccagacaccgtgtgtagcgagctgcctaagaaggtggttaagagcggaaaaggctatgagcagtggctgaccctggacaacagcttcaccttcaagtctagcctgtacccctacgatctggtgcggctcgtgaagggcaacgaggacagattcctgtacttcggcacactggacattgactccgatagactgaatttcaaggatgtgaacaagcccagcaagcagaacgagtaccggtacagcctgaaaacaatcgagaacctggaaaaatacgaggttggagtgctgggagatctgcggctggtgaaacaggagaccaggaggatctttaacaga

SEQ ID NO: 429 - DNA, 3210 bp, synthetic construct
  source 1..3210: mol_type = other DNA
atgagagagctggattaccggatcggcctggatatcgggaccaactccattggatggggcatcatcgagctgtcttggaacaaagacagagaacagtacgagaaggcaagaatcgtggacaagggcgtgagaatgttcgacaaggccgaaatacccaagaccggagccagcctggccgagcctagaagaattgccagatcaagcagaagacggctcaacagaaagtctcagagaaaaaaagatattcggaacctgctggtccagcacgagatcatcagccaaaaggaactcgctagcctgtatcctctgaccaagagcagcatggacatttgggacatcagactggacggcctggacagactgctggatagattcgagtggaccagattgctgatccacctggctcagcggagaggcttcaaaagcaaccggaagagcgagctgaaggacgtggaaaccggcaaggtgctctccagcatccaggctaatgagaagcggctgtccctgtacagaactgtgggcgagatgtggatgaagaatgaagattttagcaagtacgacaaaagaagaaatagtagcaacgaatacgtgttctctgtgtcccgggccgacctggaaaaggaaatcgtgacactgtttgaagctcagcggaagttccagagcagctatgccagcgccgatcttcaaaaaacctacctccagatctgggcccatcaactgcctttcgcctctggcaacgccatcgttaacaaggtgggctactgcagcctactgaaaggcaaggagaagagagttcctaaggccacctacaccttccaatacttcagcaccctggatcagatcaacagaaccagactgggcccaaacttccagcccttcaccaaggaacagagagatgtgatcctggacgagatgtttaaccggaccgattattacaagaagaagaccatccctgaggtgacgtactacgatatcagaaaatggctggccctggacgagacaatccagttcaagggcctgacctacgaccctaatgaagaactgaagaaaattgagctaaagtcttttatcaatctgaagcctttctacgagataaagaaggtggtgacaaactacgccaagaagacaaatgaggcctttagcacactggactatgacacctttgcctacgccctgacagtgtacaagaccgacaaggacatccgctcctacctgaaaaagagtaacaacctgtccaaatgctgctacgacgaccaactgatcgaggaattgctgacactgagctacaccaaattcggccacctgagcttcaaggctatcaaccacgttctgcctatcatgcaggagggcagaacctaccaggaggccattcaccagctcggctacgatgccacaaacctcaaaaaagagaaccggtctatgttcctgcctctgttccctgacgagatcaccaaccccatcgtgaagagggccctgacccaggccaggaaggtggtcaacgccatcatcagacgatacgggtctccaaacagcgtgcacatcgagctggccagagagctgagcaagagccacgacgagagaacaaagatcatgaaagctcatgatgaaaactacaaaaagaacaagggcgctatcagcatcctgatcgagaacggtattctgaatcctacaggttatgacatcgtccggtacaagctgtggaaggaacagggcgagagatgcgcctattctctgaaacagatccccgccaacaccttcttcaacgaaatgaagaaggagcggtccggcagccctgtgctggaaatcgatcacatcctgccctacagccagagcttcatcgacagctaccacaacaaagtgctggtgtacggggatgagaaccagaaaaagggcaatagaatcccgtacacctacttcctggaaggcaacaaggactgggagtctttcgagagctatgtgcgcctgaactcctttttcagcaagaaaaagcgaggatatctgctgaagaaggcttacctgccaagagagagtaacatgatcaaggaacggcacctcaacgacacccggtacgccagctcctacctgaagaacttcatcgagaagaatctgaagttcaaggaggtggaaggctctacccggaagaagcacgtgcaaaccgtgaacggcataatcacagcccacctgagaaagagatggggcctggaaaaggaccgccaggagacatatcttcatcacgctatggacgccatcatcgtggcatgcaccgaccaccacatggtgacaaaggtgaccgagtactaccagatcaaagaaagcaataaatctattagaaagccttacttccccctgccttgggtgggctttagagaggaaattctgtcccacctggctcggcagcctatcgccagaaagatctctgaagagctgaagatcggataccagagcttcgattacatcctcgtgtctagaatgcctaaaagatcagtgaccggcgccgcccacgagcagaccattatgaaaaagggaggcatcgacaaaaaaggaaaaaccatcatcattaagcgggtctacctgaaggatatcaagttcgacgagaatggcgatttcaagatggttggaaaggaacaggacctggctacctacgaggccatcaagcagagatacatcgagtacggcaaggaatccaagaaggccttcgagacccctctgtataagcccagcaagaaaggcaaaggcaacctgatcaagaagatcaaagtggaagtgcaaaccaagagctttgtgagagaagtcaacggcggagtggcccagaacggcgatctggtgcgggttgacctgttcgagaaggataatagatactacatgatccccatctacgtgatggataccgtgcacagcgaacttcctaacaaggccgtgaccagcagcaaaggctatgagcaatggctgaccatcgacaacagcttcaccttcaagttcagcctatacccctacgacctggtgcggctggtcaagggtaacgaggacagattcctgtacttttccaccctggatattaacagtgatagactcaacttcaaagacgtcaacaagcccagcaagcaggccgagaacagatatagcctgaagacaatcgaaaacctggaaaaatacgaggtgggcgtcctgggcgacctcagatttgtgagacaggagatcagaaagaacttc

SEQ ID NO: 430 - RNA, 21 nt, synthetic construct
  source 1..21: mol_type = other RNA
  modified_base 18: mod_base = OTHER; note = Nucleotide with phosphorothioate linkage
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
gtcatagttccattaaagcca

SEQ ID NO: 431 - RNA, 85 nt, synthetic construct; identical to SEQ ID NO: 107
  source 1..85: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 82: mod_base = OTHER; note = Nucleotide with phosphorothioate linkage
  modified_base 83: mod_base = OTHER; note = 2'-O-methyl modified nucleotide with phosphorothioate linkage
  modified_base 84: mod_base = OTHER; note = 2'-O-methyl modified nucleotide with phosphorothioate linkage
  modified_base 85: mod_base = OTHER; note = 2'-O-methyl modified nucleotide

SEQ ID NO: 432 - RNA, 16 nt, synthetic construct
  source 1..16: mol_type = other RNA
  modified_base 13: mod_base = OTHER; note = Nucleotide with phosphorothioate linkage
  modified_base 14: mod_base = OTHER; note = 2'-O-methyl modified nucleotide with phosphorothioate linkage
  modified_base 15: mod_base = OTHER; note = 2'-O-methyl modified nucleotide with phosphorothioate linkage
  modified_base 16: mod_base = OTHER; note = 2'-O-methyl modified nucleotide
gtcatagttccattaa

SEQ ID NO: 433 - RNA, 15 nt, synthetic construct
  source 1..15: mol_type = other RNA
  modified_base 12: mod_base = OTHER; note = Nucleotide with phosphorothioate linkage
  modified_base 13: mod_base = OTHER; note = 2'-O-methyl modified nucleotide with phosphorothioate linkage
  modified_base 14: mod_base = OTHER; note = 2'-O-methyl modified nucleotide with phosphorothioate linkage
  modified_base 15: mod_base = OTHER; note = 2'-O-methyl modified nucleotide
gtcatagttccatta

SEQ ID NO: 434 - RNA, 14 nt, synthetic construct
  source 1..14: mol_type = other RNA
  modified_base 11: mod_base = OTHER; note = Nucleotide with phosphorothioate linkage
  modified_base 12: mod_base = OTHER; note = 2'-O-methyl modified nucleotide with phosphorothioate linkage
  modified_base 13: mod_base = OTHER; note = 2'-O-methyl modified nucleotide with phosphorothioate linkage
  modified_base 14: mod_base = OTHER; note = 2'-O-methyl modified nucleotide
gtcatagttccatt

SEQ ID NO: 435 - RNA, 13 nt, synthetic construct
  source 1..13: mol_type = other RNA
  modified_base 10: mod_base = OTHER; note = Nucleotide with phosphorothioate linkage
  modified_base 11: mod_base = OTHER; note = 2'-O-methyl modified nucleotide with phosphorothioate linkage
  modified_base 12: mod_base = OTHER; note = 2'-O-methyl modified nucleotide with phosphorothioate linkage
  modified_base 13: mod_base = OTHER; note = 2'-O-methyl modified nucleotide
gtcatagttccat

SEQ ID NO: 436 - RNA, 11 nt, synthetic construct
  source 1..11: mol_type = other RNA
  modified_base 8: mod_base = OTHER; note = Nucleotide with phosphorothioate linkage
  modified_base 9: mod_base = OTHER; note = 2'-O-methyl modified nucleotide with phosphorothioate linkage
  modified_base 10: mod_base = OTHER; note = 2'-O-methyl modified nucleotide with phosphorothioate linkage
  modified_base 11: mod_base = OTHER; note = 2'-O-methyl modified nucleotide
gtcatagccat

SEQ ID NO: 437 - RNA, 74 nt, synthetic construct; identical to SEQ ID NO: 114
  source 1..74: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 71: mod_base = OTHER; note = Nucleotide with phosphorothioate linkage
  modified_base 72: mod_base = OTHER; note = 2'-O-methyl modified nucleotide with phosphorothioate linkage
  modified_base 73: mod_base = OTHER; note = 2'-O-methyl modified nucleotide with phosphorothioate linkage
  modified_base 74: mod_base = OTHER; note = 2'-O-methyl modified nucleotide

SEQ ID NO: 438 - RNA, 79 nt, synthetic construct; identical to SEQ ID NO: 115
  source 1..79: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 76: mod_base = OTHER; note = Nucleotide with phosphorothioate linkage
  modified_base 77: mod_base = OTHER; note = 2'-O-methyl modified nucleotide with phosphorothioate linkage
  modified_base 78: mod_base = OTHER; note = 2'-O-methyl modified nucleotide with phosphorothioate linkage
  modified_base 79: mod_base = OTHER; note = 2'-O-methyl modified nucleotide

SEQ ID NO: 439 - RNA, 73 nt, synthetic construct; identical to SEQ ID NO: 116
  source 1..73: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 70: mod_base = OTHER; note = Nucleotide with phosphorothioate linkage
  modified_base 71: mod_base = OTHER; note = 2'-O-methyl modified nucleotide with phosphorothioate linkage
  modified_base 72: mod_base = OTHER; note = 2'-O-methyl modified nucleotide with phosphorothioate linkage
  modified_base 73: mod_base = OTHER; note = 2'-O-methyl modified nucleotide

SEQ ID NO: 440 - RNA, 71 nt, synthetic construct; identical to SEQ ID NO: 117
  source 1..71: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 68: mod_base = OTHER; note = Nucleotide with phosphorothioate linkage
  modified_base 69: mod_base = OTHER; note = 2'-O-methyl modified nucleotide with phosphorothioate linkage
  modified_base 70: mod_base = OTHER; note = 2'-O-methyl modified nucleotide with phosphorothioate linkage
  modified_base 71: mod_base = OTHER; note = 2'-O-methyl modified nucleotide

SEQ ID NO: 441 - RNA, 78 nt, synthetic construct; identical to SEQ ID NO: 118
  source 1..78: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 75: mod_base = OTHER; note = Nucleotide with phosphorothioate linkage
  modified_base 76: mod_base = OTHER; note = 2'-O-methyl modified nucleotide with phosphorothioate linkage
  modified_base 77: mod_base = OTHER; note = 2'-O-methyl modified nucleotide with phosphorothioate linkage
  modified_base 78: mod_base = OTHER; note = 2'-O-methyl modified nucleotide

SEQ ID NO: 442 - RNA, 72 nt, synthetic construct; identical to SEQ ID NO: 119
  source 1..72: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 69: mod_base = OTHER; note = Nucleotide with phosphorothioate linkage
  modified_base 70: mod_base = OTHER; note = 2'-O-methyl modified nucleotide with phosphorothioate linkage
  modified_base 71: mod_base = OTHER; note = 2'-O-methyl modified nucleotide with phosphorothioate linkage
  modified_base 72: mod_base = OTHER; note = 2'-O-methyl modified nucleotide

SEQ ID NO: 443 - RNA, 70 nt, synthetic construct; identical to SEQ ID NO: 120
  source 1..70: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 67: mod_base = OTHER; note = Nucleotide with phosphorothioate linkage
  modified_base 68: mod_base = OTHER; note = 2'-O-methyl modified nucleotide with phosphorothioate linkage
  modified_base 69: mod_base = OTHER; note = 2'-O-methyl modified nucleotide with phosphorothioate linkage
  modified_base 70: mod_base = OTHER; note = 2'-O-methyl modified nucleotide

SEQ ID NO: 444 - RNA, 77 nt, synthetic construct; identical to SEQ ID NO: 121
  source 1..77: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 74: mod_base = OTHER; note = Nucleotide with phosphorothioate linkage
  modified_base 75: mod_base = OTHER; note = 2'-O-methyl modified nucleotide with phosphorothioate linkage
  modified_base 76: mod_base = OTHER; note = 2'-O-methyl modified nucleotide with phosphorothioate linkage
  modified_base 77: mod_base = OTHER; note = 2'-O-methyl modified nucleotide

SEQ ID NO: 445 - RNA, 71 nt, synthetic construct; identical to SEQ ID NO: 122
  source 1..71: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 68: mod_base = OTHER; note = Nucleotide with phosphorothioate linkage
  modified_base 69: mod_base = OTHER; note = 2'-O-methyl modified nucleotide with phosphorothioate linkage
  modified_base 70: mod_base = OTHER; note = 2'-O-methyl modified nucleotide with phosphorothioate linkage
  modified_base 71: mod_base = OTHER; note = 2'-O-methyl modified nucleotide

SEQ ID NO: 446 - RNA, 69 nt, synthetic construct; identical to SEQ ID NO: 123
  source 1..69: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 66: mod_base = OTHER; note = Nucleotide with phosphorothioate linkage
  modified_base 67: mod_base = OTHER; note = 2'-O-methyl modified nucleotide with phosphorothioate linkage
  modified_base 68: mod_base = OTHER; note = 2'-O-methyl modified nucleotide with phosphorothioate linkage
  modified_base 69: mod_base = OTHER; note = 2'-O-methyl modified nucleotide

SEQ ID NO: 447 - RNA, 94 nt, synthetic construct; identical to SEQ ID NO: 124
  source 1..94: mol_type = other RNA
  modified_base 91: mod_base = OTHER; note = Nucleotide with phosphorothioate linkage
  modified_base 92: mod_base = OTHER; note = 2'-O-methyl modified nucleotide with phosphorothioate linkage
  modified_base 93: mod_base = OTHER; note = 2'-O-methyl modified nucleotide with phosphorothioate linkage
  modified_base 94: mod_base = OTHER; note = 2'-O-methyl modified nucleotide

SEQ ID NO: 448 - RNA, 98 nt, synthetic construct; identical to SEQ ID NO: 125
  source 1..98: mol_type = other RNA
  modified_base 95: mod_base = OTHER; note = Nucleotide with phosphorothioate linkage
  modified_base 96: mod_base = OTHER; note = 2'-O-methyl modified nucleotide with phosphorothioate linkage
  modified_base 97: mod_base = OTHER; note = 2'-O-methyl modified nucleotide with phosphorothioate linkage
  modified_base 98: mod_base = OTHER; note = 2'-O-methyl modified nucleotide

SEQ ID NO: 449 - RNA, 92 nt, synthetic construct; identical to SEQ ID NO: 126
  source 1..92: mol_type = other RNA
  modified_base 89: mod_base = OTHER; note = Nucleotide with phosphorothioate linkage
  modified_base 90: mod_base = OTHER; note = 2'-O-methyl modified nucleotide with phosphorothioate linkage
  modified_base 91: mod_base = OTHER; note = 2'-O-methyl modified nucleotide with phosphorothioate linkage
  modified_base 92: mod_base = OTHER; note = 2'-O-methyl modified nucleotide

SEQ ID NO: 450 - RNA, 90 nt, synthetic construct; identical to SEQ ID NO: 127
  source 1..90: mol_type = other RNA
  modified_base 87: mod_base = OTHER; note = Nucleotide with phosphorothioate linkage
  modified_base 88: mod_base = OTHER; note = 2'-O-methyl modified nucleotide with phosphorothioate linkage
  modified_base 89: mod_base = OTHER; note = 2'-O-methyl modified nucleotide with phosphorothioate linkage
  modified_base 90: mod_base = OTHER; note = 2'-O-methyl modified nucleotide

SEQ ID NO: 451 - RNA, 96 nt, synthetic construct; identical to SEQ ID NO: 128
  source 1..96: mol_type = other RNA
  modified_base 93: mod_base = OTHER; note = Nucleotide with phosphorothioate linkage
  modified_base 94: mod_base = OTHER; note = 2'-O-methyl modified nucleotide with phosphorothioate linkage
  modified_base 95: mod_base = OTHER; note = 2'-O-methyl modified nucleotide with phosphorothioate linkage
  modified_base 96: mod_base = OTHER; note = 2'-O-methyl modified nucleotide

SEQ ID NO: 452 - RNA, 90 nt, synthetic construct; identical to SEQ ID NO: 129
  source 1..90: mol_type = other RNA
  modified_base 87: mod_base = OTHER; note = Nucleotide with phosphorothioate linkage
  modified_base 88: mod_base = OTHER; note = 2'-O-methyl modified nucleotide with phosphorothioate linkage
  modified_base 89: mod_base = OTHER; note = 2'-O-methyl modified nucleotide with phosphorothioate linkage
  modified_base 90: mod_base = OTHER; note = 2'-O-methyl modified nucleotide

SEQ ID NO: 453 - RNA, 88 nt, synthetic construct; identical to SEQ ID NO: 130
  source 1..88: mol_type = other RNA
  modified_base 85: mod_base = OTHER; note = Nucleotide with phosphorothioate linkage
  modified_base 86: mod_base = OTHER; note = 2'-O-methyl modified nucleotide with phosphorothioate linkage
  modified_base 87: mod_base = OTHER; note = 2'-O-methyl modified nucleotide with phosphorothioate linkage
  modified_base 88: mod_base = OTHER; note = 2'-O-methyl modified nucleotide

SEQ ID NO: 454 - RNA, 94 nt, synthetic construct; identical to SEQ ID NO: 131
  source 1..94: mol_type = other RNA
  modified_base 91: mod_base = OTHER; note = Nucleotide with phosphorothioate linkage
  modified_base 92: mod_base = OTHER; note = 2'-O-methyl modified nucleotide with phosphorothioate linkage
  modified_base 93: mod_base = OTHER; note = 2'-O-methyl modified nucleotide with phosphorothioate linkage
  modified_base 94: mod_base = OTHER; note = 2'-O-methyl modified nucleotide

SEQ ID NO: 455 - RNA, 88 nt, synthetic construct; identical to SEQ ID NO: 132
  source 1..88: mol_type = other RNA
  modified_base 85: mod_base = OTHER; note = Nucleotide with phosphorothioate linkage
  modified_base 86: mod_base = OTHER; note = 2'-O-methyl modified nucleotide with phosphorothioate linkage
  modified_base 87: mod_base = OTHER; note = 2'-O-methyl modified nucleotide with phosphorothioate linkage
  modified_base 88: mod_base = OTHER; note = 2'-O-methyl modified nucleotide

SEQ ID NO: 456 - RNA, 86 nt, synthetic construct; identical to SEQ ID NO: 133
  source 1..86: mol_type = other RNA
  modified_base 83: mod_base = OTHER; note = Nucleotide with phosphorothioate linkage
  modified_base 84: mod_base = OTHER; note = 2'-O-methyl modified nucleotide with phosphorothioate linkage
  modified_base 85: mod_base = OTHER; note = 2'-O-methyl modified nucleotide with phosphorothioate linkage
  modified_base 86: mod_base = OTHER; note = 2'-O-methyl modified nucleotide

SEQ ID NO: 457 - RNA, 110 nt, synthetic construct; identical to SEQ ID NO: 134
  source 1..110: mol_type = other RNA
  modified_base 107: mod_base = OTHER; note = Nucleotide with phosphorothioate linkage
  modified_base 108: mod_base = OTHER; note = 2'-O-methyl modified nucleotide with phosphorothioate linkage
  modified_base 109: mod_base = OTHER; note = 2'-O-methyl modified nucleotide with phosphorothioate linkage
  modified_base 110: mod_base = OTHER; note = 2'-O-methyl modified nucleotide

SEQ ID NO: 458 - RNA, 90 nt, synthetic construct; identical to SEQ ID NO: 135
  source 1..90: mol_type = other RNA
  modified_base 87: mod_base = OTHER; note = Nucleotide with phosphorothioate linkage
  modified_base 88: mod_base = OTHER; note = 2'-O-methyl modified nucleotide with phosphorothioate linkage
  modified_base 89: mod_base = OTHER; note = 2'-O-methyl modified nucleotide with phosphorothioate linkage
  modified_base 90: mod_base = OTHER; note = 2'-O-methyl modified nucleotide

SEQ ID NO: 459 - RNA, 46 nt, synthetic construct; identical to SEQ ID NO: 136
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 460 - RNA, 46 nt, synthetic construct; identical to SEQ ID NO: 137
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 461 - RNA, 46 nt, synthetic construct; identical to SEQ ID NO: 138
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 462 - RNA, 36 nt, synthetic construct; identical to SEQ ID NO: 139
  source 1..36: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 33: mod_base = OTHER; note = Nucleotide with phosphorothioate linkage
  modified_base 34: mod_base = OTHER; note = 2'-O-methyl modified nucleotide with phosphorothioate linkage
  modified_base 35: mod_base = OTHER; note = 2'-O-methyl modified nucleotide with phosphorothioate linkage
  modified_base 36: mod_base = OTHER; note = 2'-O-methyl modified nucleotide

SEQ ID NO: 463 - RNA, 46 nt, synthetic construct; identical to SEQ ID NO: 140
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 464 - RNA, 46 nt, synthetic construct; identical to SEQ ID NO: 141
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 465 - RNA, 37 nt, synthetic construct; identical to SEQ ID NO: 142
  source 1..37: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 34: mod_base = OTHER; note = Nucleotide with phosphorothioate linkage
  modified_base 35: mod_base = OTHER; note = 2'-O-methyl modified nucleotide with phosphorothioate linkage
  modified_base 36: mod_base = OTHER; note = 2'-O-methyl modified nucleotide with phosphorothioate linkage
  modified_base 37: mod_base = OTHER; note = 2'-O-methyl modified nucleotide

SEQ ID NO: 466 - RNA, 39 nt, synthetic construct; identical to SEQ ID NO: 143
  source 1..39: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 36: mod_base = OTHER; note = Nucleotide with phosphorothioate linkage
  modified_base 37: mod_base = OTHER; note = 2'-O-methyl modified nucleotide with phosphorothioate linkage
  modified_base 38: mod_base = OTHER; note = 2'-O-methyl modified nucleotide with phosphorothioate linkage
  modified_base 39: mod_base = OTHER; note = 2'-O-methyl modified nucleotide

SEQ ID NO: 467 - RNA, 46 nt, synthetic construct; identical to SEQ ID NO: 144
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 468 - RNA, 46 nt, synthetic construct; identical to SEQ ID NO: 145
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 469 - RNA, 46 nt, synthetic construct; identical to SEQ ID NO: 146
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 470 - RNA, 46 nt, synthetic construct; identical to SEQ ID NO: 147
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 471 - RNA, 46 nt, synthetic construct; identical to SEQ ID NO: 148
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 472 - RNA, 46 nt, synthetic construct; identical to SEQ ID NO: 149
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 473 - RNA, 46 nt, synthetic construct; identical to SEQ ID NO: 150
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 474 - RNA, 46 nt, synthetic construct; identical to SEQ ID NO: 151
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 475 - RNA, 46 nt, synthetic construct; identical to SEQ ID NO: 152
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 476 - RNA, 46 nt, synthetic construct; identical to SEQ ID NO: 153
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 477 - RNA, 46 nt, synthetic construct; identical to SEQ ID NO: 154
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 478 - RNA, 46 nt, synthetic construct; identical to SEQ ID NO: 155
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 479 - RNA, 46 nt, synthetic construct; identical to SEQ ID NO: 156
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 480 - RNA, 46 nt, synthetic construct; identical to SEQ ID NO: 157
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 481 - RNA, 46 nt, synthetic construct; identical to SEQ ID NO: 158
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 482 - RNA, 46 nt, synthetic construct; identical to SEQ ID NO: 159
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 483 - RNA, 46 nt, synthetic construct; identical to SEQ ID NO: 160
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 484 - RNA, 46 nt, synthetic construct; identical to SEQ ID NO: 161
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 485 - RNA, 46 nt, synthetic construct; identical to SEQ ID NO: 162
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 486 - RNA, 46 nt, synthetic construct; identical to SEQ ID NO: 163
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 487 - RNA, 46 nt, synthetic construct; identical to SEQ ID NO: 164
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 488 - RNA, 46 nt, synthetic construct; identical to SEQ ID NO: 165
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 489 - RNA, 46 nt, synthetic construct; identical to SEQ ID NO: 166
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 490 - RNA, 46 nt, synthetic construct; identical to SEQ ID NO: 167
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 491 - RNA, 46 nt, synthetic construct; identical to SEQ ID NO: 168
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 492 - RNA, 46 nt, synthetic construct; identical to SEQ ID NO: 169
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 493 - RNA, 46 nt, synthetic construct; identical to SEQ ID NO: 170
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 494 - RNA, 46 nt, synthetic construct; identical to SEQ ID NO: 171
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 495 - RNA, 46 nt, synthetic construct; identical to SEQ ID NO: 172
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 496 - RNA, 46 nt, synthetic construct; identical to SEQ ID NO: 173
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 497 - RNA, 46 nt, synthetic construct; identical to SEQ ID NO: 174
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 498 - RNA, 46 nt, synthetic construct; identical to SEQ ID NO: 175
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 499 - RNA, 46 nt, synthetic construct; identical to SEQ ID NO: 176
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 500 - RNA, 46 nt, synthetic construct; identical to SEQ ID NO: 177
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 501 - RNA, 46 nt, synthetic construct; identical to SEQ ID NO: 178
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 502 - RNA, 46 nt, synthetic construct; identical to SEQ ID NO: 179
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 503 - RNA, 46 nt, synthetic construct; identical to SEQ ID NO: 180
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 504 - RNA, 46 nt, synthetic construct; identical to SEQ ID NO: 181
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 505 - RNA, 46 nt, synthetic construct; identical to SEQ ID NO: 182
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 506 - RNA, 46 nt, synthetic construct; identical to SEQ ID NO: 183
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 507 - RNA, 46 nt, synthetic construct; identical to SEQ ID NO: 184
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 508 - RNA, 41 nt, synthetic construct; identical to SEQ ID NO: 185
  source 1..41: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 38: mod_base = OTHER; note = Nucleotide with phosphorothioate linkage
  modified_base 39: mod_base = OTHER; note = 2'-O-methyl modified nucleotide with phosphorothioate linkage
  modified_base 40: mod_base = OTHER; note = 2'-O-methyl modified nucleotide with phosphorothioate linkage
  modified_base 41: mod_base = OTHER; note = 2'-O-methyl modified nucleotide

SEQ ID NO: 509 - RNA, 46 nt, synthetic construct; identical to SEQ ID NO: 186
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 510 - RNA, 46 nt, synthetic construct; identical to SEQ ID NO: 187
  source 1..46: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 43: mod_base = OTHER; note = Nucleotide with phosphorothioate linkage
  modified_base 44: mod_base = OTHER; note = 2'-O-methyl modified nucleotide with phosphorothioate linkage
  modified_base 45: mod_base = OTHER; note = 2'-O-methyl modified nucleotide with phosphorothioate linkage
  modified_base 46: mod_base = OTHER; note = 2'-O-methyl modified nucleotide

SEQ ID NO: 511 - RNA, 41 nt, synthetic construct; identical to SEQ ID NO: 188
  source 1..41: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 38: mod_base = OTHER; note = Nucleotide with phosphorothioate linkage
  modified_base 39: mod_base = OTHER; note = 2'-O-methyl modified nucleotide with phosphorothioate linkage
  modified_base 40: mod_base = OTHER; note = 2'-O-methyl modified nucleotide with phosphorothioate linkage
  modified_base 41: mod_base = OTHER; note = 2'-O-methyl modified nucleotide

SEQ ID NO: 512 - RNA, 40 nt, synthetic construct; identical to SEQ ID NO: 189
  source 1..40: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 37: mod_base = OTHER; note = Nucleotide with phosphorothioate linkage
  modified_base 38: mod_base = OTHER; note = 2'-O-methyl modified nucleotide with phosphorothioate linkage
  modified_base 39: mod_base = OTHER; note = 2'-O-methyl modified nucleotide with phosphorothioate linkage
  modified_base 40: mod_base = OTHER; note = 2'-O-methyl modified nucleotide

SEQ ID NO: 513 - RNA, 39 nt, synthetic construct; identical to SEQ ID NO: 190
  source 1..39: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 36: mod_base = OTHER; note = Nucleotide with phosphorothioate linkage
  modified_base 37: mod_base = OTHER; note = 2'-O-methyl modified nucleotide with phosphorothioate linkage
  modified_base 38: mod_base = OTHER; note = 2'-O-methyl modified nucleotide with phosphorothioate linkage
  modified_base 39: mod_base = OTHER; note = 2'-O-methyl modified nucleotide

SEQ ID NO: 514 - RNA, 40 nt, synthetic construct; identical to SEQ ID NO: 191
  source 1..40: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 37: mod_base = OTHER; note = Nucleotide with phosphorothioate linkage
  modified_base 38: mod_base = OTHER; note = 2'-O-methyl modified nucleotide with phosphorothioate linkage
  modified_base 39: mod_base = OTHER; note = 2'-O-methyl modified nucleotide with phosphorothioate linkage
  modified_base 40: mod_base = OTHER; note = 2'-O-methyl modified nucleotide

SEQ ID NO: 515 - RNA, 41 nt, synthetic construct; identical to SEQ ID NO: 192
  source 1..41: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 38: mod_base = OTHER; note = Nucleotide with phosphorothioate linkage
  modified_base 39: mod_base = OTHER; note = 2'-O-methyl modified nucleotide with phosphorothioate linkage
  modified_base 40: mod_base = OTHER; note = 2'-O-methyl modified nucleotide with phosphorothioate linkage
  modified_base 41: mod_base = OTHER; note = 2'-O-methyl modified nucleotide

SEQ ID NO: 516 - RNA, 38 nt, synthetic construct; identical to SEQ ID NO: 193
  source 1..38: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 35: mod_base = OTHER; note = Nucleotide with phosphorothioate linkage
  modified_base 36: mod_base = OTHER; note = 2'-O-methyl modified nucleotide with phosphorothioate linkage
  modified_base 37: mod_base = OTHER; note = 2'-O-methyl modified nucleotide with phosphorothioate linkage
  modified_base 38: mod_base = OTHER; note = 2'-O-methyl modified nucleotide

SEQ ID NO: 517 - RNA, 36 nt, synthetic construct; identical to SEQ ID NO: 194
  source 1..36: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 33: mod_base = OTHER; note = Nucleotide with phosphorothioate linkage
  modified_base 34: mod_base = OTHER; note = 2'-O-methyl modified nucleotide with phosphorothioate linkage
  modified_base 35: mod_base = OTHER; note = 2'-O-methyl modified nucleotide with phosphorothioate linkage
  modified_base 36: mod_base = OTHER; note = 2'-O-methyl modified nucleotide

SEQ ID NO: 518 - RNA, 38 nt, synthetic construct; identical to SEQ ID NO: 195
  source 1..38: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 35: mod_base = OTHER; note = Nucleotide with phosphorothioate linkage
  modified_base 36: mod_base = OTHER; note = 2'-O-methyl modified nucleotide with phosphorothioate linkage
  modified_base 37: mod_base = OTHER; note = 2'-O-methyl modified nucleotide with phosphorothioate linkage
  modified_base 38: mod_base = OTHER; note = 2'-O-methyl modified nucleotide

SEQ ID NO: 519 - RNA, 37 nt, synthetic construct; identical to SEQ ID NO: 196
  source 1..37: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 34: mod_base = OTHER; note = Nucleotide with phosphorothioate linkage
  modified_base 35: mod_base = OTHER; note = 2'-O-methyl modified nucleotide with phosphorothioate linkage
  modified_base 36: mod_base = OTHER; note = 2'-O-methyl modified nucleotide with phosphorothioate linkage
  modified_base 37: mod_base = OTHER; note = 2'-O-methyl modified nucleotide

SEQ ID NO: 520 - RNA, 36 nt, synthetic construct; identical to SEQ ID NO: 197
  source 1..36: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 33: mod_base = OTHER; note = Nucleotide with phosphorothioate linkage
  modified_base 34: mod_base = OTHER; note = 2'-O-methyl modified nucleotide with phosphorothioate linkage
  modified_base 35: mod_base = OTHER; note = 2'-O-methyl modified nucleotide with phosphorothioate linkage
  modified_base 36: mod_base = OTHER; note = 2'-O-methyl modified nucleotide

SEQ ID NO: 521 - RNA, 135 nt, synthetic construct; identical to SEQ ID NO: 198
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 522 - RNA, 135 nt, synthetic construct; identical to SEQ ID NO: 199
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 523 - RNA, 135 nt, synthetic construct; identical to SEQ ID NO: 200
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 524 - RNA, 115 nt, synthetic construct; identical to SEQ ID NO: 201
  source 1..115: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 112: mod_base = OTHER; note = Nucleotide with phosphorothioate linkage
  modified_base 113: mod_base = OTHER; note = 2'-O-methyl modified nucleotide with phosphorothioate linkage
  modified_base 114: mod_base = OTHER; note = 2'-O-methyl modified nucleotide with phosphorothioate linkage
  modified_base 115: mod_base = OTHER; note = 2'-O-methyl modified nucleotide

SEQ ID NO: 525 - RNA, 135 nt, synthetic construct; identical to SEQ ID NO: 202
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 526 - RNA, 135 nt, synthetic construct; identical to SEQ ID NO: 203
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 527 - RNA, 118 nt, synthetic construct; identical to SEQ ID NO: 204
  source 1..118: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 115: mod_base = OTHER; note = Nucleotide with phosphorothioate linkage
  modified_base 116: mod_base = OTHER; note = 2'-O-methyl modified nucleotide with phosphorothioate linkage
  modified_base 117: mod_base = OTHER; note = 2'-O-methyl modified nucleotide with phosphorothioate linkage
  modified_base 118: mod_base = OTHER; note = 2'-O-methyl modified nucleotide

SEQ ID NO: 528 - RNA, 117 nt, synthetic construct; identical to SEQ ID NO: 205
  source 1..117: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 114: mod_base = OTHER; note = Nucleotide with phosphorothioate linkage
  modified_base 115: mod_base = OTHER; note = 2'-O-methyl modified nucleotide with phosphorothioate linkage
  modified_base 116: mod_base = OTHER; note = 2'-O-methyl modified nucleotide with phosphorothioate linkage
  modified_base 117: mod_base = OTHER; note = 2'-O-methyl modified nucleotide

SEQ ID NO: 529 - RNA, 135 nt, synthetic construct; identical to SEQ ID NO: 206
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 530 - RNA, 135 nt, synthetic construct; identical to SEQ ID NO: 207
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 531 - RNA, 135 nt, synthetic construct; identical to SEQ ID NO: 208
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 532 - RNA, 135 nt, synthetic construct; identical to SEQ ID NO: 209
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 533 - RNA, 135 nt, synthetic construct; identical to SEQ ID NO: 210
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 534 - RNA, 135 nt, synthetic construct; identical to SEQ ID NO: 211
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 535 - RNA, 135 nt, synthetic construct; identical to SEQ ID NO: 212
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 536 - RNA, 135 nt, synthetic construct; identical to SEQ ID NO: 213
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 537 - RNA, 135 nt, synthetic construct; identical to SEQ ID NO: 214
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 538 - RNA, 135 nt, synthetic construct; identical to SEQ ID NO: 215
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 539 - RNA, 135 nt, synthetic construct; identical to SEQ ID NO: 216
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 540 - RNA, 135 nt, synthetic construct; identical to SEQ ID NO: 217
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 541 - RNA, 135 nt, synthetic construct; identical to SEQ ID NO: 218
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 542 - RNA, 135 nt, synthetic construct; identical to SEQ ID NO: 219
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 543 - RNA, 135 nt, synthetic construct; identical to SEQ ID NO: 220
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 544 - RNA, 135 nt, synthetic construct; identical to SEQ ID NO: 221
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 545 - RNA, 135 nt, synthetic construct; identical to SEQ ID NO: 222
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 546 - RNA, 135 nt, synthetic construct; identical to SEQ ID NO: 223
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 547 - RNA, 135 nt, synthetic construct; identical to SEQ ID NO: 224
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 548 - RNA, 135 nt, synthetic construct; identical to SEQ ID NO: 225
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 549 - RNA, 135 nt, synthetic construct; identical to SEQ ID NO: 226
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 550 - RNA, 135 nt, synthetic construct; identical to SEQ ID NO: 227
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 551 - RNA, 135 nt, synthetic construct; identical to SEQ ID NO: 228
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 552 - RNA, 135 nt, synthetic construct; identical to SEQ ID NO: 229
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 553 - RNA, 135 nt, synthetic construct; identical to SEQ ID NO: 230
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 554 - RNA, 135 nt, synthetic construct; identical to SEQ ID NO: 231
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 555 - RNA, 135 nt, synthetic construct; identical to SEQ ID NO: 232
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 556 - RNA, 135 nt, synthetic construct; identical to SEQ ID NO: 233
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 557 - RNA, 135 nt, synthetic construct; identical to SEQ ID NO: 234
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 558 - RNA, 135 nt, synthetic construct; identical to SEQ ID NO: 235
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 559 - RNA, 135 nt, synthetic construct; identical to SEQ ID NO: 236
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 560 - RNA, 135 nt, synthetic construct; identical to SEQ ID NO: 237
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 561 - RNA, 135 nt, synthetic construct; identical to SEQ ID NO: 238
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 562 - RNA, 135 nt, synthetic construct; identical to SEQ ID NO: 239
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 563 - RNA, 135 nt, synthetic construct; identical to SEQ ID NO: 240
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 564 - RNA, 135 nt, synthetic construct; identical to SEQ ID NO: 241
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 565 - RNA, 135 nt, synthetic construct; identical to SEQ ID NO: 242
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 566 - RNA, 135 nt, synthetic construct; identical to SEQ ID NO: 243
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 567 - RNA, 135 nt, synthetic construct; identical to SEQ ID NO: 244
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 568 - RNA, 135 nt, synthetic construct; identical to SEQ ID NO: 245
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 569 - RNA, 135 nt, synthetic construct; identical to SEQ ID NO: 246
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 570 - RNA, 130 nt, synthetic construct; identical to SEQ ID NO: 247
  source 1..130: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 127: mod_base = OTHER; note = Nucleotide with phosphorothioate linkage
  modified_base 128: mod_base = OTHER; note = 2'-O-methyl modified nucleotide with phosphorothioate linkage
  modified_base 129: mod_base = OTHER; note = 2'-O-methyl modified nucleotide with phosphorothioate linkage
  modified_base 130: mod_base = OTHER; note = 2'-O-methyl modified nucleotide

SEQ ID NO: 571 - RNA, 135 nt, synthetic construct; identical to SEQ ID NO: 248
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 572 - RNA, 135 nt, synthetic construct; identical to SEQ ID NO: 249
  source 1..135: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 132: mod_base = OTHER; note = Nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide

SEQ ID NO: 573 - RNA, 119 nt, synthetic construct; identical to SEQ ID NO: 250
  source 1..119: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 116: mod_base = OTHER; note = Nucleotide with phosphorothioate linkage
  modified_base 117: mod_base = OTHER; note = 2'-O-methyl modified nucleotide with phosphorothioate linkage
  modified_base 118: mod_base = OTHER; note = 2'-O-methyl modified nucleotide with phosphorothioate linkage
  modified_base 119: mod_base = OTHER; note = 2'-O-methyl modified nucleotide

SEQ ID NO: 574 - RNA, 118 nt, synthetic construct; identical to SEQ ID NO: 251
  source 1..118: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 115: mod_base = OTHER; note = Nucleotide with phosphorothioate linkage
  modified_base 116: mod_base = OTHER; note = 2'-O-methyl modified nucleotide with phosphorothioate linkage
  modified_base 117: mod_base = OTHER; note = 2'-O-methyl modified nucleotide with phosphorothioate linkage
  modified_base 118: mod_base = OTHER; note = 2'-O-methyl modified nucleotide

SEQ ID NO: 575 - RNA, 117 nt, synthetic construct; identical to SEQ ID NO: 252
  source 1..117: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 114: mod_base = OTHER; note = Nucleotide with phosphorothioate linkage
  modified_base 115: mod_base = OTHER; note = 2'-O-methyl modified nucleotide with phosphorothioate linkage
  modified_base 116: mod_base = OTHER; note = 2'-O-methyl modified nucleotide with phosphorothioate linkage
  modified_base 117: mod_base = OTHER; note = 2'-O-methyl modified nucleotide

SEQ ID NO: 576 - RNA, 118 nt, synthetic construct; identical to SEQ ID NO: 253
  source 1..118: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 115: mod_base = OTHER; note = Nucleotide with phosphorothioate linkage
  modified_base 116: mod_base = OTHER; note = 2'-O-methyl modified nucleotide with phosphorothioate linkage
  modified_base 117: mod_base = OTHER; note = 2'-O-methyl modified nucleotide with phosphorothioate linkage
  modified_base 118: mod_base = OTHER; note = 2'-O-methyl modified nucleotide

SEQ ID NO: 577 - RNA, 119 nt, synthetic construct; identical to SEQ ID NO: 254
  source 1..119: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 116: mod_base = OTHER; note = Nucleotide with phosphorothioate linkage
  modified_base 117: mod_base = OTHER; note = 2'-O-methyl modified nucleotide with phosphorothioate linkage
  modified_base 118: mod_base = OTHER; note = 2'-O-methyl modified nucleotide with phosphorothioate linkage
  modified_base 119: mod_base = OTHER; note = 2'-O-methyl modified nucleotide

SEQ ID NO: 578 - RNA, 119 nt, synthetic construct; identical to SEQ ID NO: 255
  source 1..119: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 116: mod_base = OTHER; note = Nucleotide with phosphorothioate linkage
  modified_base 117: mod_base = OTHER; note = 2'-O-methyl modified nucleotide with phosphorothioate linkage
  modified_base 118: mod_base = OTHER; note = 2'-O-methyl modified nucleotide with phosphorothioate linkage
  modified_base 119: mod_base = OTHER; note = 2'-O-methyl modified nucleotide

SEQ ID NO: 579 - RNA, 117 nt, synthetic construct; identical to SEQ ID NO: 256
  source 1..117: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 114: mod_base = OTHER; note = Nucleotide with phosphorothioate linkage
  modified_base 115: mod_base = OTHER; note = 2'-O-methyl modified nucleotide with phosphorothioate linkage
  modified_base 116: mod_base = OTHER; note = 2'-O-methyl modified nucleotide with phosphorothioate linkage
  modified_base 117: mod_base = OTHER; note = 2'-O-methyl modified nucleotide

SEQ ID NO: 580 - RNA, 119 nt, synthetic construct; identical to SEQ ID NO: 257
  source 1..119: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 116: mod_base = OTHER; note = Nucleotide with phosphorothioate linkage
  modified_base 117: mod_base = OTHER; note = 2'-O-methyl modified nucleotide with phosphorothioate linkage
  modified_base 118: mod_base = OTHER; note = 2'-O-methyl modified nucleotide with phosphorothioate linkage
  modified_base 119: mod_base = OTHER; note = 2'-O-methyl modified nucleotide

SEQ ID NO: 581 - RNA, 118 nt, synthetic construct; identical to SEQ ID NO: 258
  source 1..118: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 115: mod_base = OTHER; note = Nucleotide with phosphorothioate linkage
  modified_base 116: mod_base = OTHER; note = 2'-O-methyl modified nucleotide with phosphorothioate linkage
  modified_base 117: mod_base = OTHER; note = 2'-O-methyl modified nucleotide with phosphorothioate linkage
  modified_base 118: mod_base = OTHER; note = 2'-O-methyl modified nucleotide

SEQ ID NO: 582 - RNA, 117 nt, synthetic construct; identical to SEQ ID NO: 259
  source 1..117: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 114: mod_base = OTHER; note = Nucleotide with phosphorothioate linkage
  modified_base 115: mod_base = OTHER; note = 2'-O-methyl modified nucleotide with phosphorothioate linkage
  modified_base 116: mod_base = OTHER; note = 2'-O-methyl modified nucleotide with phosphorothioate linkage
  modified_base 117: mod_base = OTHER; note = 2'-O-methyl modified nucleotide

SEQ ID NO: 583 - RNA, 19 nt, synthetic construct
  source 1..19: mol_type = other RNA
  modified_base 16: mod_base = OTHER; note = Nucleotide with phosphorothioate linkage
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
gtcatagttccattattgc

SEQ ID NO: 584 - RNA, 84 nt, synthetic construct; identical to SEQ ID NO: 329
  source 1..84: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 81: mod_base = OTHER; note = Nucleotide with phosphorothioate linkage
  modified_base 82: mod_base = OTHER; note = 2'-O-methyl modified nucleotide with phosphorothioate linkage
  modified_base 83: mod_base = OTHER; note = 2'-O-methyl modified nucleotide with phosphorothioate linkage
  modified_base 84: mod_base = OTHER; note = 2'-O-methyl modified nucleotide

SEQ ID NO: 585 - RNA, 21 nt, synthetic construct
  source 1..21: mol_type = other RNA
  modified_base 18: mod_base = OTHER; note = Nucleotide with phosphorothioate linkage
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
gtcatagttccatgaaagcca

SEQ ID NO: 586 - RNA, 85 nt, synthetic construct; identical to SEQ ID NO: 335
  source 1..85: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 82: mod_base = OTHER; note = Nucleotide with phosphorothioate linkage
  modified_base 83: mod_base = OTHER; note = 2'-O-methyl modified nucleotide with phosphorothioate linkage
  modified_base 84: mod_base = OTHER; note = 2'-O-methyl modified nucleotide with phosphorothioate linkage
  modified_base 85: mod_base = OTHER; note = 2'-O-methyl modified nucleotide

SEQ ID NO: 587 - RNA, 27 nt, synthetic construct
  source 1..27: mol_type = other RNA
  modified_base 24: mod_base = OTHER; note = Nucleotide with phosphorothioate linkage
  modified_base 25: mod_base = OTHER; note = 2'-O-methyl modified nucleotide with phosphorothioate linkage
  modified_base 26: mod_base = OTHER; note = 2'-O-methyl modified nucleotide with phosphorothioate linkage
  modified_base 27: mod_base = OTHER; note = 2'-O-methyl modified nucleotide
gtcatagttccattaaagccattgctg

SEQ ID NO: 588 - RNA, 93 nt, synthetic construct; identical to SEQ ID NO: 332
  source 1..93: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 90: mod_base = OTHER; note = Nucleotide with phosphorothioate linkage
  modified_base 91: mod_base = OTHER; note = 2'-O-methyl modified nucleotide with phosphorothioate linkage
  modified_base 92: mod_base = OTHER; note = 2'-O-methyl modified nucleotide with phosphorothioate linkage
  modified_base 93: mod_base = OTHER; note = 2'-O-methyl modified nucleotide